(19) United States
(12) Patent Application Publication
(10) Pub. No.: US 2025/0059174 A1
(43) Pub. Date: Feb. 20, 2025

(54) Pharmaceutical Compounds and Compositions as C-Kit Kinase Inhibitors

(71) Applicant: Enanta Pharmaceuticals, Inc., Watertown, MA (US)

(72) Inventors: Jiajun ZHANG, Cambridge, MA (US); Yuk Ming SIU, Watertown, MA (US); Peilin XU, Watertown, MA (US); Matthew C. RHODES, Boston, MA (US); Hui CAO, Belmont, MA (US); Yat Sun OR, Waltham, MA (US); Xuri GAO, Newtonville, MA (US); Wei LI, Lexington, MA (US); Xuechao XING, Wilmington, MA (US); Scott MITCHELL, Woburn, MA (US)

(21) Appl. No.: 18/797,882

(22) Filed: Aug. 8, 2024

Related U.S. Application Data
(60) Provisional application No. 63/667,539, filed on Jul. 3, 2024.
(60) Provisional application No. 63/607,506, filed on Dec. 7, 2023.
(60) Provisional application No. 63/531,637, filed on Aug. 9, 2023.

(51) Int. Cl. C07D 413/14; A61K 31/4245; A61K 31/429; A61K 31/433; A61K 31/437; A61K 31/4375; A61K 31/438; A61K 31/4439; A61K 31/444; A61K 31/4545; A61K 31/4709; A61K 31/496; A61K 31/497; A61K 31/498; A61K 31/4985; A61K 31/501; A61K 31/5025; A61K 31/506; A61K 31/519; A61K 31/53; A61K 31/5377; A61K 31/538; A61K 31/541; A61K 31/553; C07D 413/12; C07D 417/12; C07D 417/14; C07D 471/04; C07D 471/10; C07D 487/04; C07D 495/04; C07D 498/04; C07D 513/04; C07D 519/00

(52) U.S. Cl. CPC C07D 413/14; A61K 31/4245; A61K 31/429; A61K 31/433; A61K 31/437; A61K 31/4375; A61K 31/438; A61K 31/4439; A61K 31/444; A61K 31/4545; A61K 31/4709; A61K 31/496; A61K 31/497; A61K 31/498; A61K 31/4985; A61K 31/501; A61K 31/5025; A61K 31/506; A61K 31/519; A61K 31/53; A61K 31/5377; A61K 31/538; A61K 31/541; A61K 31/553; C07D 413/12; C07D 417/12; C07D 417/14; C07D 471/04; C07D 471/10; C07D 487/04; C07D 495/04; C07D 498/04; C07D 513/04; C07D 519/00

(57) ABSTRACT

The invention provides compounds of formulae (I), or pharmaceutically acceptable salts and pharmaceutical compositions thereof,

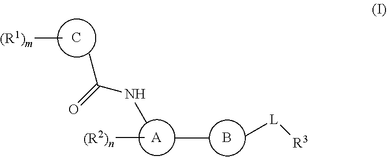

which are useful as protein kinase inhibitors; as well as methods for using such compounds to treat, ameliorate or prevent a condition associated with abnormal or deregulated kinase activity. In some embodiments, the invention provides methods for using such compounds to treat, ameliorate or prevent diseases or disorders that involve abnormal activation of c-kit or c-kit, CSF1R, and PDGFR (PDGFRα, PDGFRβ) kinases.

### RELATED APPLICATIONS

This application claims the benefit of U.S. Provisional Application No. 63/531,637, filed Aug. 9, 2023, U.S. Provisional Application No. 63/607,506, filed Dec. 7, 2023, and U.S. Provisional Application No. 63/667,539, filed Jul. 3, 2024. The entire teachings of the above applications are incorporated herein by reference.

### TECHNICAL FIELD

The present invention relates generally to compounds and pharmaceutical compositions useful as CSF1R, PDGFR and/or c-kit kinases inhibitors.

### BACKGROUND OF THE INVENTION

Protein kinases (PK) are a large set of structurally related phosphoryl transferases having highly conserved structures and catalytic functions. Protein kinases are enzymatic components of the signal transduction pathways which catalyze the transfer of the terminal phosphate from ATP to the hydroxy group of tyrosine, serine and/or threonine residues of proteins, and are therefore categorized into families by the substrates they phosphorylate: Protein Tyrosine Kinases (PTK), and Protein Serine/Threonine Kinases.

Protein kinases play a critical role in the control of cell growth and differentiation and are responsible for the control of a wide variety of cellular signal transduction processes, wherein protein kinases are key mediators of cellular signal leading to the production of growth factors and cytokines. The overexpression or inappropriate expression of normal or mutant protein kinases plays a significant role in the development of many diseases and disorders including, for example, central nervous system disorders such as Alzheimer's inflammatory disorders (such as arthritis), bone diseases such as osteoporosis, metabolic disorders such as diabetes, blood vessel proliferative disorders such as angiogenesis, autoimmune diseases such as rheumatoid arthritis, ocular diseases, cardiovascular disease, atherosclerosis, cancer, thrombosis, psoriasis, restenosis, schizophrenia, pain sensation, transplant rejection, and infectious diseases such as viral or fungal infections.

Mast cells are immune cells that reside in tissues throughout the body and release chemical mediators in response to certain stimuli. Inflammatory mediators are stored in granules within the mast cells. Activation of a mast cell leads to the process of degranulation, which releases these chemicals into the extracellular space. Dysfunction of mast cells has been implicated in a wide range of allergic and inflammatory diseases including skin and eye diseases, such as chronic urticaria systemic sclerosis, atopic dermatitis and allergic conjunctivitis; respiratory diseases such as asthma and chronic rhinosinusitis with nasal polyposis; and gastrointestinal diseases such as irritable bowel syndrome, inflammatory bowel disease, eosinophilic esophagitis and food allergy.

KIT, also known as CD117, is a receptor tyrosine kinase and is considered a critical regulator of mast cell activity. The stem cell factor, SCF, is KIT's native ligand, and activation of KIT by SCF is important in the migration, differentiation, and propagation of circulating mast cell progenitors, as well as the survival of mature mast cells within tissue. KIT is also important for mast cell activation, degranulation, and the release of downstream cytokines. Although compounds have been reported to inhibit KIT activity and some of them have been approved to treat certain types of cancer or tumor, they have not been approved as therapies to treat, ameliorate or prevent autoimmune diseases or disorders that involve abnormal activation of c-kit or c-kit, CSF1R,ne and PDGFR (PDGFRα, PDGFRβ) kinases. (Refer to WO 2013/033070, WO 2013/033116, WO 2013/033167, WO 2013/033203, WO 2013/033620, WO 2022/109595, WO 2022016021, WO 2022/182982, WO 2023/205226, WO 2023/212612, WO 2024/118887, WO 2024/123966, and WO 2024/124002).

### SUMMARY OF THE INVENTION

In its principal aspect, the present invention provides a compound of Formula (I):

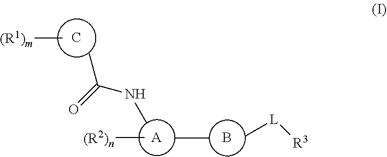

or a pharmaceutically acceptable salt thereof, wherein:
each R¹ is independently selected from the group consisting of deuterium, halogen, —CN, optionally substituted —C₁-C₆ alkyl, optionally substituted —C₁-C₆ alkoxy, optionally substituted —C₃-C₁₂ cycloalkyl, preferably optionally substituted C₃-C₈-cycloalkyl; optionally substituted —C₅-C₁₂ cycloalkenyl, preferably optionally substituted C₅-C₈-cycloalkenyl, optionally substituted 3- to 12-membered heterocycloalkyl, preferably optionally substituted 3- to 8-membered heterocycloalkyl, optionally substituted aryl, optionally substituted arylalkyl, optionally substituted heteroaryl, optionally substituted heteroarylalkyl, —C(O)R⁴, —C(O)OR⁴, —C(O)NR⁴R⁵, —C(S)NR⁴R⁵, and —NR⁴R⁵;
   alternatively, two adjacent R¹ groups are taken together with the atoms to which they are attached to form a fused ring which is optionally substituted C₅-C₈ cycloalkenyl, or optionally substituted 5- to 8-membered heterocycloalkyl;
m is selected from the group consisting of 0, 1, 2, 3, and 4;
each R⁴ and R⁵ is independently selected from the group consisting of hydrogen, optionally substituted —C₁-C₆ alkyl, optionally substituted —C₃-C₈ cycloalkyl, optionally substituted aryl, optionally substituted arylalkyl, optionally substituted heteroaryl, optionally substituted heteroarylalkyl; alternatively, R⁴ and R⁵ are taken together with the nitrogen atom to which they are attached to form an optionally substituted 3- to 12-membered heterocyclic ring, preferably a 3- to 8-membered heterocyclic ring;
each R² is independently selected from the group consisting of deuterium, halogen, —CN, optionally substituted —C₁-C₆ alkyl, optionally substituted —C₃-C₈ cycloalkyl, optionally substituted —C₁-C₆ alkoxy, and optionally substituted —C₃-C₈ cycloalkoxy;
n is 0, 1, 2, 3, or 4;
L is absent, —(CR⁶R⁷)ₚ—, —(CR⁷R⁸)_{q}O—, —(CR⁷R⁸)NR⁴—, —(CR⁷R⁸)_{q}C(O)NR⁴—, or —(CR⁷R⁸)NR⁴C(O)—;
p is selected from the group consisting of 1, 2, 3, or 4;
q is selected from the group consisting of 0, 1, 2, 3 and 4;
R⁶ is selected from the group consisting of hydrogen, optionally substituted —C₁-C₆ alkyl, optionally substituted —C₁-C₆ alkoxy, and —NHC(O)OR⁴;
R⁷ and R⁸ are each independently selected from the group consisting of hydrogen, fluorine, and optionally substituted —C₁-C₆ alkyl;
R³ is selected from the group consisting of optionally substituted —C₁-C₈ alkyl, optionally substituted —C₂-C₈ alkenyl, optionally substituted —C₃-C₁₂ cycloalkyl, optionally substituted —C₅-C₁₂ cycloalkenyl, optionally substituted 3- to 12-membered heterocycloalkyl, optionally substituted aryl, and optionally substituted heteroaryl;

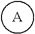

is aryl or heteroaryl;

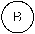

is absent or optionally substituted heteroaryl when L is not absent, and

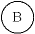

is optionally substituted heteroaryl when L is absent; alternatively, alternatively, L is absent, and R³, the atom to which it is attached and an adjacent atom of

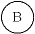

are taken together to form an optionally substituted fused 5- to 8-membered heterocycloalkyl or optionally substituted fused heteroaryl; and

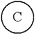

is heteroaryl, provided that

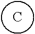

is not

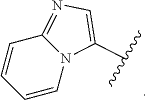 [SMILES: *c1cnc2ccccn12]

### DETAILED DESCRIPTION OF THE INVENTION

In one embodiment of the present invention is a compound of Formula (I) described above, or a pharmaceutically acceptable salt thereof. In certain embodiments of the compounds of Formula (I),

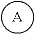

is aryl; preferably

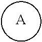

is phenyl.

In certain embodiments of the compounds of Formula (I),

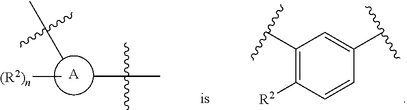

In certain embodiments, R² is methyl, difluoromethyl, or halogen. In certain embodiments, R² is methyl or halogen. In certain embodiments, R² is methyl, difluoromethyl, or chloro. In certain embodiments, R² is methyl.

In certain embodiments of the compounds of Formula (I),

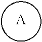

is selected from the groups below:

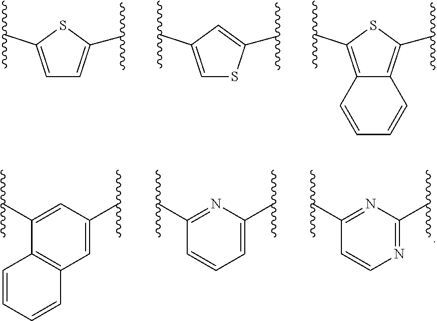

In certain embodiments of the compounds of Formula (I),

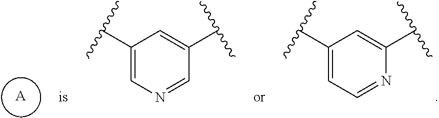

In certain embodiments of the compounds of Formula (I),

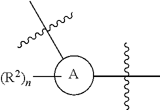

is selected from the group consisting of

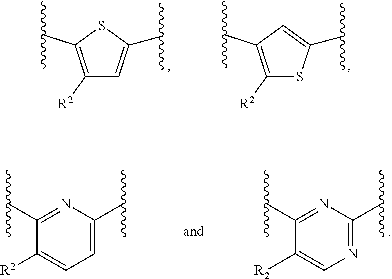

In certain embodiments of the compounds of Formula (I),

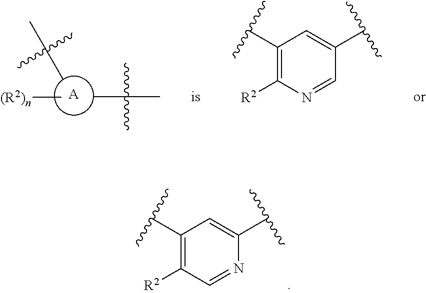

In certain embodiments, R² is methyl or halogen. In certain embodiments, R² is difluoromethyl. In certain embodiments, R² is methyl, chloro, or difluoromethyl.

In certain embodiments of the compounds of Formula (I),

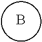

is optionally substituted 5-membered heteroaryl.

In certain embodiments of the compounds of Formula (I),

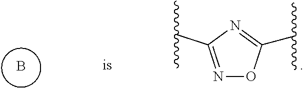

In certain embodiments of the compounds of Formula (I),

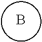

is selected from the group consisting of

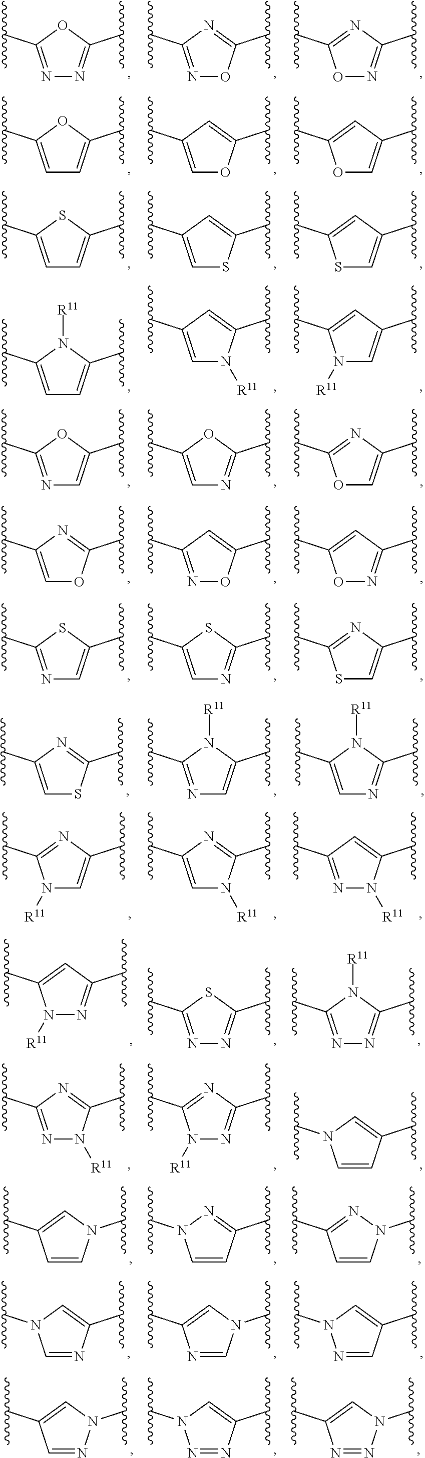

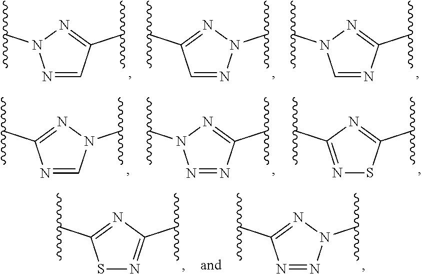

each of which is optionally substituted if possible. R¹¹ is selected from the group consisting of hydrogen, optionally substituted —C₁-C₈ alkyl, and optionally substituted —C₃-C₈ cycloalkyl. Alternatively,

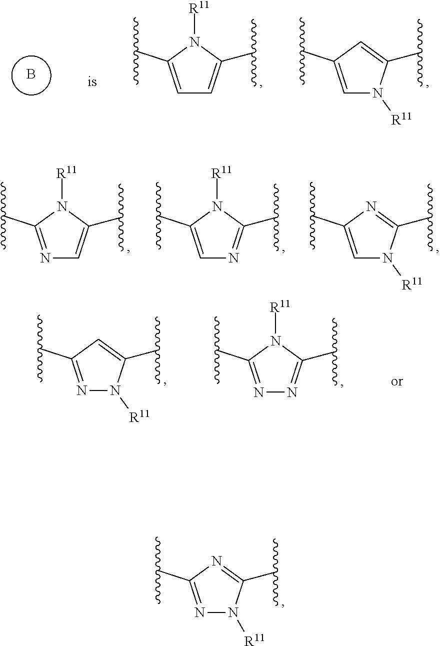

L is absent, and R¹¹ and R³ are taken together with the nitrogen and carbon atoms to which they are respectively attached to form an optionally substituted fused 5- to 8-membered heterocycyl or an optionally substituted fused heteroaryl.

In certain embodiments of the compounds of Formula (I),

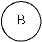

is optionally substituted 6-membered heteroaryl.

In certain embodiments of the compounds of Formula (I),

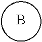

is selected from the group consisting of

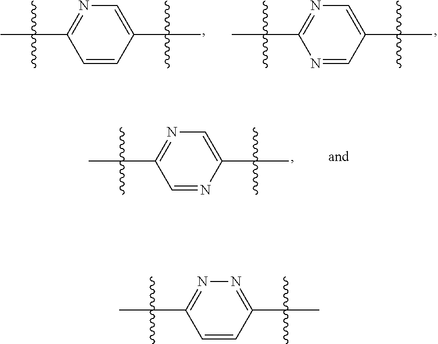 [SMILES: *c1ccc(*)nc1 ; *c1cnc(*)nc1 ; *c1cnc(*)cn1 ; *c1ccc(*)nn1]

and is optionally substituted.

In certain embodiments of the compounds of Formula (I),

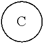

is fused bicyclic heteroaryl, for example, an 8- to 10-membered fused bicyclic heteroaryl.

In certain embodiments of the compounds of Formula (I),

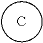

is selected from the group below:

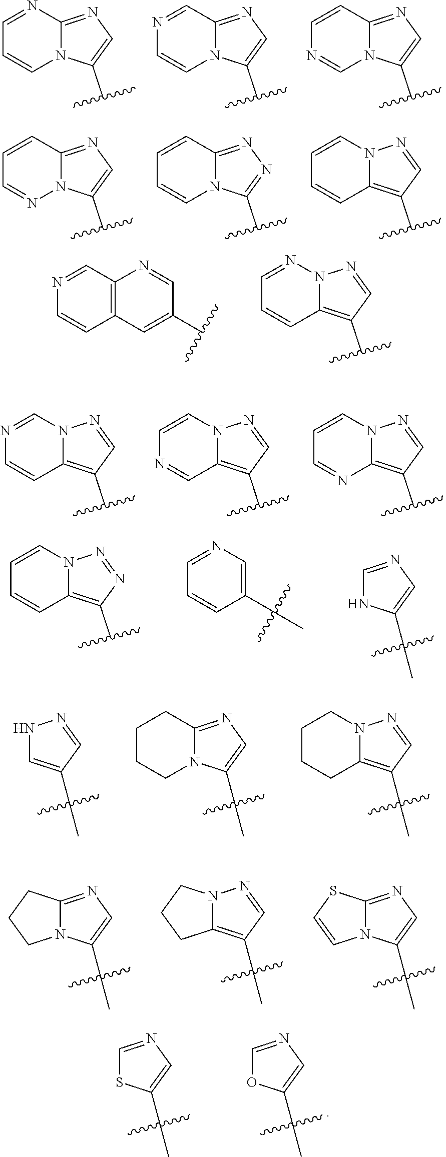

In certain embodiments of the compounds of Formula (I), m is 0.

In certain embodiments of the compounds of Formula (I), m is 1 or m is 2.

In certain embodiments of the compounds of Formula (I), m is 1, 2, 3 or 4 and at least one R¹ is halogen, optionally substituted —C₁-C₈ alkyl, or —CN. In these embodiments, m is preferably 1 or 2. More preferably m is 1.

In certain embodiments of the compounds of Formula (I), m is 1, 2, 3 or 4 and at least one R¹ is —NR⁴R⁵, wherein R⁴ and R⁵ as previously defind; alternatively, R⁴ and R⁵ are taken together with the nitrogen atom to which they are attached to form an optionally substituted -3 to 12-membered heterocyclic ring, preferably an optionally substituted 3 to 8-membered heterocyclic ring. In these embodiments, m is preferably 1 or 2. More preferably m is 1.

In certain embodiments of the compounds of Formula (I), m is 1, 2, 3, or 4 and at least one R¹ is optionally substituted —C₅-C₈ cycloalkenyl, optionally substituted aryl, or optionally substituted heteroaryl. In these embodiments, m is preferably 1 or 2. More preferably m is 1.

In certain embodiments of the compounds of Formula (I), m is 1, 2, 3 or 4, and at least one R¹ is

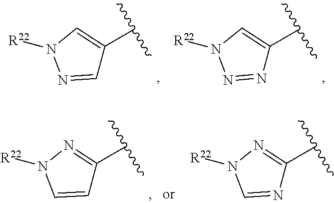

or at least one R¹ is

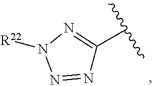

wherein R²² is hydrogen, optionally substituted —C₁-C₆ alkyl, or optionally substituted —C₃-C₈ cycloalkyl. In certain embodiments, R²² is hydrogen, optionally substituted C₁-C₄-alkyl or optionally substituted C₃-C₆-cycloalkyl. Preferably R²² is hydrogen, methyl, fluoromethyl, difluoromethyl, trifluoromethyl, or fluorocyclopropyl, or R²² is oxetanyl or 2-hydroxy-2-methylpropyl. In these embodiments, m is preferably 1 or 2. More preferably m is 1.

In certain embodiments of the compounds of Formula (I), m is 1 or m is 2, and each R¹ is independently —F, —CN, or optionally substituted —CH₃, preferably CH₂F, CHF₂ or CF₃.

In certain embodiments of the compounds of Formula (I), m is 0.

In certain embodiments of the compounds of Formula (I), R¹ is optionally substituted 3- to 12-membered heterocycloalkyl, optionally substituted aryl, or optionally substituted heteroaryl. Preferably R¹ is optionally substituted 3- to 12-membered spiro-heterocycloalkyl or optionally substituted heteroaryl.

In certain embodiments of the compounds of Formula (I), R¹ is selected from the groups below, and R¹ is optionally substituted when possible:

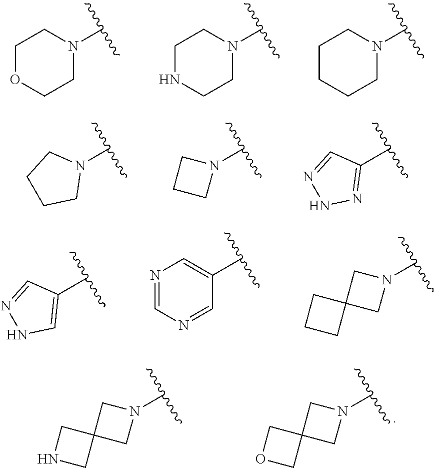

In certain embodiments of the compounds of Formula (I), R¹ is selected from the groups below, and R¹ is optionally substituted when possible:

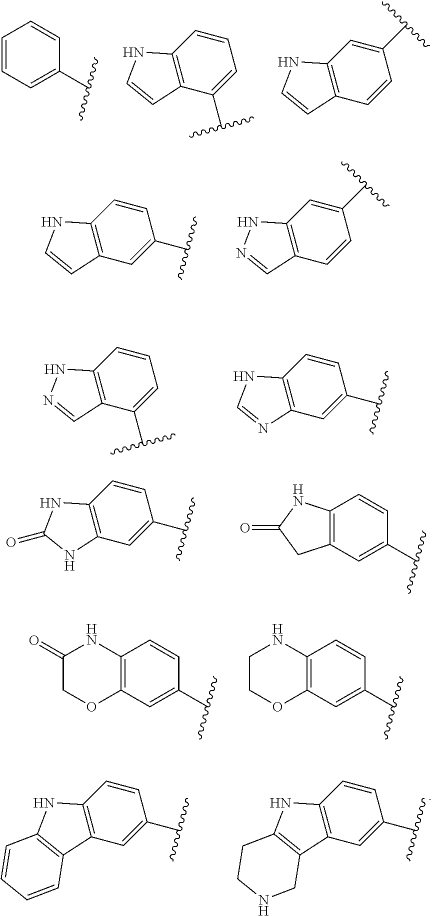

In certain embodiments of the compounds of Formula (I), R¹ is selected from the groups below, and R¹ is optionally substituted when possible:

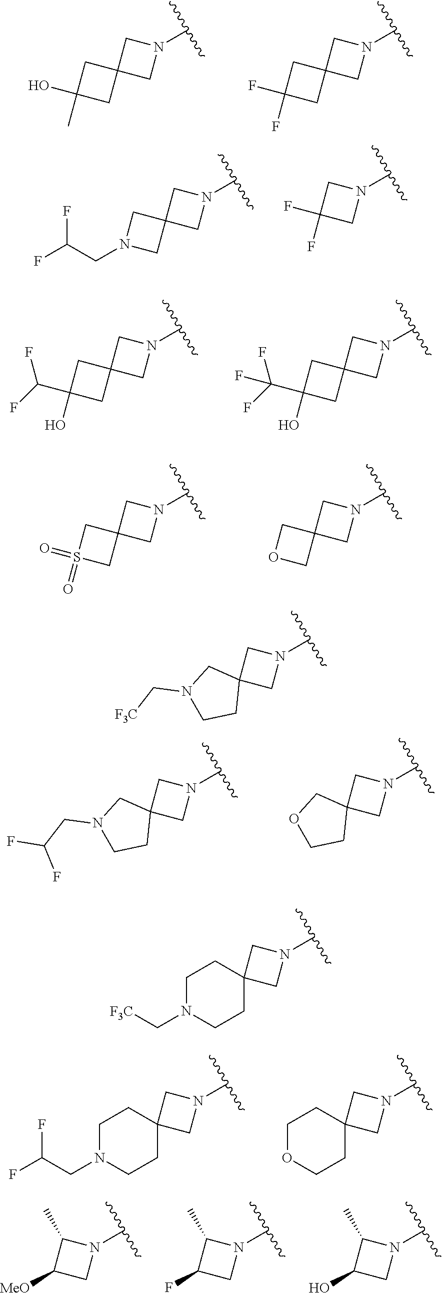

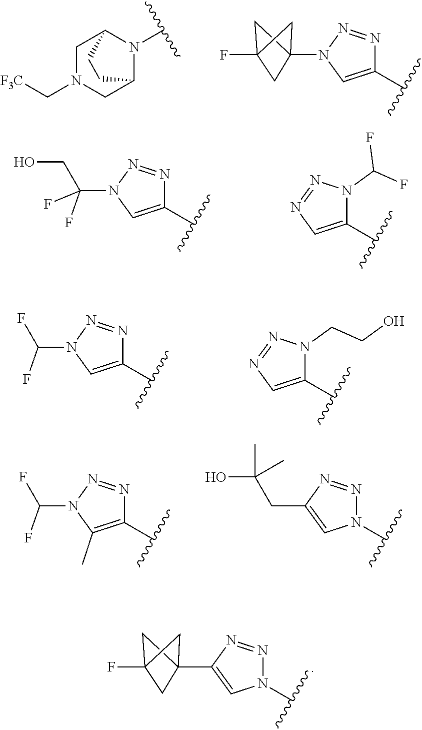

In certain embodiments of the compounds of Formula (I), L is —(CR⁶R⁷)ₚ—, p is 1 or 2, and R⁶ and R⁷ are as previously defined. Preferably each R⁶ and R⁷ is independently hydrogen or halogen; more preferably each R⁶ and R⁷ is hydrogen.

In certain embodiments of the compounds of Formula (I), L is absent.

In certain embodiments of the compounds of Formula (I), L is —C(O)NH— or —NHC(O)— and B is absent.

In certain embodiments of the compounds of Formula (I), n is 1, 2, 3, or 4 and at least one R² is optionally substituted —C₁-C₈ alkyl and halogen. In these embodiments, n is preferably 1 or 2. More preferably n is 1.

In certain embodiments of the compounds of Formula (I), n is 1, 2, 3, or 4 and at least one R² is —CH₃, —CD₃, —CF₃, —Cl, or —F. In these embodiments, n is preferably 1 or 2. More preferably n is 1.

In certain embodiments of the compounds of Formula (I), R³ is optionally substituted —C₃-C₁₂ cycloalkyl, optionally substituted —C₅-C₁₂ cycloalkenyl, or optionally substituted 3- to 12-membered heterocycloalkyl. In certain embodiments, R³ is optionally substituted —C₃-C₆ cycloalkyl, optionally substituted —C₅-C₆ cycloalkenyl, or optionally substituted 3- to 6-membered heterocycloalkyl.

In certain embodiments of the compounds of Formula (I), R³ is selected from the groups below:

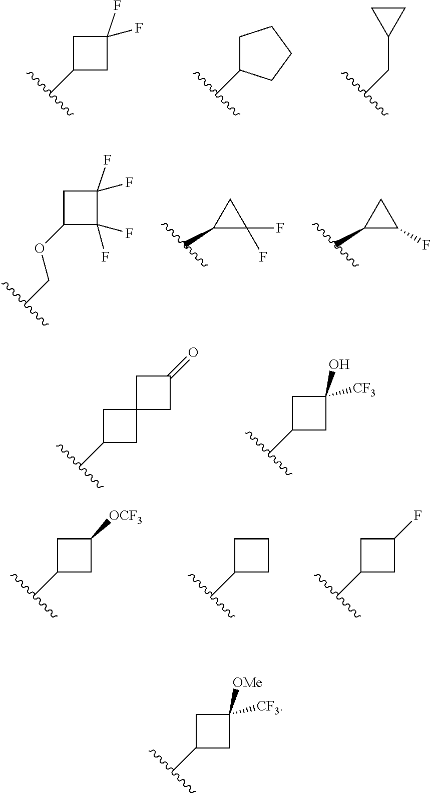

In certain embodiments of the compounds of Formula (I), R³ is

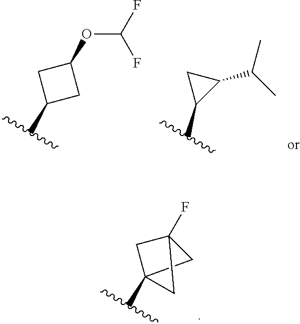

In certain embodiments of the compounds of Formula (I), R³ is select from the groups below, and R³ is optionally substituted:

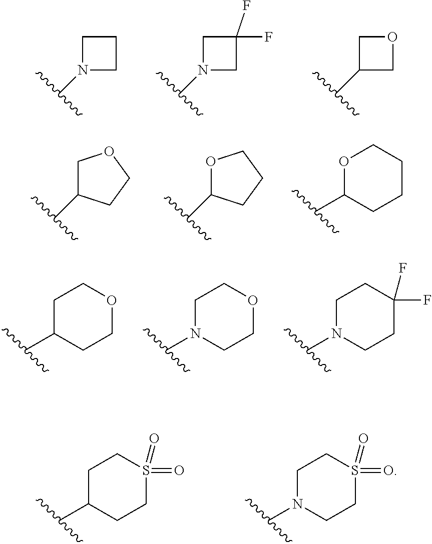

In certain embodiments of the compounds of Formula (I),

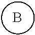

is selected from the group consisting of H

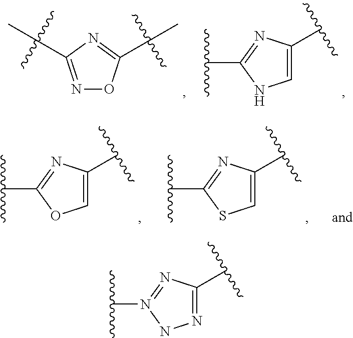

In certain embodiments of the compounds of Formula (I),

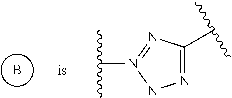

In certain embodiments of the compounds of Formula (I),

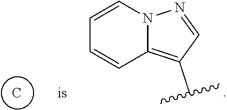

In certain embodiments of the compounds of Formula (I),

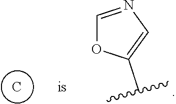

In certain embodiments of the compounds of Formula (I), m is 1, and R¹ is optionally substituted —C₃-C₈ cycloalkyl, optionally substituted —C₅-C₈ cycloalkenyl, optionally substituted 3- to 8-membered heterocycloalkyl, optionally substituted aryl, or optionally substituted heteroaryl.

In certain embodiments of the compounds of Formula (I), L is absent, and R³ is optionally substituted —C₃-C₁₂ cycloalkyl, optionally substituted —C₅-C₁₂ cycloalkenyl, or optionally substituted 3- to 12-membered heterocycloalkyl.

In certain embodiments, the compound of Formula (I) is represented by Formula (II):

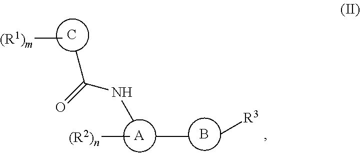

wherein R¹, R², R³, m, n,

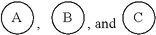

are as previously defined.

In certain embodiments, the compound of Formula (I) is represented by Formula (III):

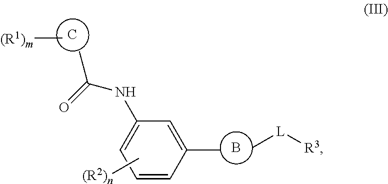

wherein R¹, R², R³, m, n,

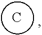

and L are as previously defined.

In certain embodiments, the compound of Formula (I) is represented by Formula (IV):

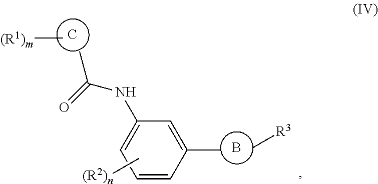

wherein R¹, R², R³, m, n,

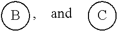

are as previously defined.

In certain embodiments, the compound of Formula (I) is represented by Formula (V):

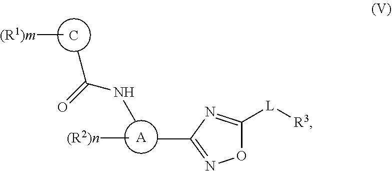

wherein R¹, R², R³, m, n,

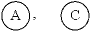

and L are as previously defined.

In certain embodiments, the compound of Formula (I) is represented by Formula (VI):

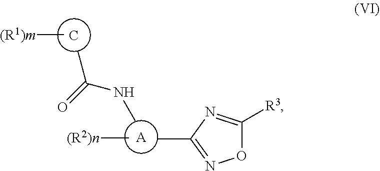

wherein R¹, R², R³, m, n,

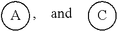

are as previously defined.

In certain embodiments, the compound of Formula (I) is represented by one of Formulae (VII-1)˜(VII-3):

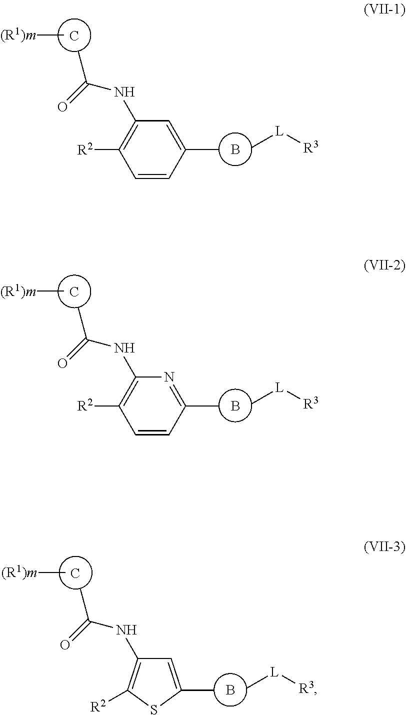

wherein R¹, R², R³, m,

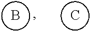

and L are as previously defined.

In certain embodiments, the compound of Formula (I) is represented by one of Formulae (VIII-1)˜(VIII-3):

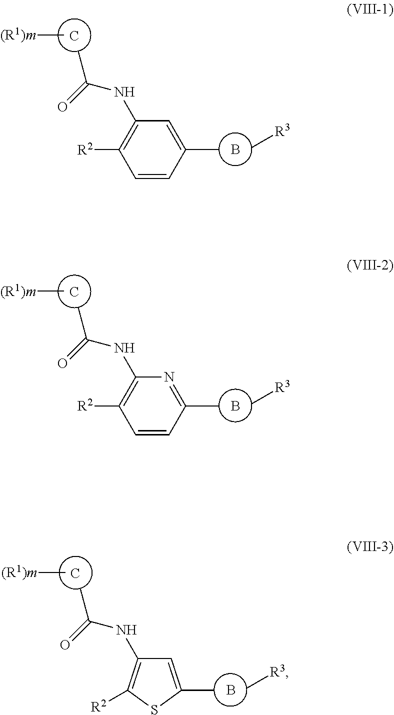

wherein R¹, R², R³, m,

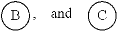

are as previously defined.

In certain embodiments, the compound of Formula (I) is represented by one of Formulae (IX-1)˜(IX-16):

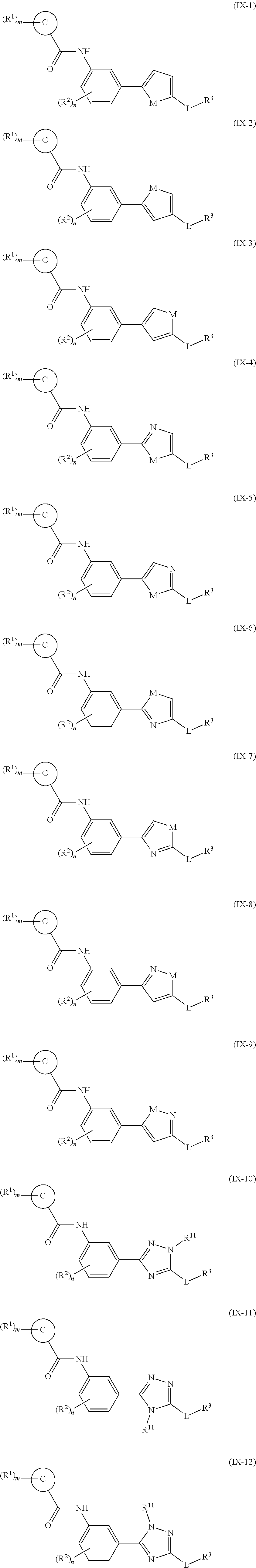

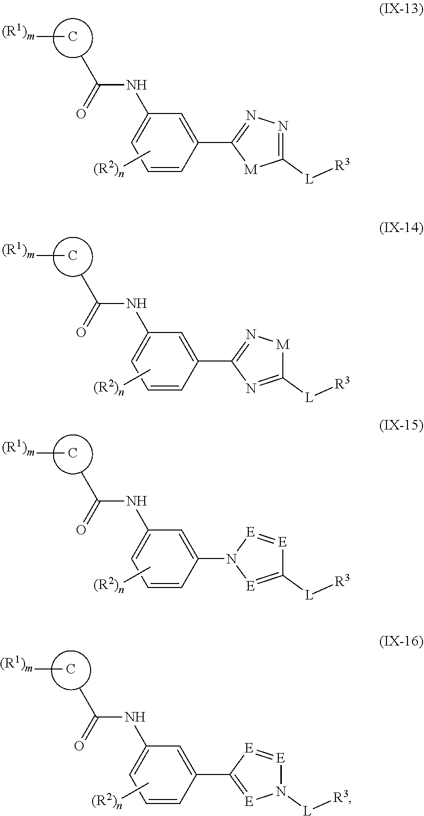

wherein each M is O, S, or N—R¹¹; each E is independently CH or N; and R¹, R², R³, m, n, R¹¹,

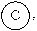

and L are as previously defined.

In certain embodiments, the compound of Formula (I) is represented by one of Formulae (X-1)˜(X-16):

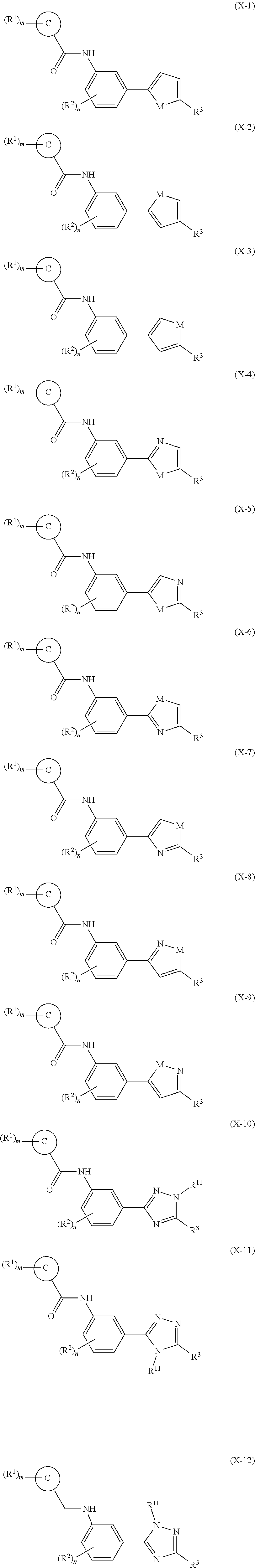

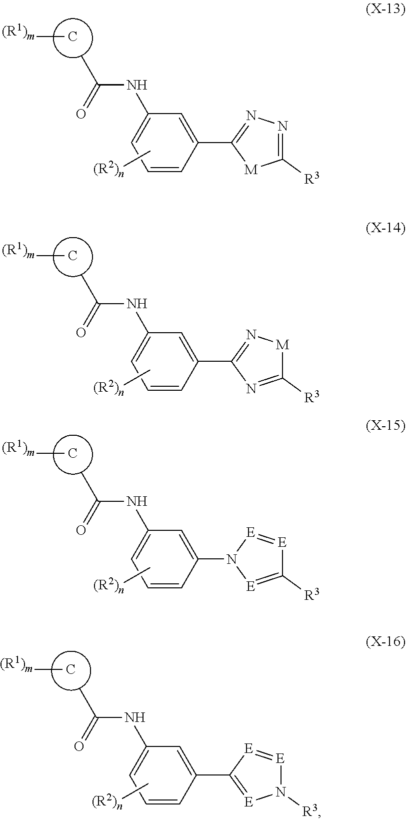

wherein M, E, R¹, R², R³, m, n, R¹¹, and

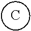

are as previously defined.

In certain embodiments, the compound of Formula (I) is represented by one of Formulae (XI-1)˜(XI-6):

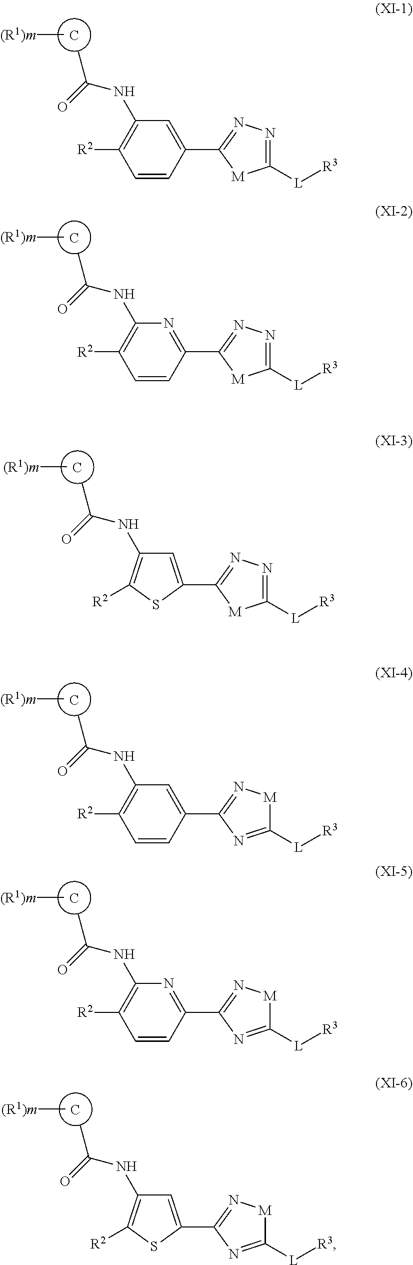

wherein R¹, R², R³, m, M,

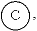

and L are as previously defined.

In certain embodiments, the compound of Formula (I) is represented by one of Formulae (XII-1)˜(XII-6):

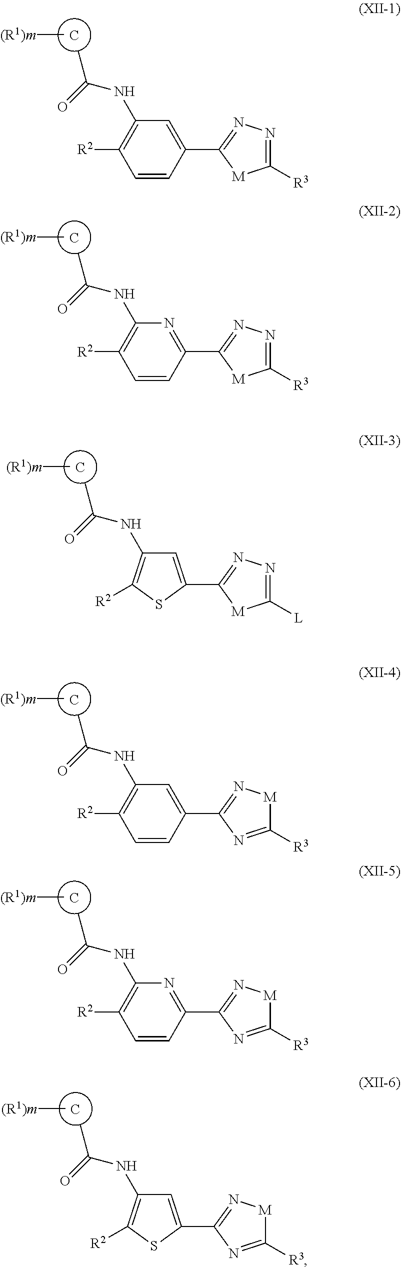

wherein R¹, R², R³, m, M, and

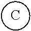

are as previously defined.

In certain embodiments, the compound of Formula (I) is represented by Formula (XIII):

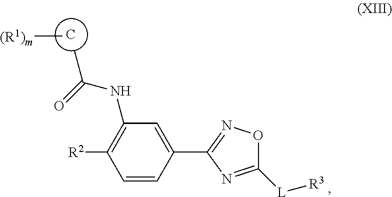

wherein R¹, R², R³, m, L, and

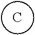

are as previously defined, preferably R² is optionally substituted methyl, such as methyl, fluoromethyl, difluoromethyl or trifluoromethyl.

In certain embodiments, the compound of Formula (I) is represented by Formula (XIV):

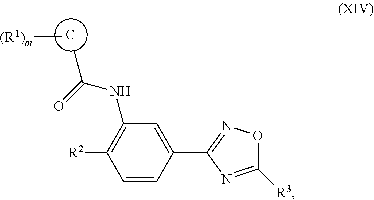

wherein R¹, R², R³, m, and

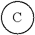

are as previously defined, preferably R² is optionally substituted methyl, such as methyl, fluoromethyl, difluoromethyl or trifluoromethyl.

In certain embodiments, the compound of Formula (I) is represented by Formula (XIII) or Formula (XIV), wherein

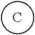

is selected from the groups below:

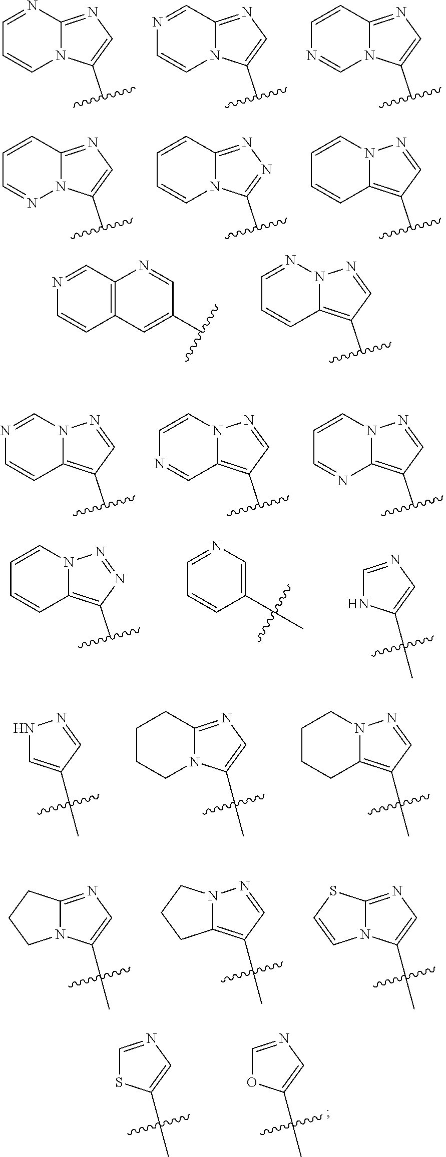

R² is optionally substituted methyl or halogen; and R³ is selected from the groups below, and is optionally substituted when possible:

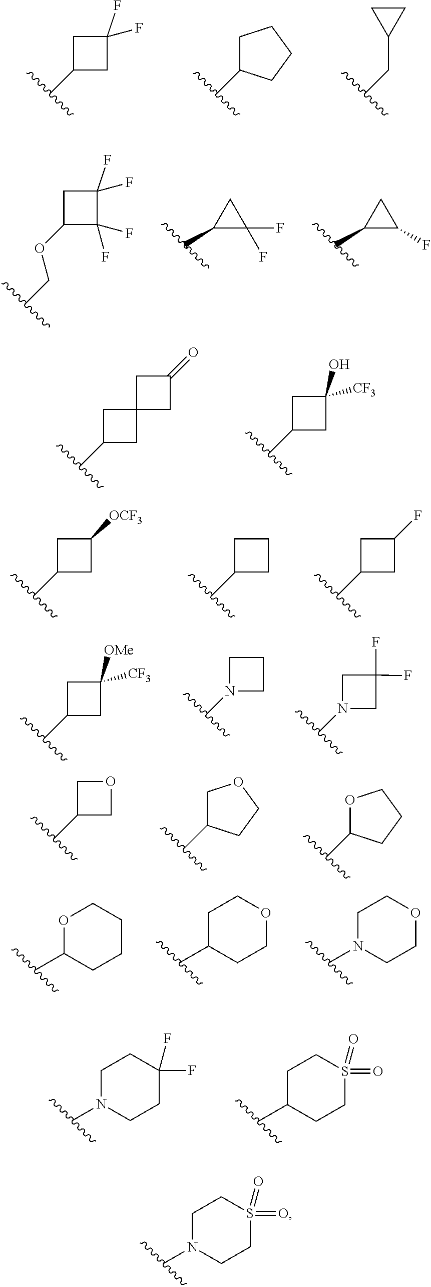

### or R³ is

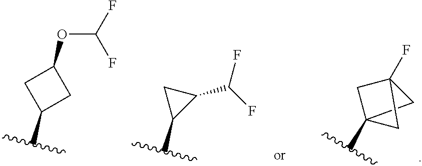

In certain embodiments, the compound of Formula (I) is represented by one of Formulae (XV-1)˜(XV-4):

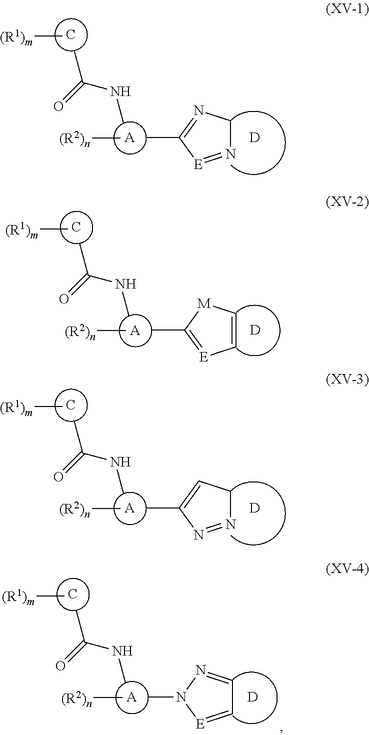

wherein

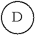

is optionally substituted cycloalkenyl, optionally substituted heterocycloalkyl, optionally substituted aryl, or optionally substituted heteroaryl; R¹, R², m, n, E, M,

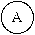

and

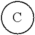

are as previously defined. In certain embodiments of the compounds of Formulas (XV-1) and (XV-4),

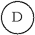

is not optionally substituted cycloalkenyl or optionally substituted aryl.

In certain embodiments, the compound of Formula (I) is represented by one of Formulae (XVI-1)˜(XVI-4):

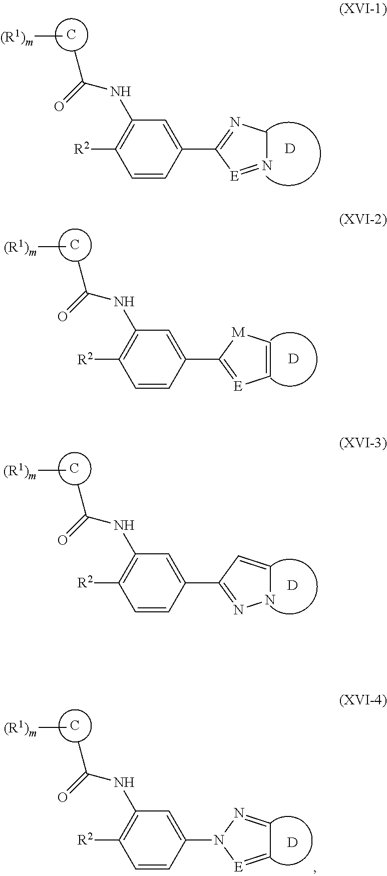

wherein R¹, R², m, E, M,

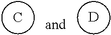

are as previously defined, and

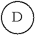

is optionally substituted.

In certain embodiments, the compound of Formula (I) is represented by Formula (XVII):

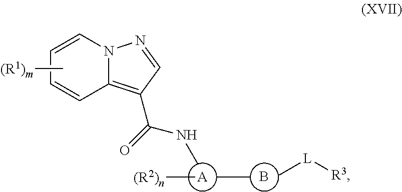

wherein L, R¹, R², R³, m, n,

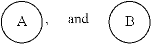

are as previously defined.

In certain embodiments, the compound of Formula (I) is represented by Formula (XVII-1) or Formula (XVII-2):

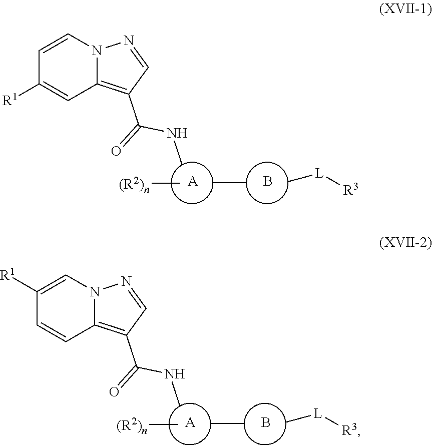

wherein L, R¹, R², R³, n,

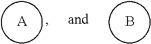

are as previously defined.

In certain embodiments, the compound of Formula (I) is represented by Formula (XVIII):

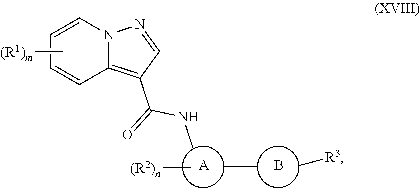

wherein R¹, R², R³, m, n,

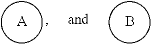

are as previously defined.

In certain embodiments, the compound of Formula (I) is represented by Formula (XVIII-1) or Formula XVIII-2):

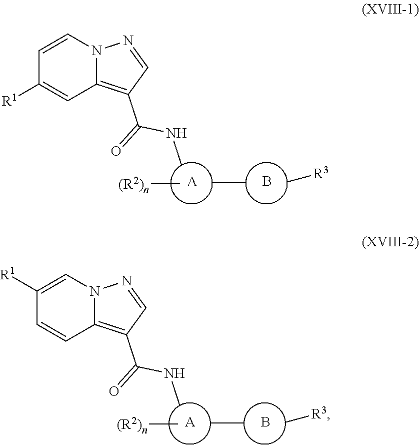

wherein R¹, R², R³, n,

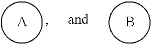

are as previously defined.

In certain embodiments, the compound of Formula (I) is represented by one of Formulae (XIX-1)˜(XIX-4):

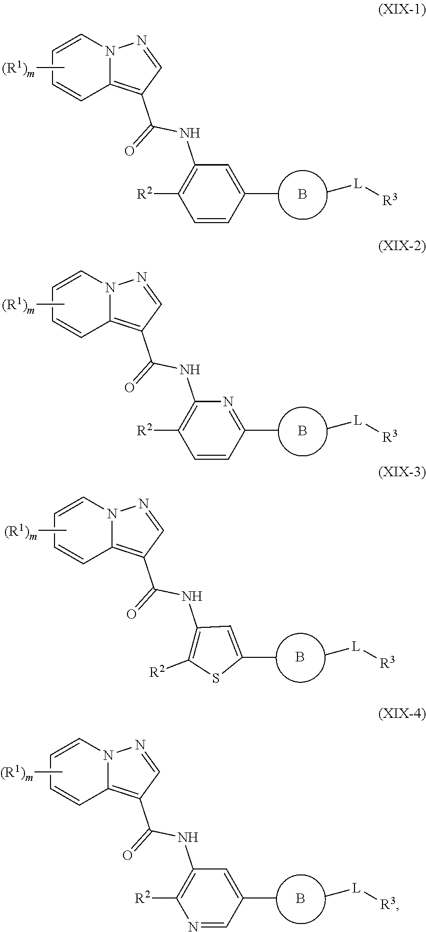

wherein L, R¹, R², R³, m, and

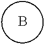

are as previously defined. In certain embodiments, R² is methyl or halogen, preferably methyl.

In certain embodiments, the compound of Formula (I) is represented by one of Formulae (XX-1)˜(XX-8):

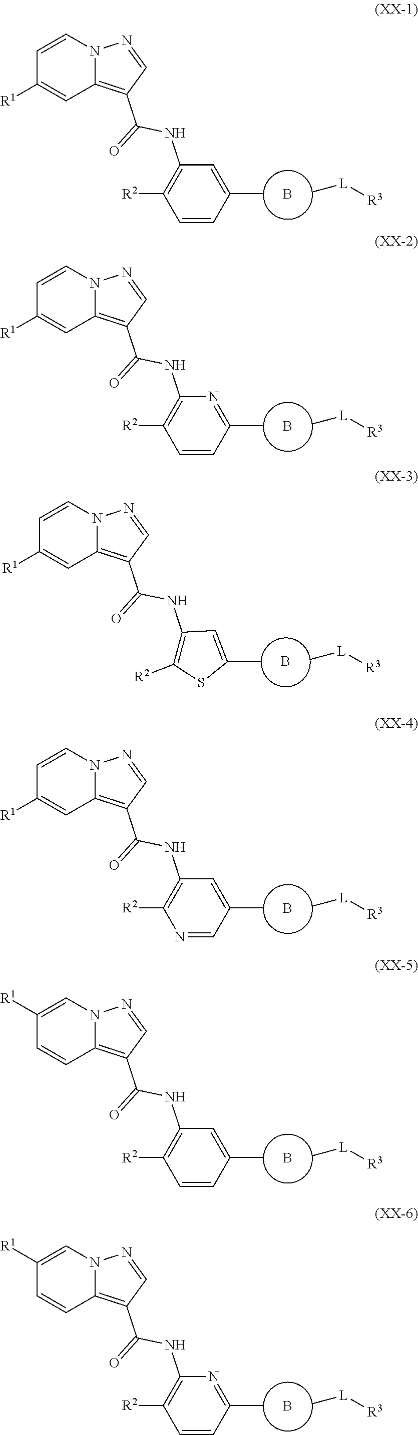

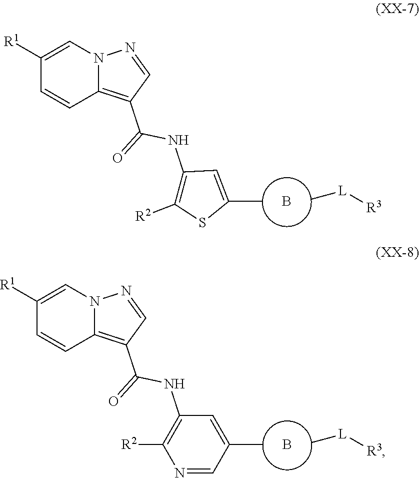

wherein L, R¹, R², R³, and

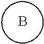

are as previously defined. In certain embodiments, R² is methyl or halogen, preferably methyl.

In certain embodiments, the compound of Formula (I) is represented by one of Formulae (XXI-1)˜(XXI-4):

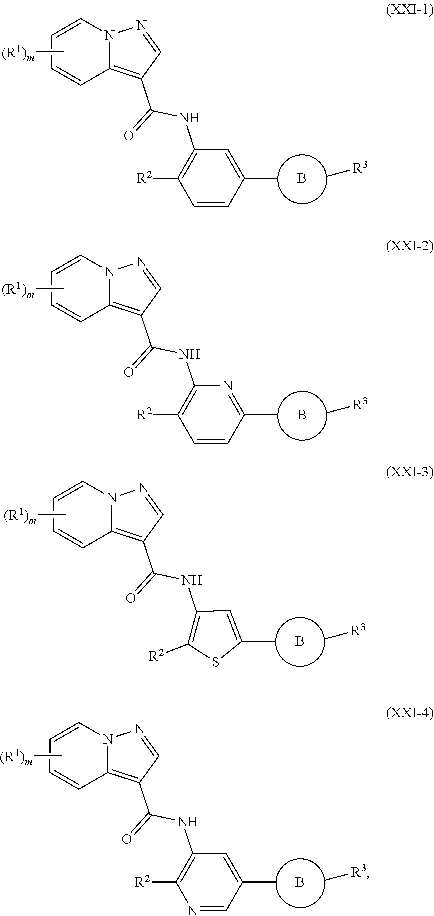

wherein R¹, R², R³, m, and

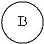

are as previously defined. In certain embodiments, R² is methyl or halogen, preferably methyl.

In certain embodiments, the compound of Formula (I) is represented by one of Formulae (XXII-1)˜(XXII-8):

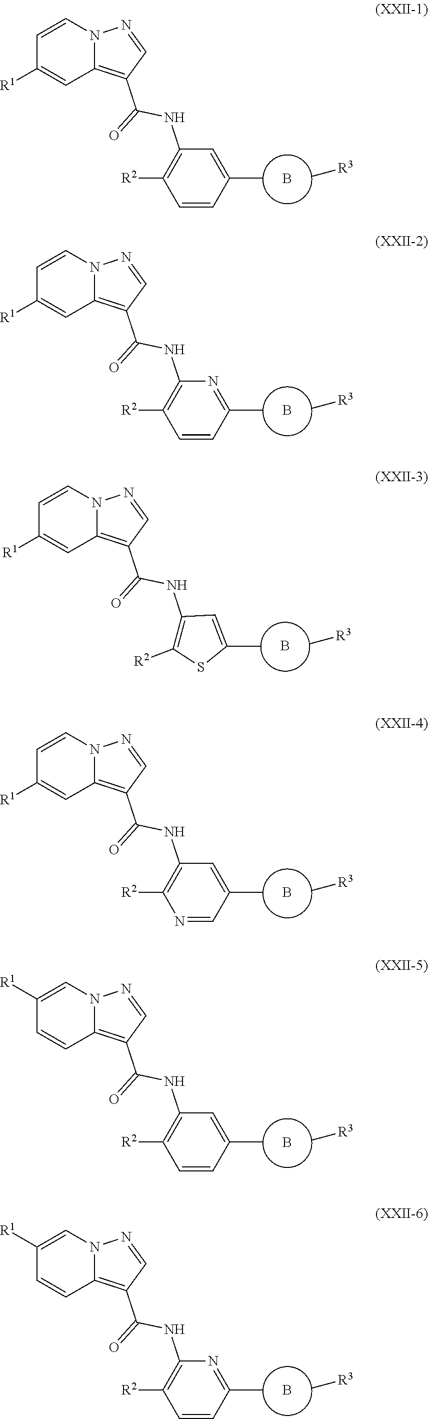

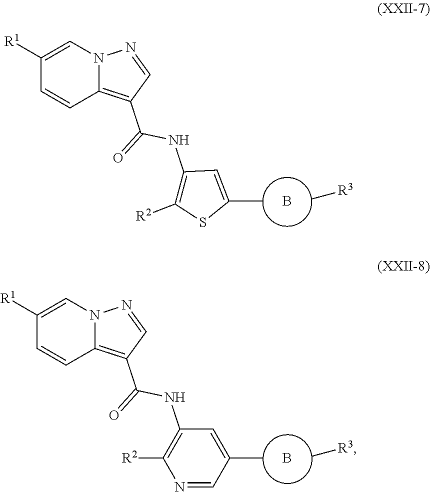

wherein R¹, R², R³, and

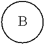

are as previously defined. In certain embodiments, R² is methyl or halogen, preferably methyl.

In certain embodiments, the compound of Formula (I) is represented by one of Formulae (XIX-1)˜(XX-4), (XX-1)˜(XX-8), (XXI-1)˜(XXI-4), and (XXII-1)˜(XXII-8), wherein R¹ is optionally substituted —C₃-C₁₂ cycloalkyl, optionally substituted —C₅-C₁₂ cycloalkenyl, optionally substituted 3- to 12-membered heterocycloalkyl, optionally substituted aryl, or optionally substituted heteroaryl; R² is optionally substituted methyl or halogen; R³ is optionally substituted —C₃-C₁₂ cycloalkyl, optionally substituted —C₅-C₁₂ cycloalkenyl, or optionally substituted 3- to 12-membered heterocycloalkyl; and

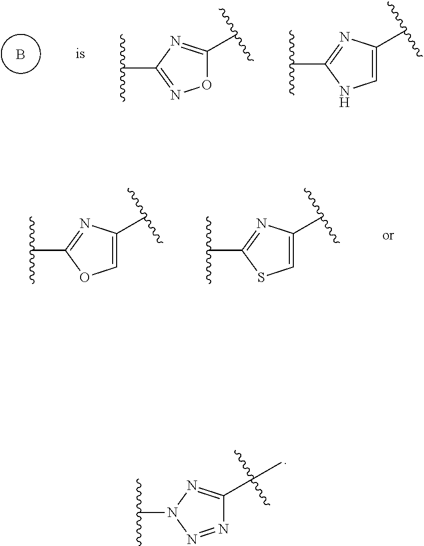

In certain embodiments, the compound of Formula (I) is represented by Formula (XXIII-1) or Formula (XXIII-2):

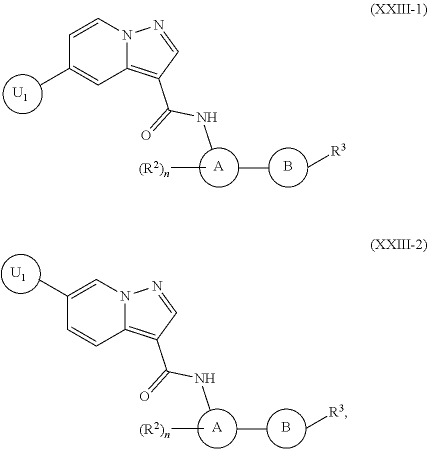

wherein U₁ is optionally substituted 3- to 12-membered heterocycloalkyl, or optionally substituted heteroaryl; R², R³, n,

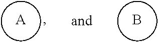

are as previously defined.

In certain embodiments, the compound of Formula (I) is represented by Formula (XXIII-1) or Formula (XXIII-2), U₁ is selected from the groups below, and is optionally substituted when possible:

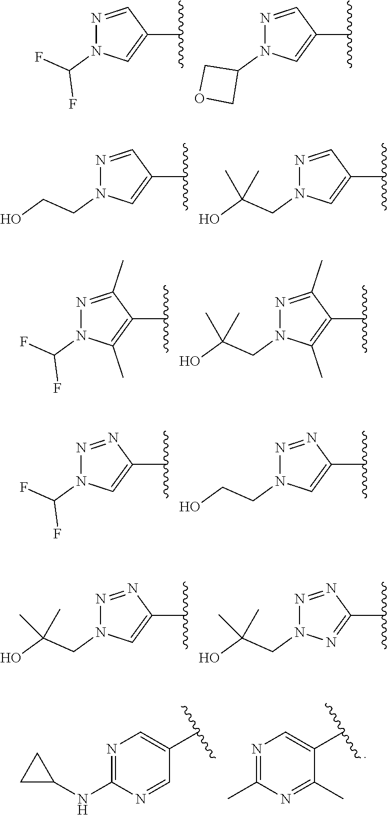

In certain embodiments, the compound of Formula (I) is represented by Formula (XXIII-1a) or Formula (XXIII-2a):

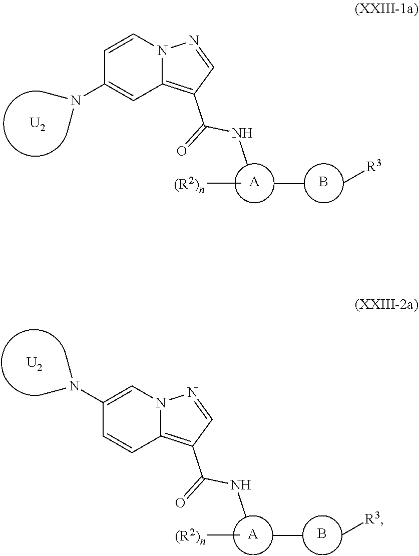

wherein U₂ is optionally substituted 3- to 12-membered heterocycloalkyl; R², R³, n,

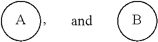

are as previously defined. Preferably, U₂ is optionally substituted 3- to 12-membered spiro heterocycloalkyl.

In certain embodiments, the compound of Formula (I) is represented by Formula (XXIII-1a) or Formula (XXIII-2a), U₂ is selected from the groups below, and is optionally substituted when possible:

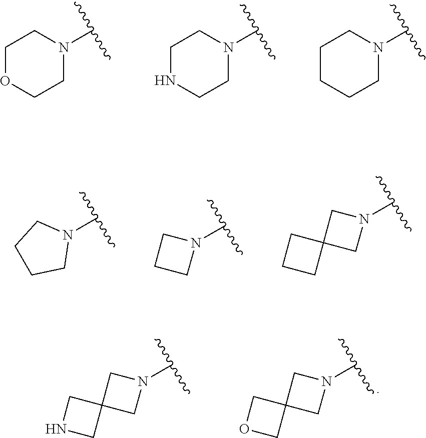

In another embodiment, the compound of Formula (I) is represented by Formula (XXIII-1a) or Formula (XXIII-2a), U₂ is selected from the groups below, and is optionally substituted when possible:

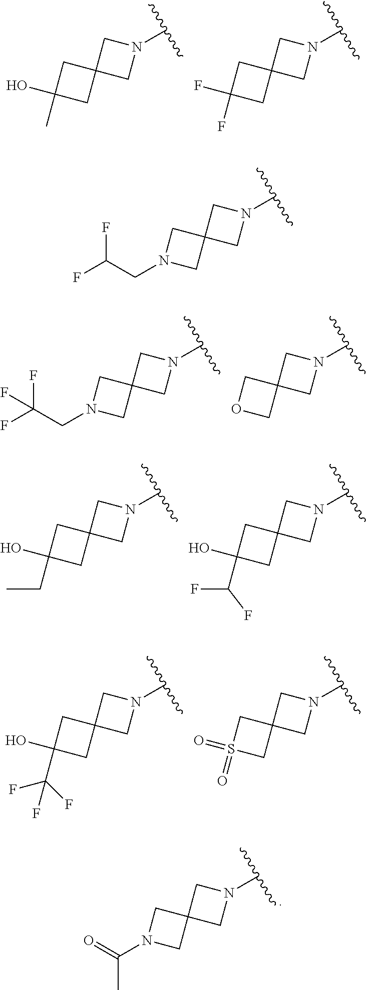

In certain embodiments, the compound of Formula (I) is represented by Formula (XXIV-1) or Formula (XXIV-2):

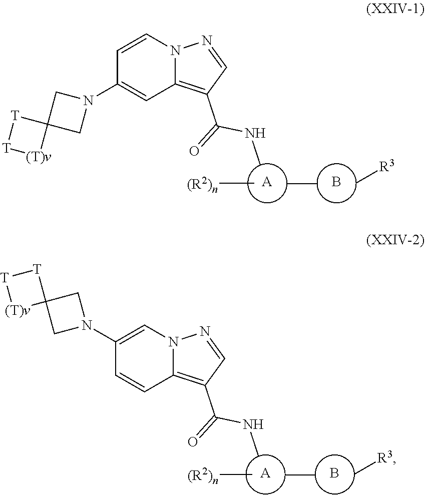

wherein at least one T is CR₂₁R₂₂, and the other Ts are independently O, NR₂₃, —SO₂—, or CR₂₁R₂₂; R₂₁ and R₂₂ are each independently selected from the group consisting of hydrogen, OH, optionally substituted —C₁-C₆ alkyl, optionally substituted —C₁-C₆ alkoxyl, and optionally substituted —C₃-C₈ cycloalkyl; R₂₃ is hydrogen, optionally substituted —C₁-C₆ alkyl, optionally substituted —C₃-C₈ cycloalkyl, —C(O)R⁴, —C(O)OR⁴, or —C(O)NR⁴R⁵, wherein R⁴ and R⁵ are as previously defined; v is 1, 2, 3 or 4, preferably, v is 1; R², R³, n,

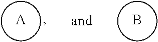

are as previously defined.

In certain embodiments, the compound of Formula (I) is represented by Formula (XXV):

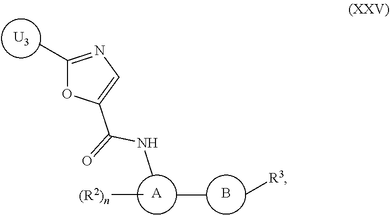

wherein U₃ is optionally substituted 3- to 12-membered heterocycloalkyl, optionally substituted aryl, or optionally substituted heteroaryl; R², R³, n,

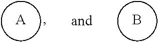

are as previously defined. Preferably, U₃ is selected from the groups below and is optionally substituted:

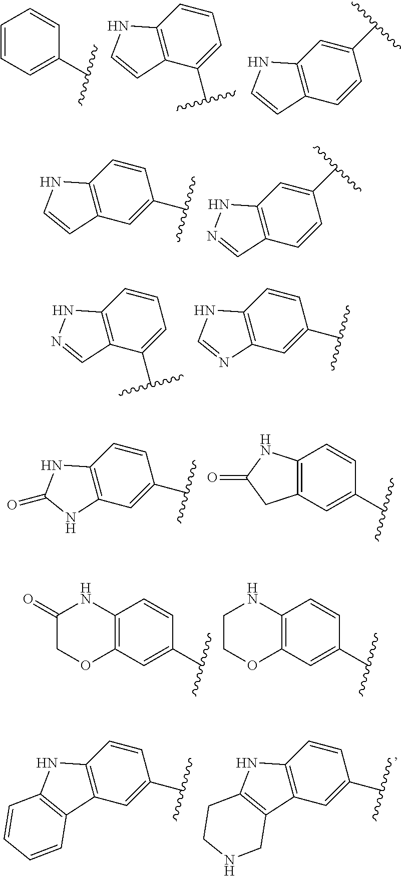

or U₃ is selected from the groups below and is optionally substituted

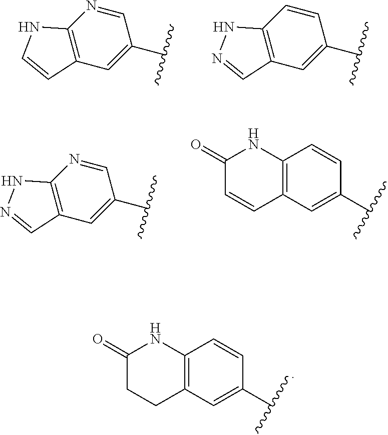 [SMILES: *c1cnc2[nH]ccc2c1 ; *c1ccc2[nH]ncc2c1 ; *c1cnc2[nH]ncc2c1 ; *c1ccc2[nH]c(=O)ccc2c1 ; *c1ccc2c(c1)CCC(=O)N2]

In certain embodiments, the compound of Formula (I) is represented by one of Formulae (XXIII-1), (XXIII-2), (XXIII-1a), (XXIII-2a), (XXIV-1), (XXIV-2), and (XXV), wherein

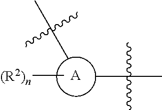

is selected from the groups below.

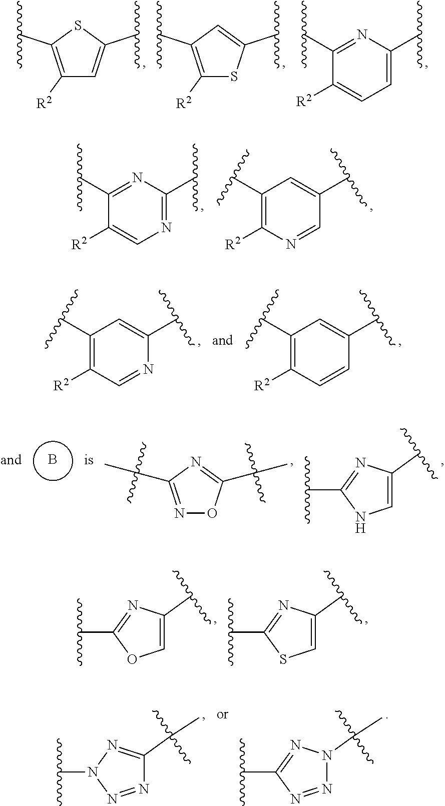

### Preferably,

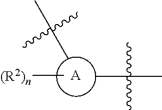

is selected from the group below:

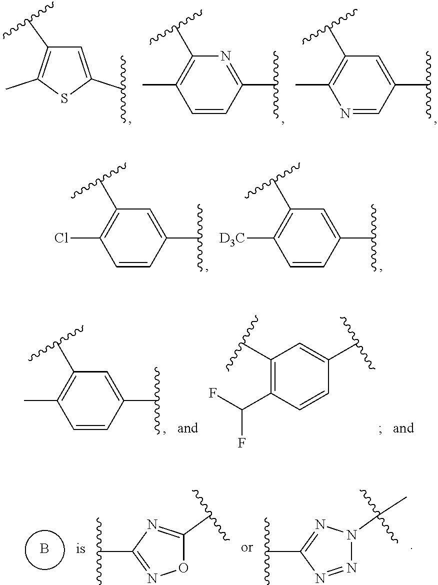

In certain embodiments, the compound of Formula (I) is represented by one of Formulae (XXIII-1), (XXIII-2), (XXIII-1a), (XXIII-2a), (XXIV-1), (XXIV-2), and (XXV), wherein

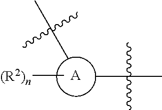

is selected from the groups below:

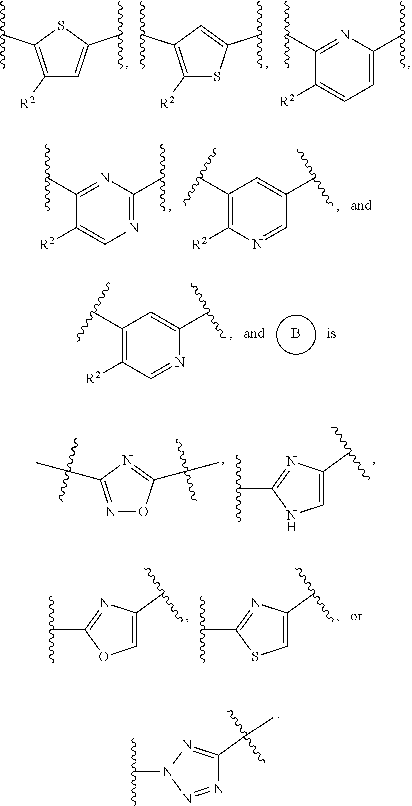

In certain embodiments, the compound of Formula (I) is represented by one of Formulae (XXVI-1)˜(XXVI-4):

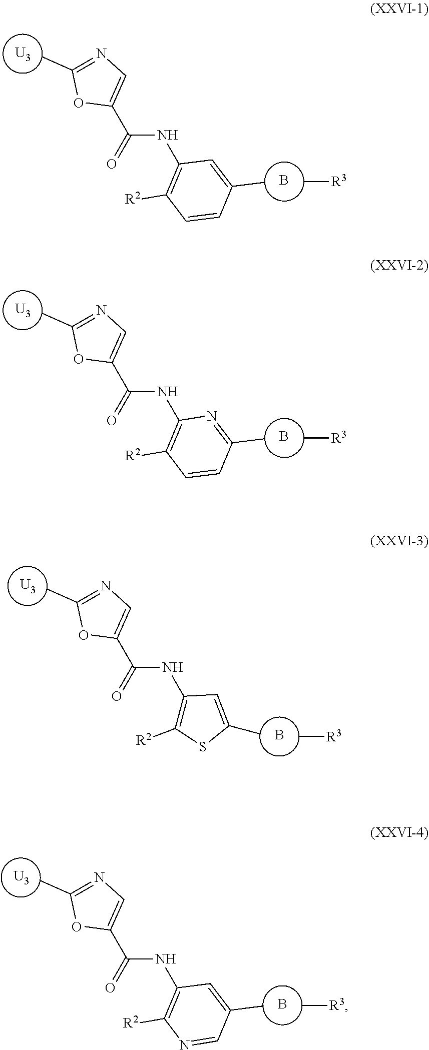

wherein U₃, R², R³, and

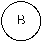

are as previously defined. Preferably, U₃ is selected from the group below and is optionally substituted:

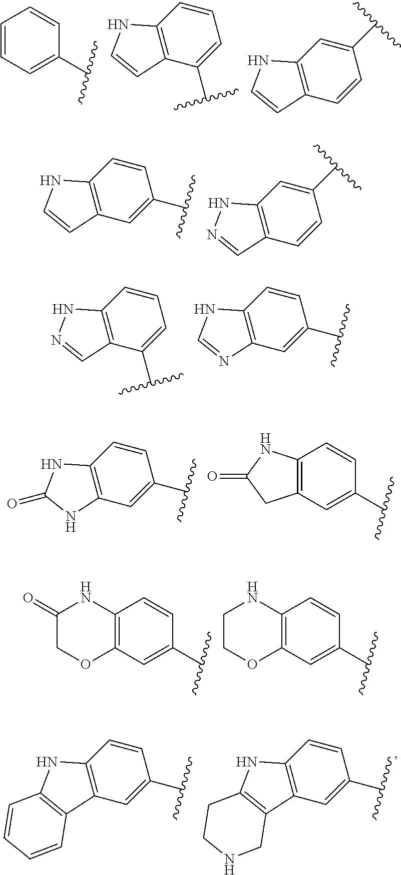

or U₃ is selected from the groups below and is optionally substituted

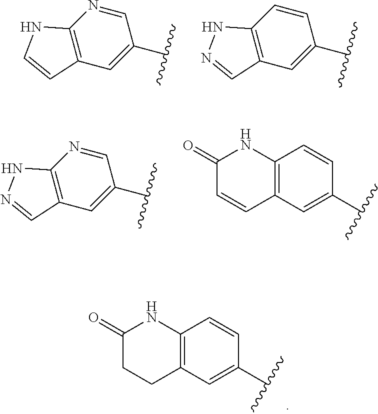

In certain embodiments, the compound of Formula (I) is represented by by one of Formulae (XXVII-1)˜(XXVII-8):

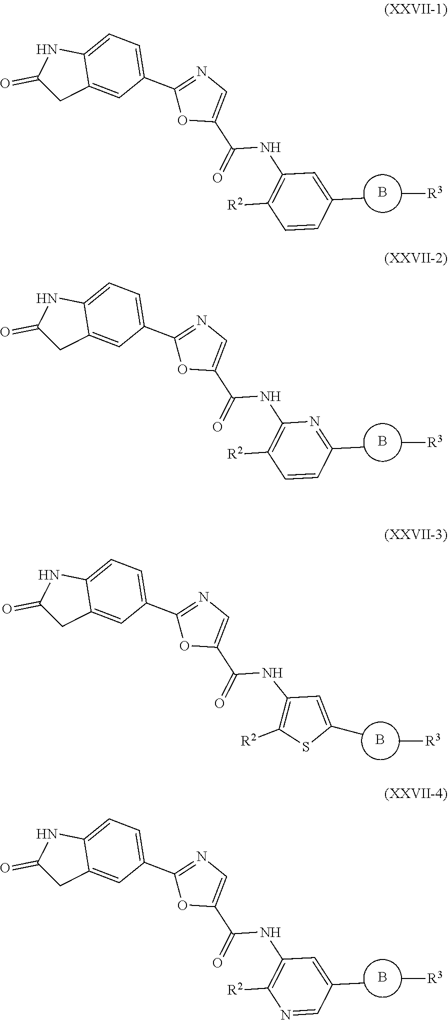

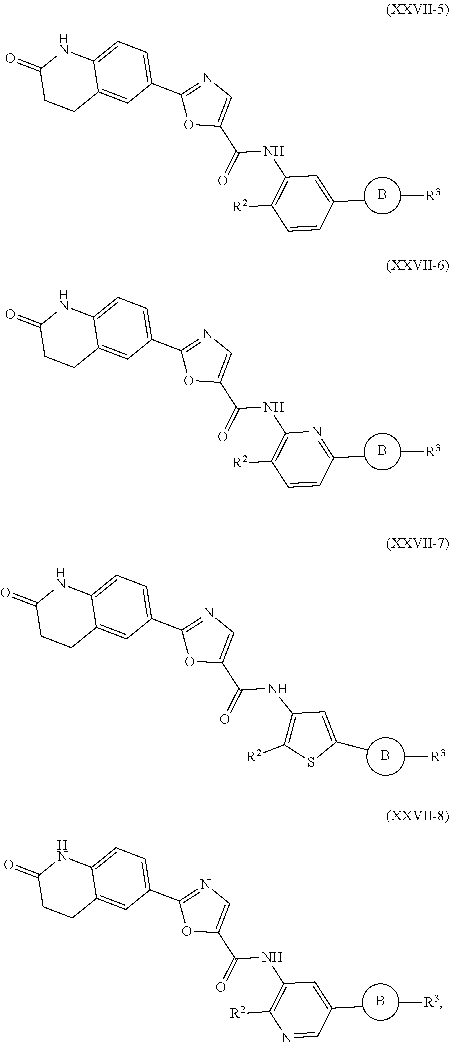

wherein R², R³, and

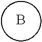

are as previously defined.

In certain embodiments, the compound of Formula (I) is represented by one of Formula (XXVIII-1) to Formula (XXVIII-8):

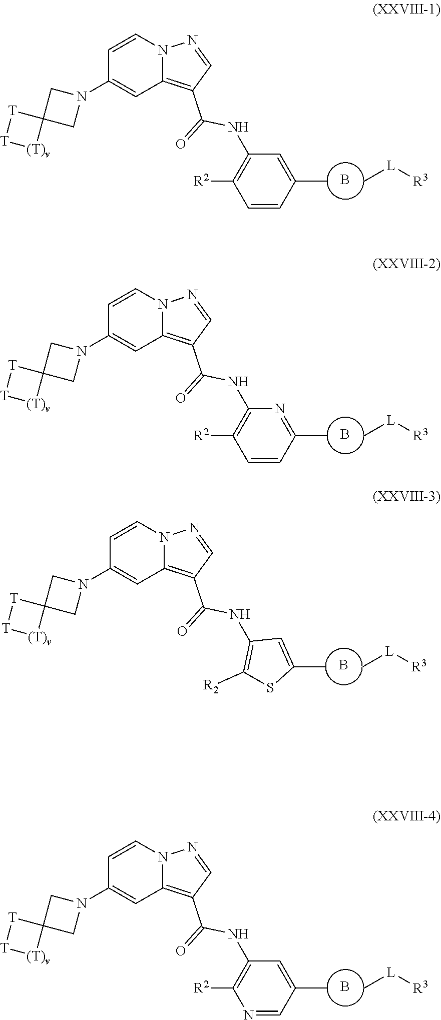

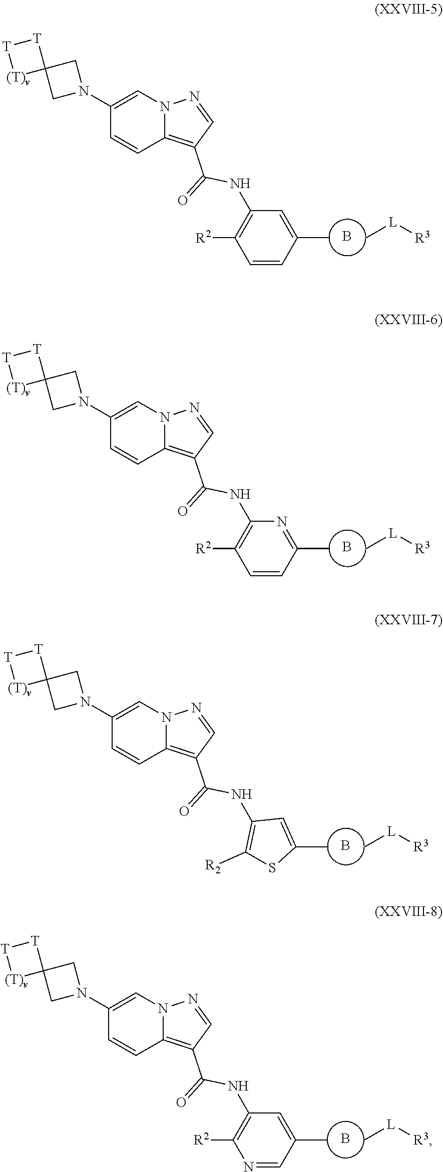

wherein T, v, L, R², R³, and

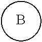

are as previously defined. In certain embodiments, R² is methyl or halogen, preferably methyl.

In certain embodiments, the compound of Formula (I) is represented by one of Formula (XXIX-1) to Formula (XXIX-8):

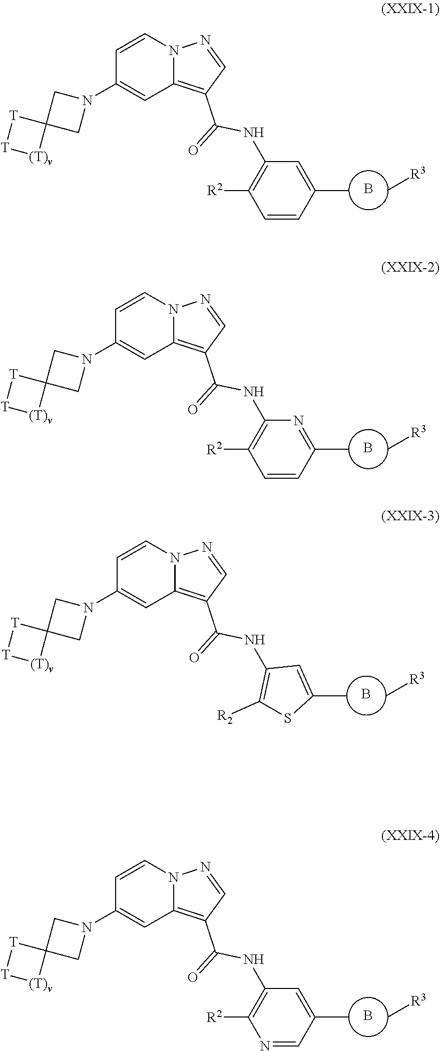

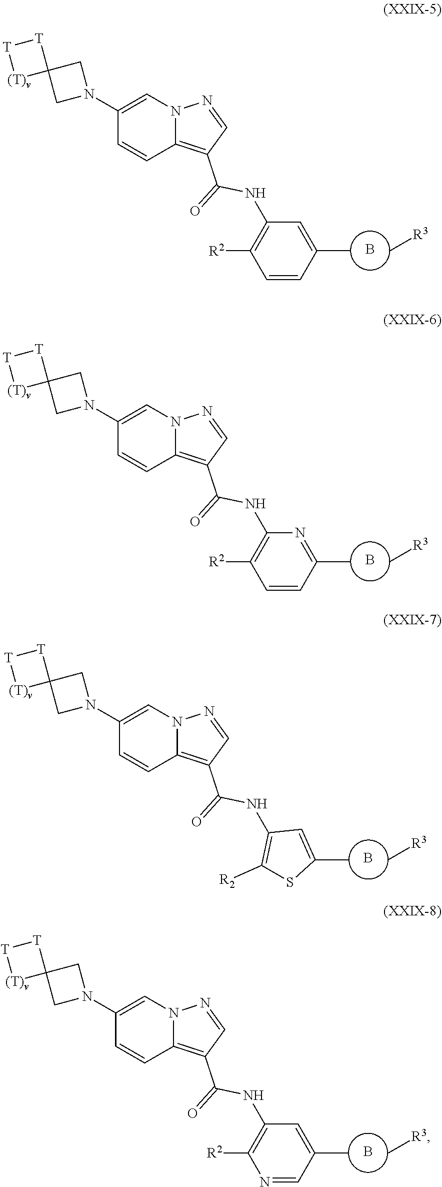

wherein T, v, R², R³, and

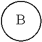

are as previously defined. In certain embodiments, R² is methyl or halogen, preferably methyl.

In certain embodiments, the compound of Formula (I) is represented by Formula (XXX-1) to Formula (XXX-8).

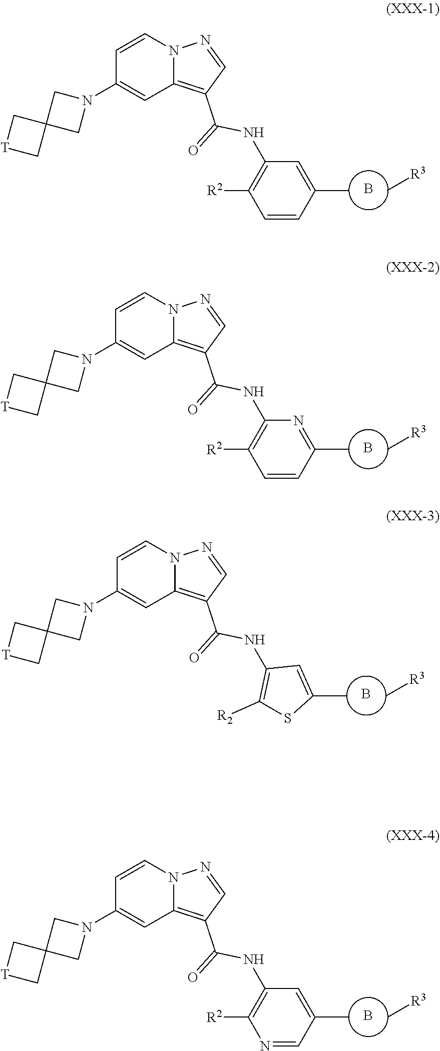

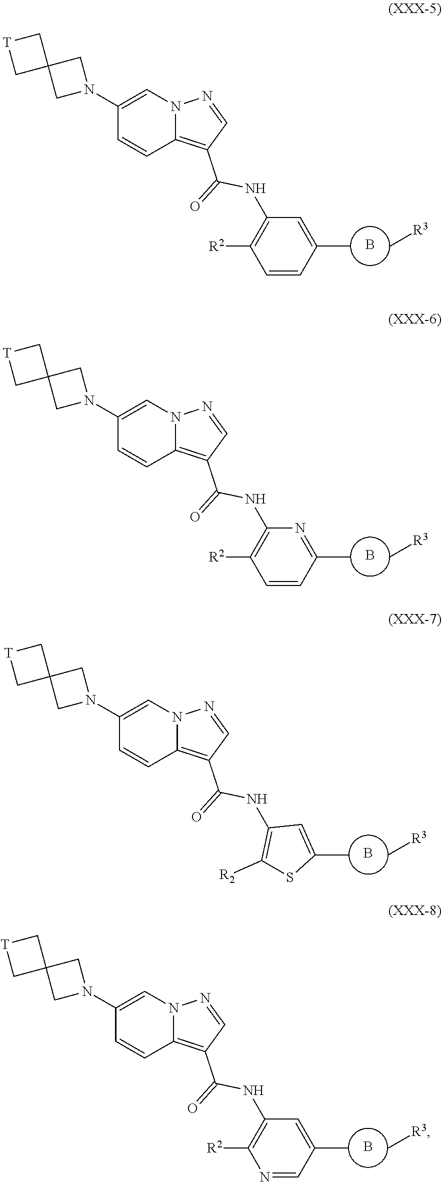

wherein T, R², R³, and

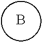

are as previously defined. In certain embodiments, R² is methyl or halogen; T is O or CR₂₁R₂₂;

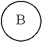

is optionally substituted and is selected from the group consisting of

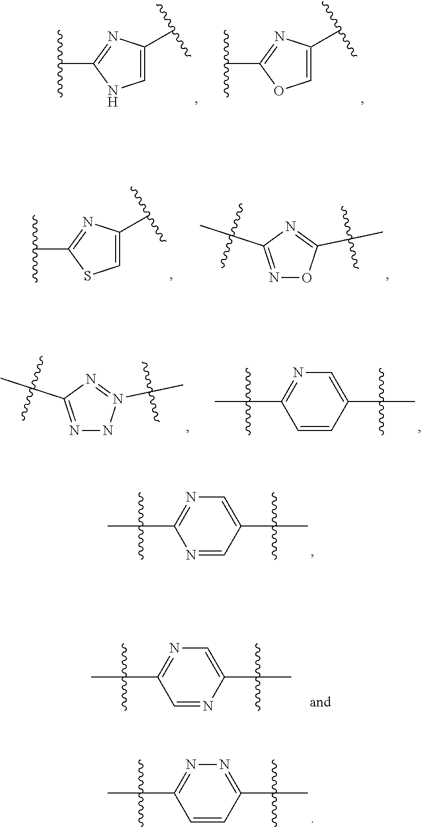

Each preferred group stated above can be taken in combination with one, any or all other preferred groups.

It will be appreciated that the description of the present invention herein should be construed in congruity with the laws and principles of chemical bonding. In some instances, it may be necessary to remove a hydrogen atom in order to accommodate a substituent at any given location.

It will be appreciated that the compounds of the present invention may contain one or more asymmetric carbon atoms and may exist in racemic, diastereoisomeric, and optically active forms. It will still be appreciated that certain compounds of the present invention may exist in different tautomeric forms. All tautomers are contemplated to be within the scope of the present invention.

The compounds of the present invention and any other pharmaceutically active agent(s) may be administered together or separately and, when administered separately, administration may occur simultaneously or sequentially, in any order. The amounts of the compounds of the present invention and the other pharmaceutically active agent(s) and the relative timings of administration will be selected in order to achieve the desired combined therapeutic effect. The administration in combination of a compound of the present invention and salts, solvates, or other pharmaceutically acceptable derivatives thereof with other treatment agents may be achieved by concomitant administration in: (1) a unitary pharmaceutical composition including both compounds; or (2) separate pharmaceutical compositions each including one of the compounds.

In certain embodiments of the combination therapy, the additional therapeutic agent is administered at a lower dose and/or dosing frequency as compared to dose and/or dosing frequency of the additional therapeutic agent required to achieve similar results in treating or preventing as PDGFR and/or c-kit kinases inhibitors.

It should be understood that the compounds encompassed by the present invention are those that are suitably stable for use as a pharmaceutical agent.

### Definitions

Listed below are definitions of various terms used to describe this invention. These definitions apply to the terms as they are used throughout this specification and claims, unless otherwise limited in specific instances, either individually or as part of a larger group.

The term “aryl,” as used herein, refers to a mono- or polycyclic carbocyclic ring system comprising at least one aromatic ring. Preferred aryl groups are C₆-C₁₂-aryl groups, including, but not limited to, phenyl, naphthyl, tetrahydronaphthyl, indanyl, and indenyl. A polycyclic aryl is a polycyclic ring system that comprises at least one aromatic ring. Polycyclic aryls can comprise fused rings, covalently attached rings or a combination thereof.

The term “heteroaryl,” as used herein, refers to a mono- or polycyclic aromatic radical having one or more ring atom selected from S, O and N; and the remaining ring atoms are carbon, wherein any N or S contained within the ring may be optionally oxidized. In certain embodiments, a heteroaryl group is a 5- to 10-membered heteroaryl, such as a 5- or 6-membered monocyclic heteroaryl or an 8- to 10-membered bicyclic heteroaryl. Heteroaryl groups include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzoxazolyl, quinoxalinyl. A polycyclic heteroaryl can comprise fused rings, covalently attached rings or a combination thereof. A heteroaryl group can be C-attached or N-attached where possible.

In accordance with the invention, aryl and heteroaryl groups can be substituted or unsubstituted.

The term “bicyclic aryl” or “bicyclic heteroaryl” refers to a ring system consisting of two rings wherein at least one ring is aromatic; and the two rings can be fused or covalently attached.

The term “alkyl” as used herein, refers to saturated, straight- or branched-chain hydrocarbon radicals. “C₁-C₄ alkyl,” “C₁-C₆ alkyl,” “C₁-C₈ alkyl,” “C₁-C₁₂ alkyl,” “C₂-C₄ alkyl,” and “C₃-C₆ alkyl,” refer to alkyl groups containing from 1 to 4, 1 to 6, 1 to 8, 1 to 12, 2 to 4 and 3 to 6 carbon atoms respectively. Examples of alkyl groups include, but are not limited to, methyl, ethyl, propyl, isopropyl, n-butyl, sec-butyl, tert-butyl, n-pentyl, neopentyl, n-hexyl, n-heptyl and n-octyl radicals.

The term “alkenyl” as used herein, refers to straight- or branched-chain hydrocarbon radicals having at least one carbon-carbon double bond. “C₂-C₈ alkenyl,” “C₂-C₁₂ alkenyl,” “C₂-C₄ alkenyl,” “C₃-C₄ alkenyl,” and “C₃-C₆ alkenyl,” refer to alkenyl groups containing from 2 to 8, 2 to 12, 2 to 4, 3 to 4 or 3 to 6 carbon atoms respectively. Alkenyl groups include, but are not limited to, ethenyl, propenyl, butenyl, 2-methyl-2-buten-2-yl, heptenyl, octenyl, and the like.

The term “alkynyl” as used herein, refers to straight- or branched-chain hydrocarbon radicals having at least one carbon-carbon triple bond. “C₂-C₈ alkynyl,” “C₂-C₁₂ alkynyl,” “C₂-C₄ alkynyl,” “C₃-C₄ alkynyl,” and “C₃-C₆ alkynyl,” refer to alkynyl groups containing from 2 to 8, 2 to 12, 2 to 4, 3 to 4 or 3 to 6 carbon atoms respectively. Representative alkynyl groups include, but are not limited to, ethynyl, 2-propynyl, 2-butynyl, heptynyl, octynyl, and the like.

The term “cycloalkyl”, as used herein, refers to a monocyclic or polycyclic saturated carbocyclic ring, such as a bi- or tri-cyclic fused, bridged or spiro system. The ring carbon atoms are optionally oxo-substituted or optionally substituted with an exocyclic olefinic double bond. Preferred cycloalkyl groups include C₃-C₁₂ cycloalkyl, C₃-C₆ cycloalkyl, C₃-C₈ cycloalkyl and C₄-C₇ cycloalkyl. Examples of cycloalkyl include, but are not limited to, cyclopropyl, cyclobutyl, cyclopentyl, cyclohexyl, cyclopentyl, cyclooctyl, 4-methylene-cyclohexyl, bicyclo[2.2.1]heptyl, bicyclo[3.1.0]hexyl, spiro[2.5]octyl, 3-methylenebicyclo[3.2.1]octyl, spiro[4.4]nonanyl, and the like.

The term “cycloalkenyl”, as used herein, refers to monocyclic or polycyclic carbocyclic ring, such as a bi- or tri-cyclic fused, bridged or spiro system having at least one carbon-carbon double bond. The ring carbon atoms are optionally oxo-substituted or optionally substituted with an exocyclic olefinic double bond. Preferred cycloalkenyl groups include C₃-C₁₂ cycloalkenyl, C₄-C₁₂-cycloalkenyl, C₃-C₈ cycloalkenyl, C₄-C₈ cycloalkenyl and C₅-C₇ cycloalkenyl groups. Examples of cycloalkenyl include, but are not limited to, cyclopropenyl, cyclobutenyl, cyclopentenyl, cyclohexenyl, cycloheptenyl, cyclooctenyl, bicyclo[2.2.1]hept-2-enyl, bicyclo[3.1.0]hex-2-enyl, spiro[2.5]oct-4-enyl, spiro[4.4]non-2-enyl, bicyclo[4.2.1]non-3-en-12-yl, and the like.

As used herein, the term “arylalkyl” means a functional group wherein an alkylene chain is attached to an aryl group, e.g., —(CH₂)ₙ-phenyl, where n is 1 to 12, preferably 1 to 6 and more preferably 1 or 2. The term “substituted arylalkyl” means an arylalkyl functional group in which the aryl group is substituted. Similarly, the term “heteroarylalkyl” means a functional group wherein an alkylene chain, is attached to a heteroaryl group, e.g., —(CH₂)ₙ-heteroaryl, where n is 1 to 12, preferably 1 to 6 and more preferably 1 or 2. The term “substituted heteroarylalkyl” means a heteroarylalkyl functional group in which the heteroaryl group is substituted.

As used herein, the term “alkoxy” refers to a radical in which an alkyl group having the designated number of carbon atoms is connected to the rest of the molecule via an oxygen atom. Alkoxy groups include C₁-C₁₂-alkoxy, C₁-C₈-alkoxy, C₁-C₆-alkoxy, C₁-C₄-alkoxy and C₁-C₃-alkoxy groups. Examples of alkoxy groups includes, but are not limited to, methoxy, ethoxy, n-propoxy, 2-propoxy (isopropoxy) and the higher homologs and isomers. Preferred alkoxy is C₁-C₃alkoxy.

An “aliphatic” group is a non-aromatic moiety comprised of any combination of carbon atoms, hydrogen atoms, halogen atoms, oxygen, nitrogen or other atoms, and optionally contains one or more units of unsaturation, e.g., double and/or triple bonds. Examples of aliphatic groups are functional groups, such as alkyl, alkenyl, alkynyl, O, OH, NH, NH₂, C(O), S(O)₂, C(O)O, C(O)NH, OC(O)O, OC(O)NH, OC(O)NH₂, S(O)₂NH, S(O)₂NH₂, NHC(O)NH₂, NHC(O)C(O)NH, NHS(O)₂NH, NHS(O)₂NH₂, C(O)NHS(O)₂, C(O)NHS(O)₂NH or C(O)NHS(O)₂NH₂, and the like, groups comprising one or more functional groups, non-aromatic hydrocarbons (optionally substituted), and groups wherein one or more carbons of a non-aromatic hydrocarbon (optionally substituted) is replaced by a functional group. Carbon atoms of an aliphatic group can be optionally oxo-substituted. An aliphatic group may be straight chained, branched, cyclic, or a combination thereof and preferably contains between about 1 and about 24 carbon atoms, more typically between about 1 and about 12 carbon atoms. In addition to aliphatic hydrocarbon groups, as used herein, aliphatic groups expressly include, for example, alkoxyalkyls, polyalkoxyalkyls, such as polyalkylene glycols, polyamines, and polyimines, for example. Aliphatic groups may be optionally substituted.

The terms “heterocyclic” and “heterocycloalkyl” can be used interchangeably and refer to a non-aromatic ring or a polycyclic ring system, such as a bi- or tri-cyclic fused, bridged or spiro system, where (i) each ring system contains at least one heteroatom independently selected from oxygen, sulfur and nitrogen, (ii) each ring system can be saturated or unsaturated (iii) the nitrogen and sulfur heteroatoms may optionally be oxidized, (iv) the nitrogen heteroatom may optionally be quaternized, (v) any of the above rings may be fused to an aromatic ring, and (vi) the remaining ring atoms are carbon atoms which may be optionally oxo-substituted or optionally substituted with exocyclic olefinic double bond. Representative heterocycloalkyl groups include, but are not limited to, 1,3-dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, quinoxalinyl, pyridazinonyl, 2-azabicyclo[2.2.1]-heptyl, 8-azabicyclo[3.2.1]octyl, 5-azaspiro[2.5]octyl, 2-oxa-7-azaspiro[4.4]nonanyl, 7-oxooxepan-4-yl, and tetrahydrofuryl. Such heterocyclic or heterocycloalkyl groups may be further substituted. A heterocycloalkyl or heterocyclic group can be C-attached or N-attached where possible.

It is understood that any alkyl, alkenyl, alkynyl, alicyclic, cycloalkyl, cycloalkenyl, aryl, heteroaryl, heterocyclic, aliphatic moiety or the like described herein can also be a divalent or multivalent group when used as a linkage to connect two or more groups or substituents, which can be at the same or different atom(s). One skilled in the art can readily determine the valence of any such group from the context in which it occurs.

The term “substituted” refers to substitution by independent replacement of one, two, or three or more of the hydrogen atoms with substituents including, but not limited to, —F, —Cl, —Br, —I, —OH, C₁-C₁₂-alkyl; C₂-C₁₂-alkenyl, C₂-C₁₂-alkynyl, —C₃-C₁₂-cycloalkyl, protected hydroxy, —NO₂, —N₃, —CN, —NH₂, protected amino, oxo, thioxo, —NH—C₁-C₁₂-alkyl, —NH—C₂-C₈-alkenyl, —NH—C₂-C₈-alkynyl, —NH—C₃-C₁₂-cycloalkyl, —NH-aryl, —NH-heteroaryl, —NH— heterocycloalkyl, -dialkylamino, -diarylamino, -diheteroarylamino, —O—C₁-C₁₂-alkyl, —O—C₂-C₈-alkenyl, —O—C₂-C₈-alkynyl, —O—C₃-C₁₂-cycloalkyl, —O-aryl, —O-heteroaryl, —O— heterocycloalkyl, —C(O)—C₁-C₁₂-alkyl, —C(O)—C₂-C₈-alkenyl, —C(O)—C₂-C₈-alkynyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₈-alkenyl, —CONH—C₂-C₈-alkynyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₈-alkenyl, —OCO₂—C₂-C₈-alkynyl, —OCO₂—C₃-C₁₂-cycloalkyl, —OCO₂-aryl, —OCO₂-heteroaryl, —OCO₂-heterocycloalkyl, —CO₂—C₁-C₁₂ alkyl, —CO₂—C₂-C₈ alkenyl, —CO₂—C₂-C₈ alkynyl, —CO₂—C₃-C₁₂-cycloalkyl, —CO₂-aryl, —CO₂-heteroaryl, —CO₂-heterocyloalkyl, —OCONH₂, —OCONH—C₁-C₁₂-alkyl, —OCONH—C₂-C₈-alkenyl, —OCONH—C₂-C₈-alkynyl, —OCONH—C₃-C₁₂-cycloalkyl, —OCONH-aryl, —OCONH-heteroaryl, —OCONH-heterocycloalkyl, —NHC(O)H, —NHC(O)—C₁-C₁₂-alkyl, —NHC(O)—C₂-C₈-alkenyl, —NHC(O)—C₂-C₈-alkynyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₈-alkenyl, —NHCO₂—C₂-C₈-alkynyl, —NHCO₂—C₃-C₁₂-cycloalkyl, —NHCO₂-aryl, —NHCO₂-heteroaryl, —NHCO₂— heterocycloalkyl, —NHC(O)NH₂, —NHC(O)NH—C₁-C₁₂-alkyl, —NHC(O)NH—C₂-C₈-alkenyl, —NHC(O)NH—C₂-C₈-alkynyl, —NHC(O)NH—C₃-C₁₂-cycloalkyl, —NHC(O)NH-aryl, —NHC(O)NH-heteroaryl, —NHC(O)NH-heterocycloalkyl, —NHC(S)NH₂, —NHC(S)NH—C₁-C₁₂-alkyl, —NHC(S)NH—C₂-C₈-alkenyl, —NHC(S)NH—C₂-C₈-alkynyl, —NHC(S)NH—C₃-C₁₂-cycloalkyl, —NHC(S)NH-aryl, —NHC(S)NH-heteroaryl, —NHC(S)NH— heterocycloalkyl, —NHC(NH)NH₂, —NHC(NH)NH—C₁-C₁₂-alkyl, —NHC(NH)NH—C₂-C₈-alkenyl, —NHC(NH)NH—C₂-C₈-alkynyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₈-alkenyl, —NHC(NH)—C₂-C₈-alkynyl, —NHC(NH)—C₃-C₁₂-cycloalkyl, —NHC(NH)-aryl, —NHC(NH)-heteroaryl, —NHC(NH)-heterocycloalkyl, —C(NH)NH₂, —C(NH)NH—C₁-C₁₂-alkyl, —C(NH)NH—C₂-C₈-alkenyl, —C(NH)NH—C₂-C₈-alkynyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₈-alkenyl, —S(O)—C₂-C₈-alkynyl, —S(O)—C₃-C₁₂-cycloalkyl, —S(O)-aryl, —S(O)-heteroaryl, —S(O)-heterocycloalkyl, —SO₂H₂, —SO₂NH—C₁-C₁₂-alkyl, —SO₂NH—C₂-C₈-alkenyl, —SO₂NH—C₂-C₈-alkynyl, —SO₂—C₁-C₁₂-alkyl, —SO₂—C₂-C₈-alkenyl, —SO₂—C₂-C₈-alkynyl, —SO₂—C₃-C₁₂-cycloalkyl, —SO₂-aryl, —SO₂-heteroaryl, —SO₂-heterocycloalkyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₈-alkenyl, —NHSO₂—C₂-C₈-alkynyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₈-alkenyl, —S—C₂-C₈-alkynyl, —S—C₃-C₁₂-cycloalkyl, —S-aryl, —S-heteroaryl, —S-heterocycloalkyl, or methylthio-methyl. In certain embodiments, the substituents are independently selected from halo, preferably C₁ and F; C₁-C₄-alkyl, preferably methyl and ethyl; halo-C₁-C₄-alkyl, such as fluoromethyl, difluoromethyl, and trifluoromethyl; C₂-C₄-alkenyl; halo-C₂-C₄-alkenyl; C₃-C₆-cycloalkyl, such as cyclopropyl; C₁-C₄-alkoxy, such as methoxy and ethoxy; halo-C₁-C₄-alkoxy, such as fluoromethoxy, difluoromethoxy, and trifluoromethoxy; —CN; —OH; NH₂; C₁-C₄-alkylamino; di(C₁-C₄-alkyl)amino; and NO₂. It is understood that an aryl, heteroaryl, alkyl, alkenyl, alkynyl, cycloalkyl, or heterocycloalkyl in a substituent can be further substituted. In certain embodiments, a substituent in a substituted moiety is additionally optionally substituted with one or more groups, each group being independently selected from C₁-C₄-alkyl; —CF₃, —OCH₃, —OCF₃, —F, —Cl, —Br, —I, —OH, —NO₂, —CN, and —NH₂. Preferably, a substituted alkyl group is substituted with one or more halogen atoms, more preferably one or more fluorine or chlorine atoms.

The term “halo” or halogen” alone or as part of another substituent, as used herein, refers to a fluorine, chlorine, bromine, or iodine atom.

The term “optionally substituted”, as used herein, means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

The term “hydrogen” includes hydrogen and deuterium. In addition, the recitation of an element includes all isotopes of that element so long as the resulting compound is pharmaceutically acceptable. In certain embodiments, the isotopes of an element are present at a particular position according to their natural abundance. In other embodiments, one or more isotopes of an element at a particular position are enriched beyond their natural abundance.

The term “hydroxy activating group,” as used herein, refers to a labile chemical moiety which is known in the art to activate a hydroxyl group so that it will depart during synthetic procedures such as in a substitution or an elimination reaction. Examples of hydroxyl activating groups include, but not limited to, mesylate, tosylate, triflate, p-nitrobenzoate, phosphonate and the like.

The term “activated hydroxyl,” as used herein, refers to a hydroxy group activated with a hydroxyl activating group, as defined above, including, but not limited to mesylate, tosylate, triflate, p-nitrobenzoate, phosphonate groups.

The term “hydroxy protecting group,” as used herein, refers to a labile chemical moiety which is known in the art to protect a hydroxyl group against undesired reactions during synthetic procedures. After said synthetic procedure(s) the hydroxy protecting group as described herein may be selectively removed. Hydroxy protecting groups as known in the art are described generally in P. G. M. Wuts, Greene's Protective Groups in Organic Synthesis, 5th edition, John Wiley & Sons, Hoboken, NJ (2014). Examples of hydroxyl protecting groups include, but are not limited to, benzyloxycarbonyl, 4-methoxybenzyloxycarbonyl, tert-butoxy-carbonyl, isopropoxycarbonyl, diphenylmethoxycarbonyl, 2,2,2-trichloroethoxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, allyl, benzyl, triphenyl-methyl (trityl), methoxymethyl, methylthiomethyl, benzyloxymethyl, 2-(trimethylsilyl)-ethoxymethyl, methanesulfonyl, trimethylsilyl, triisopropylsilyl, and the like.

The term “protected hydroxy,” as used herein, refers to a hydroxy group protected with a hydroxy protecting group, as defined above, including but not limited to, benzoyl, acetyl, trimethylsilyl, triethylsilyl, methoxymethyl groups, for example.

The term “hydroxy prodrug group,” as used herein, refers to a promoiety group which is known in the art to change the physicochemical, and hence the biological properties of a parent drug in a transient manner by covering or masking the hydroxy group. After said synthetic procedure(s), the hydroxy prodrug group as described herein must be capable of reverting back to hydroxy group in vivo. Hydroxy prodrug groups as known in the art are described generally in Kenneth B. Sloan, Prodrugs, Topical and Ocular Drug Delivery, (Drugs and the Pharmaceutical Sciences; Volume 53), Marcel Dekker, Inc., New York (1992).

The term “amino protecting group,” as used herein, refers to a labile chemical moiety which is known in the art to protect an amino group against undesired reactions during synthetic procedures. After said synthetic procedure(s) the amino protecting group as described herein may be selectively removed. Amino protecting groups as known in the art are described generally in P. G. M. Wuts, Greene's Protective Groups in Organic Synthesis, 5th edition, John Wiley & Sons, Hoboken, NJ (2014). Examples of amino protecting groups include, but are not limited to, methoxycarbonyl, t-butoxycarbonyl, 12-fluorenyl-methoxycarbonyl, benzyloxycarbonyl, and the like.

The term “protected amino,” as used herein, refers to an amino group protected with an amino protecting group as defined above.

The term “leaving group” means a functional group or atom which can be displaced by another functional group or atom in a substitution reaction, such as a nucleophilic substitution reaction. By way of example, representative leaving groups include chloro, bromo and iodo groups; sulfonic ester groups, such as mesylate, tosylate, brosylate, nosylate and the like; and acyloxy groups, such as acetoxy, trifluoroacetoxy and the like.

The term “aprotic solvent,” as used herein, refers to a solvent that is relatively inert to proton activity, i.e., not acting as a proton-donor. Examples include, but are not limited to, hydrocarbons, such as hexane and toluene, for example, halogenated hydrocarbons, such as, for example, methylene chloride, ethylene chloride, chloroform, and the like, heterocyclic compounds, such as, for example, tetrahydrofuran and N-methylpyrrolidinone, and ethers such as diethyl ether, bis-methoxymethyl ether. Such compounds are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of aprotic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

The term “protic solvent,” as used herein, refers to a solvent that tends to provide protons, such as alcohol, for example, methanol, ethanol, propanol, isopropanol, butanol, t-butanol, and the like. Such solvents are well known to those skilled in the art, and it will be obvious to those skilled in the art that individual solvents or mixtures thereof may be preferred for specific compounds and reaction conditions, depending upon such factors as the solubility of reagents, reactivity of reagents and preferred temperature ranges, for example. Further discussions of protogenic solvents may be found in organic chemistry textbooks or in specialized monographs, for example: Organic Solvents Physical Properties and Methods of Purification, 4th ed., edited by John A. Riddick et al., Vol. II, in the Techniques of Chemistry Series, John Wiley & Sons, NY, 1986.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

The synthesized compounds can be separated from a reaction mixture and further purified by a method such as column chromatography, high pressure liquid chromatography, or recrystallization. As can be appreciated by the skilled artisan, further methods of synthesizing the compounds of the Formula herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2^{nd} Ed. Wiley-VCH (1999); P. G. M. Wuts, Greene's Protective Groups in Organic Synthesis, 5th edition, John Wiley & Sons, Hoboken, NJ (2014); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The term “subject,” as used herein, refers to an animal. Preferably, the animal is a mammal. More preferably, the mammal is a human. A subject also refers to, for example, a dog, cat, horse, cow, pig, guinea pig, fish, bird and the like.

The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers or cis- and trans-isomers. Likewise, all tautomeric forms are also intended to be included. Tautomers may be in cyclic or acyclic. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

Certain compounds of the present invention may also exist in different stable conformational forms which may be separable. Torsional asymmetry due to restricted rotation about an asymmetric single bond, for example because of steric hindrance or ring strain, may permit separation of different conformers. The present invention includes each conformational isomer of these compounds and mixtures thereof.

As used herein, the term “pharmaceutically acceptable salt,” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 2-19 (1977). The salts can be prepared in situ during the final isolation and purification of the compounds of the invention, or separately by reacting the free base function with a suitable organic acid. Examples of pharmaceutically acceptable salts include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentane-propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intra-arterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable formulations.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is mixed under sterile conditions with a pharmaceutically acceptable carrier and any needed preservatives or buffers as may be required. Ophthalmic formulations, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to Van Devanter et al., U.S. Pat. No. 5,508,269 to Smith et al., and WO 98/43650 by Montgomery, all of which are incorporated herein by reference).

### Pharmacology and Administration

Protein tyrosine kinases (PTK) play a central role in the regulation of a wide variety of cellular processes and maintaining control over cellular function. Protein kinases catalyze and regulate the process of phosphorylation, whereby the kinases covalently attach phosphate groups to proteins or lipid targets in response to a variety of extracellular signals. Examples of such stimuli include hormones, neurotransmitters, growth and differentiation factors, cell cycle events, environmental stresses and nutritional stresses. An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events. These diseases include, but are not limited to, autoimmune diseases, inflammatory diseases, bone diseases, metabolic diseases, neurological and neurodegenerative diseases, cancer, cardiovascular diseases, respiratory diseases, allergies and asthma, Alzheimer's disease, and hormone-related diseases.

Examples of protein-tyrosine kinases include, but are not limited to,
(a) tyrosine kinases such as Irk, IGFR-1, Zap-70, Bmx, Btk, CHK (Csk homologous kinase), CSK (C-terminal Src Kinase), ltk-1, Src (c-Src, Lyn, Fyn, Lek, Syk, Hck, Yes, Blk, Fgr and Frk), Tee, Txk/Rlk, Abl, EGFR (EGFR-1/ErbB-1, ErbB-2/NEU/IER-2, ErbB-3 and ErbB-4), FAK, FGF1 R (also FGFR1 or FGR-1), FGF2R (also FGR-2), MET (also Met-1 or c-MET), PDGFR (α and β), Tie-1, Tie-2 (also Tek-1 or Tek), VEGFR1 (also FLT-1), VEGFR2 (also KDR), FLT-3, FLT-4, c-KIT, JAK1, JAK2, JAK3, TYK2, LOK, ET, TRKA, PYK2, ALK (Anaplastic Lymphoma Kinase), EPHA (1-8), EPHB (1-6), RON, Fes, Fer or EPHB4 (also EPHB4-1), and
(b) and serine/threonine kinases such as Aurora, c-RAF, SGK, MAP kinases (e.g., MKK4, MKK6, etc.), SAPK2a, SAPK20, Ark, ATM (1-3), CamK (1-IV), CamKK, Chkl and 2 (Checkpoint kinases), CKI, CK2, Erk, IKK-I (also IKK-α or CHUK), IKK-2 (also IKK-β), Ilk, Jnk (1-3), LimK (1 and 2), MLK3Raf (A, B, and C), CDK (1-10), PKC (including all PKC subtypes), Plk (1-3), NIK, Pak (1-3), PDK1, PKR, RhoK, RIP, RIP-2, GSK3 (α and β), KA, P38, Erk (1-3), PKB (including all PKB subtypes) (also AKT-1, AKT-2, AKT-3 or AKT3-1), IRAK1, FRK, SGK, TAK1 and Tpl-2 (also COT).

Phosphorylation modulates or regulates a variety of cellular processes such as proliferation, growth, differentiation, metabolism, apoptosis, motility, transcription, translation and other signaling processes. Aberrant or excessive PTK activity has been observed in many disease states including, but not limited to, benign and malignant proliferative disorders, diseases resulting from inappropriate activation of the immune system and diseases resulting from inappropriate activation of the nervous systems. Specific diseases and disease conditions include, but are not limited to, autoimmune disorders, allograft rejection, graft vs. host disease, diabetic retinopathy, choroidal neovascularization due to age-related macular degeneration, psoriasis, arthritis, osteoarthritis, rheumatoid arthritis, synovial pannus invasion in arthritis, multiple sclerosis, myasthenia gravis, diabetes mellitus, diabetic angiopathy, retinopathy of prematurity, infantile hemangiomas, non-small cell lung, bladder and head and neck cancers, prostate cancer, breast cancer, ovarian cancer, gastric and pancreatic cancer, psoriasis, fibrosis, rheumatoid arthritis, atherosclerosis, restenosis, autoimmune disease, allergy, respiratory diseases, asthma, transplantation rejection, inflammation, thrombosis, retinal vessel proliferation, inflammatory bowel disease, Crohn's disease, ulcerative colitis, bone diseases, transplant or bone marrow transplant rejection, lupus, chronic pancreatitis, cachexia, septic shock, fibroproliferative and differentiative skin diseases or disorders, central nervous system diseases, neurodegenerative diseases, disorders or conditions related to nerve damage and axon degeneration subsequent to a brain or spinal cord injury, acute or chronic cancer, ocular diseases, viral infections, heart disease, lung or pulmonary diseases or kidney or renal diseases and bronchitis.

Tyrosine kinases can be broadly classified as receptor-type (having extracellular, transmembrane and intracellular domains) or the non-receptor type (being wholly intracellular) protein tyrosine kinases. Tyrosine kinases transfer the terminal phosphate of ATP to tyrosine residues of proteins thereby activating or inactivating signal transduction pathways. Inappropriate or uncontrolled activation of many of these kinase (aberrant protein tyrosine kinase activity), for example by over-expression or mutation, results in uncontrolled cell growth. Many of the protein tyrosine kinases, whether a receptor or non-receptor tyrosine kinase have been found to be involved in cellular signaling pathways involved in numerous pathogenic conditions, including, but not limited to, immunomodulation, inflammation, or proliferative disorders such as cancer.

c-Kit

Mast cells are tissue elements derived from a particular subset of hematopoietic stem cells that express CD34, c-kit and CD13 antigens. Mast cells are characterized by their heterogeneity, not only regarding tissue location and structure but also at the functional and histochemical levels. Immature mast cell progenitors circulate in the bloodstream and differentiate into various tissues. These differentiation and proliferation processes are under the influence of cytokines, one of importance being Stem Cell Factor (SCF), also termed c-Kit ligand, Steel factor or Mast Cell Growth Factor. The Stem Cell Factor receptor is encoded by the protooncogene, c-kit, which is expressed in hematopoietic progenitor cells, mast cells, germ cells, interstitial cells of Cajal (ICC), and some human tumors, and is also expressed by non hematopoietic cells.

Stem cell factor (SCF), also known as c-kit ligand, is the primary regulating factor for human mast cell growth and function. The SCF receptor, c-kit receptor, is a Type III transmembrane receptor protein tyrosine kinase which initiates cell growth and proliferation signal transduction cascades in response to SCF binding. Ligation of c-kit receptor by SCF induces its dimerization followed by its transphorylation, leading to the recruitment and activation of various intracytoplasmic substrates. These activated substrates induce multiple intracellular signaling pathways responsible for cell proliferation and activation. These proteins are known to be involved in many cellular mechanisms, which in case of disruption, lead to disorders such as abnormal cell proliferation and migration, as well as inflammation.

The relationship between mast cells, SCF and c-kit receptor is discussed in the following references: Huang, E. et al., “The hematopoietic growth factor Kt is encoded by the SI locus and is the ligand of the c-kit receptor, the gene product of the W locus”, Cell, 63, 225-233, 1990; Zseb0, K. M. et al., “Stem cell factor is encoded at the Sl locus of the mouse and is the ligand for the c-kit tyrosine kinase receptor”, Gell, 63, 213-224, 1990; Zhang, S. et al.,” Cytokine production by cell cultures from bronchial subepithelial myofibroblasts”, J. Path0l., 180, 95-10, 1996; Zhang, S. et al., “Human mast cells express stem cell factor”, J. Pathol., 186, 59-66, 1998; Kassel, O. et al., “Up and down regulation by glucocorticoids of the constitutive expression of the mast cell growth factor stem cell factor by human tung fibroblasts in culture”, Mol. Fharmacol., 54, 1073-1079, 1998; Kasset, O. et al., “Human bronchial smooth muscle cells in culture produce Stem Cell Factor”, Eur. Bespir. J., 13, 951-954, 1999; Kassel, O. et at., “The Stem Cel Factor, Stem cell factor, its Properties and Potential Role in the Airways”, Pulmonary Pharmacology & Therapeutics”, 14, 227-288, 2001; de Paulis, A. et al, “Stem cell factor is localized in, released trom, and cleaved by human mast cells”, J. Immunol, 163, 2799-2808, 1999; Mot, C. D. et al., “Structure of a c-kit product complex reveals the basis for kinase transactivation”, J. Biol. Chem., 278, 31461-31464, 2003; temura, A. et al., “The c-kit ligand, stem cell actor, promotes mast cell survival by suppressing apoptosis”, Am. J. Path0l., 144,321-328, 1994; Nilsson, G. et al., “Stem cell factor is a chemotactic factor for human mast cells”, A Ammunol., 153, 3717-3723, 1994; Meininger, C. J. et al., “The c-kit receptor ligand functions as a mast cell chemoattractant”, Blood, 79, 958-963, 1992, and Kinashi, T. et al., “Steel factor and c-kit regulate cell-matrix adhesion”, Blood, 83, 1033-1038, 1994.

The following references discuss the c-kit signaling pathway and its relationship with various downstream pathways and the relationship with diseases associated with mast cells: Thommes, K. et al., “Identification of Tyr-703 and Tyr-936 as the primary association sites for Gr2 and Grb?in the c-Kit/stem cel factor receptor”, Biochem., d. 341,211-216, 1999; ishizuka, T. et al., Stem cell factor augments Fc epsilon Rlmediated TNF-alpha production and stimulates MAP kinases via a different pathway in MC/9 mast cells”, J. Immunol., 161, 3624-3630, 1998; Timokhina, I. et al., “Kit signaling through Pi 3-kinase and Sr kinase pathways: an essential role for Fact and INK activation in mast cell proliferation”, EMBO 1, 17, 6250-6262, 1998; Tang, B. et al., “Tec kinase associates with c-kit and is tyrosine phosphorylated and activated following stem cell factor binding”, Mot. Cell. Biol., 14, 8432-8437, 1994, and Ueda, S. et al., “Critical roles of c-Kit tyrosine residues 567 and 719 in stem cell factor-induced chemotaxis: contribution of src tamily kinase and PI3-kinase on calcium mobilization and cell migration”, Blood, 99, 3342-3349, 2002.

Mast cells are the primary effector cells in allergic inflammation. Mast cells are also involved in other pathogenic processes such as acute inflammation and fibrosis. Mast cells present in tissues of patients are implicated in or contribute to the genesis of diseases such as autoimmune diseases (multiple sclerosis, rheumatoid arthritis, inflammatory bowel diseases (IBD)), allergic diseases (allergic rhinitis, allergic sinusitis, anaphylactic syndrome, urticaria, angioedema, atopic dermatitis, allergic contact dermatitis, erythema nodosum, erythema multiforme, cutaneous necrotizing venulitis and insect bite skin inflammation and bronchial asthma), tumor angiogenesis, germ cell tumors, mast cell tumors, gastrointestinal stromal tumors, small-cell lung cancer, melanoma, breast cancer, acute myelogenous leukemia, glioblastoma, neuroblastoma and mastocytosis, inflammatory diseases, diabetes, type I diabetes, type II diabetes, irritable bowel syndrome (IBS), CNS disorders and interstitial cystitis. In these diseases, mast cells participate in the destruction of tissues by releasing a cocktail of different proteases and mediators categorized into three groups: preformed granule-associated mediators (histamine, proteoglycans, and neutral proteases), lipid-derived mediators (prostaglandins, thromboxanes and leucotrienes), and various cytokines (IL-1, IL-2, IL-3, IL-4, IL-5, IL-6, IL-8, TNF-α, GM-CSF, MIP-Lα, MIP-Iβ, MIP-2 and IFN-x). The liberation by activated mast cells of mediators (TNF-α, histamine, leukotrienes, prostaglandins etc.) as well as proteases may i) induce inflammation and vasodilatation and ii) participate in the tissue destruction process.

In addition, mast cell activation induces diverse effector responses, such as secretion of allergic mediators, proteases, chemokines such as MCP-1 and RANTES, leukotrienes, prostaglandins and neurotrophins; and induction of cytokine gene transcription (IL-4, IL-5, IL-6, IL-13, TNF-α and GM-CSF). These mediators contribute to creating the asthmatic phenotype by their effects on endothelial cells, smooth muscle cells and fibroblasts and on extracellular matrix, and by recruiting other inflammatory cells.

Asthma is characterized by airflow obstruction, bronchial hyper responsiveness and airway inflammation. Airway inflammation is the major factor in the development and perpetuation of asthma. In allergic asthma, allergens are thought to initiate the inflammatory process by inducing a T-lymphocyte mediated response (TH2) that results in the production of allergen-specific lgE. lgE binds to its high-affinity receptor FcεRI on pulmonary mast cells, triggering a type I (lgE-mediated) immediate allergic response. Thus, mast cells play a role in asthma.

The activation of mast cells by different stimuli such as stress, trauma, infection and neurotransmitters, also participate in the exacerbation of the chemical imbalance causing CNS disorders. More specifically, mast cell degranulation is stimulated by common neurotransmitters such as neurotensin, somatostatin, substance P and acetylcholine, by growth or survival factors, notably such as NGF. Mast cells involved in the response to such stimulus can be brain mast cells but also other mast cells releasing the content of their granules in the blood stream that ultimately reach sensory, motor or brain neurons. Following mast cells activation, released granules liberate various factors capable of modulating and altering neurotransmission and neurons survival. Among such factors, serotonin is important since an increase of the level of free serotonin has been observed in depressed patients. Alternatively, the sudden burst of serotonin may be followed by a period of serotonin shortage, leading to pain and migraine. As a consequence, it is believed that mast cells exacerbate in autocrine or paracrine manner the deregulation of neurotransmission. For example, anxiety or stress-induced release of neurotransmitters such as serotonin activates mast cells, which in turn release the content of their granules, further contributing to the chemical imbalance in the brain leading to CNS disorders.

Other mediators released by mast cells can be categorized into vasoactive, nociceptive, proinflammatory and other neurotransmitters. Taken together, these factors are able to induce disturbance in the activity of neurons, whether they are sensory, motor, or CNS neurons. In addition, patients afflicted with mastocytosis are more inclined to develop CNS disorders than the normal population. This can be explained by the presence of activating mutations in the c-kit receptor, which induce degranulation of mast cells and a burst of factors contributing to chemical imbalance and neurotransmission alteration.

The activation of mast cells by different drugs, including, but not limited to, salicylic derivatives, morphine derivatives, opioids, heroin, amphetamines, alcohol, nicotine, analgesics, anesthetics, and anxyolitics results in the degranulation of mast cells, which participate in the exacerbation of the chemical imbalance responsible for drug habituation and withdrawal syndrome. Following mast cells activation, released granules liberate various factors capable of modulating and altering neurotransmission. Among such factors is morphine which is bound or stored in mast cells granules. Tobacco smoke also induces the release of mediators from canine mast cells and modulates prostaglandin production leading to asthma. In addition, patients afflicted with mastocytosis are more inclined to develop substance use disorders than the normal population. This can be explained by the presence of activating mutations in the c-kit receptor, which induce degranulation of mast cells and a burst of factors contributing to chemical imbalance and neurotransmission alteration.

Mast cells have also been identified to be involved in or to contribute to drug dependence and withdrawal symptoms.

The relationship between mast cells, SCF and c-kit kinase in various diseases is discussed in the following fereterces: Oliveira et al., “Stem Cell Factor: A Hemopoietic Cytokine with Important Targets in Asthma”, Current Drug Targets, 2: 313-318, 2003; Puxeddu et al., “Mast cells in allergy and beyond”, The International Journal of Biochemistry & Cell Biology, 35: 1601-1607, 2003; Rottem et al., “Mast cells and autoimmunity”, Autoimmunity Reviews, 4: 21-27, 2005; Woolley, D. E. et al., “The mast cell in inflammatory arthritis”, N. Engl. J. Med., 348:1709-1711, 2003; Benoist, C. et al., “Mast cells in autoimmune disease”, Nature, 420:875-878, 2002; Nigrovic, P. A. et al., “Mast cells in inflammatory arthritis”, Arthritis es. Ther., 7:1-11, 2005; Wang, H. W. et al., “Mast cell accumulation and cytokine expression in the tight skin mouse model of scleroderma”, Exp. Dernatol., 14, 295-302, 2005; Olsson, N. et al., “Demonstration of mast cell chemotactic activity in bronchoalveolar lavage fluid collected from asthmatic patients before and during pollen season”, J. Allergy Clin. immunol., 105, 455-461, 2000; Ma, Y. et al., “Idolinone derivatives inhibit constitutively activated KTT mutants and kilt neoplastic mast cells”, J. Invest. Dermatol., 114, 392-394, 2000; Kobayashi, Y. et al., “Mst Cells as a Target ot Rheumatoid Arthritis Treatment”, Jpn. J. Pharmacol., 7-11, 2002, and Ai-Muhsen, S. Z. et al., The expression of stem cell factor and c-kit receptor in human asthmatic airways”, C#in. Exp. Allergy, 34, 911-916, 2004.

In addition, the treatment of asthma and arthritis with administration of a c-kit inhibitor is presented in the following references: Takeuchi et al., “ST1571 inhibits growth and adhesion of human mast cells in culture”, Journal of Leukocyte Biology, 74: 1026-1034, 2003; Berlin et al., “Treatment of Cockroach Allergen Asthma Model with Imatinib Attenuates Airway Responses”, American dourat of Respiratory and Critical care Medicine, 171: 35-39, 2005; Ekland et al., “Treatment of rheumatoid arthritis with imatinib mesylate: clinical improvement in three refractory cases”, Annals of Medicine, 35: 362-367, 2003; Miyachi et al., “Efficacy of imatinib mesylate (ST1571) treatment for a patient with rheumatoid arthritis developing chronic myelogenous leukemia”, Clinical Rheumatology, 22: 329-332, 2003; Juurikivi et al., “Inhibition of c-kit tyrosine kinase by imatinib mesylate induces apoptosis in mast cells in rheumatoid synovial: a potential approach to the treatment of arthritis”, Ann. Rheum. Dis., 64: 1126-1131, 2005; Wolf, A. A M., et al., “The kinase inhibitor iratinib mnesylate inhibits TN˜-alpha production in vitro and prevents TNF-dependent acute hepatic inflammation”, Proo. Natl. Acad. Sci. U.S.A 102:13622-13627, 2005; Leath et al., “Novel and emerging therapies for asthma”, Drug Discovery Today, 10(23/24): 1647-1655, 2005; Berlin et al., “Inhibition of SCF attenuates peribronchial remodeling in chronic cockroach allergen-induced asthma”, Laboratory Investigations, 86: 557-565, 2006; Paniagua et al., “Selective tyrosine kinase inhibition by imatinib mesylate for the treatment of autoimmune arthritis”, The Journal of Clinical Investigation, 116(10): 2633-2642, 2006; Wenzel et al., “Update in Asthma”, American Journal of Respiratory and Critical care Medicine, 173: 698-706, 2006; Chaudhary et al., “Pharmacological Differentiation of Inflammation and Fibrosis in the Bleomycin Model”, American Journal of Respiratory and Critical care Medicine, 173: 769-776, 2006, and Reber et al., “Review: Stem cell factor and its receptor c-Kit as targets for inflammatory diseases”, European Journal of Pharmacology, 533: 327-340, 2006.

The activity of the c-kit receptor is regulated in normal cells, and the normal functional activity of this c-kit gene product is important for the maintenance of normal hematopoeisis, melanogenesis, genetogensis, and growth and differentiation of mast cells. Inhibition of c-kit kinase activity reduces the growth and differentiation of mast cells and thereby mediates the diseases and/or conditions associated with mast cells, such as autoimmune diseases, multiple sclerosis, rheumatoid arthritis, inflammatory bowel diseases (IBD), respiratory diseases, allergic diseases, allergic rhinitis, allergic sinusitis, anaphylactic syndrome, urticaria, angioedema, atopic dermatitis, allergic contact dermatitis, erythema nodosum, erythema multiforme, cutaneous necrotizing venulitis and insect bite skin inflammation, bronchial asthma, tumor angiogenesis, germ cell tumors, mast cell tumors, gastrointestinal stromal tumors, small-cell lung cancer, melanoma, breast cancer, acute myelogenous leukemia, glioblastoma, neuroblastoma and mastocytosis, inflammatory diseases, diabetes, type I diabetes, type II diabetes, irritable bowel syndrome (IBS), CNS disorders and interstitial cystitis.

In addition to its importance in normal cellular physiologic activities, c-kit kinase plays a role in the biological aspects of certain human cancers, and unregulated c-kit kinase activity is implicated in the pathogenesis of human cancers, and in certain tumors types. Proliferation of tumor cell growth mediated by c-kit can occur by a specific mutation of the c-kit polypeptide that results in ligand independent activation or by autocrine stimulation of the receptor. In the former case, mutations that cause constitutive activation of c-kit kinase activity in the absence of SCF binding are implicated in malignant human cancers, including germ cell tumors, mast cell tumors, gastrointestinal stromal tumors, small-cell lung cancer, melanoma, breast cancer, acute myelogenous leukemia, glioblastoma, neuroblastoma and mastocytosis.

A proliferation assay for the evaluation of the efficacy of c-kit inhibitors and PDGF inhibitors is given in Kuriu et al., “Proliteration of human myeloid leukemia cell line associated with the tyrosine-phosphorylation and activation of the proto-oncogene c-kit product”, Blood, 78(11): 2834-2840, 1991; Heinrich et al., “Inhibition of c-kit receptor tyrosine kinase activity by ST1571, a selective tyrosine kinase inhibitor”, Blood, 96(3): 925-932, 2000; Buchdunger et al., “Abl Protein-Tyrosine Kinase Inhibitor ST1571 Inhibits In Vitro Signal Transduction Mediated by c-Kit and Platelet-Derived Growth Factor Receptors”, The Journal of Pharmacology and Experimental Therapeutics, 295(1): 139-145, 2000; and Smolich et al., The antiangiogenic protein kinase inhibitors SU5416 and SU6668 inhibit the SCF receptor (c-kit) in a human myeloid leukemia cell line and in acute myeloid leukemia blasts”, Blood, 97(5): 1413-1421, 2001. This assay uses MO7E cells, which are a human promegakaryocytic leukemia cell line that depend on SCF for proliferation. These references in combination with Berlin et al., Ekland et al., and Miyachi et al., (cited above) show that that a c-kit kinase inhibitor screened via this proliferation assay was later found to treat rheumatoid arthritis and asthma.

In addition, a compound that was initially evaluated for its efficacy as a c-kit inhibitor using a proliferation assay based on Ba/F3 cells and Ba/F3-derived cells (see WO 2004/01903) was later found to be effective in the treatment of mast cell tumours and asthma (see Bellamy F. et al., “Pharmacokinetics of masitinib in cats”, Vet. Res. Commun., June 16 (epub) 2009; Hahn K. A. et al., “Mastinib is safe and effective for treatment of canine mact cell tumours’, J. Vet. intern. Med., 22, 1301-1309, 2008 and Humbert M. et al., “Mastinib, a &-kit/PDGF receptor tyrosine kinase inhibitor, improves disease control in severe corticosteroid-dependent asthmatics”, 64, 1194-1201, 2009. c-kit receptor has a substantial homology to the PDGF receptor and to the CSF-1 receptor (c-Fms).

### Platelet-Derived Growth Factor (PDGF) Receptor Family

PDGF (Platelet-derived Growth Factor) is a commonly occurring growth factor which plays an important role both in normal growth and in pathological cell proliferation. By way of example, such as that observed in carcinogenesis and in diseases of the smooth muscle cells of blood vessels, for example in atherosclerosis and thrombosis. The PDGF growth factor family consists of PDGF-A, PDGF-B, PDGF-C and PDGF-D, which form either homo- or heterodimers (AA, AB, BB, CC, DD) that bind to the protein tyrosine kinase receptors PDGFR-α and PDGFR-β. Dimerization of the growth factors is a prerequisite for activation of the kinase, as the monomeric forms are inactive. The two receptor isoforms dimerize upon binding resulting in three possible receptor combinations, PDGFR-αα, PDGFR-ββ and PDGFR-αβ. Growth factor AA binds only to aa, growth factor BB can bind with -αα, -ββ and -αβ, growth factors CC and AB specifically interact with -aa and -a˜, and growth factor DD binds to -33. The PDGF-receptor plays an important role in the maintenance, growth and development of hematopoietic and non-hematopoietic cells.

Key downstream mediators of PDGFR signaling are Ras/mitogen-activated protein kinase (MAPK), P1-3 kinase and phospholipase-y (PLC) pathways. MAPK family members regulate various biological functions by phosphorylation of target molecules (transcription factors and other kinases) and thus contribute to regulation of cellular processes such as proliferation, differentiation, apoptosis and immunoresponses. P1-3 kinase activation generated PIP3 which functions as a second messenger to activate downstream tyrosine kinases Btk and ltk, the Ser/Thr kinases PDK1 and Akt (PKB). Akt activation is involved in survival, proliferation, and cell growth. After activation PLC hydolyses its substrate, Ptdlns(4,5)P2, and forms two secondary messengers, diacylglycerol and lns(1,4,5)P3 which stimulates intracellular processes such as proliferation, angiogenesis and cell motility.

PDGFR is expressed on early stem cells, mast cells, myeloid cells, mesenchymal cells and smooth muscle cells. Only PDGFR-0 is implicated in myeloid leukemias usually as a translocation partner with Tel, Huntingtin interacting protein (HIP1) or Rabaptin5. Activation mutations in PDGFR-α kinase domain are associated with gastrointestinal stromal tumors (GIST).

Certain embodiments of compounds of Formula (I) and Formula (II) provided herein inhibit PDGF receptor (PDGFRα and PDGFRβ) activity and c-kit kinase activity, and are useful for the treatment of diseases, which respond to an inhibition of the PDGF receptor kinase. Therefore, certain compounds of Formula (I) provided herein are useful for the treatment of tumor diseases, such as gliomas, sarcomas, prostate tumors, small cell lung cancer and tumors of the colon, breast, and ovary. In addition, certain embodiments of compounds of Formula (I) provided herein are useful to treat disorders, such as thrombosis, psoriasis, scleroderma, fibrosis, asthma, metabolic diseases and hypereosinophilia. Compounds of Formula (I) and Formula (II) provided herein are also effective against diseases associated with vascular smooth-muscle cell migration and proliferation, such as restenosis and atherosclerosis.

Patients with obliterative bronchiolitis (OB), a chronic rejection of allogenic lung transplants, often show an elevated PDGF concentration in bronchoalveolar lavage fluids. In certain embodiments, compounds of Formula (I) provided herein exhibit useful effects in the treatment of disorders arising as a result of transplantation, for example, allogenic ransplantation, especially tissue rejection, such as obliterative bronchiolitis (OB).

In certain embodiments, compounds of Formula (I) provided herein are useful for the protection of stem cells, for example to combat the hemotoxic effect of chemotherapeutic agents, such as 5-fluorouracil.

The compounds of Formula (I) provided herein, and the pharmaceutically acceptable salts, pharmaceutically acceptable solvates (e.g. hydrates), the N-oxide derivatives, protected derivatives, individual isomers and mixture of isomers thereof, are inhibitors of c-kit kinase activity or are inhibitors of c-kit kinase activity and PDGFRR (α and β) kinase activity. In certain embodiments, the compounds of Formula (I) provided herein, and the pharmaceutically acceptable salts, pharmaceutically acceptable solvates (e.g. hydrates), the N-oxide derivatives, protected derivatives, individual isomers and mixture of isomers thereof, are inhibitors of c-kit kinase activity and PDGFRR (a and 3) kinase activity. In other embodiments, the compounds of Formula (I) provided herein, and the pharmaceutically acceptable salts, pharmaceutically acceptable solvates (e.g. hydrates), the N-oxide derivatives, protected derivatives, individual isomers and mixture of isomers thereof, are inhibitors of either c-kit kinase activity. Such compounds of Formula (I) provided herein, and the pharmaceutically acceptable salts, pharmaceutically acceptable solvates (e.g. hydrates), the N-oxide derivatives, protected derivatives, individual isomers and mixture of isomers thereof, are useful for treating diseases or disorders in which c-kit kinase, or c-kit and PDGFR (a and/or β) kinase, contributes to the pathology and/or symptomology of a disease or disorder. Such diseases or disorders include, but are not limited to, a mast cell associated disease, inflammatory diseases, respiratory diseases, an allergy disorder, fibrosis diseases, metabolic diseases, autoimmune diseases, a CNS related disorder, a neurodegenerative disorder, neurological diseases, dermatoligical diseases, a graft-versus-host disease, a pain condition, a neoplastic disorder, a cardiovascular disease and cancer.

Non-limiting examples of such diseases include asthma, allergic rhinitis, allergic sinusitis, bronchial asthma, irritable bowel syndrome (IBS), inflammatory bowel disease (IBD), pulmonary arterial hypertension (PAH), idiopathic arterial hypertension (IPAH), primary pulmonary hypertension (PPH), pulmonary fibrosis, liver fibrosis, cardiac fibrosis, scleroderma, urticaria, dermatoses, atopic dermatitis, allergic contact dermatitis, diabetes, type I diabetes, type II diabetes, rheumatoid arthritis, multiple scherosis, cytopenias (by way of example only, anemia, leucopenia, neutropenia, thrombocytopenia, granuloctopenia, pancytoia and idiopathic thrombocytopenic purpura), systemic lupus erythematosus, chronic obstructive pulmonary disease (COPD), adult respiratory distress syndrome (ARDS), ulcerative colitis, Crohns disease, psoriasis, lymphomas (by way of example only, B and T cell lymphomas), myelodysplasic syndrome, breast cancer, pancreatic cancer, papillary thyroid carcinoma, ovarian carcinoma, human adenoid cystic carcinoma, non small cell lung cancer, secretory breast carcinoma, congenital fibrosarcoma, congenital mesoblastic nephroma, acute myelogenous leukemia, chronic myeloid leukemia metastasis, cancer-related pain, neuroblastoma, osteosarcoma, melanoma, bone metastases, a tumor of breast, renal, lung, prostate, pancreas, colon, ovary, thyroid, colorectal tumors, neuronal tumors, uterine tumors, gastrointestinal stromal tumors (GIST), gliomas, sarcomas, tumor angiogenesis, germ cell tumors, mast cell tumors, glioblastoma, neuroblastoma, mastocytosis, osteoporosis, hypereosinophilia, restenosis, atherosclerosis, anaphylactic syndrome, angioedema, erythema nodosum, erythema multiforme, cutaneous necrotizing venulitis, insect bite skin inflammation, CNS disorders and interstitial cystitis.

In certain embodiments, the compounds of Formula (I) provided herein, and the pharmaceutically acceptable salts, pharmaceutically acceptable solvates (e.g. hydrates), the N-oxide derivatives, protected derivatives, individual isomers and mixture of isomers thereof, are useful for treating diseases or disorders in which c-kit kinase contributes to the pathology and/or symptomology of a disease or disorder. Non-limiting examples of such diseases include asthma, allergic rhinitis, allergic sinusitis, bronchial asthma, irritable bowel syndrome (IBS), inflammatory bowel disease (IBD), pulmonary arterial hypertension (PAH), pulmonary fibrosis, liver fibrosis, cardiac fibrosis, scleroderma, urticaria, dermatoses, atopic dermatitis, allergic contact dermatitis, diabetes, type I diabetes, type II diabetes, rheumatoid arthritis, multiple scherosis, cytopenias (by way of example only, anemia, leucopenia, neutropenia, thrombocytopenia, granuloctopenia, pancytoia and idiopathic thrombocytopenic purpura), systemic lupus erythematosus, chronic obstructive pulmonary disease (COPD), adult respiratory distress syndrome (ARDS), ulcerative colitis, Crohns disease, psoriasis, lymphomas (by way of example only, Band T cell lymphomas), myelodysplasic syndrome, breast cancer, pancreatic cancer, papillary thyroid carcinoma, ovarian carcinoma, human adenoid cystic carcinoma, non small cell lung cancer, secretory breast carcinoma, congenital fibrosarcoma, congenital mesoblastic nephroma, acute myelogenous leukemia, chronic myeloid leukemia metastasis, cancer-related pain, neuroblastoma, osteosarcoma, melanoma, bone metastases, a tumor of breast, renal, lung, prostate, pancreas, colon, ovary, thyroid, colorectal tumors, neuronal tumors, uterine tumors, gastrointestinal stromal tumors (GIST), gliomas, sarcomas, tumor angiogenesis, germ cell tumors, mast cell tumors, glioblastoma, neuroblastoma, mastocytosis, osteoporosis, hypereosinophilia, restenosis, atherosclerosis, anaphylactic syndrome, angioedema, erythema nodosum, erythema multiforme, cutaneous necrotizing venulitis, insect bite skin inflammation, CNS disorders and interstitial cystitis.

In certain embodiments, the compounds of Formula (I) provided herein, and the pharmaceutically acceptable salts, pharmaceutically acceptable solvates (e.g. hydrates), the N-oxide derivatives, protected derivatives, individual isomers and mixture of isomers thereof, are useful for treating diseases or disorders in which c-kit kinase and PDGFR (a and/or 3) kinase contribute to the pathology and/or symptomology of a disease or disorder. Non-limiting examples of such diseases include asthma, allergic rhinitis, allergic sinusitis, bronchial asthma, irritable bowel syndrome (IBS), inflammatory bowel disease (IBD), pulmonary arterial hypertension (PAH), pulmonary fibrosis, liver fibrosis, cardiac fibrosis, scleroderma, urticaria, dermatoses, atopic dermatitis, allergic contact dermatitis, diabetes, type I diabetes, type 11 diabetes, rheumatoid arthritis, multiple scherosis, cytopenias (by way of example only, anemia, leucopenia, neutropenia, thrombocytopenia, granuloctopenia, pancytoia and idiopathic thrombocytopenic purpura), systemic lupus erythematosus, chronic obstructive pulmonary disease (COPD), adult respiratory distress syndrome (ARDS), ulcerative colitis, Crohns disease, psoriasis, lymphomas (by way of example only, B and T cell lymphomas), myelodysplasic syndrome, breast cancer, pancreatic cancer, papillary thyroid carcinoma, ovarian carcinoma, human adenoid cystic carcinoma, non small cell lung cancer, secretory breast carcinoma, congenital fibrosarcoma, congenital mesoblastic nephroma, acute myelogenous leukemia, chronic myeloid leukemia metastasis, cancer-related pain, neuroblastoma, osteosarcoma, melanoma, bone metastases, a tumor of breast, renal, lung, prostate, pancreas, colon, ovary, thyroid, colorectal tumors, neuronal tumors, uterine tumors, gastrointestinal stromal tumors (GIST), gliomas, sarcomas, tumor angiogenesis, germ cell tumors, mast cell tumors, glioblastoma, neuroblastoma, mastocytosis, osteoporosis, hypereosinophilia, restenosis, atherosclerosis, anaphylactic syndrome, angioedema, erythema nodosum, erythema multiforme, cutaneous necrotizing venulitis, insect bite skin inflammation, CNS disorders and interstitial cystitis.

Another aspect provided herein includes methods for treating a cell-proliferative disease, comprising administering to a system or subject in need of such treatment an effective amount of a compound of Formula (I), or pharmaceutically acceptable salts or pharmaceutical compositions thereof, wherein the cell-proliferative disease is lymphoma, osteosarcoma, melanoma, or a tumor of breast, renal, prostate, colorectal, thyroid, ovarian, pancreatic, neuronal, lung, uterine or gastrointestinal tumor.

In certain embodiments, the compounds of Formula (I), pharmaceutically acceptable salts, solvates, N-oxides and isomers thereof, pharmaceutical compositions, and/or combinations provided herein are used in the treatment diseases and/or disorders including, but not limited to, asthma, bronchial asthma, allergic asthma, intrinsic asthma, extrinsic asthma, exercise-induced asthma, drug-induced asthma (including aspirin and NSAID-induced) and dust-induced asthma, chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus.

In certain embodiments, the compounds of Formula (I), pharmaceutically acceptable salts, solvates, N-oxides and isomers thereof, pharmaceutical compositions, and/or combinations provided herein are used in the treatment of dermatological disorders including, but not limited to, psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, basal cell carcinoma, actinic keratosis, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions.

In certain embodiments, the compounds of Formula (I), pharmaceutically acceptable salts, solvates, N-oxides and isomers thereof, pharmaceutical compositions, and/or combinations provided herein are used in the treatment of rheumatoid arthritis, irritable bowel syndrome, systemic lupus erythematosus, multiple sclerosis, Hashimoto's thyroiditis, Crohns disease, inflammatory bowel disease (IBD), Graves' disease, Addison's disease, diabetes mellitus, idiopathic thrombocytopaenic purpura, eosinophilic fasciitis, hyper-lgE syndrome, antiphospholipid syndrome and Sazary syndrome.

In certain embodiments, the compounds of Formula (I), pharmaceutically acceptable salts, solvates, N-oxides and isomers thereof, and pharmaceutical compositions provided herein are used in the treatment of cancer including, but not limited to, prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumor recurrences, and paraneoplastic syndromes.

Provided herein are compounds of Formula (I), pharmaceutically acceptable salts, pharmaceutically acceptable solvates (e.g. hydrates), the N-oxide derivatives, protected derivatives, individual isomers and mixture of isomers thereof, and pharmaceutical compositions containing at least one compound of Formula (I), or pharmaceutically acceptable salts, pharmaceutically acceptable solvates (e.g. hydrates), the N-oxide derivatives, protected derivatives, individual isomers or mixture of isomers thereof, for use in activating c-kit kinase activity, or c-kit kinase and PDGFRR (a and/or 3) kinase activity, and thereby are used to in the prevention or treatment of diseases and/or disorders associated with c-kit kinase activity, or c-kit kinase and PDGFRR (a and/or 3) kinase activity.

Also provided herein are methods for the treatment of a subject suffering from a disease and/or disorder associated with c-kit kinase activity, wherein the method includes administering to the subject in need thereof, an effective amount of a compound of Formula (I), or pharmaceutically acceptable salts, pharmaceutically acceptable solvates (e.g. hydrates), the N-oxide derivatives, protected derivatives, individual isomers or mixture of isomers thereof, either alone or as part of a pharmaceutical composition as described herein.

Also provided herein are methods for the treatment of a subject suffering from a disease and/or disorder associated with c-kit kinase activity and PDGFR (a and/or 3) kinase activity, wherein the method includes administering to the subject in need thereof, an effective amount of a compound of Formula (I), or pharmaceutically acceptable salts, pharmaceutically acceptable solvates (e.g. hydrates), the N-oxide derivatives, protected derivatives, individual isomers or mixture of isomers thereof, either alone or as part of a pharmaceutical composition as described herein.

Provided herein is the use of a compound of Formula (I), or pharmaceutically acceptable salts, pharmaceutically acceptable solvates (e.g. hydrates), the N-oxide derivatives, protected derivatives, individual isomers or mixture of isomers thereof, in the manufacture of a medicament for the treatment of a disease or disorder associated with c-kit kinase activity. Also provided herein is the use of a compound of Formula (I), or pharmaceutically acceptable salts, pharmaceutically acceptable solvates (e.g. hydrates), the N-oxide derivatives, protected derivatives, individual isomers or mixture of isomers thereof, in the manufacture of a medicament for the treatment of a disease or disorder associated with c-kit kinase activity and PDGFR (α and/or β) kinase activity.

Furthermore, provided herein is the use of a compound having Formula (I), or pharmaceutically acceptable salts or pharmaceutical compositions thereof, and optionally in combination with a therapeutically effective amount of a second agent, in the manufacture of a medicament for treating a disease or condition modulated by kinase activity, particularly c-kit, or c-kit and PDGFRR (α and/or β).

In accordance with the foregoing, the present invention further provides a method for preventing or treating any of the diseases or disorders described above in a subject in need of such treatment, which method comprises administering to said subject a therapeutically effective amount of a compound of Formula (I), or a pharmaceutically acceptable salt thereof. For any of the above uses, the required dosage will vary depending on the mode of administration, the particular condition to be treated and the effect desired. (See, “Administration and Pharmaceutical Compositions,” infra).

An inhibitory amount or dose of the compounds of the present invention may range from about 0.01 mg/Kg to about 500 mg/Kg, alternatively from about 1 to about 50 mg/Kg. Inhibitory amounts or doses will also vary depending on route of administration, as well as the possibility of co-usage with other agents.

According to the methods of treatment of the present invention, conditions are treated or prevented in a patient such as a human or another animal by administering to the patient a therapeutically effective amount of a compound of the invention, in such amounts and for such time as is necessary to achieve the desired result.

By a “therapeutically effective amount” of a compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). An effective amount of the compound described above may range from about 0.1 mg/Kg to about 500 mg/Kg, preferably from about 1 to about 50 mg/Kg. Effective doses will also vary depending on route of administration, as well as the possibility of co-usage with other agents. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

The total daily dose of the compounds of this invention administered to a human or other animal in single or in divided doses can be in amounts, for example, from 0.01 to 50 mg/kg body weight or more usually from 0.1 to 25 mg/kg body weight. Single dose compositions may contain such amounts or submultiples thereof to make up the daily dose. In general, treatment regimens according to the present invention comprise administration to a patient in need of such treatment from about 10 mg to about 1000 mg of the compound(s) of this invention per day in single or multiple doses.

The compounds of the present invention described herein can, for example, be administered by injection, intravenously, intra-arterial, subdermally, intraperitoneally, intramuscularly, or subcutaneously; or orally, buccally, nasally, transmucosally, topically, in an ophthalmic preparation, or by inhalation, with a dosage ranging from about 0.1 to about 500 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with pharmaceutically excipients or carriers to produce a single dosage form will vary depending upon the host treated and the particular mode of administration. A typical preparation will contain from about 5% to about 95% active compound (w/w). Alternatively, such preparations may contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

When the compositions of this invention comprise a combination of a compound of the Formula described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The said “additional therapeutic or prophylactic agents” includes but not limited to, immune therapies (e.g. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (e.g. N-acetylcysteine), cytokine agonists, cytokine antagonists, lung surfactants and/or antimicrobial and anti-viral agents (e.g. ribavirin and amantidine). The compositions according to the invention may also be used in combination with gene replacement therapy.

### Abbreviations

Abbreviations which may be used in the descriptions of the scheme and the examples that follow are: Ac for acetyl; AcOH for acetic acid; ACN or MeCN or CH₃CN for acetonitrile; AlMe₃ for trimethylaluminum; BINAP for 2,2′-bis(diphenylphosphino)-1,1′-binaphthalene; Boc for t-butoxycarbonyl; B₂(Pin)₂ for bis(pinacolato)diboron; Brine for sodium chloride solution in water; Burgess reagent for 1-methoxy-N-triethylammoniosulfonyl-methanimidate; Cbz for benzyloxycarbonyl; ClCOCOCl for oxalyl chloride; CDI for carbonyldiimidazole; cHex for cyclohexane; Cs₂CO₃ for cesium carbonate; CuBr₂ for copper (II) bromide; CuI for copper (I) iodide; CuSO₄-5H₂O for copper (II) sulfate pentahydrate; DBAD for di-tert-butyl azodicarboxylate; DCE for 1,2-dichloroethane; DCM or CH₂Cl₂ for dichloromethane; DDC for N,N′-dicyclohexylcarbodiimide; CH₃ for methyl; DIPEA or (i-Pr)₂EtN for N,N-diisopropylethyl amine; DME for 1,2-dimethoxyethane; DMF for N,N-dimethylformamide; DMSO for dimethyl sulfoxide; EDC for 1-ethyl-3-(3-dimethylaminopropyl)carbodiimide; EtOAc or EA for ethyl acetate; EtOH for ethanol; Fe for iron; Fmoc for fluorenylmethyloxycarbonyl; Ghosez's reagent for 1-chloro-N,N,2-trimethyl-1-propenylamine; HATU for O-(7-azabenzotriazol-2-yl)-N,N,N′,N′-tetramethyluronium Hexafluoro-phosphate; HCl for hydrogen chloride; H₂O for water; K₂CO₃ for potassium carbonate; KOAc for potassium acetate; MeMgBr for methylmagnesium bromide; K₃PO₄ for tripotassium phosphate; Lawesson's reagent for 2,4-bis(4-methoxyphenyl)-2,4-dithioxo-1,3,2,4-dithiadiphosphetane; MeOH for methanol; N₂ for nitrogen; NaCl for sodium chloride; NaHCO₃ for sodium bicarbonate or sodium hydrogen carbonate; Na₂CO₃ sodium carbonate; NaOH for sodium hydroxide; Na₂SO₄ for sodium sulfate; NH₃ for ammonia; NH₄Cl for ammonium chloride; NH₄OH for ammonium hydroxide; NH₄HCO₃ for ammonium bicarbonate; NH₂OH for hydroxyamine; NMP for N-methyl-2-pyrrolidone; NO₂ for nitro; Ph₃P for triphenylphosphine; PTSA for p-toluenesulfonic acid; Pd/C for palladium on carbon; PdCl₂(dppf) for [1,1′-bis(diphenylphosphino)ferrocene]dichloropalladium(II); PdCl₂(dppf)-CH₂Cl₂ for [1,1′-bis(diphenylphosphino)ferrocene]dichloropalladium(II), complex with dichloromethane; PdCl₂(PPh₃)₂ for bis(triphenylphosphine)palladium(II) dichloride; Pd(OAc)₂ for Palladium(II) acetate; Pd(OH)₂ for palladium (II) hydroxide; Pd(Ph₃P)₄ for tetrakis(triphenylphosphine)palladium(0); Pd-PEPPSI-SIPr for (1,3-bis(2,6-diisopropylphenyl)imidazolidene) (3-chloropyridyl) palladium(II) dichloride; SnCl₂ for tin (II) chloride; SOCl₂ for thionyl chloride; TEA or Et₃N for triethylamine; TFA for trifluoroacetic acid; TFAA for trifluoroacetic anhydride; THE for tetrahydrofuran; T₃P for propanephosphonic acid anhydride; OTf for triflate; Ph for phenyl; rt for room temperature; TBS for tert-butyl dimethylsilyl; TMS for trimethylsilyl; or TMSOTf for trimethylsilyl trifluoromethanesulfonate; XPhos Pd G3 for (2-dicyclohexylphosphino-2′,4′,6′-triisopropyl-1,1′-biphenyl)[2-(2′-amino-1,1′-biphenyl)]palladium(II) methanesulfonate; Zn for zinc.

### Synthetic Methods

The compounds and processes of the present invention will be better understood in connection with the following synthetic schemes that illustrate the methods by which the compounds of the invention may be prepared, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, and/or methods of the invention may be made without departing from the spirit of the invention and the scope of the appended claims.

Non-limiting examples of synthetic schemes demonstrating the making of compounds of the invention are illustrated in Schemes 1 to 6.

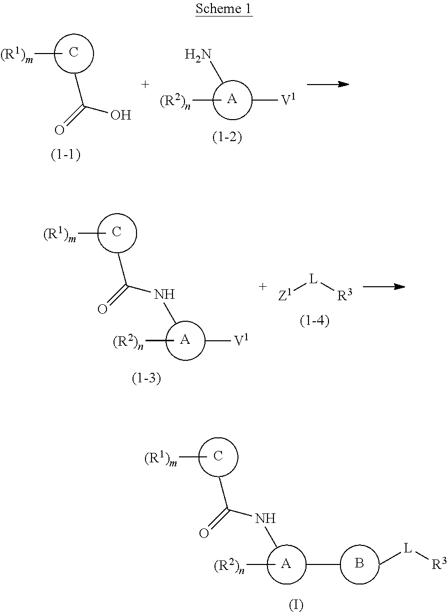

Scheme 1 illustrates a general method to prepare the compound of Formula (I). The carboxylic acid (1-1), wherein R¹, C and m are as previously defined, is condensed with amine (1-2) wherein, A, R² and n are previously defined and V¹ is, without limitation, hydroxylamine, an azide, an alkyne, a carboxylic acid, a hydrazide or a nitrile, under amide coupling conditions (e.g. HATU, EDC, DCC, T3P, etc.) or other acid activating conditions such as acyl halide (SOCl₂, ClCOCOCl and Ghosez's reagent), or acyl imidazole (carbonyl diimidazole) provides amide (1-3). The amide (1-3) is reacted with compound (1-4), wherein L and R³ are previously defined and Z¹ is, without limitation, a carboxylic acid, an alkyne, an amine, or an azide, using relevant heterocyclic synthetic methods found in the literature (e.g. Y. Ishihara, A. Montero, P. S. Baran, The Portable Chemist's Consultant: A Survival Guide for Discovery, Process, and Radiolabeling, Apple Publishing Group, New York City, NY, 2013, or J. A. Joule, K. Mills. Heterocyclic Chemistry, 5th Edition, Wiley-Blackwell, Hoboken, NJ, 2010, etc.) to form the corresponding heterocyclic ring B and to provide the compound with Formula (I), wherein B is as previously defined.

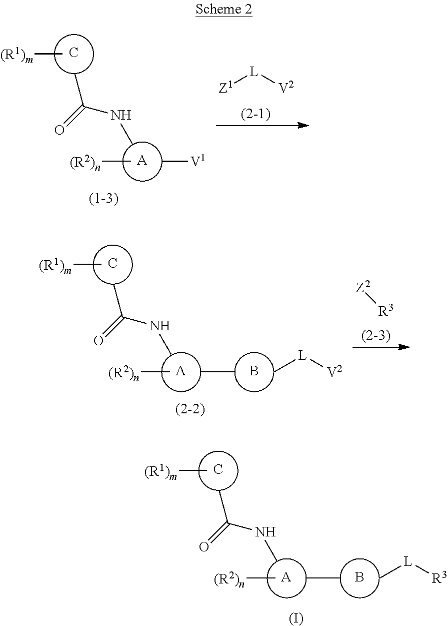

Alternatively, as demonstrated in Scheme 2, the compound of Formula (I) can be prepared from the compound (1-3), wherein R¹, R², m, n, A, C, and V¹ are as previously defined. Compound (1-3) is reacted with compound (2-1), wherein L and Z¹ are as previously defined, V² is selected from, but not limited to, hydrogen, halogen, triflate, boronic acid, boronic ester, amine, etc., using the heterocyclic synthetic method previously described to form heterocyclic ring B and to provide compound (2-2). Treatment of compound (2-2) with compound (2-3), wherein R³ is as previously defined and Z² is selected from, but not limited to, halogen, triflate, boronic acid, boronic ester, amine, etc., affords the compound of Formula (I).

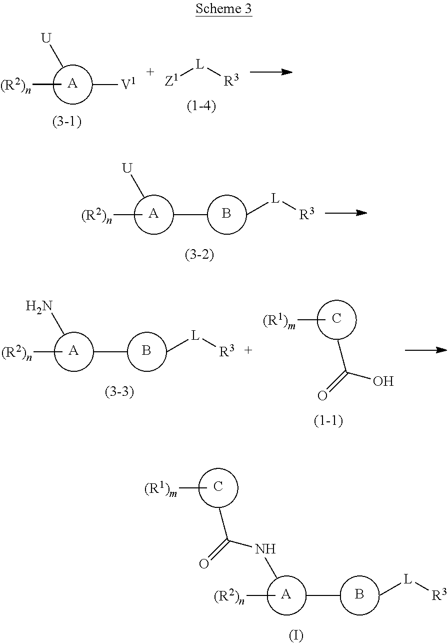

Alternatively, as demonstrated in Scheme 3, the compound of Formula (I) can be prepared from Compound (3-1), wherein A, R², n and V¹ are as previously defined, and U is —NO₂, or a protected amino group (e.g. —NHBoc, NHCbz, NHFmoc, etc.). Compound (3-1) is reacted with compound (1-4), wherein Z¹, L, and R³ are as previously defined, using the heterocyclic synthetic methods previously described to form heterocyclic ring B and to provide compound (3-2), wherein B is as previously defined. U in (3-2) is converted to the amine in (3-3) by following procedures: when U is —NO₂, either hydrogenation under proper hydrogen pressure in the presence of a catalyst such as but not limited to Pd/C, Pd(OH)₂, Raney Nickel, or treatment with a metal reducing reagent such as but not limited to, Zn, Fe, SnCl₂, etc. can provide amine (3-3); when U is a protected amino group, the amino protecting group is removed (e.g. hydrogenation for Cbz in the presence of a proper catalyst such as Pd/C, or an acid such HCl, TFA, PTSA, TMSOTf for Boc) to afford amine (3-3). The amine (3-3) is condensed with the carboxylic acid (1-1), wherein R¹, C, and m are as previously defined, under previously described amide bond formation conditions to provide the compound with Formula (I).

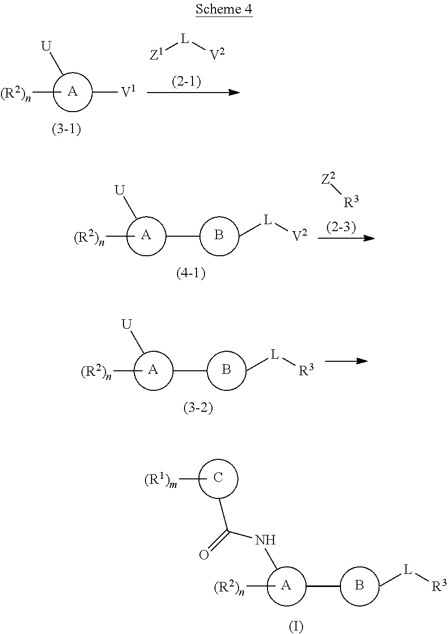

Alternatively, as demonstrated in Scheme 4, the compound of Formula (I) can be prepared from Compound (3-1), wherein U, A, R², n, and V¹ are as previously defined. Compound (3-1) is reacted with compound (2-1), wherein Z¹, L, and V² are as previously defined, using the heterocyclic synthetic methods previously described to form heterocyclic ring B and to provide compound (4-1), wherein B is as previously defined. Treatment of compound (4-1) with compound (2-3), wherein R³ and Z² are as previously defined, affords compound (3-2), which can be further elaborated to the compound of Formula (I), wherein R¹, m, and C are as previously defined, using the reaction sequence as shown in Scheme 3 and as described above.

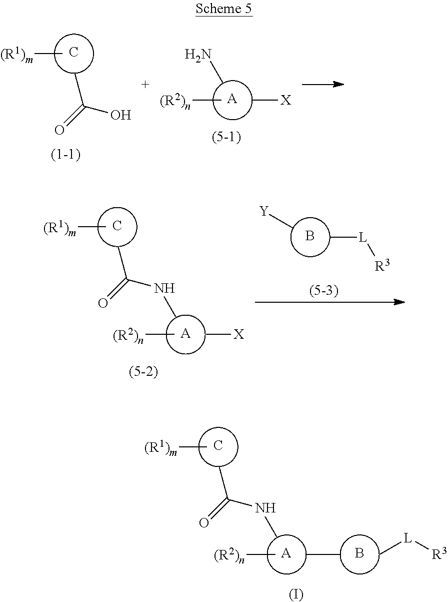

Alternatively, as demonstrated in Scheme 5, the compound of Formula (I) can be prepared from the amine (5-1), wherein A, R², and n are as previously defined, and X is hydrogen, halogen, boronic acid, boronic ester, or triflate. Compound (1-1), wherein R¹, C, and m are as previously defined, is condensed with compound (5-1) under previously described amide bond formation conditions to form the amide (5-2). Compound (5-2) is reacted using transition metal catalyzed reactions (e.g. Suzuki coupling, Stille coupling, Sonogashira coupling, Negishi coupling, Buchwald-Hartwig coupling, Ullmann coupling, C—H activation coupling, photoredox-mediated coupling, etc.) with (5-3), wherein L, B, and R³ are previously defined, and Y is, without limitation, halogen, boronic acid, boronic ester, organotin, organozinc, magnesium halide, organosilane, or hydrogen, to provide the compound of Formula (I).

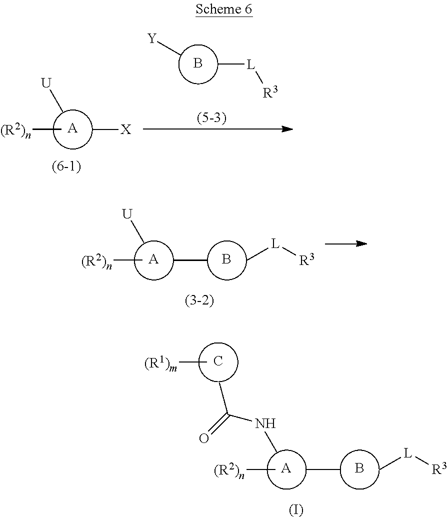

Alternatively, as demonstrated in Scheme 6, the compound of Formula (I) can be prepared from Compound (6-1), wherein U, A, R², n, and X are as previously defined. Compound (6-1) is reacted with Compound (5-3), wherein Y, B, L, and R³ are as previously defined, using the previously described transition metal catalyzed reactions to form compound (3-2), which can be further elaborated to the compound of Formula (I), wherein R¹, C, and m are as previously defined, using the reaction sequence as shown in Scheme 3 and as described above.

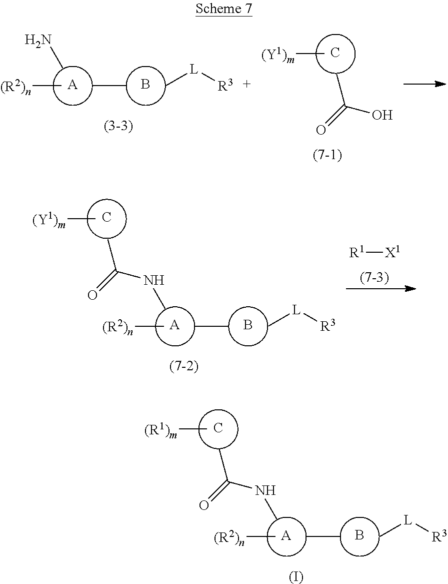

Alternatively, as demonstrated in Scheme 7, the compound of Formula (I) can be prepared from Compound (3-3), wherein A, B, L, R², R³, and n are as previously defined. Compound (3-3) is condensed with Compound (7-1), wherein C and m are as previously defined, and Y¹ is halogen, organotin, triflate, boronic acid, or boronic ester under previously described amide bond formation conditions to form the amide (7-2). Compound (7-2) is coupled with Compound (7-3), wherein R¹ is as previously defined and X¹ is hydrogen, halogen, organotin, triflate, boronic acid, or boronic ester, to form the compound of Formula (I) by using transition metal catalyzed reactions (e.g. Suzuki coupling, Stille coupling, Sonogashira coupling, Negishi coupling, Buchwald-Hartwig coupling, Ullmann coupling, C—H activation coupling, photoredox-mediated coupling, etc.)

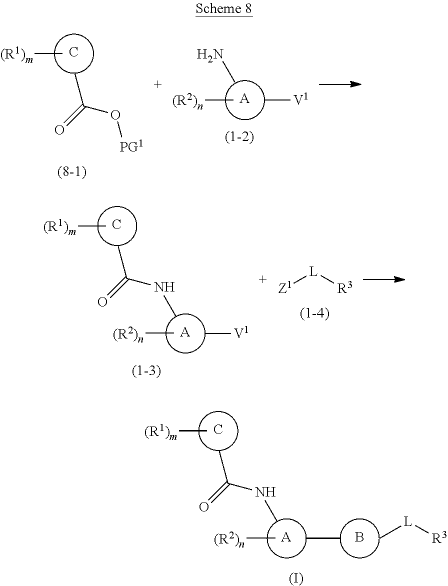

Alternatively, as demonstrated in Scheme 8, the compound of Formula (I) can be prepared from Compound (8-1), wherein R¹, C, and m are as previously defined and PG¹ is methyl, ethyl, propyl, difluoromethyl, benzyl, phenyl, or pentafluorophenyl. The transamidation of Compound (8-1) and Compound (1-2), wherein R², A, V¹, and n are as previously defined, could afford Compound (1-3) in the presence of Lewis acid, e.g. trimethylaluminum, diethyl aluminum chloride, boron trifluoride etherate, etc. or in the presence of a base, e.g. LiHMDS, NaHMDS, KHMDS, triethylamine, DIPEA, N-methylmorpholine, etc. The compound of Formula (I) could be further obtained by using the similar approach as shown in Scheme 1.

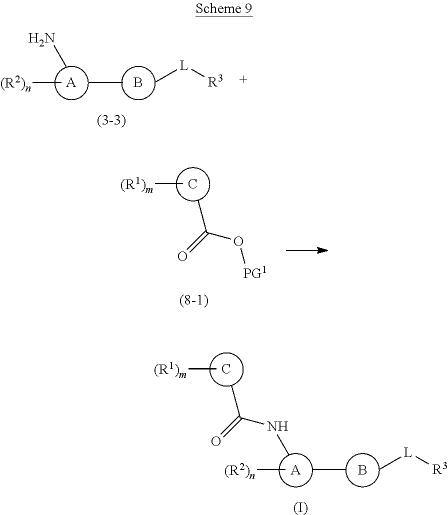

Alternatively, as demonstrated in Scheme 9, the compound of Formula (I) can be prepared by the transamidation of Compound (3-3), wherein R², A, B, L, R³, and n are as previously defined, with Compound (8-1), wherein RI, C, PG¹ and m are as previously defined, in the presence of Lewis acid, e.g. trimethylaluminum, diethyl aluminum chloride, boron trifluoride etherate, etc. or in the presence of a base, e.g. LiHMDS, NaHMDS, KHMDS, triethylamine, DIPEA, N-methyl morpholine, etc.

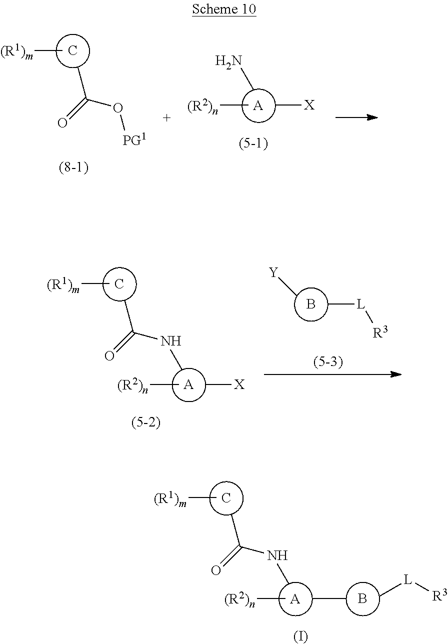

Alternatively, as demonstrated in Scheme 10, the compound of Formula (I) can be prepared from Compound (8-1), wherein RI, C, PG¹, and m are as previously defined. The transamidation of Compound (8-1) and Compound (5-1), wherein R², A, X, and n are as previously defined, could afford Compound (5-2) in the presence of Lewis acid, e.g. trimethylaluminum, diethyl aluminum chloride, boron trifluoride etherate, etc. or in the presence of a base, e.g. LiHMDS, NaHMDS, KHMDS, triethylamine, DIPEA, N-methyl morpholine, etc. The compound of Formula (I) could be further obtained by using the similar approach as shown in Scheme 5.

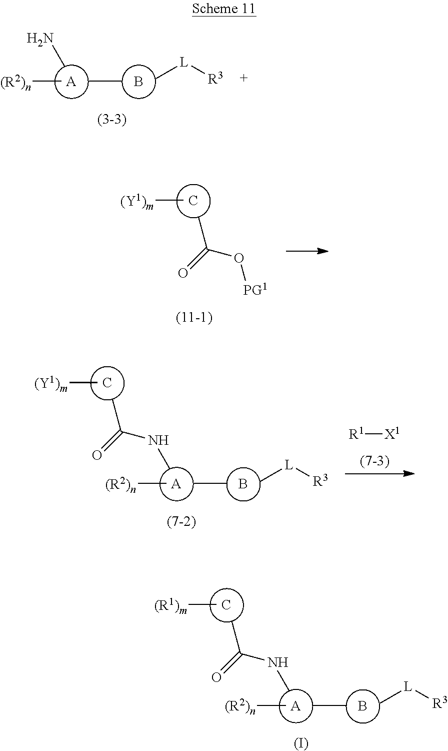

Alternatively, as demonstrated in Scheme 11, the compound of Formula (I) can be prepared from Compound (11-1), wherein R¹, C, Y¹, PG¹, and m are as previously defined. The transamidation of Compound (11-1) and Compound (3-3), wherein R², A, B, L, R³, and n are as previously defined, could afford Compound (7-2) in the presence of Lewis acid, e.g. trimethylaluminum, diethyl aluminum chloride, boron trifluoride etherate, etc. or in the presence of a base, e.g. LiHMDS, NaHMDS, KHMDS, triethylamine, DIPEA, N-methyl morpholine, etc. The compound of Formula (I) could be further obtained by using the similar approach as shown in Scheme 7.

### EXAMPLES

The compounds and processes of the present invention will be better understood in connection with the following examples, which are intended as an illustration only and not limiting the scope of the invention. Starting materials were either available from a commercial vendor or produced by methods well known to those skilled in the art.

### General Conditions:

Mass spectra were run on LC-MS systems using electrospray ionization. These were Agilent 1290 Infinity II systems with an Agilent 6120 Quadrupole detector. Spectra were obtained using a ZORBAX Eclipse XDB-C18 column (4.6×30 mm, 1.8 micron). Spectra were obtained at 298K using a mobile phase of 0.1% formic acid in water (A) and 0.1% formic acid in acetonitrile (B). Spectra were obtained with the following solvent gradient: 5% (B) from 0-1.5 min, 5-95% (B) from 1.5-4.5 min, and 95% (B) from 4.5-6 min. The solvent flowrate was 1.2 mL/min. Compounds were detected at 210 nm and 254 nm wavelengths. [M+H]⁺ refers to mono-isotopic molecular weights.

NMR spectra were run on a Bruker 400 MHz spectrometer. Spectra were measured at 298K and referenced using the solvent peak. Chemical shifts for ¹H NMR were reported in parts per million (ppm).

Compounds were purified via reverse-phase high-performance liquid chromatography (RPHPLC) using a Gilson GX-281 automated liquid handling system. Compounds were purified on a Phenomenex Kinetex EVO C18 column (250×21.2 mm, 5 micron), unless otherwise specified. Compounds were purified at 298K using a mobile phase of water (A) and acetonitrile (B) using gradient elution between 0% and 100% (B), unless otherwise specified. The solvent flowrate was 20 mL/min and compounds were detected at 254 nm wavelength.

Alternatively, compounds were purified via normal-phase liquid chromatography (NPLC) using a Teledyne ISCO Combiflash purification system. Compounds were purified on a REDISEP silica gel cartridge. Compounds were purified at 298K and detected at 254 nm wavelength.

### Example 1

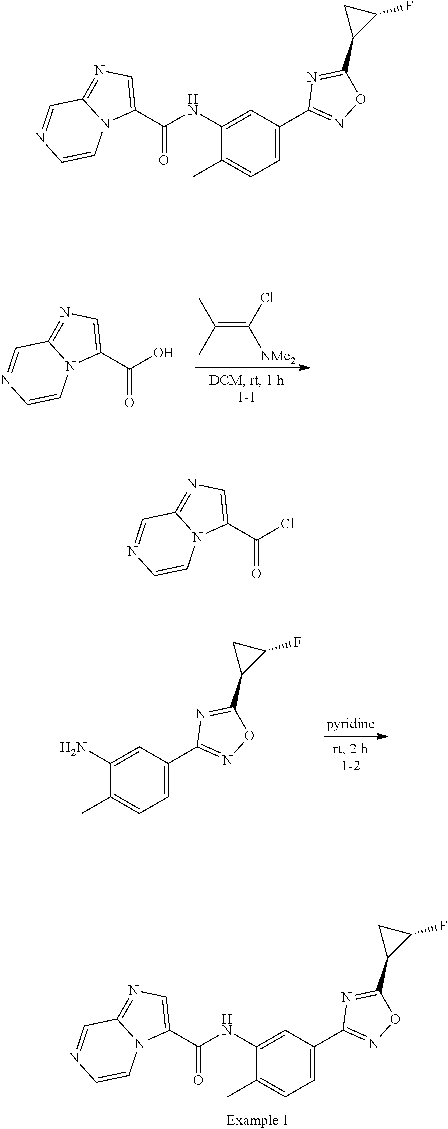

Step 1-1: A suspension of imidazo[1,2-a]pyrazine-3-carboxylic acid (33 mg, 0.202 mmol) in CH₂Cl₂ (0.5 ml) was treated with 1-chloro-N,N,2-trimethylprop-1-en-1-amine (32 μl, 0.242 mmol). The reaction was stirred at room temperature for 1 hr and the mixture turned to a clear solution. The freshly prepared desired product (37 mg, 0.204 mmol, 101% yield) in CH₂Cl₂ was directly used in the next step.
Step 1-2: A solution of 5-(5-((1R,2S)-2-fluorocyclopropyl)-1,2,4-oxadiazol-3-yl)-2-methylaniline (18 mg, 0.077 mmol) in pyridine (0.5 ml) was treated with a freshly prepared solution of the compound from Step 1-1 (17 mg, 0.094 mmol) in DCM. The reaction was stirred at room temperature for 2 hrs. The mixture was concentrated in vacuo. The crude was added to a 4 g silica gel column and eluted by acetone/cyclohexane from 0% to 100% to give Example 1 (15 mg, 0.040 mmol, 51.4% yield) as a white solid. LC-MS, ES+: 379.21 [M+H]⁺. ¹H NMR (400 MHz, CDCl₃) δ 9.48 (dd, J=4.7, 1.5 Hz, 1H), 9.35 (d, J=1.5 Hz, 1H), 8.73 (s, 1H), 8.37 (d, J=1.8 Hz, 2H), 8.25 (d, J=4.6 Hz, 1H), 7.84 (dd, J=7.9, 1.7 Hz, 1H), 7.38 (d, J=8.0 Hz, 1H), 5.05 (dddd, J=62.7, 6.3, 3.5, 1.8 Hz, 1H), 2.72 (dddd, J=16.1, 11.0, 7.0, 1.8 Hz, 1H), 2.43 (s, 3H), 1.84 (dddd, J=21.5, 10.9, 7.2, 3.6 Hz, 1H), 1.61 (dq, J=13.3, 6.8 Hz, 1H).

The following examples were prepared using procedures similar to those described above:

| Example | Structure | ESI-MS |
|---|---|---|
| 2 | 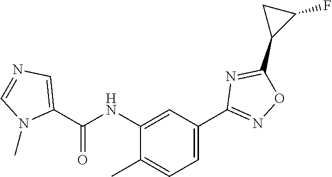 [SMILES: Cc1ccc(-c2noc([C@H]3C[C@@H]3F)n2)cc1NC(=O)c1cncn1C] | (M + H)⁺: 342.21 |
| 3 | 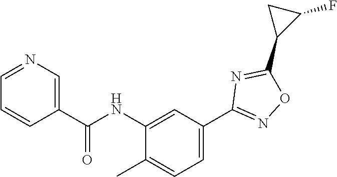 [SMILES: Cc1ccc(-c2noc([C@H]3C[C@@H]3F)n2)cc1NC(=O)c1cccnc1] | (M + H)⁺: 339.17 |
| 4 | 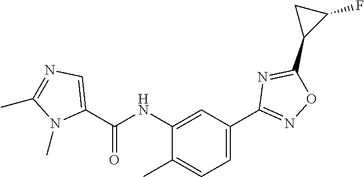 [SMILES: Cc1ccc(-c2noc([C@H]3C[C@@H]3F)n2)cc1NC(=O)c1cnc(C)n1C] | (M + H)⁺: 356.22 |
| 5 | 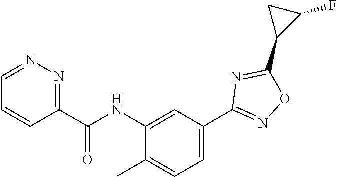 [SMILES: Cc1ccc(-c2noc([C@H]3C[C@@H]3F)n2)cc1NC(=O)c1cccnn1] | (M + H)⁺: 340.17 |
| 6 | 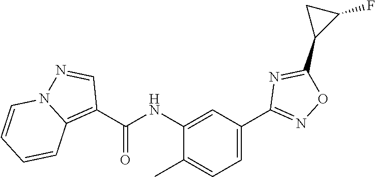 [SMILES: Cc1ccc(-c2noc([C@H]3C[C@@H]3F)n2)cc1NC(=O)c1cnn2ccccc12] | (M + H)⁺: 378.19 |
| 7 | 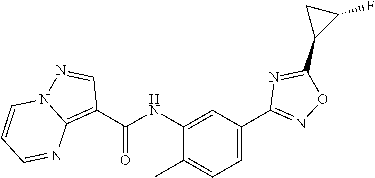 [SMILES: Cc1ccc(-c2noc([C@H]3C[C@@H]3F)n2)cc1NC(=O)c1cnn2cccnc12] | (M + H)⁺: 379.15 |
| 8 | 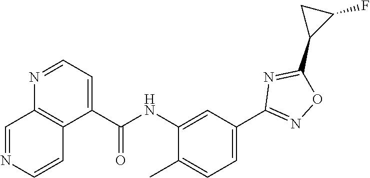 [SMILES: Cc1ccc(-c2noc([C@H]3C[C@@H]3F)n2)cc1NC(=O)c1ccnc2cnccc12] | (M + H)⁺: 390.24 |
| 9 | 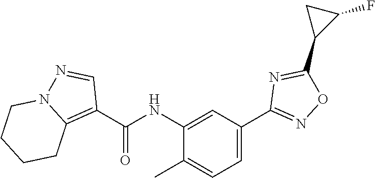 [SMILES: Cc1ccc(-c2noc([C@H]3C[C@@H]3F)n2)cc1NC(=O)c1cnn2c1CCCC2] | (M + H)⁺: 382.26 |
| 10 | 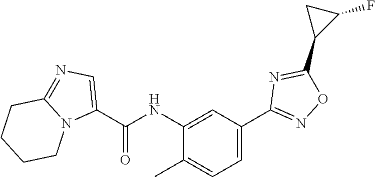 [SMILES: Cc1ccc(-c2noc([C@H]3C[C@@H]3F)n2)cc1NC(=O)c1cnc2n1CCCC2] | (M + H)⁺: 382.23 |
| 11 | 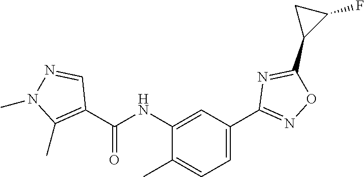 [SMILES: Cc1ccc(-c2noc([C@H]3C[C@@H]3F)n2)cc1NC(=O)c1cnn(C)c1C] | (M + H)⁺: 356.22 |
| 12 | 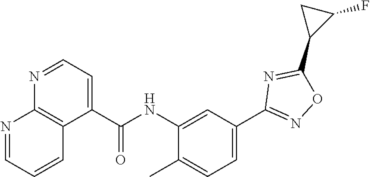 [SMILES: Cc1ccc(-c2noc([C@H]3C[C@@H]3F)n2)cc1NC(=O)c1ccnc2ncccc12] | (M + H)⁺: 390.24 |
| 13 | 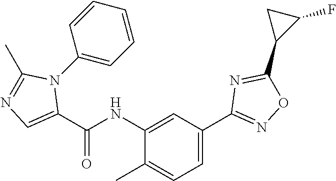 [SMILES: Cc1ccc(-c2noc([C@H]3C[C@@H]3F)n2)cc1NC(=O)c1cnc(C)n1-c1ccccc1] | (M + H)⁺: 418.36 |
| 14 | 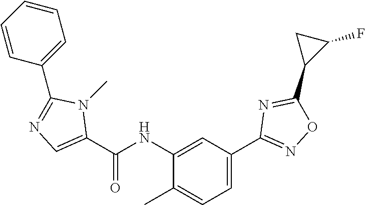 [SMILES: Cc1ccc(-c2noc([C@H]3C[C@@H]3F)n2)cc1NC(=O)c1cnc(-c2ccccc2)n1C] | (M + H)⁺: 418.46 |
| 15 | 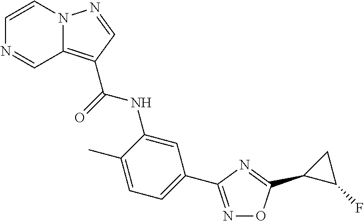 [SMILES: Cc1ccc(-c2noc([C@H]3C[C@@H]3F)n2)cc1NC(=O)c1cnn2ccncc12] | (M + H)⁺: 379.19 |
| 16 | 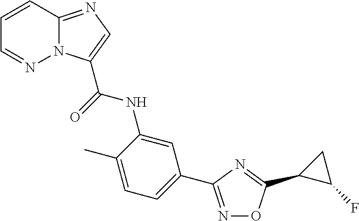 | (M + H)⁺: 379.19 |
| 17 | 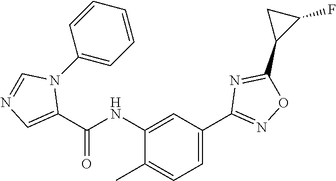 [SMILES: Cc1ccc(-c2noc([C@H]3C[C@@H]3F)n2)cc1NC(=O)c1cncn1-c1ccccc1] | (M + H)⁺: 404.35 |
| 18 | 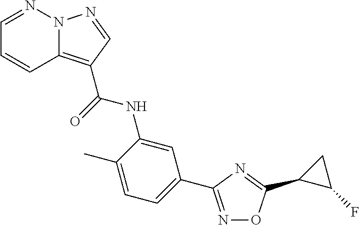 [SMILES: Cc1ccc(-c2noc([C@H]3C[C@@H]3F)n2)cc1NC(=O)c1cnn2ncccc12] | (M + H)⁺: 379.29 |
| 19 | 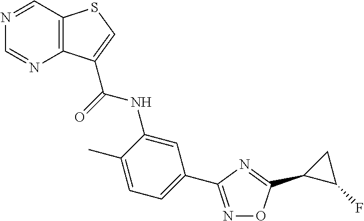 [SMILES: Cc1ccc(-c2noc([C@H]3C[C@@H]3F)n2)cc1NC(=O)c1csc2cncnc12] | (M + H)⁺: 396.19 |
| 20 | 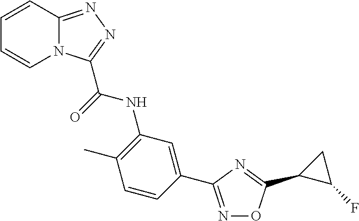 [SMILES: Cc1ccc(-c2noc([C@H]3C[C@@H]3F)n2)cc1NC(=O)c1nnc2ccccn12] | (M + H)⁺: 379.19 |
| 21 | 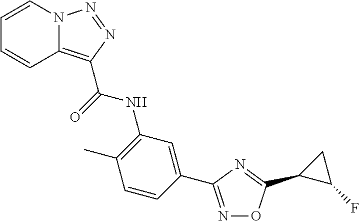 [SMILES: Cc1ccc(-c2noc([C@H]3C[C@@H]3F)n2)cc1NC(=O)c1nnn2ccccc12] | (M + H)⁺: 379.19 |
| 22 | 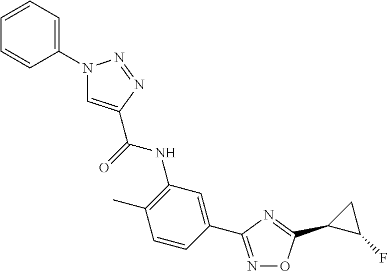 [SMILES: Cc1ccc(-c2noc([C@H]3C[C@@H]3F)n2)cc1NC(=O)c1cn(-c2ccccc2)nn1] | (M + H)⁺: 405.36 |
| 23 | 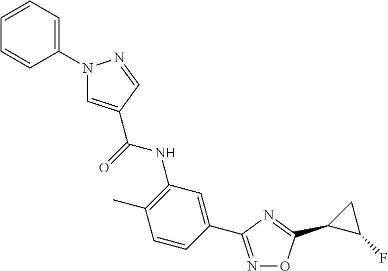 [SMILES: Cc1ccc(-c2noc([C@H]3C[C@@H]3F)n2)cc1NC(=O)c1cnn(-c2ccccc2)c1] | (M + H)⁺: 404.28 |
| 24 | 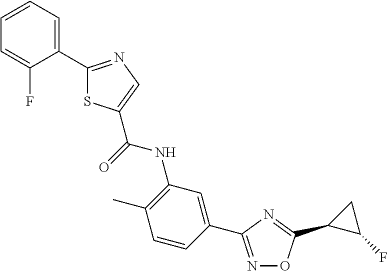 [SMILES: Cc1ccc(-c2noc([C@H]3C[C@@H]3F)n2)cc1NC(=O)c1cnc(-c2ccccc2F)s1] | (M + H)⁺: 439.20 |
| 25 | 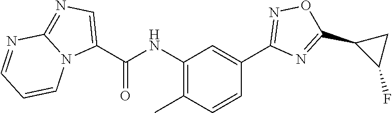 [SMILES: Cc1ccc(-c2noc([C@H]3C[C@@H]3F)n2)cc1NC(=O)c1cnc2ncccn12] | (M + H)⁺: 379.2 |
| 26 | 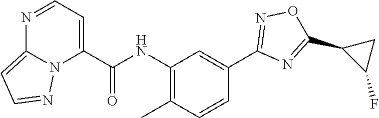 [SMILES: Cc1ccc(-c2noc([C@H]3C[C@@H]3F)n2)cc1NC(=O)c1ccnc2ccnn12] | (M + H)⁺: 379.1 |
| 27 | 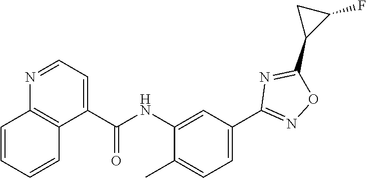 [SMILES: Cc1ccc(-c2noc([C@H]3C[C@@H]3F)n2)cc1NC(=O)c1ccnc2ccccc12] | (M + H)⁺: 389.3 |
| 28 | 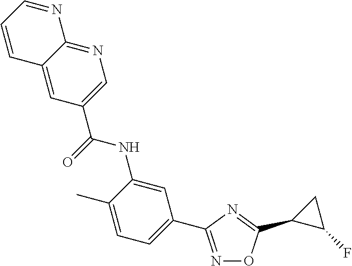 [SMILES: Cc1ccc(-c2noc([C@H]3C[C@@H]3F)n2)cc1NC(=O)c1cnc2ncccc2c1] | (M + H)⁺: 390.3 |
| 29 | 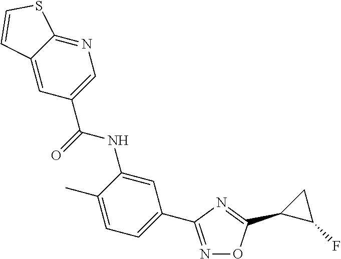 [SMILES: Cc1ccc(-c2noc([C@H]3C[C@@H]3F)n2)cc1NC(=O)c1cnc2sccc2c1] | (M + H)⁺: 395.1 |
| 30 | 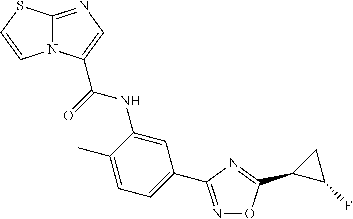 [SMILES: Cc1ccc(-c2noc([C@H]3C[C@@H]3F)n2)cc1NC(=O)c1cnc2sccn12] | (M + H)⁺: 384.3 |
| 31 | 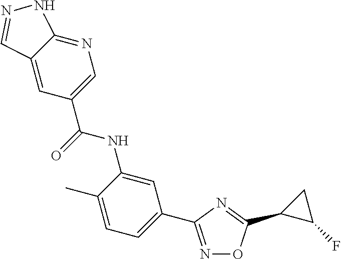 [SMILES: Cc1ccc(-c2noc([C@H]3C[C@@H]3F)n2)cc1NC(=O)c1cnc2[nH]ncc2c1] | (M + H)⁺: 379.1 |
| 32 | 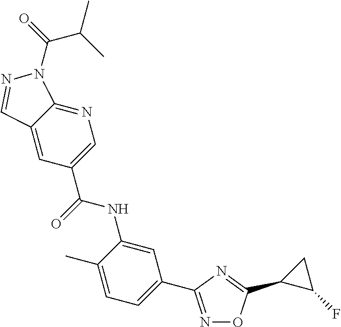 [SMILES: Cc1ccc(-c2noc([C@H]3C[C@@H]3F)n2)cc1NC(=O)c1cnc2c(cnn2C(=O)C(C)C)c1] | (M + H)⁺: 449.1 |
| 33 | 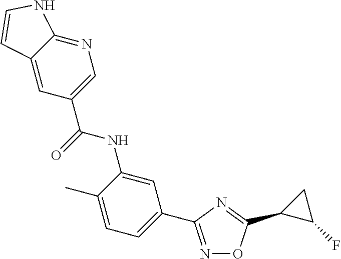 [SMILES: Cc1ccc(-c2noc([C@H]3C[C@@H]3F)n2)cc1NC(=O)c1cnc2[nH]ccc2c1] | (M + H)⁺: 378.3 |
| 34 | 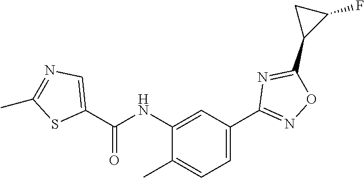 [SMILES: Cc1ncc(C(=O)Nc2cc(-c3noc([C@H]4C[C@@H]4F)n3)ccc2C)s1] | (M + H)⁺: 359.19 |
| 35 | 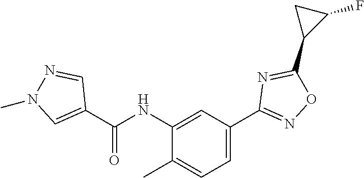 [SMILES: Cc1ccc(-c2noc([C@H]3C[C@@H]3F)n2)cc1NC(=O)c1cnn(C)c1] | (M + H)⁺: 342.18 |
| 36 | 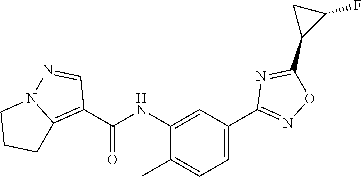 [SMILES: Cc1ccc(-c2noc([C@H]3C[C@@H]3F)n2)cc1NC(=O)c1cnn2c1CCC2] | (M + H)⁺: 368.23 |
| 37 | 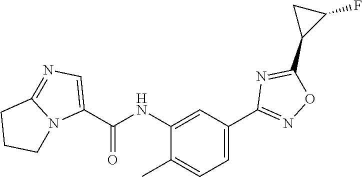 [SMILES: Cc1ccc(-c2noc([C@H]3C[C@@H]3F)n2)cc1NC(=O)c1cnc2n1CCC2] | (M + H)⁺: 368.24 |
| 38 | 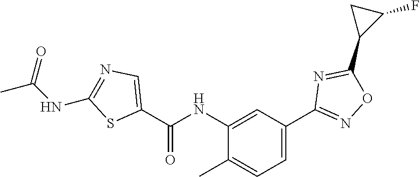 [SMILES: CC(=O)Nc1ncc(C(=O)Nc2cc(-c3noc([C@H]4C[C@@H]4F)n3)ccc2C)s1] | (M + H)⁺: 402.17 |
| 39 | 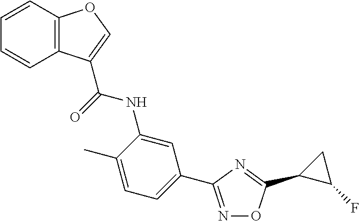 [SMILES: Cc1ccc(-c2noc([C@H]3C[C@@H]3F)n2)cc1NC(=O)c1coc2ccccc12] | (M + H)⁺: 378.23 |
| 40 | 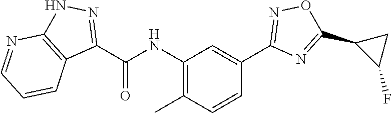 [SMILES: Cc1ccc(-c2noc([C@H]3C[C@@H]3F)n2)cc1NC(=O)c1n[nH]c2ncccc12] | (M + H)⁺: 379.05 |
| 41 | 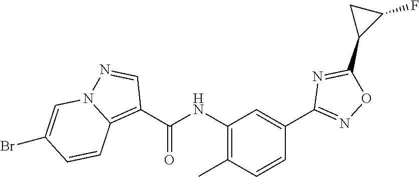 [SMILES: Cc1ccc(-c2noc([C@H]3C[C@@H]3F)n2)cc1NC(=O)c1cnn2cc(Br)ccc12] | (M + H)⁺: 456.08, 458.06 |
| 42 | 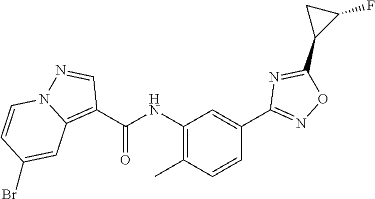 [SMILES: Cc1ccc(-c2noc([C@H]3C[C@@H]3F)n2)cc1NC(=O)c1cnn2ccc(Br)cc12] | (M + H)⁺: 456.09, 458.11 |
| 43 | 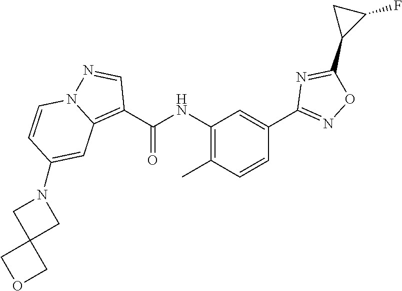 [SMILES: Cc1ccc(-c2noc([C@H]3C[C@@H]3F)n2)cc1NC(=O)c1cnn2ccc(N3CC4(COC4)C3)cc12] | (M + H)⁺: 475.24 |
| 44 | 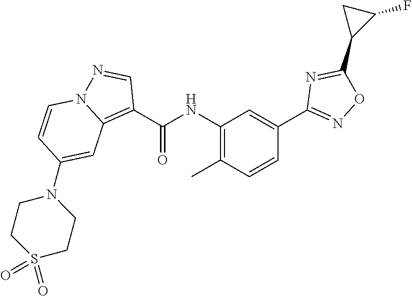 [SMILES: Cc1ccc(-c2noc([C@H]3C[C@@H]3F)n2)cc1NC(=O)c1cnn2ccc(N3CCS(=O)(=O)CC3)cc12] | (M + H)⁺: 511.29 |
| 45 | 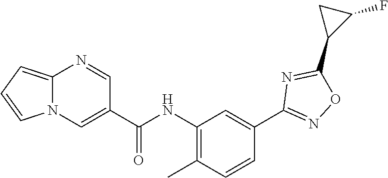 [SMILES: Cc1ccc(-c2noc([C@H]3C[C@@H]3F)n2)cc1NC(=O)c1cnc2cccn2c1] | (M + H)⁺: 378.09 |
| 46 | 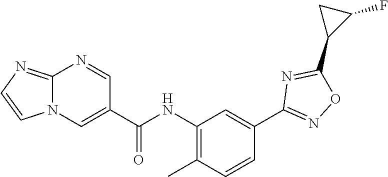 [SMILES: Cc1ccc(-c2noc([C@H]3C[C@@H]3F)n2)cc1NC(=O)c1cnc2nccn2c1] | (M + H)⁺: 379.05 |
| 47 | 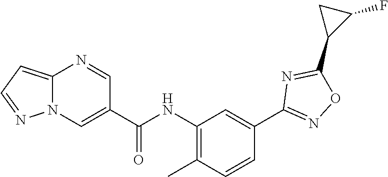 [SMILES: Cc1ccc(-c2noc([C@H]3C[C@@H]3F)n2)cc1NC(=O)c1cnc2ccnn2c1] | (M + H)⁺: 379.10 |
| 48 | 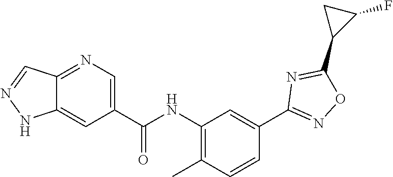 [SMILES: Cc1ccc(-c2noc([C@H]3C[C@@H]3F)n2)cc1NC(=O)c1cnc2cn[nH]c2c1] | (M + H)⁺: 379.15 |
| 49 | 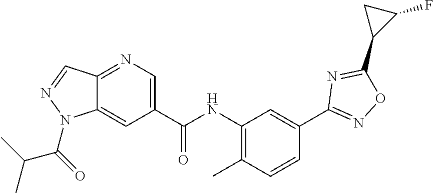 [SMILES: Cc1ccc(-c2noc([C@H]3C[C@@H]3F)n2)cc1NC(=O)c1cnc2cnn(C(=O)C(C)C)c2c1] | (M + H)⁺: 449.11 |
| 50 | 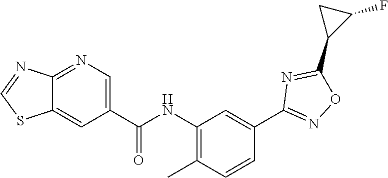 [SMILES: Cc1ccc(-c2noc([C@H]3C[C@@H]3F)n2)cc1NC(=O)c1cnc2ncsc2c1] | (M + H)⁺: 396.09 |
| 51 | 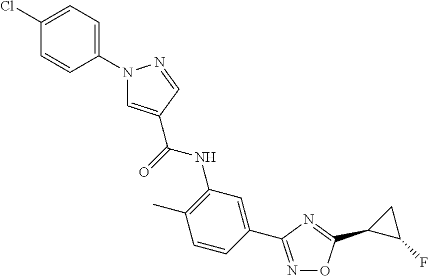 [SMILES: Cc1ccc(-c2noc([C@H]3C[C@@H]3F)n2)cc1NC(=O)c1cnn(-c2ccc(Cl)cc2)c1] | (M + H)⁺: 438.23 |
| 52 | 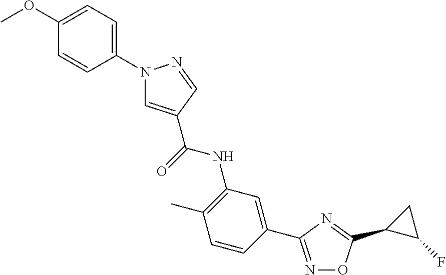 [SMILES: COc1ccc(-n2cc(C(=O)Nc3cc(-c4noc([C@H]5C[C@@H]5F)n4)ccc3C)cn2)cc1] | (M + H)⁺: 434.28 |
| 53 | 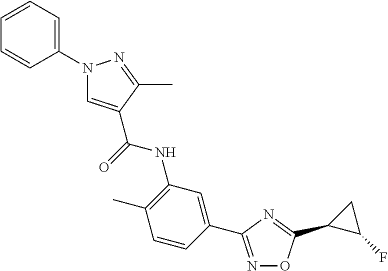 [SMILES: Cc1ccc(-c2noc([C@H]3C[C@@H]3F)n2)cc1NC(=O)c1cn(-c2ccccc2)nc1C] | (M + H)⁺: 418.26 |
| 54 | 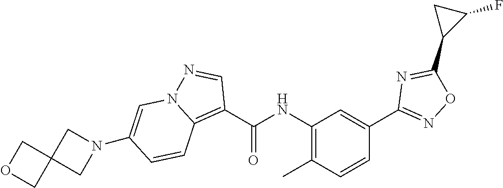 [SMILES: Cc1ccc(-c2noc([C@H]3C[C@@H]3F)n2)cc1NC(=O)c1cnn2cc(N3CC4(COC4)C3)ccc12] | (M + H)⁺: 475.29 |
| 1a | 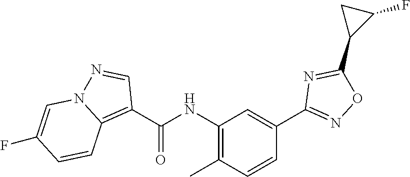 [SMILES: Cc1ccc(-c2noc([C@H]3C[C@@H]3F)n2)cc1NC(=O)c1cnn2cc(F)ccc12] | (M + H)⁺: 396.14 |
| 55 | 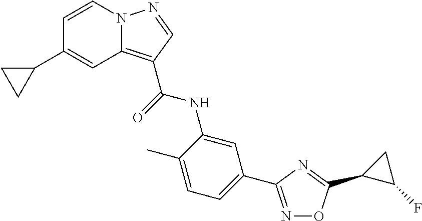 [SMILES: Cc1ccc(-c2noc([C@H]3C[C@@H]3F)n2)cc1NC(=O)c1cnn2ccc(C3CC3)cc12] | (M + H)⁺: 418.314 |
| 56 | 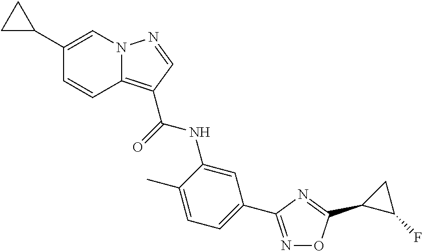 [SMILES: Cc1ccc(-c2noc([C@H]3C[C@@H]3F)n2)cc1NC(=O)c1cnn2cc(C3CC3)ccc12] | (M + H)⁺: 418.218 |
| 57 | 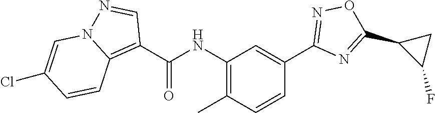 [SMILES: Cc1ccc(-c2noc([C@H]3C[C@@H]3F)n2)cc1NC(=O)c1cnn2cc(Cl)ccc12] | (M + H)⁺: 412.03 |
| 58 | 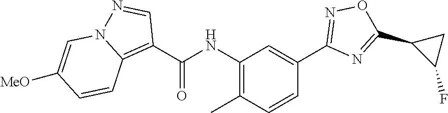 [SMILES: COc1ccc2c(C(=O)Nc3cc(-c4noc([C@H]5C[C@@H]5F)n4)ccc3C)cnn2c1] | (M + H)⁺: 408.09 |
| 59 | 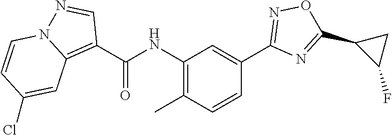 [SMILES: Cc1ccc(-c2noc([C@H]3C[C@@H]3F)n2)cc1NC(=O)c1cnn2ccc(Cl)cc12] | (M + H)⁺: 412.07 |
| 60 | 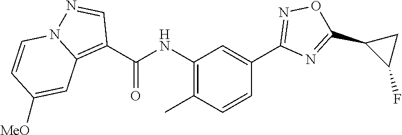 [SMILES: COc1ccn2ncc(C(=O)Nc3cc(-c4noc([C@H]5C[C@@H]5F)n4)ccc3C)c2c1] | (M + H)⁺: 408.14 |
| 61 | 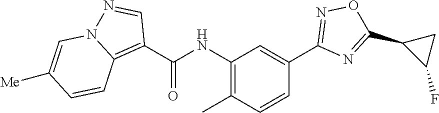 [SMILES: Cc1ccc2c(C(=O)Nc3cc(-c4noc([C@H]5C[C@@H]5F)n4)ccc3C)cnn2c1] | (M + H)⁺: 392.34 |
| 62 | 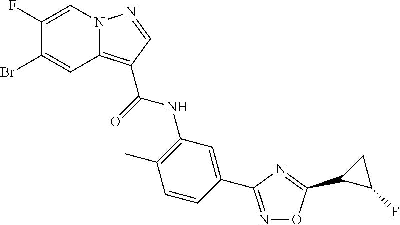 | (M + H)⁺: 473.946, 475.866 |
| 63 | 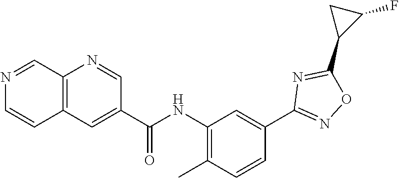 [SMILES: Cc1ccc(-c2noc([C@H]3C[C@@H]3F)n2)cc1NC(=O)c1cnc2cnccc2c1] | (M + H)⁺: 390.2 |
| 64 | 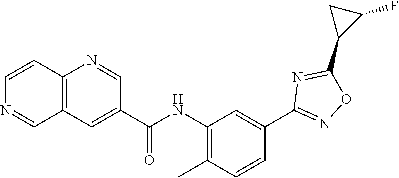 [SMILES: Cc1ccc(-c2noc([C@H]3C[C@@H]3F)n2)cc1NC(=O)c1cnc2ccncc2c1] | (M + H)⁺: 390.1 |
| 65 | 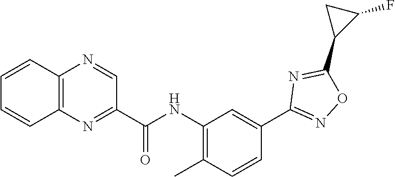 [SMILES: Cc1ccc(-c2noc([C@H]3C[C@@H]3F)n2)cc1NC(=O)c1cnc2ccccc2n1] | (M + H)⁺: 390.2 |
| 66 | 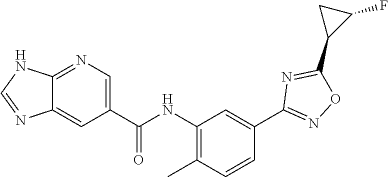 [SMILES: Cc1ccc(-c2noc([C@H]3C[C@@H]3F)n2)cc1NC(=O)c1cnc2[nH]cnc2c1] | (M + H)⁺: 379.2 |
| 67 | 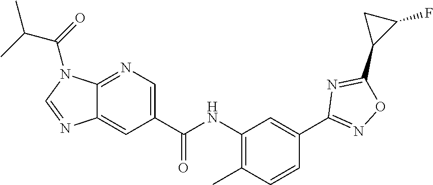 [SMILES: Cc1ccc(-c2noc([C@H]3C[C@@H]3F)n2)cc1NC(=O)c1cnc2c(c1)ncn2C(=O)C(C)C] | (M + H)⁺: 449.2 |
| 68 | 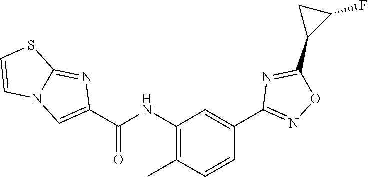 [SMILES: Cc1ccc(-c2noc([C@H]3C[C@@H]3F)n2)cc1NC(=O)c1cn2ccsc2n1] | (M + H)⁺: 384.1 |
| 69 | 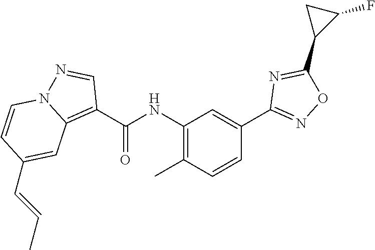 [SMILES: C/C=C/c1ccn2ncc(C(=O)Nc3cc(-c4noc([C@H]5C[C@@H]5F)n4)ccc3C)c2c1] | (M + H)⁺: 418.2 |
| 70 | 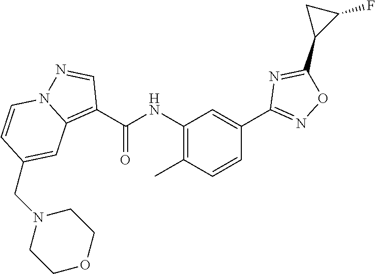 [SMILES: Cc1ccc(-c2noc([C@H]3C[C@@H]3F)n2)cc1NC(=O)c1cnn2ccc(CN3CCOCC3)cc12] | (M + H)⁺: 477.1 |
| 71 | 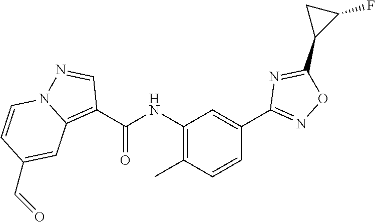 [SMILES: Cc1ccc(-c2noc([C@H]3C[C@@H]3F)n2)cc1NC(=O)c1cnn2ccc(C=O)cc12] | (M + H)⁺: 406.1 |
| 72 | 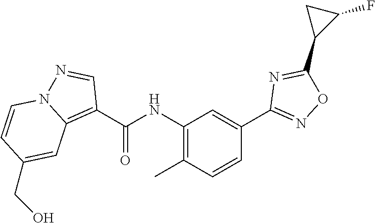 [SMILES: Cc1ccc(-c2noc([C@H]3C[C@@H]3F)n2)cc1NC(=O)c1cnn2ccc(CO)cc12] | (M + H)⁺: 408.1 |
| 73 | 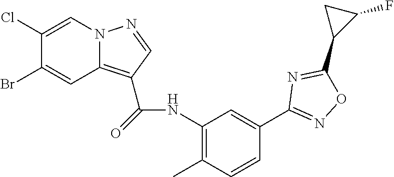 [SMILES: Cc1ccc(-c2noc([C@H]3C[C@@H]3F)n2)cc1NC(=O)c1cnn2cc(Cl)c(Br)cc12] | (M + H)⁺: 489.8, 491.7 |
| 74 | 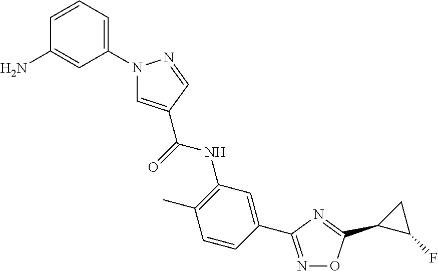 [SMILES: Cc1ccc(-c2noc([C@H]3C[C@@H]3F)n2)cc1NC(=O)c1cnn(-c2cccc(N)c2)c1] | (M + H)⁺: 419.51 |
| 75 | 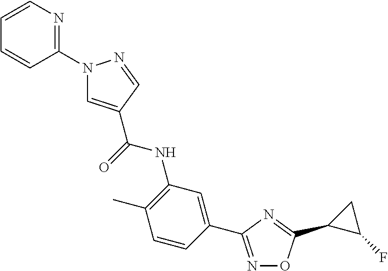 [SMILES: Cc1ccc(-c2noc([C@H]3C[C@@H]3F)n2)cc1NC(=O)c1cnn(-c2ccccn2)c1] | (M + H)⁺: 405.21 |
| 76 | 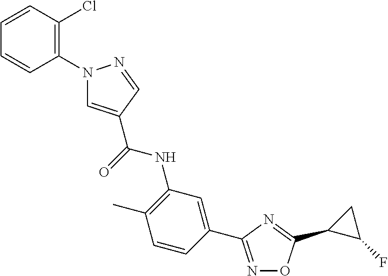 [SMILES: Cc1ccc(-c2noc([C@H]3C[C@@H]3F)n2)cc1NC(=O)c1cnn(-c2ccccc2Cl)c1] | (M + H)⁺: 438.13 |
| 77 | 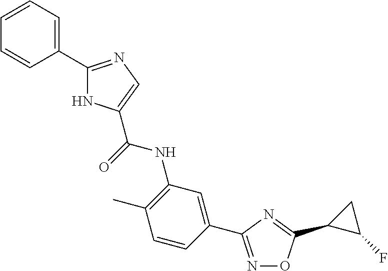 [SMILES: Cc1ccc(-c2noc([C@H]3C[C@@H]3F)n2)cc1NC(=O)c1cnc(-c2ccccc2)[nH]1] | (M + H)⁺: 404.25 |
| 78 | 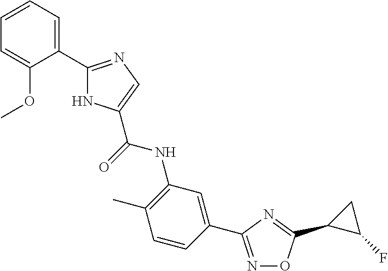 [SMILES: COc1ccccc1-c1ncc(C(=O)Nc2cc(-c3noc([C@H]4C[C@@H]4F)n3)ccc2C)[nH]1] | (M + H)⁺: 434.25 |
| 79 | 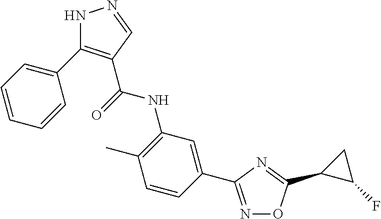 [SMILES: Cc1ccc(-c2noc([C@H]3C[C@@H]3F)n2)cc1NC(=O)c1cn[nH]c1-c1ccccc1] | (M + H)⁺: 404.23 |
| 80 | 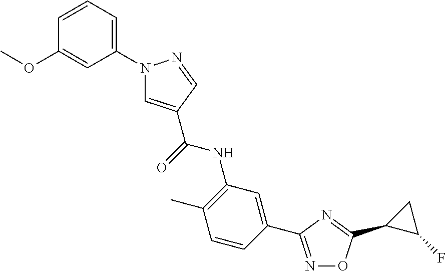 [SMILES: COc1cccc(-n2cc(C(=O)Nc3cc(-c4noc([C@H]5C[C@@H]5F)n4)ccc3C)cn2)c1] | (M + H)⁺: 434.26 |
| 81 | 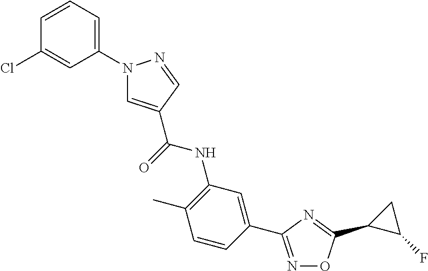 [SMILES: Cc1ccc(-c2noc([C@H]3C[C@@H]3F)n2)cc1NC(=O)c1cnn(-c2cccc(Cl)c2)c1] | (M + H)⁺: 438.19, 440.11 |
| 82 | 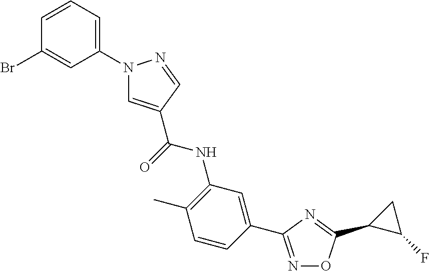 [SMILES: Cc1ccc(-c2noc([C@H]3C[C@@H]3F)n2)cc1NC(=O)c1cnn(-c2cccc(Br)c2)c1] | (M + H)⁺: 482.15, 484.11 |
| 83 | 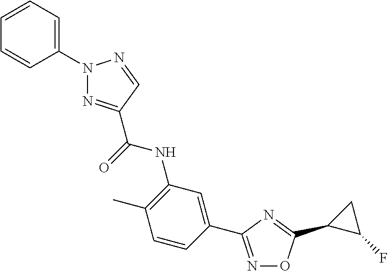 [SMILES: Cc1ccc(-c2noc([C@H]3C[C@@H]3F)n2)cc1NC(=O)c1cnn(-c2ccccc2)n1] | (M + H)⁺: 405.24 |
| 84 | 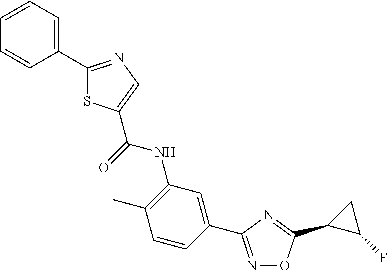 [SMILES: Cc1ccc(-c2noc([C@H]3C[C@@H]3F)n2)cc1NC(=O)c1cnc(-c2ccccc2)s1] | (M + H)⁺: 421.24 |
| 85 | 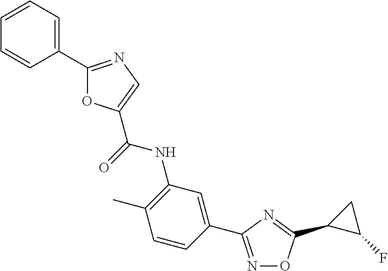 [SMILES: Cc1ccc(-c2noc([C@H]3C[C@@H]3F)n2)cc1NC(=O)c1cnc(-c2ccccc2)o1] | (M + H)⁺: 405.27 |
| 86 | 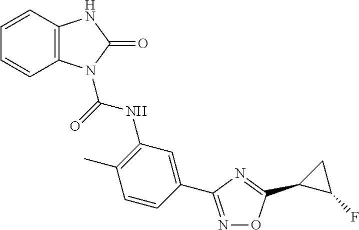 [SMILES: Cc1ccc(-c2noc([C@H]3C[C@@H]3F)n2)cc1NC(=O)n1c(=O)[nH]c2ccccc21] | (M + H)⁺: 394.13 |
| 87 | 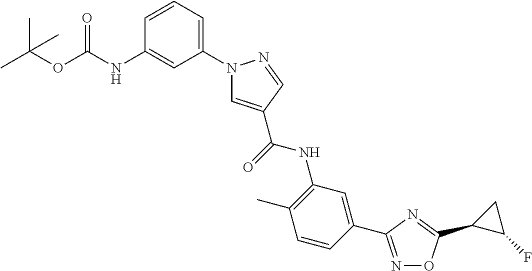 [SMILES: Cc1ccc(-c2noc([C@H]3C[C@@H]3F)n2)cc1NC(=O)c1cnn(-c2cccc(NC(=O)OC(C)(C)C)c2)c1] | (M + H)⁺: 519.31 |
| 88 | 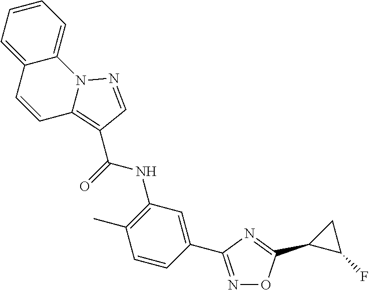 [SMILES: Cc1ccc(-c2noc([C@H]3C[C@@H]3F)n2)cc1NC(=O)c1cnn2c1ccc1ccccc12] | (M + H)⁺: 428.23 |
| 89 | 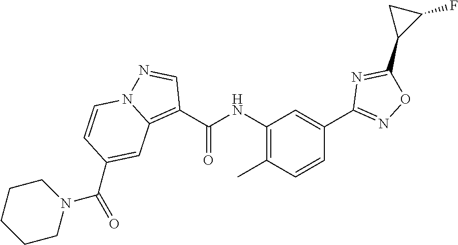 [SMILES: Cc1ccc(-c2noc([C@H]3C[C@@H]3F)n2)cc1NC(=O)c1cnn2ccc(C(=O)N3CCCCC3)cc12] | (M + H)⁺: 489.32 |
| 90 | 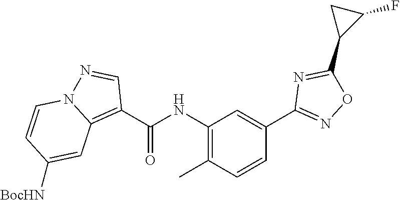 [SMILES: Cc1ccc(-c2noc([C@H]3C[C@@H]3F)n2)cc1NC(=O)c1cnn2ccc(NC(=O)OC(C)(C)C)cc12] | (M + H)⁺: 493.31 |
| 91 | 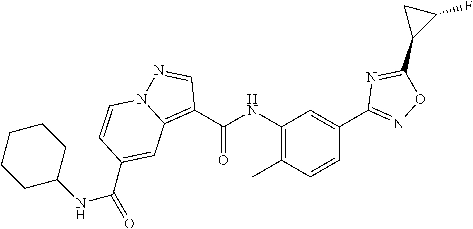 [SMILES: Cc1ccc(-c2noc([C@H]3C[C@@H]3F)n2)cc1NC(=O)c1cnn2ccc(C(=O)NC3CCCCC3)cc12] | (M + H)⁺: 503.22 |
| 92 | 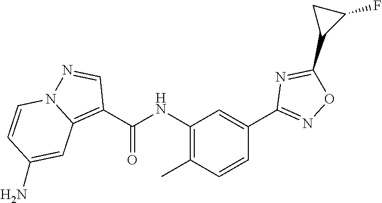 | (M + H)⁺: 393.34 |
| 93 | 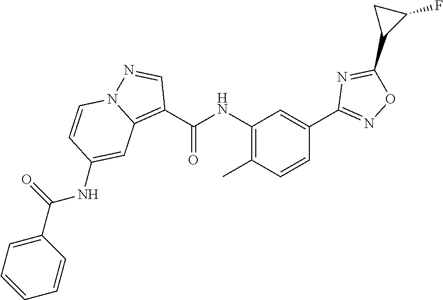 | (M + H)⁺: 497.17 |
| 94 | 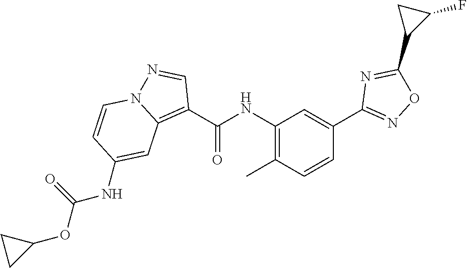 | (M + H)⁺: 477.22 |
| 95 | 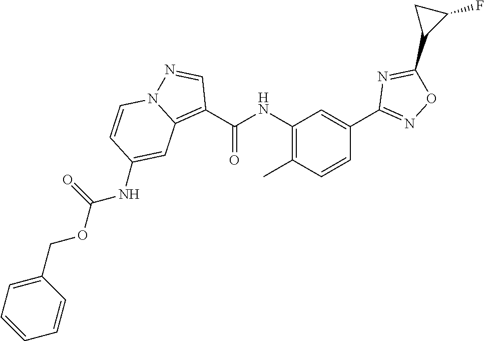 | (M + H)⁺: 527.23 |
| 96 | 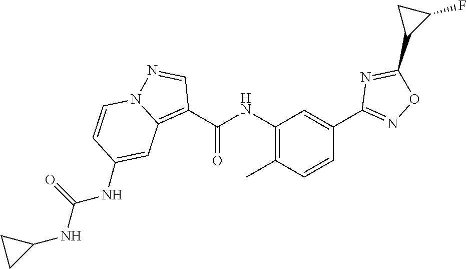 | (M + H)⁺: 476.29 |
| 97 | 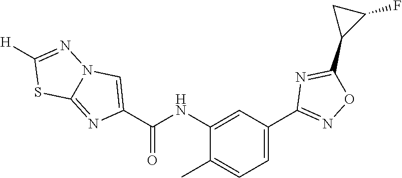 [SMILES: Cc1ccc(-c2noc([C@H]3C[C@@H]3F)n2)cc1NC(=O)c1cn2ncsc2n1] | (M + H)⁺: 385.10 |
| 98 | 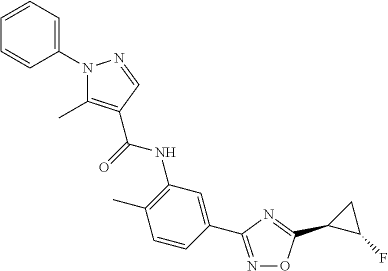 [SMILES: Cc1ccc(-c2noc([C@H]3C[C@@H]3F)n2)cc1NC(=O)c1cnn(-c2ccccc2)c1C] | (M + H)⁺: 418.15 |
| 98-1 | 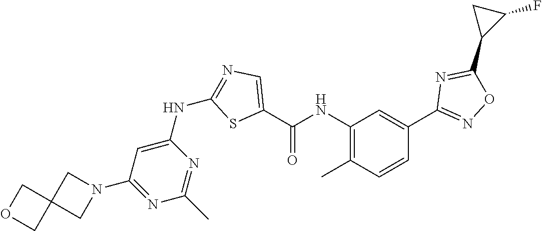 [SMILES: Cc1nc(Nc2ncc(C(=O)Nc3cc(-c4noc([C@H]5C[C@@H]5F)n4)ccc3C)s2)cc(N2CC3(COC3)C2)n1] | (M + H)⁺: 549.37 |
| 98-2 | 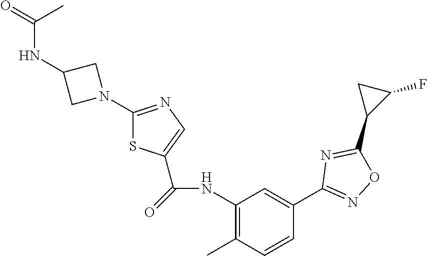 [SMILES: CC(=O)NC1CN(c2ncc(C(=O)Nc3cc(-c4noc([C@H]5C[C@@H]5F)n4)ccc3C)s2)C1] | (M + H)⁺: 457.47 |
| 98-3 | 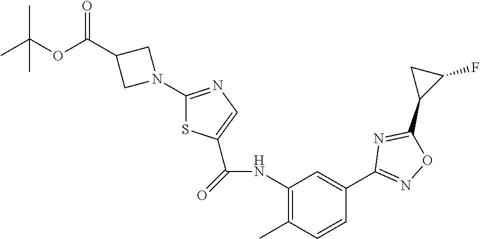 [SMILES: Cc1ccc(-c2noc([C@H]3C[C@@H]3F)n2)cc1NC(=O)c1cnc(N2CC(C(=O)OC(C)(C)C)C2)s1] | (M + H)⁺: 500.43 |
| 98-4 | 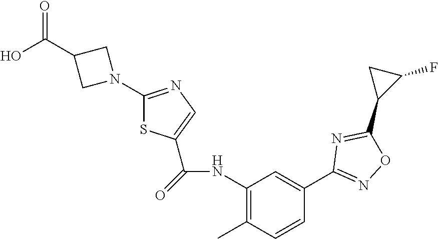 [SMILES: Cc1ccc(-c2noc([C@H]3C[C@@H]3F)n2)cc1NC(=O)c1cnc(N2CC(C(=O)O)C2)s1] | (M + H)⁺: 443.45 |
| 98-5 | 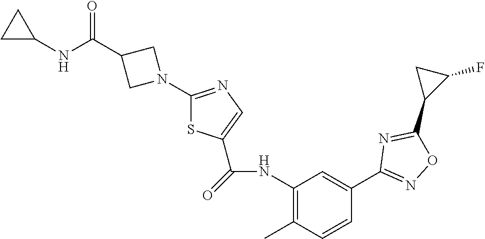 [SMILES: Cc1ccc(-c2noc([C@H]3C[C@@H]3F)n2)cc1NC(=O)c1cnc(N2CC(C(=O)NC3CC3)C2)s1] | (M + H)⁺: 482.87 |
| 98-6 | 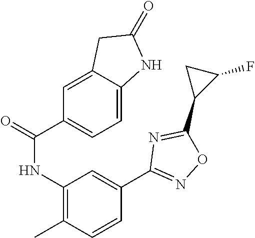 [SMILES: Cc1ccc(-c2noc([C@H]3C[C@@H]3F)n2)cc1NC(=O)c1ccc2c(c1)CC(=O)N2] | (M + H)⁺: 393.066 |
| 98-7 | 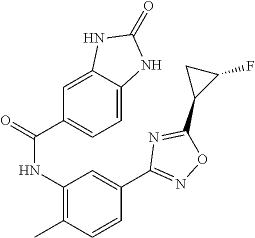 [SMILES: Cc1ccc(-c2noc([C@H]3C[C@@H]3F)n2)cc1NC(=O)c1ccc2[nH]c(=O)[nH]c2c1] | (M + H)⁺: 394.074 |
| 98-8 | 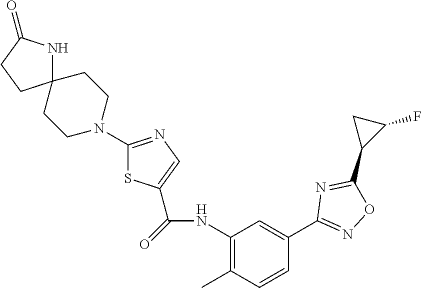 [SMILES: Cc1ccc(-c2noc([C@H]3C[C@@H]3F)n2)cc1NC(=O)c1cnc(N2CCC3(CCC(=O)N3)CC2)s1] | (M + H)⁺: 497.31 |
| 98-9 | 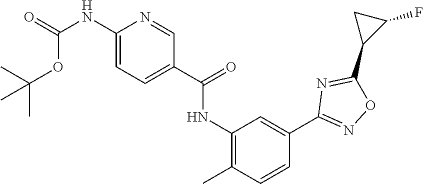 [SMILES: Cc1ccc(-c2noc([C@H]3C[C@@H]3F)n2)cc1NC(=O)c1ccc(NC(=O)OC(C)(C)C)nc1] | (M + H)⁺: 454.218 |

### Example 99

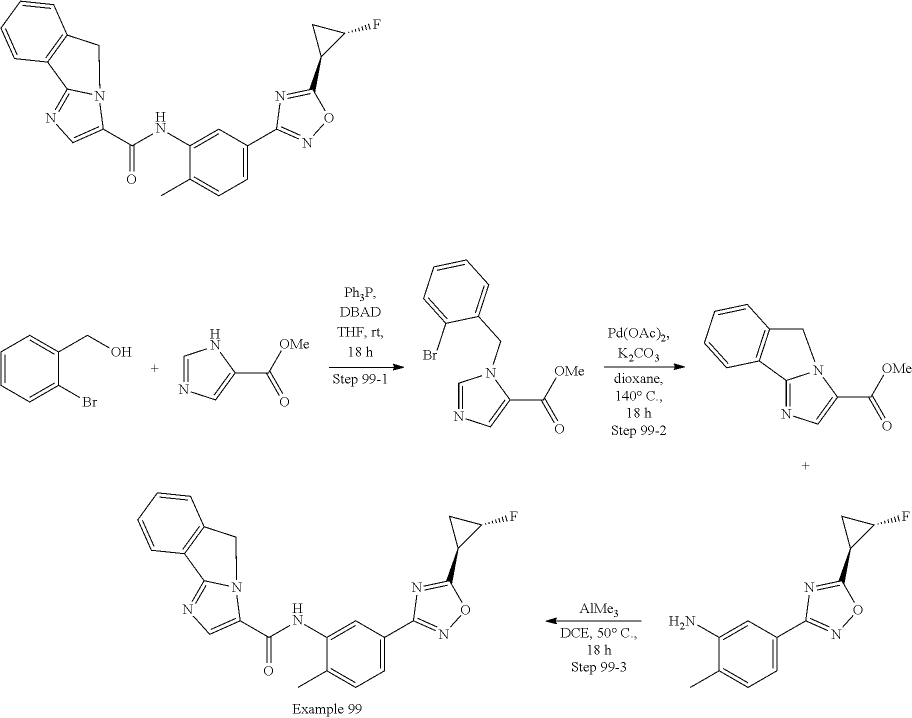

Step 99-1: To a solution of o-bromobenzyl alcohol (1.50 g, 8.0 mmol), Methyl 3H-imidazole-4-carboxylate (1.0 g, 8.0 mmol) and triphenylphosphine (2.73 g, 10.4 mmol) in anhydrous THF (20 mL) at −40° C. was added di-tert-butyl azodicarboxylate (2.49 g, 10.8 mmol) in anhydrous THF (15 mL) dropwise. After being stirred at −40° C. for 30 minutes, the mixture was warmed up to room temperature and stirred overnight. The mixture was evaporated to dryness and purified by flash chromatography to provide the desired product (1.20 g, 51%). ESI-MS (M+H)³⁰: 295.00, 297.00.
Step 99-2: A mixture of potassium carbonate (187 mg, 1.36 mmol), palladium(II) acetate (11.4 mg, 0.051 mmol), Di-tert-butyl(methyl)phosphonium tetrafluoroborate (25.2 mg, 0.102 mmol) and the compound from Step 99-1 (200 mg, 0.678 mmol) was in dioxane (4 mL) under N₂ in a sealed tube was stirred at 140° C. for 18 h. It was cooled to rt, diluted with EtOAc, filtered through celite, conc. purified by flash chromatography to give the desired product (52 mg, 35.8% yield). ESI-MS (M+H)⁺: 215.08.
Step 99-3: A mixture of the compound from Step 99-2 (52 mg, 0.243 mmol), 5-(5-((1R,2S)-2-fluorocyclopropyl)-1,2,4-oxadiazol-3-yl)-2-methylaniline (63 mg, 0.267 mmol) and trimethylaluminum (0.37 mL 2M solution in toluene, 0.73 mmol) in DCE (1 mL) was stirred at 50° C. for 18 h. It was cooled to rt, diluted with DCM, quenched with water and a small amount of 1N HCl, filtered through celite, the filtrate was separated, and the organic layer was concentrated. The crude mixture was purified by flash chromatography on silica gel to give Example 99 (52 mg, 52%) as a white solid. ESI-MS (M+H)³⁰: 416.52.

The following examples were prepared using procedures similar to those described above:

| Example | Structure | ESI-MS |
|---|---|---|
| 100 | 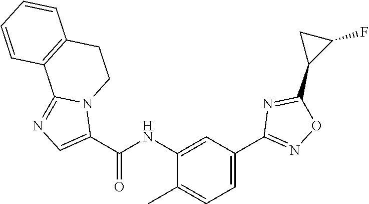 [SMILES: Cc1ccc(-c2noc([C@H]3C[C@@H]3F)n2)cc1NC(=O)c1cnc2n1CCc1ccccc1-2] | (M + H)⁺: 430.60 |
| 101 | 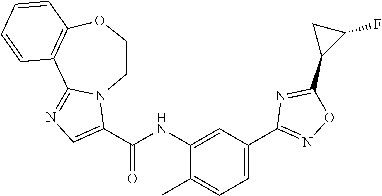 [SMILES: Cc1ccc(-c2noc([C@H]3C[C@@H]3F)n2)cc1NC(=O)c1cnc2n1CCOc1ccccc1-2] | (M + H)⁺: 446.53 |
| 102 | 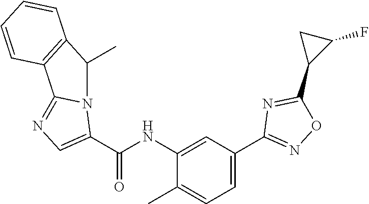 [SMILES: Cc1ccc(-c2noc([C@H]3C[C@@H]3F)n2)cc1NC(=O)c1cnc2n1C(C)c1ccccc1-2] | (M + H)⁺: 430.60 |
| 103 | 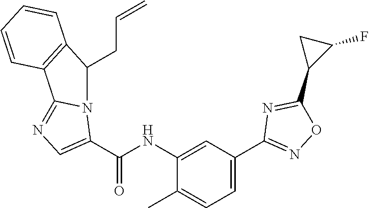 [SMILES: C=CCC1c2ccccc2-c2ncc(C(=O)Nc3cc(-c4noc([C@H]5C[C@@H]5F)n4)ccc3C)n21] | (M + H)⁺: 456.57 |
| 104 | 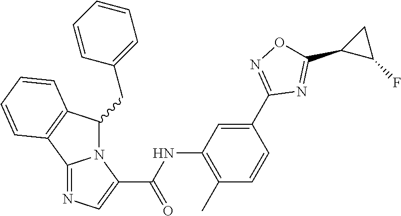 [SMILES: Cc1ccc(-c2noc([C@H]3C[C@@H]3F)n2)cc1NC(=O)c1cnc2n1C(Cc1ccccc1)c1ccccc1-2] | (M + H)⁺: 506.54 |
| 105 | 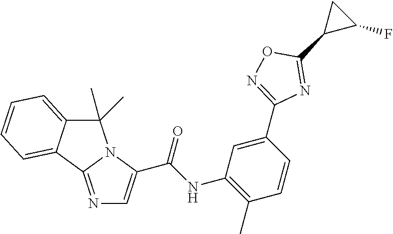 [SMILES: Cc1ccc(-c2noc([C@H]3C[C@@H]3F)n2)cc1NC(=O)c1cnc2n1C(C)(C)c1ccccc1-2] | (M + H)⁺: 444.51 |

### Example 106 & Example 107

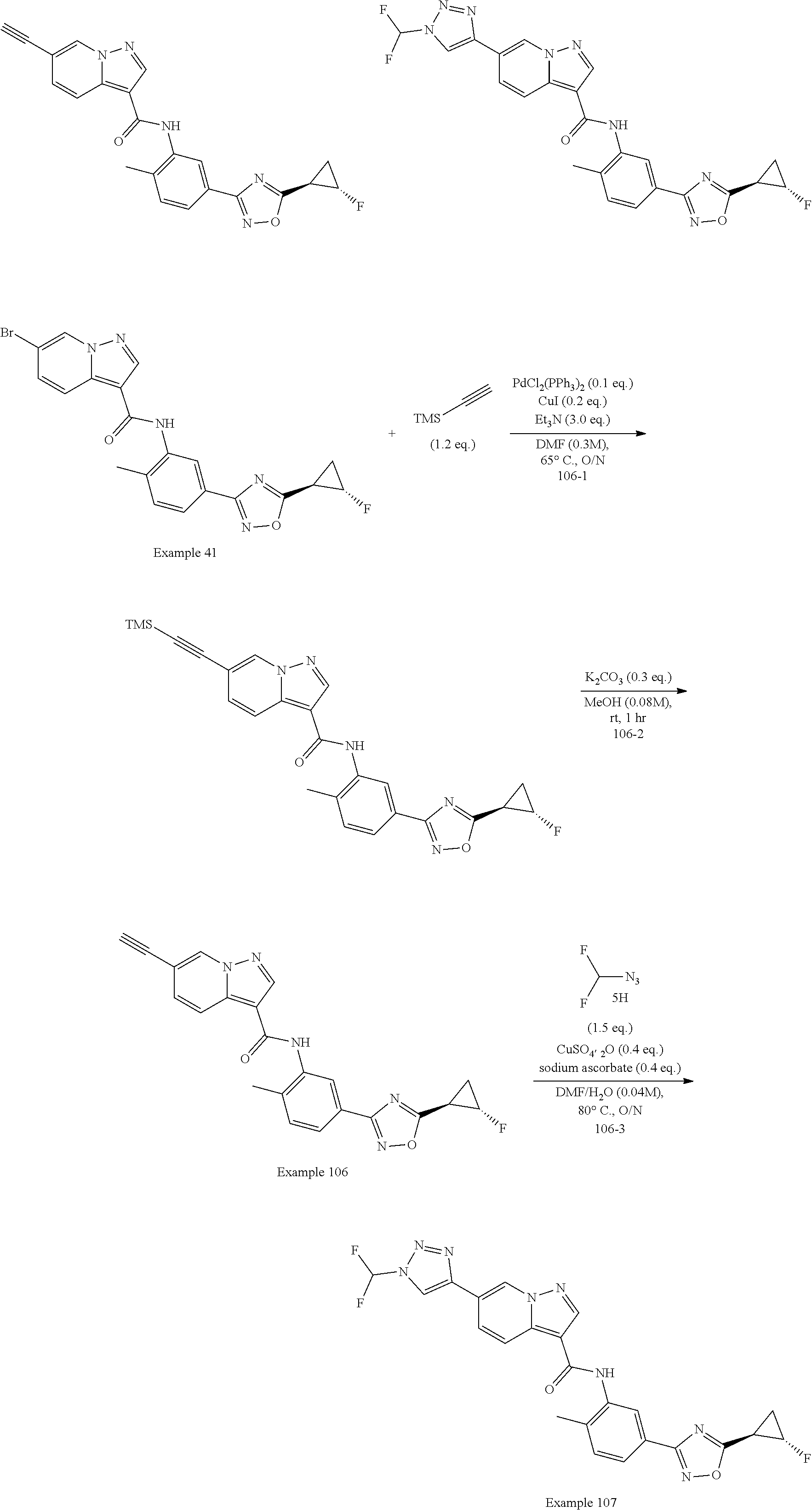

Step 106-1: Example 41: 6-bromo-N-(5-(5-((1R,2S)-2-fluorocyclopropyl)-1,2,4-oxadiazol-3-yl)-2-methylphenyl)pyrazolo [1,5-a]pyridine-3-carboxamide (500 mg, 1.1 mmol, 1.0 eq.), CuI (42 mg, 0.22 mmol, 0.2 eq.), PdCl₂(PPh₃)₂ (77 mg, 0.11 mmol, 0.1 eq.) was loaded in a 20 mL vial equipped with a magnetic stirbar. The vial was sealed, and then evacuated and refilled with nitrogen (3 times). DMF (4 mL) was added through a syringe, followed by Et₃N (0.46 mL, 3.3 mmol, 3.0 eq.) and ethynyltrimethylsilane (0.19 mL, 1.3 mmol, 1.2 eq.). The mixture was heated at 65° C. overnight. After the reaction was complete, water (10 mL) was added, and the mixture was extracted with ethyl acetate (20 mL*3). The combined organic layers were dried over anhydrous sodium sulfate, and concentrated in vacuo. The crude was purified using silica gel column chromatography (0-50% EtOAc in cHex) to afford the desired product (390 mg, 75%) as a colorless oil.
Step 106-2: To a solution of the compound from Step 106-1 (390 mg, 0.82 mmol, 1.0 eq.) in methanol (10 mL) was added K₂CO₃ (34 mg, 0.25 mmol, 0.3 eq.). The mixture was stirred at room temperature over 1 hour. After the reaction was complete, the mixture was concentrated in vacuo, and the crude was purified using silica gel column chromatography (0-50% EtOAc in cHex) to afford Example 106 (260 mg, 79%) as a white solid. LC-MS, ES+: 402.19 [M+H]⁺.
Step 106-3: Example 106 (20 mg, 0.05 mmol, 1.0 eq.), copper sulfate pentahydrate (5.0 mg, 0.02 mmol, 0.4 eq.) and sodium ascorbate (3.9 mg, 0.02 mmol, 0.4 eq.) was loaded in a 20 mL vial equipped with a magnetic stirbar. DMF (0.8 mL) and water (0.4 mL) was added, followed by azidodifluoromethane (0.15 mL, 1.5 eq., 0.5 M solution in DIVE). The mixture was heated at 80° C. overnight. After the reaction was complete, the mixture was filtered through celite, and purified over preparative TIPLC to afford Example 107 (11.2 mg, 450%) as a white solid. LC-MS, ES+: 495.11 [M+H]⁺.

The following examples were prepared using procedures similar to those described above:

| Example | Structure | ESI-MS |
|---|---|---|
| 108 | 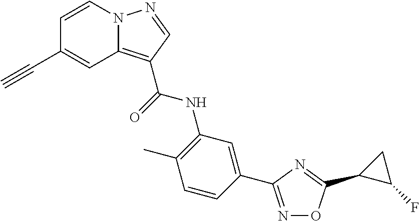 [SMILES: C#Cc1ccn2ncc(C(=O)Nc3cc(-c4noc([C@H]5C[C@@H]5F)n4)ccc3C)c2c1] | (M + H)⁺: 402.24 |
| 109 | 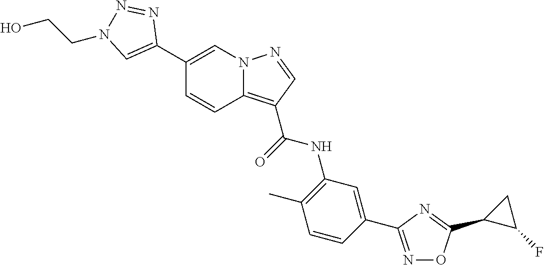 [SMILES: Cc1ccc(-c2noc([C@H]3C[C@@H]3F)n2)cc1NC(=O)c1cnn2cc(-c3cn(CCO)nn3)ccc12] | (M + H)⁺: 489.11 |
| 110 | 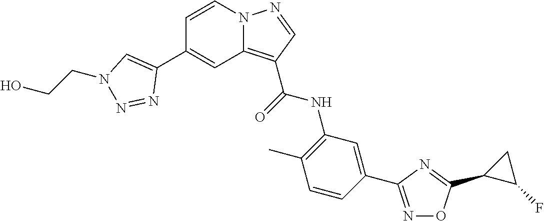 [SMILES: Cc1ccc(-c2noc([C@H]3C[C@@H]3F)n2)cc1NC(=O)c1cnn2ccc(-c3cn(CCO)nn3)cc12] | (M + H)⁺: 489.11 |
| 111 | 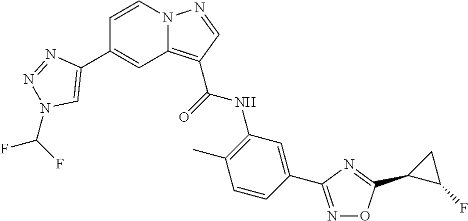 [SMILES: Cc1ccc(-c2noc([C@H]3C[C@@H]3F)n2)cc1NC(=O)c1cnn2ccc(-c3cn(C(F)F)nn3)cc12] | (M + H)⁺: 495.15 |
| 112 | 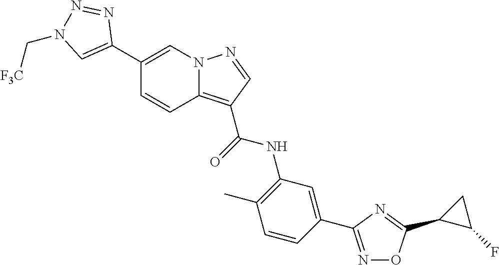 [SMILES: Cc1ccc(-c2noc([C@H]3C[C@@H]3F)n2)cc1NC(=O)c1cnn2cc(-c3cn(CC(F)(F)F)nn3)ccc12] | (M + H)⁺: 527.16 |
| 113 | 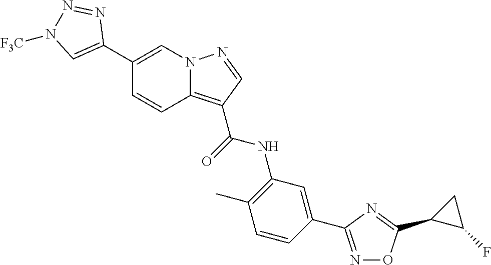 [SMILES: Cc1ccc(-c2noc([C@H]3C[C@@H]3F)n2)cc1NC(=O)c1cnn2cc(-c3cn(C(F)(F)F)nn3)ccc12] | (M + H)⁺: 513.10 |
| 114 | 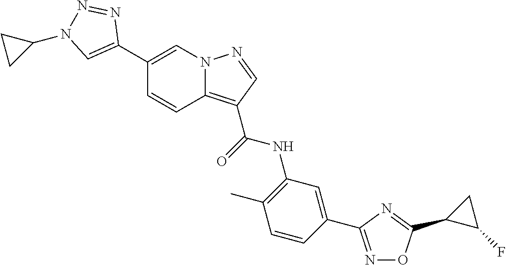 [SMILES: Cc1ccc(-c2noc([C@H]3C[C@@H]3F)n2)cc1NC(=O)c1cnn2cc(-c3cn(C4CC4)nn3)ccc12] | (M + H)⁺: 485.17 |
| 115 | 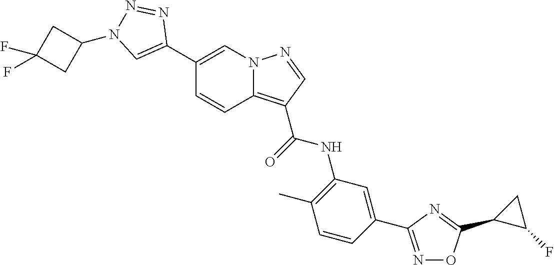 [SMILES: Cc1ccc(-c2noc([C@H]3C[C@@H]3F)n2)cc1NC(=O)c1cnn2cc(-c3cn(C4CC(F)(F)C4)nn3)ccc12] | (M + H)⁺: 535.14 |
| 116 | 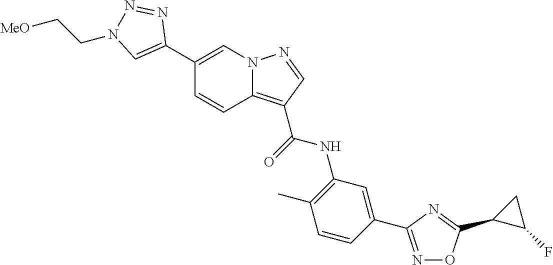 [SMILES: COCCn1cc(-c2ccc3c(C(=O)Nc4cc(-c5noc([C@H]6C[C@@H]6F)n5)ccc4C)cnn3c2)nn1] | (M + H)⁺: 503.12 |
| 117 | 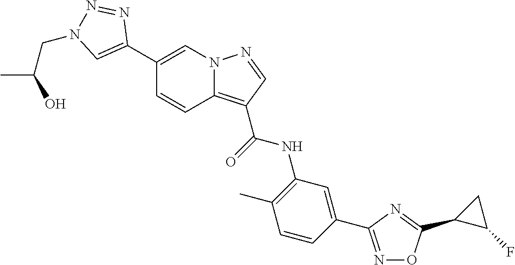 [SMILES: Cc1ccc(-c2noc([C@H]3C[C@@H]3F)n2)cc1NC(=O)c1cnn2cc(-c3cn(C[C@H](C)O)nn3)ccc12] | (M + H)⁺: 503.12 |
| 118 | 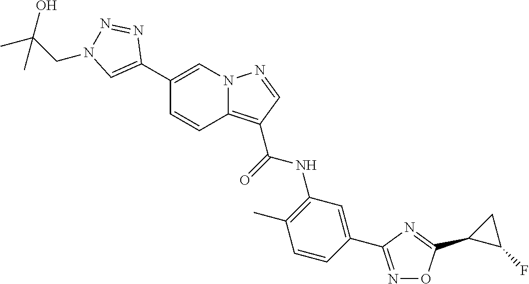 [SMILES: Cc1ccc(-c2noc([C@H]3C[C@@H]3F)n2)cc1NC(=O)c1cnn2cc(-c3cn(CC(C)(C)O)nn3)ccc12] | (M + H)⁺: 517.14 |
| 119 | 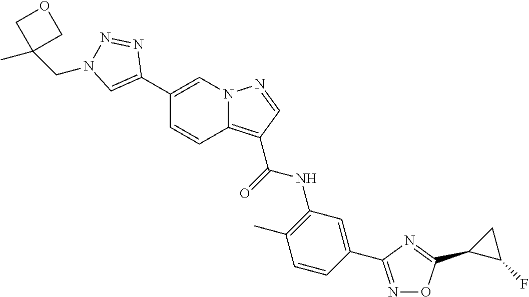 [SMILES: Cc1ccc(-c2noc([C@H]3C[C@@H]3F)n2)cc1NC(=O)c1cnn2cc(-c3cn(CC4(C)COC4)nn3)ccc12] | (M + H)⁺: 529.09 |
| 120 | 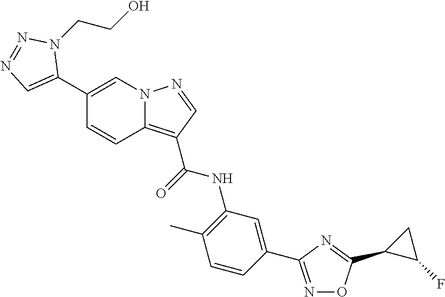 [SMILES: Cc1ccc(-c2noc([C@H]3C[C@@H]3F)n2)cc1NC(=O)c1cnn2cc(-c3cnnn3CCO)ccc12] | (M + H)⁺: 489.06 |
| 121 | 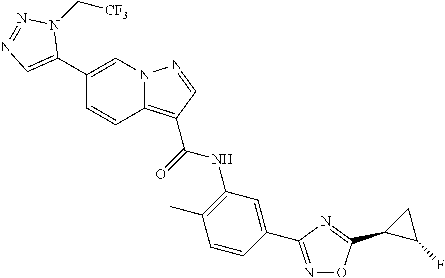 [SMILES: Cc1ccc(-c2noc([C@H]3C[C@@H]3F)n2)cc1NC(=O)c1cnn2cc(-c3cnnn3CC(F)(F)F)ccc12] | (M + H)⁺: 527.12 |
| 122 | 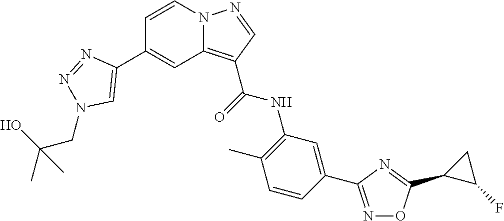 [SMILES: Cc1ccc(-c2noc([C@H]3C[C@@H]3F)n2)cc1NC(=O)c1cnn2ccc(-c3cn(CC(C)(C)O)nn3)cc12] | (M + H)⁺: 517.1 |
| 123 | 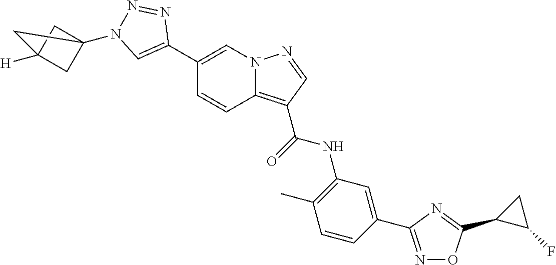 [SMILES: Cc1ccc(-c2noc([C@H]3C[C@@H]3F)n2)cc1NC(=O)c1cnn2cc(-c3cn(C45CC(C4)C5)nn3)ccc12] | (M + H)⁺: 511.0 |
| 124 | 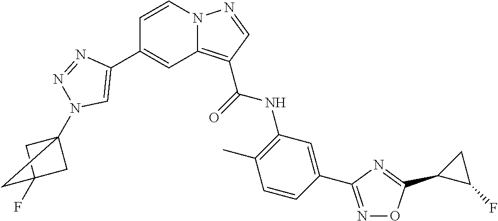 [SMILES: Cc1ccc(-c2noc([C@H]3C[C@@H]3F)n2)cc1NC(=O)c1cnn2ccc(-c3cn(C45CC(F)(C4)C5)nn3)cc12] | (M + H)⁺: 529.1 |
| 125 | 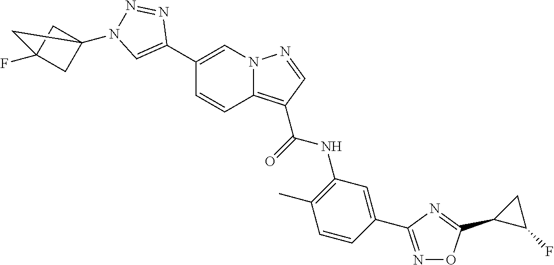 [SMILES: Cc1ccc(-c2noc([C@H]3C[C@@H]3F)n2)cc1NC(=O)c1cnn2cc(-c3cn(C45CC(F)(C4)C5)nn3)ccc12] | (M + H)⁺: 529.1 |
| 126 | 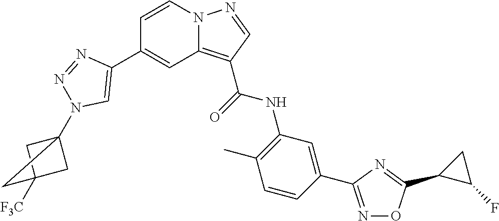 [SMILES: Cc1ccc(-c2noc([C@H]3C[C@@H]3F)n2)cc1NC(=O)c1cnn2ccc(-c3cn(C45CC(C(F)(F)F)(C4)C5)nn3)cc12] | (M + H)⁺: 579.1 |
| 127 | 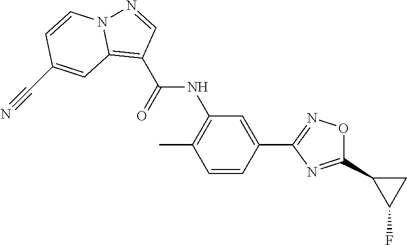 [SMILES: Cc1ccc(-c2noc([C@H]3C[C@@H]3F)n2)cc1NC(=O)c1cnn2ccc(C#N)cc12] | (M + H)⁺: 403.26 |
| 128 | 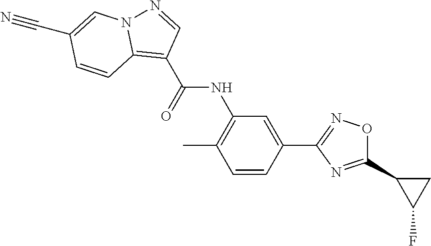 [SMILES: Cc1ccc(-c2noc([C@H]3C[C@@H]3F)n2)cc1NC(=O)c1cnn2cc(C#N)ccc12] | (M + H)⁺: 403.31 |
| 129 | 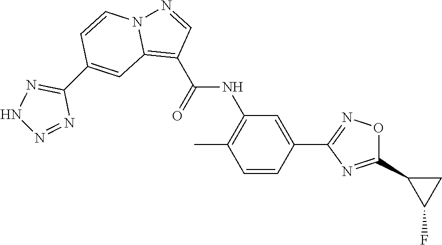 [SMILES: Cc1ccc(-c2noc([C@H]3C[C@@H]3F)n2)cc1NC(=O)c1cnn2ccc(-c3nn[nH]n3)cc12] | (M + H)⁺: 446.16 |
| 130 | 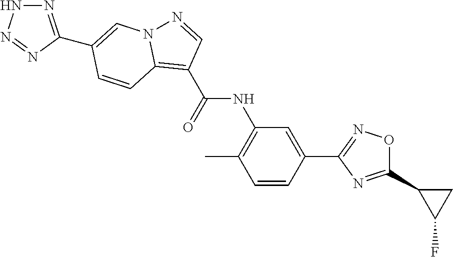 [SMILES: Cc1ccc(-c2noc([C@H]3C[C@@H]3F)n2)cc1NC(=O)c1cnn2cc(-c3nn[nH]n3)ccc12] | (M + H)⁺: 446.14 |
| 131 | 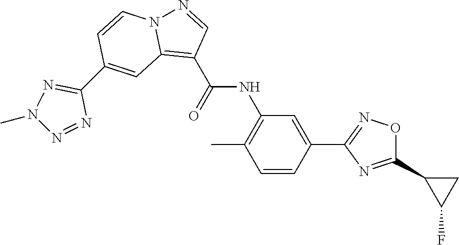 [SMILES: Cc1ccc(-c2noc([C@H]3C[C@@H]3F)n2)cc1NC(=O)c1cnn2ccc(-c3nnn(C)n3)cc12] | (M + H)⁺: 460.29 |
| 132 | 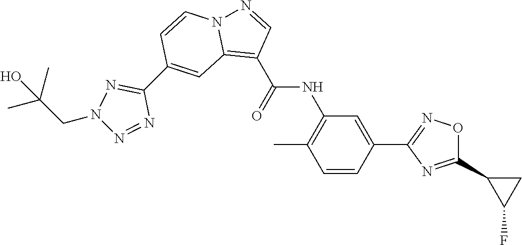 [SMILES: Cc1ccc(-c2noc([C@H]3C[C@@H]3F)n2)cc1NC(=O)c1cnn2ccc(-c3nnn(CC(C)(C)O)n3)cc12] | (M + H)⁺: 518.26 |
| 132-1 | 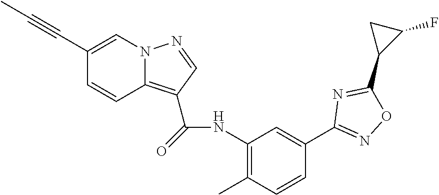 [SMILES: CC#Cc1ccc2c(C(=O)Nc3cc(-c4noc([C@H]5C[C@@H]5F)n4)ccc3C)cnn2c1] | (M + H)⁺: 416.1 |
| 132-2 | 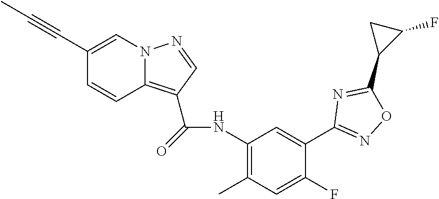 [SMILES: CC#Cc1ccc2c(C(=O)Nc3cc(-c4noc([C@H]5C[C@@H]5F)n4)c(F)cc3C)cnn2c1] | (M + H)⁺: 434.1 |
| 132-3 | 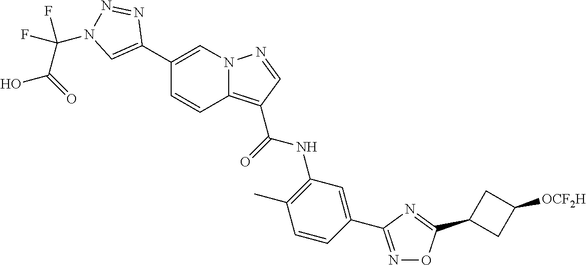 | (M + H)⁺: 601.09 |
| 132-4 | 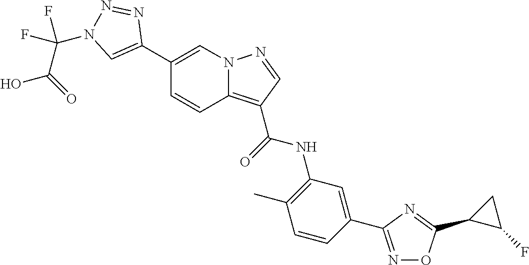 [SMILES: Cc1ccc(-c2noc([C@H]3C[C@@H]3F)n2)cc1NC(=O)c1cnn2cc(-c3cn(C(F)(F)C(=O)O)nn3)ccc12] | (M + H)⁺: 538.97 |
| 132-5 | 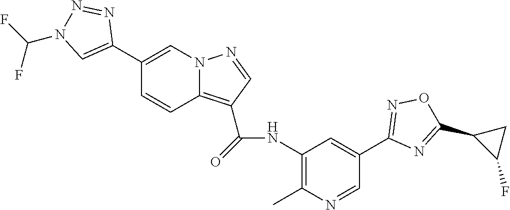 [SMILES: Cc1ncc(-c2noc([C@H]3C[C@@H]3F)n2)cc1NC(=O)c1cnn2cc(-c3cn(C(F)F)nn3)ccc12] | (M + H)⁺: 496.07 |
| 132-6 | 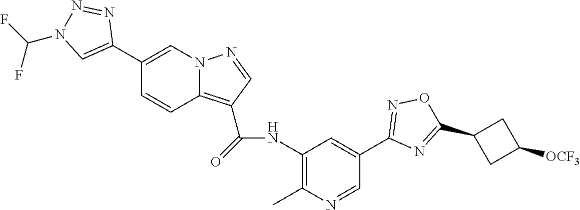 [SMILES: Cc1ncc(-c2noc([C@H]3C[C@@H](OC(F)(F)F)C3)n2)cc1NC(=O)c1cnn2cc(-c3cn(C(F)F)nn3)ccc12] | (M + H)⁺: 576.08 |
| 132-7 | 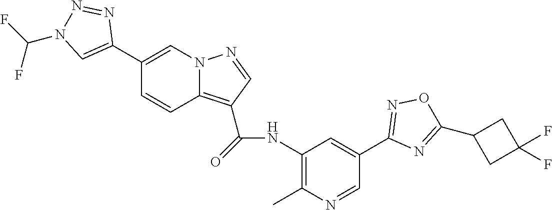 [SMILES: Cc1ncc(-c2noc(C3CC(F)(F)C3)n2)cc1NC(=O)c1cnn2cc(-c3cn(C(F)F)nn3)ccc12] | (M + H)⁺: 528.13 |
| 132-8 | 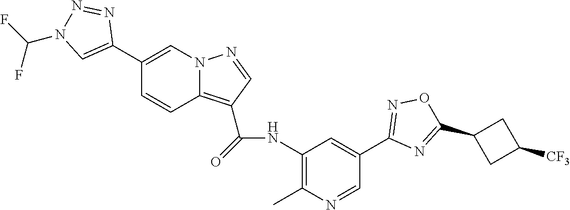 [SMILES: Cc1ncc(-c2noc([C@H]3C[C@@H](C(F)(F)F)C3)n2)cc1NC(=O)c1cnn2cc(-c3cn(C(F)F)nn3)ccc12] | (M + H)⁺: 560.14 |
| 132-9 | 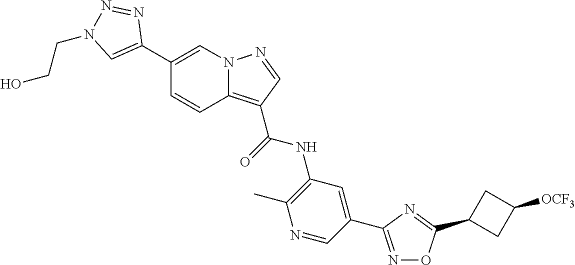 [SMILES: Cc1ncc(-c2noc([C@H]3C[C@@H](OC(F)(F)F)C3)n2)cc1NC(=O)c1cnn2cc(-c3cn(CCO)nn3)ccc12] | (M + H)⁺: 570.17 |
| 132-10 | 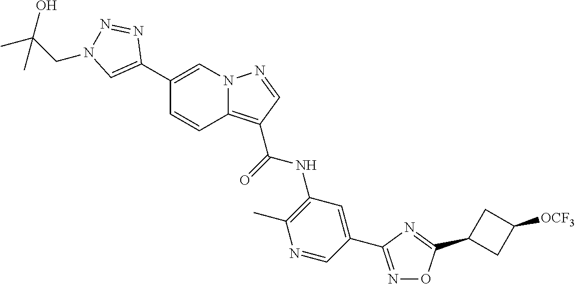 [SMILES: Cc1ncc(-c2noc([C@H]3C[C@@H](OC(F)(F)F)C3)n2)cc1NC(=O)c1cnn2cc(-c3cn(CC(C)(C)O)nn3)ccc12] | (M + H)⁺: 598.21 |
| 132-11 | 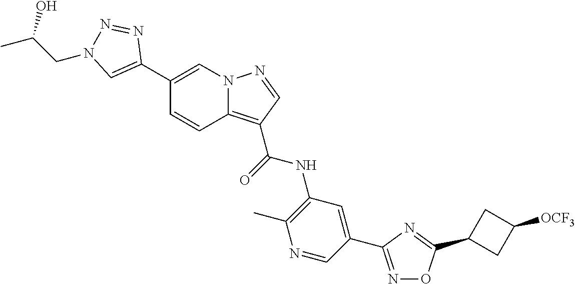 [SMILES: Cc1ncc(-c2noc([C@H]3C[C@@H](OC(F)(F)F)C3)n2)cc1NC(=O)c1cnn2cc(-c3cn(C[C@H](C)O)nn3)ccc12] | (M + H)⁺: 583.53 |
| 132-12 | 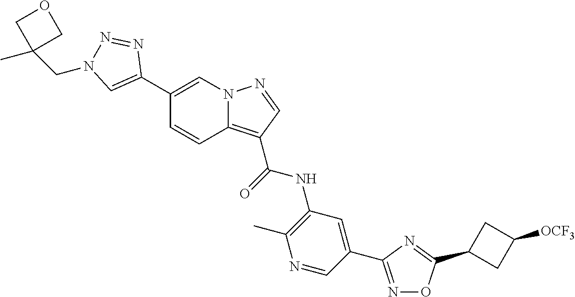 [SMILES: Cc1ncc(-c2noc([C@H]3C[C@@H](OC(F)(F)F)C3)n2)cc1NC(=O)c1cnn2cc(-c3cn(CC4(C)COC4)nn3)ccc12] | (M + H)⁺: 610.25 |
| 132-13 | 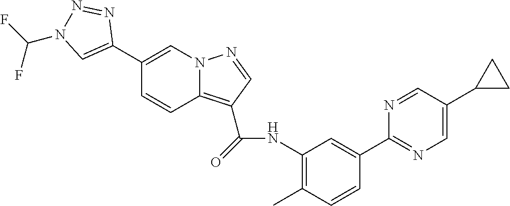 [SMILES: Cc1ccc(-c2ncc(C3CC3)cn2)cc1NC(=O)c1cnn2cc(-c3cn(C(F)F)nn3)ccc12] | (M + H)⁺: 487.28 |
| 132-14 | 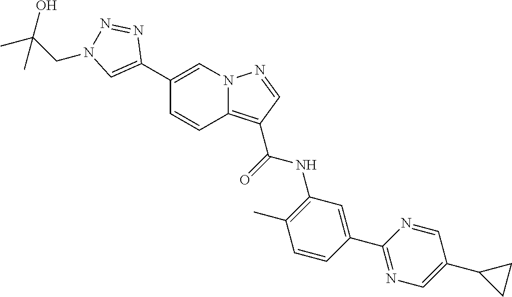 [SMILES: Cc1ccc(-c2ncc(C3CC3)cn2)cc1NC(=O)c1cnn2cc(-c3cn(CC(C)(C)O)nn3)ccc12] | (M + H)⁺: 509.41 |
| 132-15 | 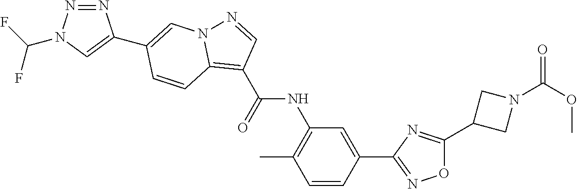 [SMILES: COC(=O)N1CC(c2nc(-c3ccc(C)c(NC(=O)c4cnn5cc(-c6cn(C(F)F)nn6)ccc45)c3)no2)C1] | (M + H)⁺: 550.15 |
| 132-16 | 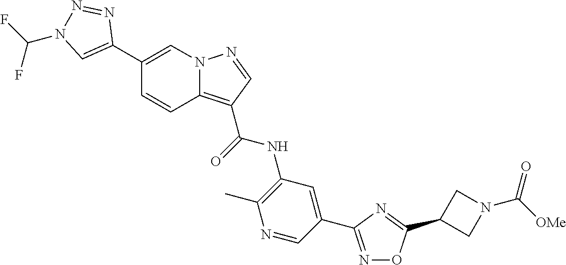 | (M + H)⁺: 551.15 |
| 132-17 | 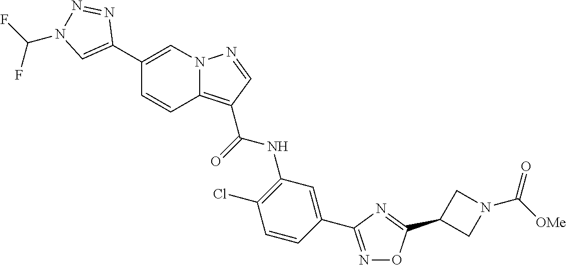 | (M + H)⁺: 570.10 |

### Example 2a

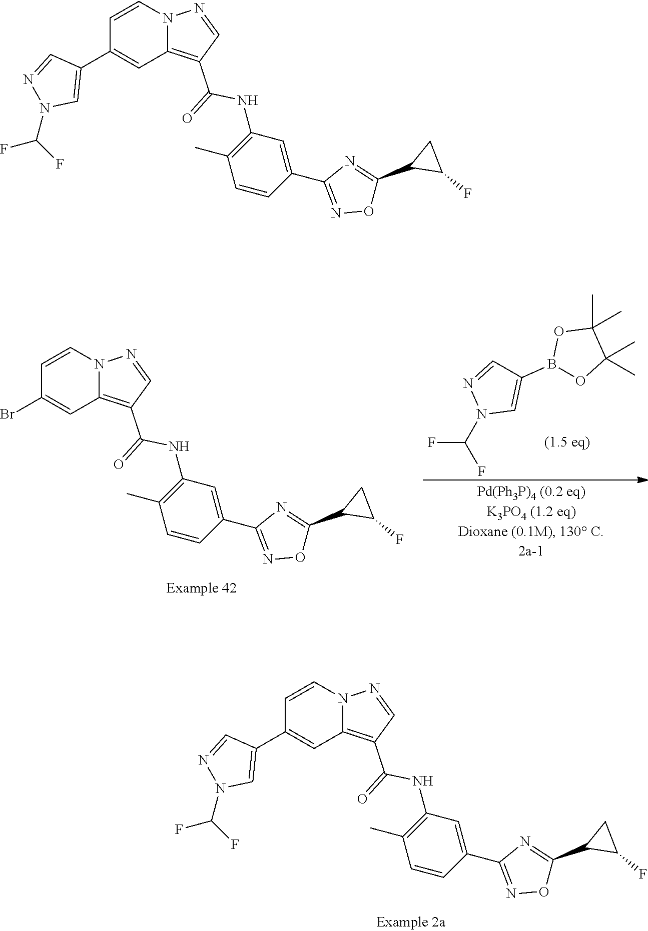

Step 2a-1: To the vial were added potassium phosphate (15.07 mg, 1.2 Eq, 71 mol), Pd(Ph₃P)₄ (13.68 mg, 0.2 Eq, 12 mol), 1-(difluoromethyl)-4-(4,4,5,5-tetramethyl-1,3,2-dioxaborolan-2-yl)-1H-pyrazolel (21.66 mg, 1.5 Eq, 89 mol) and Example 42: 5-bromo-6-fluoro-N-(5-(5-((1S,2S)-2-fluorocyclopropyl)-1,2,4-oxadiazol-3-yl)-2-methylphenyl)pyrazolo[1,5-a]pyridine-3-carboxamide (27 mg, 1 Eq, 59 mol). The vial was then purged with nitrogen 3 times before 1,4-Dioxane (592 μL) was added. The reaction mixture was then heated to 130° C. for 4 hours. The reaction mixture was diluted with DMSO (2.5 mL) and the crude material was subjected to TIPLC to isolate the desired product Example 2a (18 mg, 35 μmol, 58.9%) as a white solid. LC-MS, ES+: 494.298 [M+H]⁺.

The following examples were prepared using procedures similar to those described above:

| Example | Structure | ESI-MS |
|---|---|---|
| 133 | 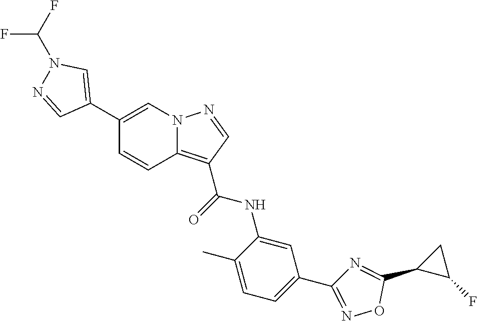 [SMILES: Cc1ccc(-c2noc([C@H]3C[C@@H]3F)n2)cc1NC(=O)c1cnn2cc(-c3cnn(C(F)F)c3)ccc12] | (M + H)⁺: 494.250 |
| 134 | 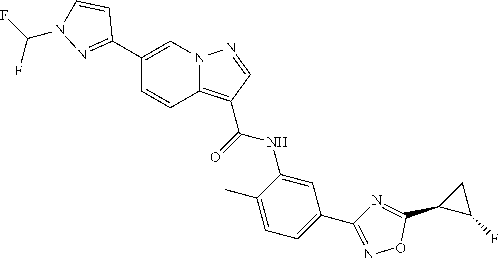 [SMILES: Cc1ccc(-c2noc([C@H]3C[C@@H]3F)n2)cc1NC(=O)c1cnn2cc(-c3ccn(C(F)F)n3)ccc12] | (M + H)⁺: 494.250 |
| 135 | 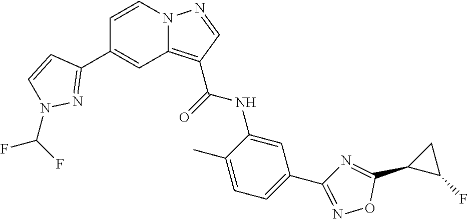 [SMILES: Cc1ccc(-c2noc([C@H]3C[C@@H]3F)n2)cc1NC(=O)c1cnn2ccc(-c3ccn(C(F)F)n3)cc12] | (M + H)⁺: 494.250 |
| 136 | 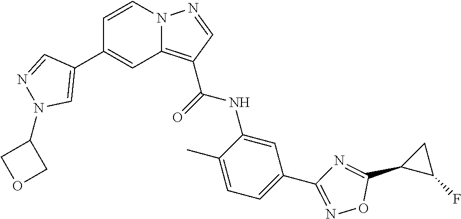 [SMILES: Cc1ccc(-c2noc([C@H]3C[C@@H]3F)n2)cc1NC(=O)c1cnn2ccc(-c3cnn(C4COC4)c3)cc12] | (M + H)⁺: 500.394 |
| 137 | 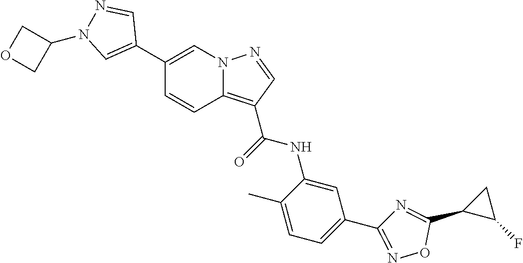 [SMILES: Cc1ccc(-c2noc([C@H]3C[C@@H]3F)n2)cc1NC(=O)c1cnn2cc(-c3cnn(C4COC4)c3)ccc12] | (M + H)⁺: 500.394 |
| 138 | 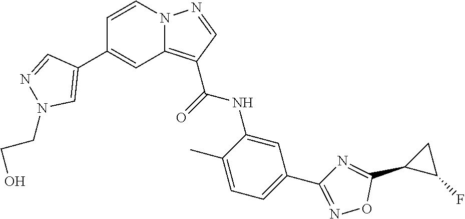 [SMILES: Cc1ccc(-c2noc([C@H]3C[C@@H]3F)n2)cc1NC(=O)c1cnn2ccc(-c3cnn(CCO)c3)cc12] | (M + H)⁺: 488.394 |
| 139 | 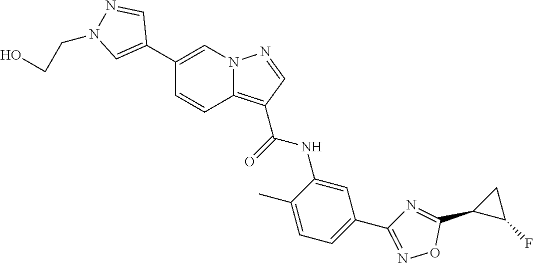 [SMILES: Cc1ccc(-c2noc([C@H]3C[C@@H]3F)n2)cc1NC(=O)c1cnn2cc(-c3cnn(CCO)c3)ccc12] | (M + H)⁺: 488.442 |
| 140 | 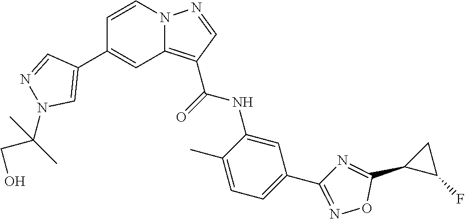 [SMILES: Cc1ccc(-c2noc([C@H]3C[C@@H]3F)n2)cc1NC(=O)c1cnn2ccc(-c3cnn(C(C)(C)CO)c3)cc12] | (M + H)⁺: 516.475 |
| 141 | 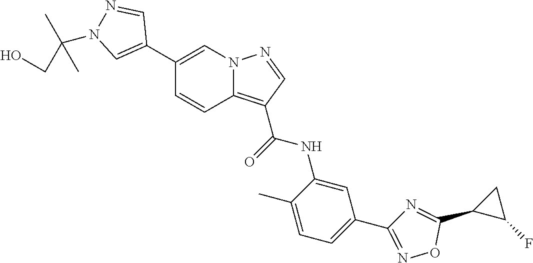 [SMILES: Cc1ccc(-c2noc([C@H]3C[C@@H]3F)n2)cc1NC(=O)c1cnn2cc(-c3cnn(C(C)(C)CO)c3)ccc12] | (M + H)⁺: 516.523 |
| 142 | 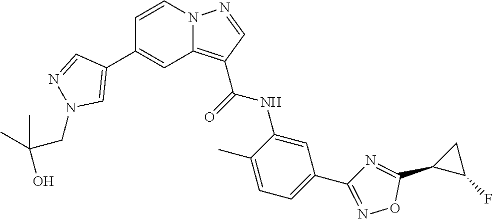 [SMILES: Cc1ccc(-c2noc([C@H]3C[C@@H]3F)n2)cc1NC(=O)c1cnn2ccc(-c3cnn(CC(C)(C)O)c3)cc12] | (M + H)⁺: 516.475 |
| 143 | 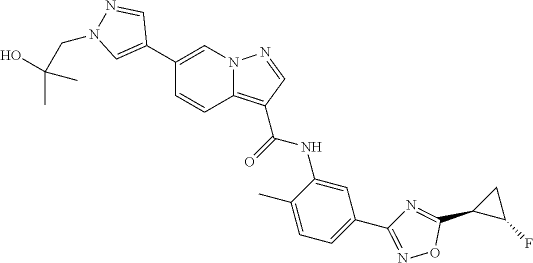 [SMILES: Cc1ccc(-c2noc([C@H]3C[C@@H]3F)n2)cc1NC(=O)c1cnn2cc(-c3cnn(CC(C)(C)O)c3)ccc12] | (M + H)⁺: 516.427 |
| 144 | 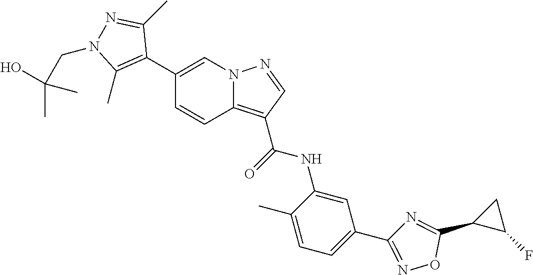 [SMILES: Cc1ccc(-c2noc([C@H]3C[C@@H]3F)n2)cc1NC(=O)c1cnn2cc(-c3c(C)nn(CC(C)(C)O)c3C)ccc12] | (M + H)⁺: 544.603 |
| 145 | 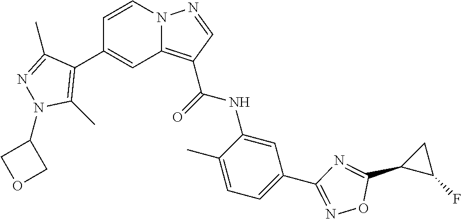 [SMILES: Cc1ccc(-c2noc([C@H]3C[C@@H]3F)n2)cc1NC(=O)c1cnn2ccc(-c3c(C)nn(C4COC4)c3C)cc12] | (M + H)⁺: 528.571 |
| 146 | 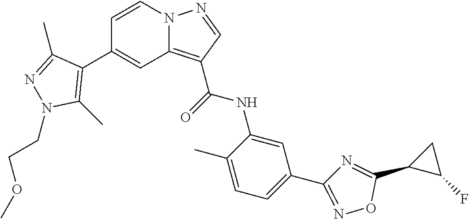 [SMILES: COCCn1nc(C)c(-c2ccn3ncc(C(=O)Nc4cc(-c5noc([C@H]6C[C@@H]6F)n5)ccc4C)c3c2)c1C] | (M + H)⁺: 530.443 |
| 147 | 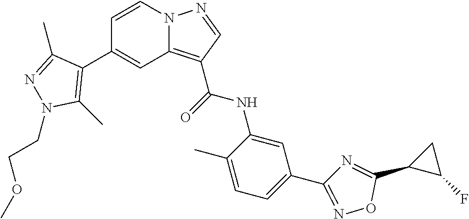 [SMILES: COCCn1nc(C)c(-c2ccn3ncc(C(=O)Nc4cc(-c5noc([C@H]6C[C@@H]6F)n5)ccc4C)c3c2)c1C] | (M + H)⁺: 530.443 |
| 148 | 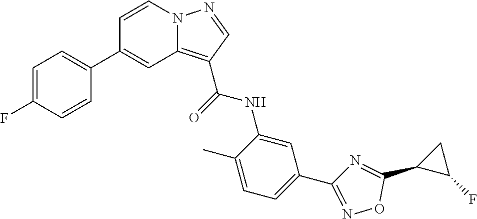 [SMILES: Cc1ccc(-c2noc([C@H]3C[C@@H]3F)n2)cc1NC(=O)c1cnn2ccc(-c3ccc(F)cc3)cc12] | (M + H)⁺: 472.362 |
| 149 | 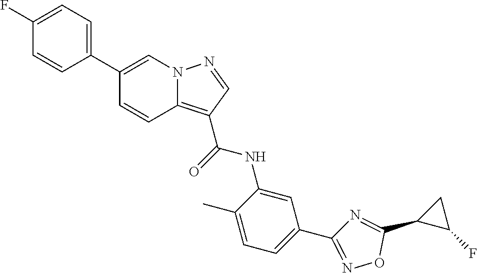 [SMILES: Cc1ccc(-c2noc([C@H]3C[C@@H]3F)n2)cc1NC(=O)c1cnn2cc(-c3ccc(F)cc3)ccc12] | (M + H)⁺: 472.362 |
| 150 | 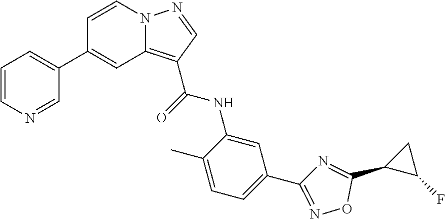 [SMILES: Cc1ccc(-c2noc([C@H]3C[C@@H]3F)n2)cc1NC(=O)c1cnn2ccc(-c3cccnc3)cc12] | (M + H)⁺: 455.370 |
| 151 | 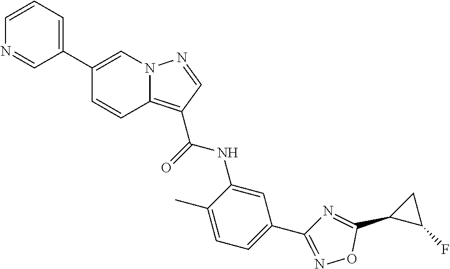 [SMILES: Cc1ccc(-c2noc([C@H]3C[C@@H]3F)n2)cc1NC(=O)c1cnn2cc(-c3cccnc3)ccc12] | (M + H)⁺: 455.514 |
| 152 | 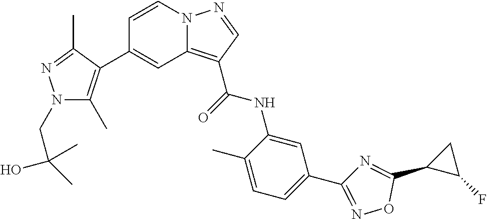 [SMILES: Cc1ccc(-c2noc([C@H]3C[C@@H]3F)n2)cc1NC(=O)c1cnn2ccc(-c3c(C)nn(CC(C)(C)O)c3C)cc12] | (M + H)⁺: 544.555 |
| 153 | 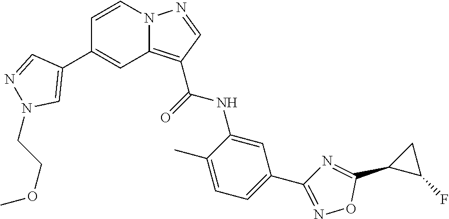 [SMILES: COCCn1cc(-c2ccn3ncc(C(=O)Nc4cc(-c5noc([C@H]6C[C@@H]6F)n5)ccc4C)c3c2)cn1] | (M + H)⁺: 502.362 |
| 154 | 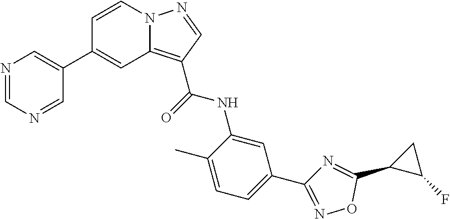 [SMILES: Cc1ccc(-c2noc([C@H]3C[C@@H]3F)n2)cc1NC(=O)c1cnn2ccc(-c3cncnc3)cc12] | (M + H)⁺: 456.282 |
| 155 | 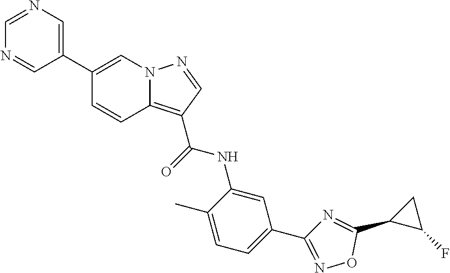 [SMILES: Cc1ccc(-c2noc([C@H]3C[C@@H]3F)n2)cc1NC(=O)c1cnn2cc(-c3cncnc3)ccc12] | (M + H)⁺: 456.186 |
| 156 | 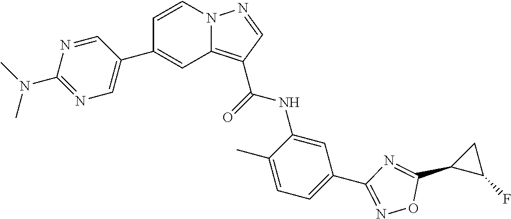 [SMILES: Cc1ccc(-c2noc([C@H]3C[C@@H]3F)n2)cc1NC(=O)c1cnn2ccc(-c3cnc(N(C)C)nc3)cc12] | (M + H)⁺: 499.202 |
| 157 | 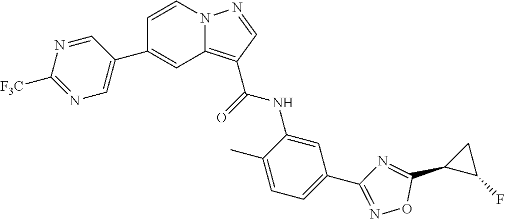 [SMILES: Cc1ccc(-c2noc([C@H]3C[C@@H]3F)n2)cc1NC(=O)c1cnn2ccc(-c3cnc(C(F)(F)F)nc3)cc12] | (M + H)⁺: 524.144 |
| 158 | 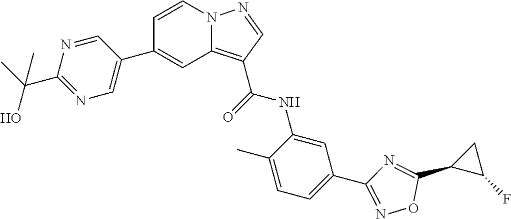 [SMILES: Cc1ccc(-c2noc([C@H]3C[C@@H]3F)n2)cc1NC(=O)c1cnn2ccc(-c3cnc(C(C)(C)O)nc3)cc12] | (M + H)⁺: 514.202 |
| 159 | 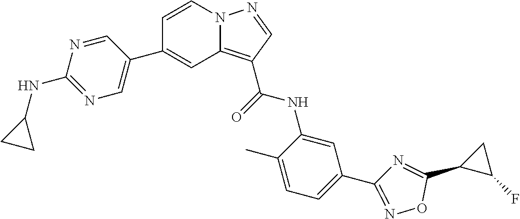 [SMILES: Cc1ccc(-c2noc([C@H]3C[C@@H]3F)n2)cc1NC(=O)c1cnn2ccc(-c3cnc(NC4CC4)nc3)cc12] | (M + H)⁺: 511.206 |
| 160 | 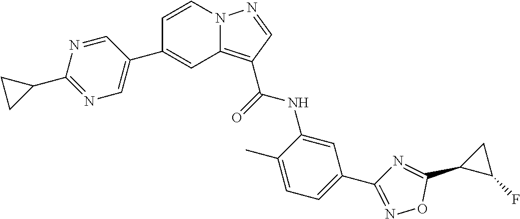 [SMILES: Cc1ccc(-c2noc([C@H]3C[C@@H]3F)n2)cc1NC(=O)c1cnn2ccc(-c3cnc(C4CC4)nc3)cc12] | (M + H)⁺: 496.192 |
| 161 | 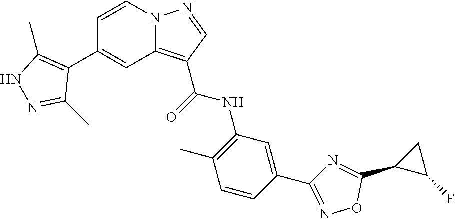 [SMILES: Cc1ccc(-c2noc([C@H]3C[C@@H]3F)n2)cc1NC(=O)c1cnn2ccc(-c3c(C)n[nH]c3C)cc12] | (M + H)⁺: 472.5 |
| 162 | 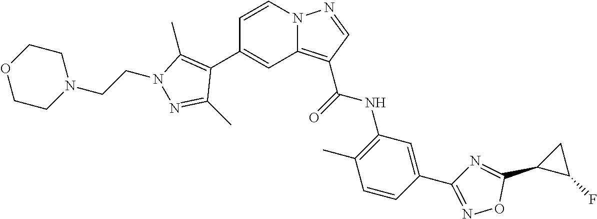 [SMILES: Cc1ccc(-c2noc([C@H]3C[C@@H]3F)n2)cc1NC(=O)c1cnn2ccc(-c3c(C)nn(CCN4CCOCC4)c3C)cc12] | (M + H)⁺: 585.6 |
| 163 | 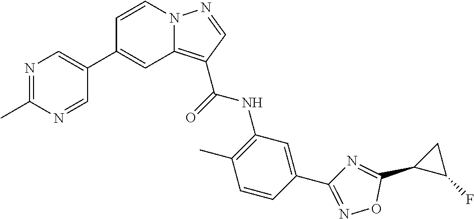 [SMILES: Cc1ncc(-c2ccn3ncc(C(=O)Nc4cc(-c5noc([C@H]6C[C@@H]6F)n5)ccc4C)c3c2)cn1] | (M + H)⁺: 470.3 |
| 164 | 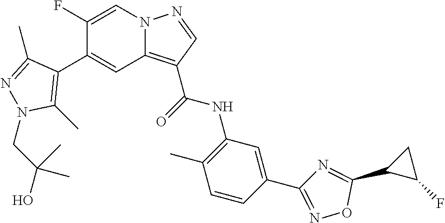 | (M + H)⁺: 562.316 |
| 165 | 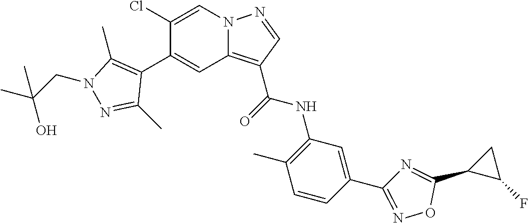 [SMILES: Cc1ccc(-c2noc([C@H]3C[C@@H]3F)n2)cc1NC(=O)c1cnn2cc(Cl)c(-c3c(C)nn(CC(C)(C)O)c3C)cc12] | (M + H)⁺: 580.2 |
| 166 | 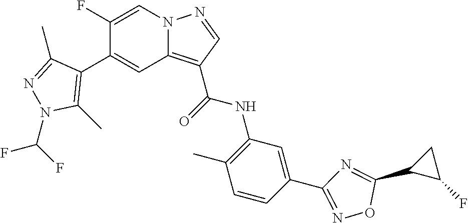 | (M + H)⁺: 540.139 |
| 167 | 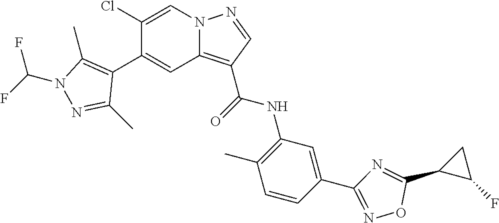 [SMILES: Cc1ccc(-c2noc([C@H]3C[C@@H]3F)n2)cc1NC(=O)c1cnn2cc(Cl)c(-c3c(C)nn(C(F)F)c3C)cc12] | (M + H)⁺: 556.1 |
| 167-1 | 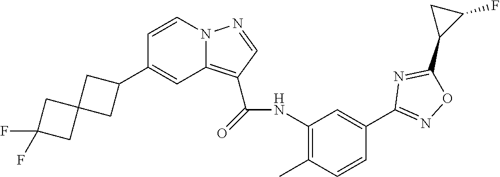 [SMILES: Cc1ccc(-c2noc([C@H]3C[C@@H]3F)n2)cc1NC(=O)c1cnn2ccc(C3CC4(C3)CC(F)(F)C4)cc12] | (M + H)⁺: 508.02 |

### Example 168

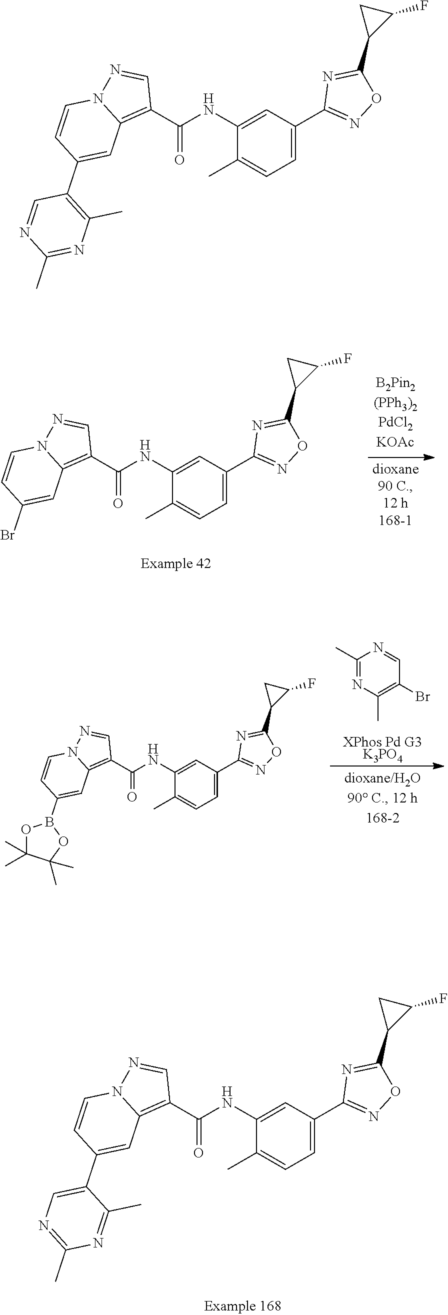

Step 168-1: A suspension of Example 42: 5-bromo-N-(5-(5-((1R,2S)-2-fluorocyclopropyl)-1,2,4-oxadiazol-3-yl)-2-methylphenyl)pyrazolo[1,5-a]pyridine-3-carboxamide (100 mg, 0.219 mmol) in 1,4-dioxane (2.0 ml) was treated with 4,4,4′,4′,5,5,5′,5′-octamethyl-2,2′-bi(1,3,2-dioxaborolane) (67 mg, 0.263 mmol), (PPh₃)₂PdCl₂ (16 mg, 0.022 mmol), and KOAc (91 mg, 0.657 mmol). The reaction was stirred at 90 C for 12 hr. The mixture was concentrated in vacuo. The crude was added to a 4 g silica gel column and eluted by acetone/cyclohexane from 0% to 100% to give the desired product (61 mg, 0.120 mmol, 55% yield) as a white solid.
Step 168-2: A solution of 5-bromo-2,4-dimethylpyrimidine (10 mg, 0.052 mmol), XPhos Pd G3 (4 mg, 0.004 mmol), and K₃PO₄ (28 mg, 0.130 mmol) in dioxane/H₂O (1.0 ml, 9:1) was treated with a freshly prepared solution of the compound from Step 168-1 (22 mg, 0.044 mmol). The reaction was stirred at 80° C. for 12 hrs. The mixture was concentrated in vacuo. The crude was added to a 4 g silica gel column and eluted by acetone/cyclohexane from 0% to 100% to give Example 168 (17 mg, 0.035 mmol, 80% yield) as a white solid. LC-MS, ES+: 484.31 [M+H]⁺.

The following examples were prepared using procedures similar to those described above:

| Example | Structure | ESI-MS |
|---|---|---|
| 169 | 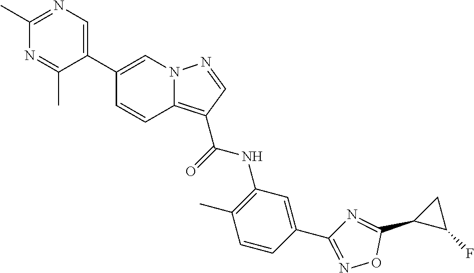 [SMILES: Cc1ncc(-c2ccc3c(C(=O)Nc4cc(-c5noc([C@H]6C[C@@H]6F)n5)ccc4C)cnn3c2)c(C)n1] | (M + H)⁺: 484.4 |
| 170 | 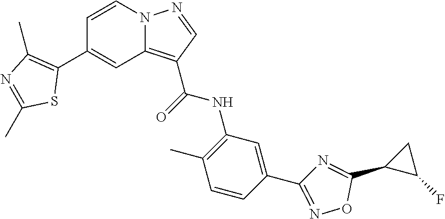 [SMILES: Cc1nc(C)c(-c2ccn3ncc(C(=O)Nc4cc(-c5noc([C@H]6C[C@@H]6F)n5)ccc4C)c3c2)s1] | (M + H)⁺: 489.4 |
| 171 | 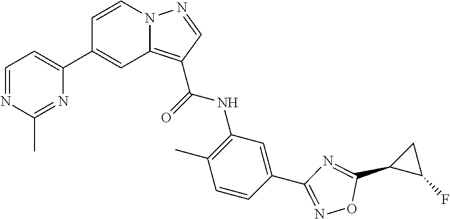 [SMILES: Cc1nccc(-c2ccn3ncc(C(=O)Nc4cc(-c5noc([C@H]6C[C@@H]6F)n5)ccc4C)c3c2)n1] | (M + H)⁺: 470.4 |
| 171-1 | 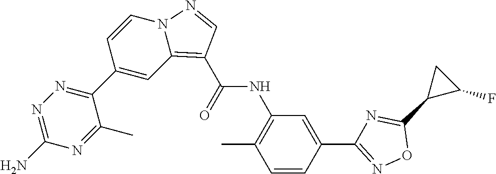 [SMILES: Cc1ccc(-c2noc([C@H]3C[C@@H]3F)n2)cc1NC(=O)c1cnn2ccc(-c3nnc(N)nc3C)cc12] | (M + H)⁺: 486.19 |
| 171-2 | 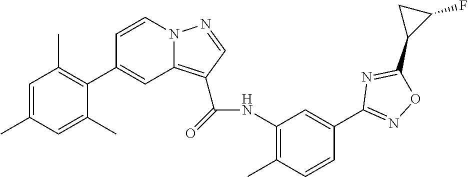 [SMILES: Cc1cc(C)c(-c2ccn3ncc(C(=O)Nc4cc(-c5noc([C@H]6C[C@@H]6F)n5)ccc4C)c3c2)c(C)c1] | (M + H)⁺: 496.1 |
| 171-3 | 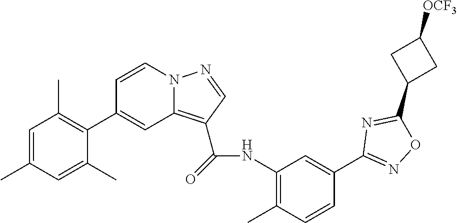 [SMILES: Cc1cc(C)c(-c2ccn3ncc(C(=O)Nc4cc(-c5noc([C@H]6C[C@@H](OC(F)(F)F)C6)n5)ccc4C)c3c2)c(C)c1] | (M + H)⁺: 575.9 |
| 171-4 | 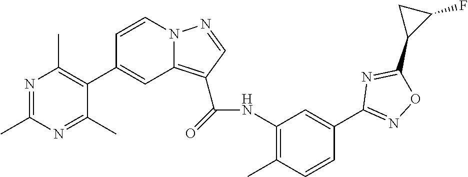 [SMILES: Cc1nc(C)c(-c2ccn3ncc(C(=O)Nc4cc(-c5noc([C@H]6C[C@@H]6F)n5)ccc4C)c3c2)c(C)n1] | (M + H)⁺: 498.2 |
| 171-5 | 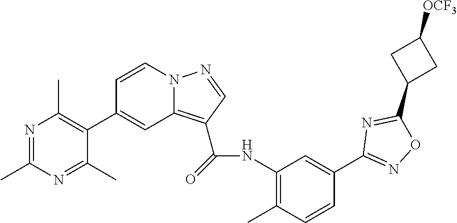 [SMILES: Cc1nc(C)c(-c2ccn3ncc(C(=O)Nc4cc(-c5noc([C@H]6C[C@@H](OC(F)(F)F)C6)n5)ccc4C)c3c2)c(C)n1] | (M + H)⁺: 578.2 |
| 171-6 | 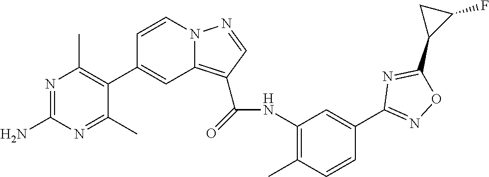 [SMILES: Cc1ccc(-c2noc([C@H]3C[C@@H]3F)n2)cc1NC(=O)c1cnn2ccc(-c3c(C)nc(N)nc3C)cc12] | (M + H)⁺: 499.2 |
| 171-7 | 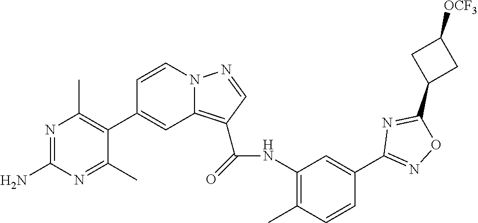 [SMILES: Cc1ccc(-c2noc([C@H]3C[C@@H](OC(F)(F)F)C3)n2)cc1NC(=O)c1cnn2ccc(-c3c(C)nc(N)nc3C)cc12] | (M + H)⁺: 579.2 |
| 171-8 | 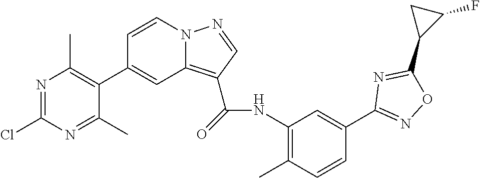 [SMILES: Cc1ccc(-c2noc([C@H]3C[C@@H]3F)n2)cc1NC(=O)c1cnn2ccc(-c3c(C)nc(Cl)nc3C)cc12] | (M + H)⁺: 517.8 |
| 171-9 | 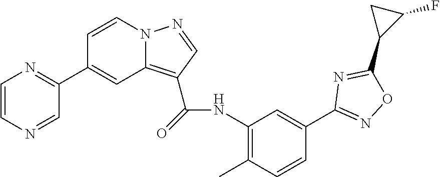 [SMILES: Cc1ccc(-c2noc([C@H]3C[C@@H]3F)n2)cc1NC(=O)c1cnn2ccc(-c3cnccn3)cc12] | (M + H)⁺: 456.1 |
| 171-10 | 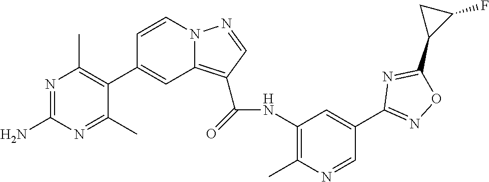 [SMILES: Cc1ncc(-c2noc([C@H]3C[C@@H]3F)n2)cc1NC(=O)c1cnn2ccc(-c3c(C)nc(N)nc3C)cc12] | (M + H)⁺: 500.1 |
| 171-11 | 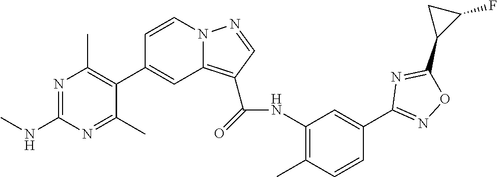 [SMILES: CNc1nc(C)c(-c2ccn3ncc(C(=O)Nc4cc(-c5noc([C@H]6C[C@@H]6F)n5)ccc4C)c3c2)c(C)n1] | (M + H)⁺: 514.5 |
| 171-12 | 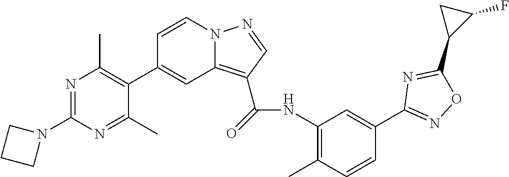 [SMILES: Cc1ccc(-c2noc([C@H]3C[C@@H]3F)n2)cc1NC(=O)c1cnn2ccc(-c3c(C)nc(N4CCC4)nc3C)cc12] | (M + H)⁺: 539.5 |
| 171-13 | 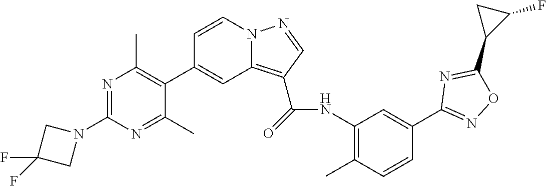 [SMILES: Cc1ccc(-c2noc([C@H]3C[C@@H]3F)n2)cc1NC(=O)c1cnn2ccc(-c3c(C)nc(N4CC(F)(F)C4)nc3C)cc12] | (M + H)⁺: 575.5 |
| 171-14 | 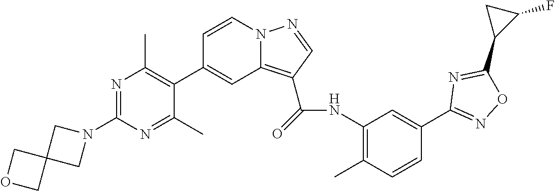 [SMILES: Cc1ccc(-c2noc([C@H]3C[C@@H]3F)n2)cc1NC(=O)c1cnn2ccc(-c3c(C)nc(N4CC5(COC5)C4)nc3C)cc12] | (M + H)⁺: 581.5 |
| 171-15 | 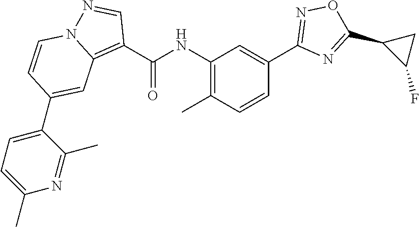 [SMILES: Cc1ccc(-c2ccn3ncc(C(=O)Nc4cc(-c5noc([C@H]6C[C@@H]6F)n5)ccc4C)c3c2)c(C)n1] | (M + H)⁺: 483.44 |
| 171-16 | 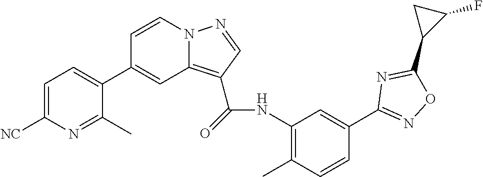 [SMILES: Cc1ccc(-c2noc([C@H]3C[C@@H]3F)n2)cc1NC(=O)c1cnn2ccc(-c3ccc(C#N)nc3C)cc12] | (M + H)⁺: 493.91 |
| 171-17 | 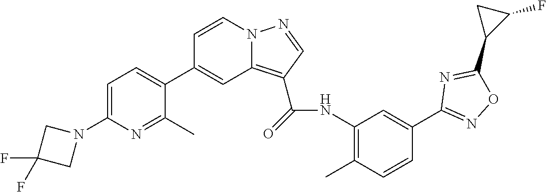 [SMILES: Cc1ccc(-c2noc([C@H]3C[C@@H]3F)n2)cc1NC(=O)c1cnn2ccc(-c3ccc(N4CC(F)(F)C4)nc3C)cc12] | (M + H)⁺: 560.00 |
| 171-18 | 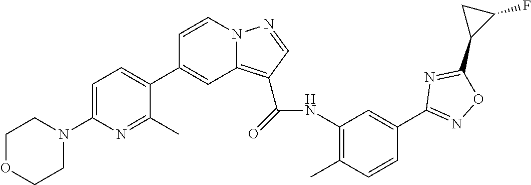 [SMILES: Cc1ccc(-c2noc([C@H]3C[C@@H]3F)n2)cc1NC(=O)c1cnn2ccc(-c3ccc(N4CCOCC4)nc3C)cc12] | (M + H)⁺: 554.09 |
| 171-19 | 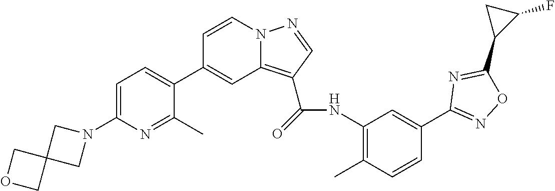 [SMILES: Cc1ccc(-c2noc([C@H]3C[C@@H]3F)n2)cc1NC(=O)c1cnn2ccc(-c3ccc(N4CC5(COC5)C4)nc3C)cc12] | (M + H)⁺: 566.00 |
| 171-20 | 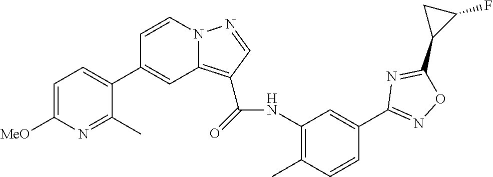 [SMILES: COc1ccc(-c2ccn3ncc(C(=O)Nc4cc(-c5noc([C@H]6C[C@@H]6F)n5)ccc4C)c3c2)c(C)n1] | (M + H)⁺: 499.23 |
| 171-21 | 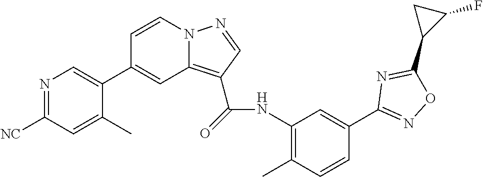 | (M + H)⁺: 554.91 |
| 171-22 | 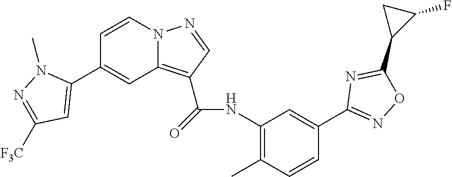 [SMILES: Cc1ccc(-c2noc([C@H]3C[C@@H]3F)n2)cc1NC(=O)c1cnn2ccc(-c3cc(C(F)(F)F)nn3C)cc12] | (M + H)⁺: 525.87 |
| 171-23 | 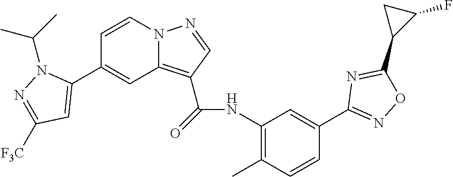 [SMILES: Cc1ccc(-c2noc([C@H]3C[C@@H]3F)n2)cc1NC(=O)c1cnn2ccc(-c3cc(C(F)(F)F)nn3C(C)C)cc12] | (M + H)⁺: 553.95 |
| 171-24 | 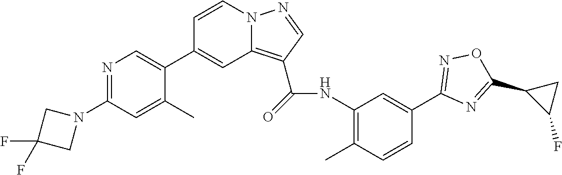 [SMILES: Cc1ccc(-c2noc([C@H]3C[C@@H]3F)n2)cc1NC(=O)c1cnn2ccc(-c3cnc(N4CC(F)(F)C4)cc3C)cc12] | (M + H)⁺: 560.19 |
| 171-25 | 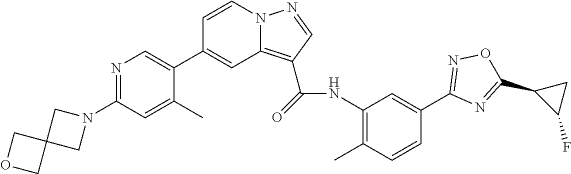 [SMILES: Cc1ccc(-c2noc([C@H]3C[C@@H]3F)n2)cc1NC(=O)c1cnn2ccc(-c3cnc(N4CC5(COC5)C4)cc3C)cc12] | (M + H)⁺: 566.05 |
| 171-26 | 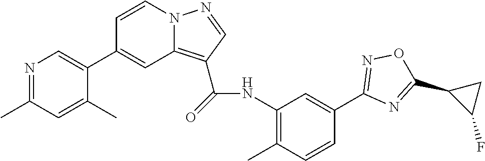 [SMILES: Cc1cc(C)c(-c2ccn3ncc(C(=O)Nc4cc(-c5noc([C@H]6C[C@@H]6F)n5)ccc4C)c3c2)cn1] | (M + H)⁺: 483.35 |
| 171-28 | 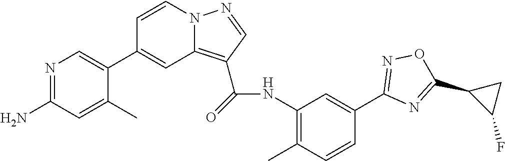 [SMILES: Cc1ccc(-c2noc([C@H]3C[C@@H]3F)n2)cc1NC(=O)c1cnn2ccc(-c3cnc(N)cc3C)cc12] | (M + H)⁺: 484.17 |
| 171-29 | 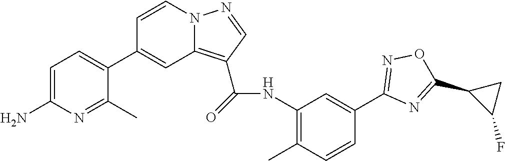 [SMILES: Cc1ccc(-c2noc([C@H]3C[C@@H]3F)n2)cc1NC(=O)c1cnn2ccc(-c3ccc(N)nc3C)cc12] | (M + H)⁺: 484.17 |
| 171-30 | 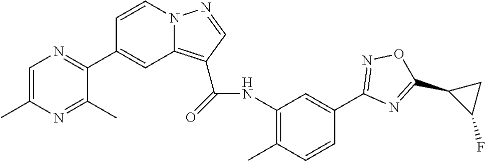 [SMILES: Cc1cnc(-c2ccn3ncc(C(=O)Nc4cc(-c5noc([C@H]6C[C@@H]6F)n5)ccc4C)c3c2)c(C)n1] | (M + H)⁺: 484.17 |
| 171-31 | 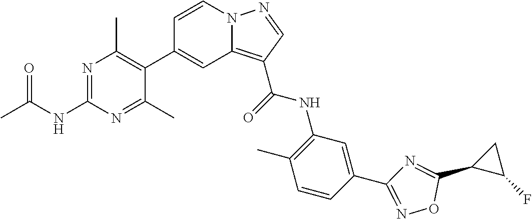 [SMILES: CC(=O)Nc1nc(C)c(-c2ccn3ncc(C(=O)Nc4cc(-c5noc([C@H]6C[C@@H]6F)n5)ccc4C)c3c2)c(C)n1] | (M + H)⁺: 541.4 |

### Example 172

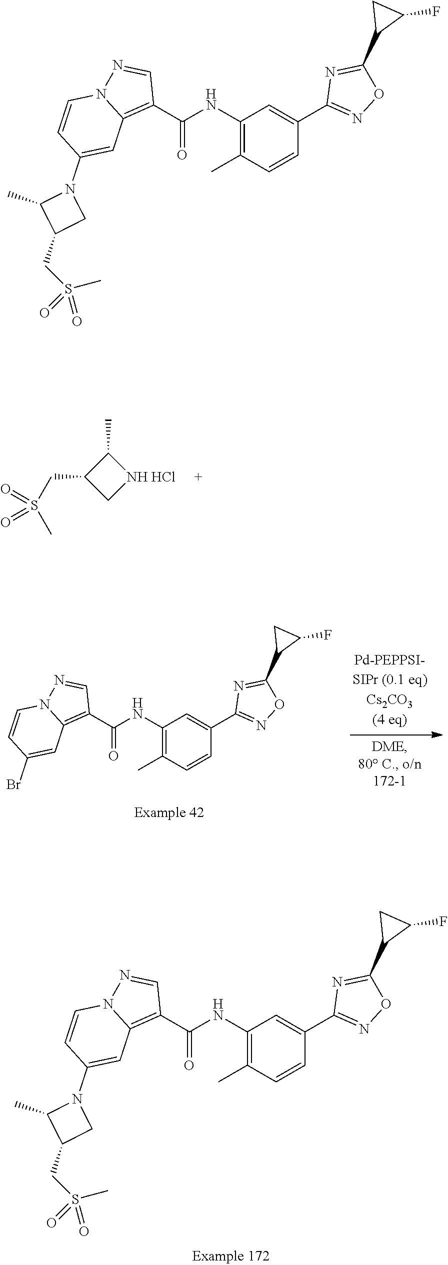

Step 172-1: A solution of (2 S, 3S)-2-methyl-3-((methyl sulfonyl)methyl)azetidine, HCl (37 mg, 1.4 Eq, 0.19 mmol) and Example 42: 5-bromo-N-(5-(5-((1R,2M)-2-fluorocyclopropyl)-1,2,4-oxadiazol-3-yl)-2-methylphenyl)pyrazolo[1,5-a]pyridine-3-carboxamide (60 mg, 1 Eq, 0.13 mmol) in 1,2-Dimethoxyethane (0.5 mL) was treated with Cs₂CO₃ (178 mg, 4.2 Eq, 546 μmol) and Pd-PEPPSI-STPr catalyst (12 mg, 0.13 Eq, 18 μmol) under N₂. The reaction was warmed to 80° C. and stirred overnight. The mixture was filtered through celite and concentrated in vacuo. The crude was purified by HPLC to give the desired product Example 172 (39 mg, 69 μmol, 52% Yield, 9500 Purity) as a white solid. LC-MS, ES+: 539.19 [M+H]⁺.

The following examples were prepared using procedures similar to those described above:

| Example | Structure | ESI-MS |
|---|---|---|
| 173 | 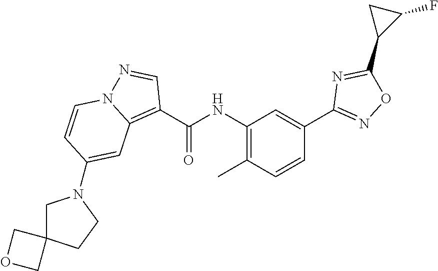 [SMILES: Cc1ccc(-c2noc([C@H]3C[C@@H]3F)n2)cc1NC(=O)c1cnn2ccc(N3CCC4(COC4)C3)cc12] | (M + H)⁺: 489.28 |
| 174 | 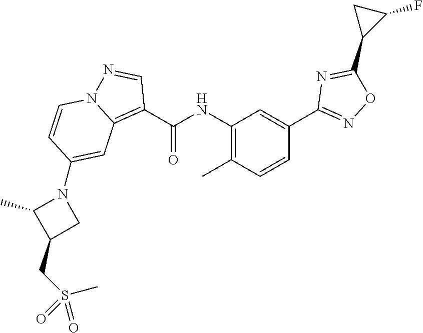 [SMILES: Cc1ccc(-c2noc([C@H]3C[C@@H]3F)n2)cc1NC(=O)c1cnn2ccc(N3C[C@@H](CS(C)(=O)=O)[C@@H]3C)cc12] | (M + H)⁺: 539.29 |
| 175 | 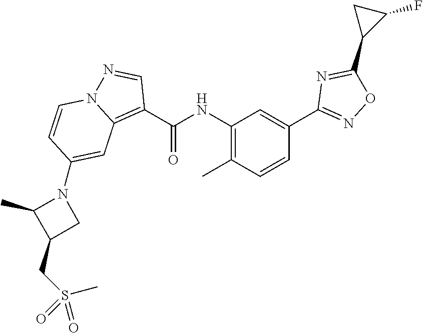 [SMILES: Cc1ccc(-c2noc([C@H]3C[C@@H]3F)n2)cc1NC(=O)c1cnn2ccc(N3C[C@@H](CS(C)(=O)=O)[C@H]3C)cc12] | (M + H)⁺: 539.19 |
| 176 | 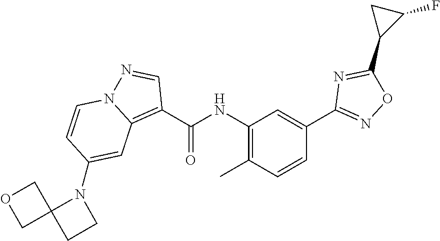 [SMILES: Cc1ccc(-c2noc([C@H]3C[C@@H]3F)n2)cc1NC(=O)c1cnn2ccc(N3CCC34COC4)cc12] | (M + H)⁺: 475.19 |
| 177 | 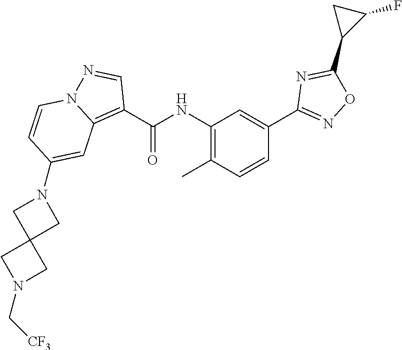 [SMILES: Cc1ccc(-c2noc([C@H]3C[C@@H]3F)n2)cc1NC(=O)c1cnn2ccc(N3CC4(CN(CC(F)(F)F)C4)C3)cc12] | (M + H)⁺: 556.45 |
| 178 | 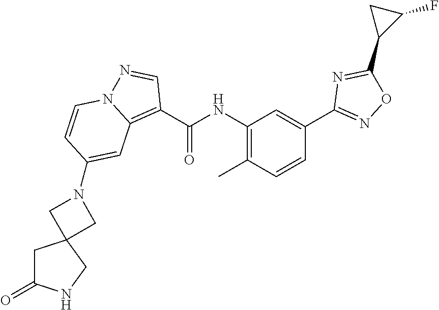 [SMILES: Cc1ccc(-c2noc([C@H]3C[C@@H]3F)n2)cc1NC(=O)c1cnn2ccc(N3CC4(CNC(=O)C4)C3)cc12] | (M + H)⁺: 502.23 |
| 179 | 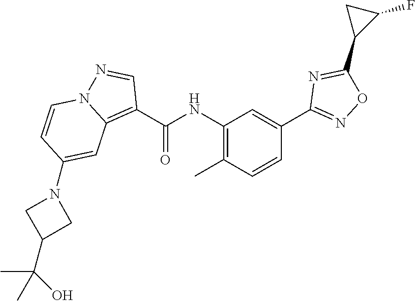 [SMILES: Cc1ccc(-c2noc([C@H]3C[C@@H]3F)n2)cc1NC(=O)c1cnn2ccc(N3CC(C(C)(C)O)C3)cc12] | (M + H)⁺: 491.24 |
| 180 | 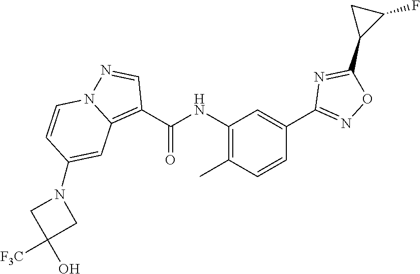 [SMILES: Cc1ccc(-c2noc([C@H]3C[C@@H]3F)n2)cc1NC(=O)c1cnn2ccc(N3CC(O)(C(F)(F)F)C3)cc12] | (M + H)⁺: 517.17 |
| 181 | 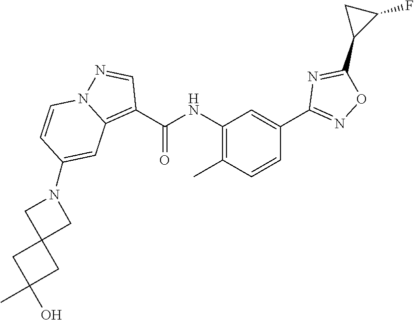 [SMILES: Cc1ccc(-c2noc([C@H]3C[C@@H]3F)n2)cc1NC(=O)c1cnn2ccc(N3CC4(C3)CC(C)(O)C4)cc12] | (M + H)⁺: 503.24 |
| 182 | 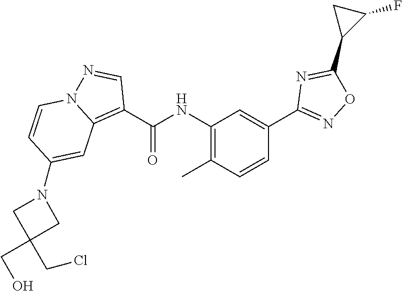 [SMILES: Cc1ccc(-c2noc([C@H]3C[C@@H]3F)n2)cc1NC(=O)c1cnn2ccc(N3CC(CO)(CCl)C3)cc12] | (M + H)⁺: 511.14 513.09 |
| 183 | 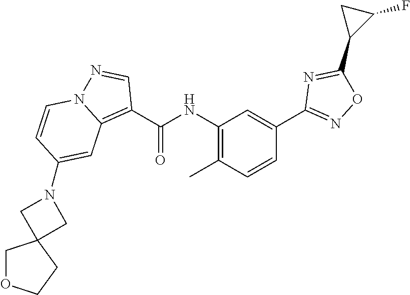 [SMILES: Cc1ccc(-c2noc([C@H]3C[C@@H]3F)n2)cc1NC(=O)c1cnn2ccc(N3CC4(CCOC4)C3)cc12] | (M + H)⁺: 489.26 |
| 184 | 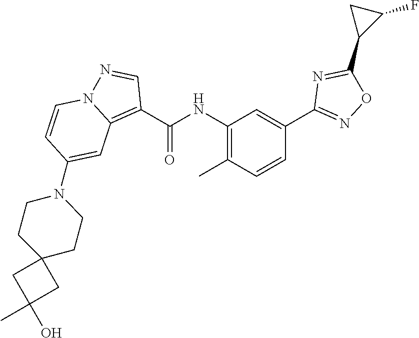 [SMILES: Cc1ccc(-c2noc([C@H]3C[C@@H]3F)n2)cc1NC(=O)c1cnn2ccc(N3CCC4(CC3)CC(C)(O)C4)cc12] | (M + H)⁺: 531.37 |
| 185 | 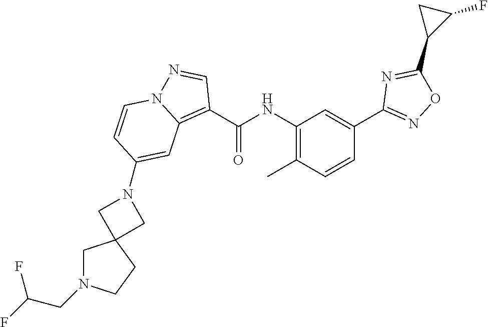 [SMILES: Cc1ccc(-c2noc([C@H]3C[C@@H]3F)n2)cc1NC(=O)c1cnn2ccc(N3CC4(CCN(CC(F)F)C4)C3)cc12] | (M + H)⁺: 552.44 |
| 186 | 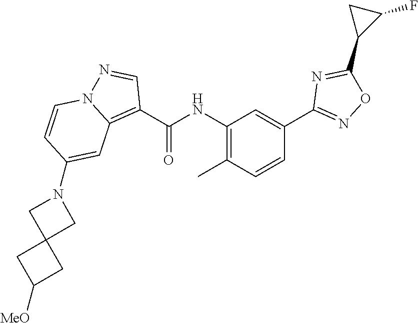 [SMILES: COC1CC2(C1)CN(c1ccn3ncc(C(=O)Nc4cc(-c5noc([C@H]6C[C@@H]6F)n5)ccc4C)c3c1)C2] | (M + H)⁺: 503.25 |
| 187 | 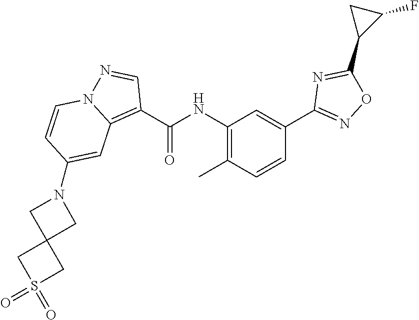 [SMILES: Cc1ccc(-c2noc([C@H]3C[C@@H]3F)n2)cc1NC(=O)c1cnn2ccc(N3CC4(C3)CS(=O)(=O)C4)cc12] | (M + H)⁺: 523.08 |
| 188 | 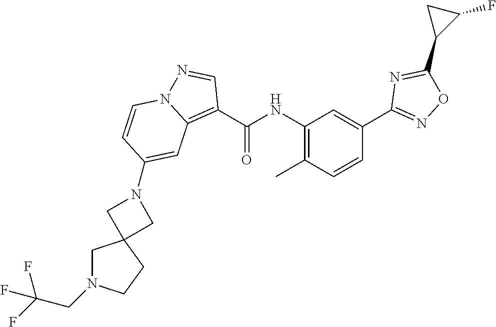 [SMILES: Cc1ccc(-c2noc([C@H]3C[C@@H]3F)n2)cc1NC(=O)c1cnn2ccc(N3CC4(CCN(CC(F)(F)F)C4)C3)cc12] | (M + H)⁺: 570.24 |
| 189 | 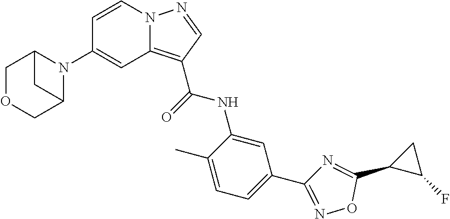 [SMILES: Cc1ccc(-c2noc([C@H]3C[C@@H]3F)n2)cc1NC(=O)c1cnn2ccc(N3C4COCC3C4)cc12] | (M + H)⁺: 475.290 |
| 190 | 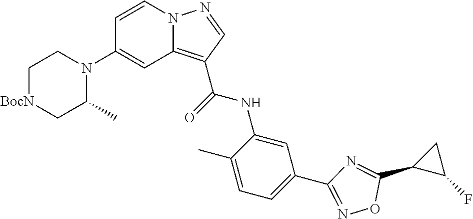 [SMILES: Cc1ccc(-c2noc([C@H]3C[C@@H]3F)n2)cc1NC(=O)c1cnn2ccc(N3CCN(C(=O)OC(C)(C)C)C[C@H]3C)cc12] | (M + H)⁺: 576.271 |
| 191 | 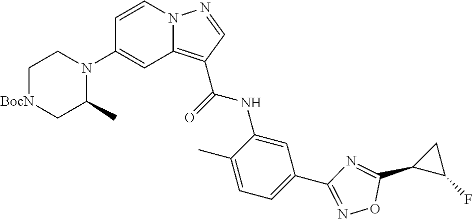 [SMILES: Cc1ccc(-c2noc([C@H]3C[C@@H]3F)n2)cc1NC(=O)c1cnn2ccc(N3CCN(C(=O)OC(C)(C)C)C[C@@H]3C)cc12] | (M + H)⁺: 576.273 |
| 192 | 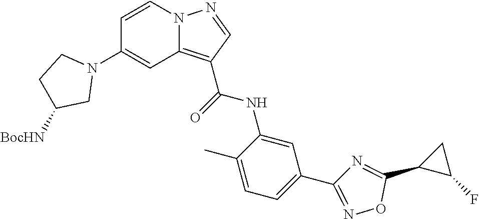 [SMILES: Cc1ccc(-c2noc([C@H]3C[C@@H]3F)n2)cc1NC(=O)c1cnn2ccc(N3CC[C@@H](NC(=O)OC(C)(C)C)C3)cc12] | (M + H)⁺: 562.256 |
| 193 | 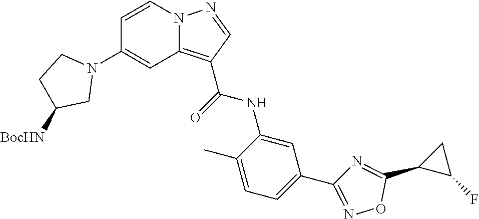 [SMILES: Cc1ccc(-c2noc([C@H]3C[C@@H]3F)n2)cc1NC(=O)c1cnn2ccc(N3CC[C@H](NC(=O)OC(C)(C)C)C3)cc12] | (M + H)⁺: 562.316 |
| 194 | 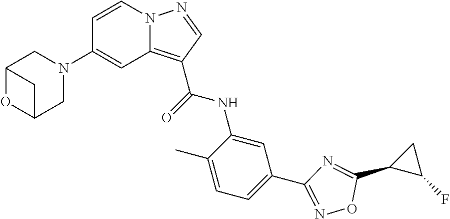 [SMILES: Cc1ccc(-c2noc([C@H]3C[C@@H]3F)n2)cc1NC(=O)c1cnn2ccc(N3CC4CC(C3)O4)cc12] | (M + H)⁺: 475.187 |
| 195 | 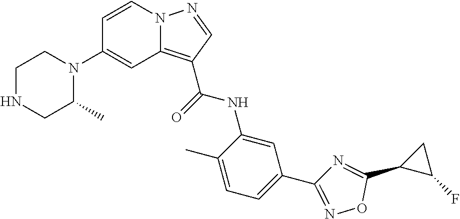 [SMILES: Cc1ccc(-c2noc([C@H]3C[C@@H]3F)n2)cc1NC(=O)c1cnn2ccc(N3CCNC[C@H]3C)cc12] | (M + H)⁺: 476.198 |
| 196 | 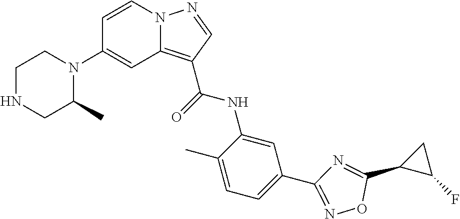 [SMILES: Cc1ccc(-c2noc([C@H]3C[C@@H]3F)n2)cc1NC(=O)c1cnn2ccc(N3CCNC[C@@H]3C)cc12] | (M + H)⁺: 476.227 |
| 197 | 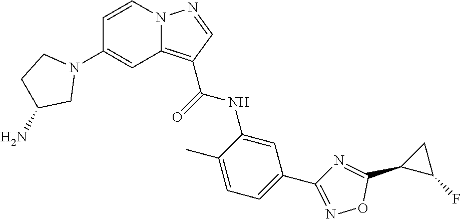 [SMILES: Cc1ccc(-c2noc([C@H]3C[C@@H]3F)n2)cc1NC(=O)c1cnn2ccc(N3CC[C@@H](N)C3)cc12] | (M + H)⁺: 462.183 |
| 198 | 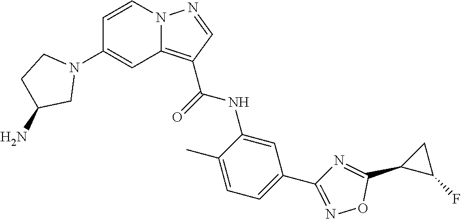 [SMILES: Cc1ccc(-c2noc([C@H]3C[C@@H]3F)n2)cc1NC(=O)c1cnn2ccc(N3CC[C@H](N)C3)cc12] | (M + H)⁺: 462.207 |
| 199 | 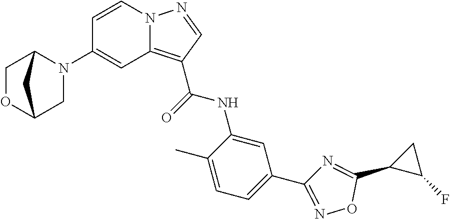 [SMILES: Cc1ccc(-c2noc([C@H]3C[C@@H]3F)n2)cc1NC(=O)c1cnn2ccc(N3C[C@H]4C[C@@H]3CO4)cc12] | (M + H)⁺: 475.290 |
| 200 | 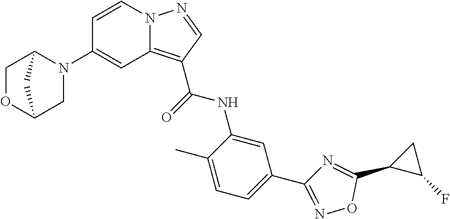 [SMILES: Cc1ccc(-c2noc([C@H]3C[C@@H]3F)n2)cc1NC(=O)c1cnn2ccc(N3C[C@@H]4C[C@H]3CO4)cc12] | (M + H)⁺: 475.290 |
| 201 | 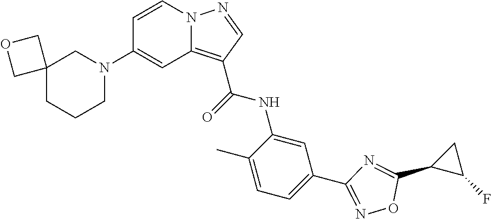 [SMILES: Cc1ccc(-c2noc([C@H]3C[C@@H]3F)n2)cc1NC(=O)c1cnn2ccc(N3CCCC4(COC4)C3)cc12] | (M + H)⁺: 503.221 |
| 202 | 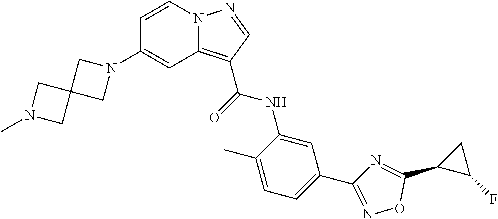 [SMILES: Cc1ccc(-c2noc([C@H]3C[C@@H]3F)n2)cc1NC(=O)c1cnn2ccc(N3CC4(CN(C)C4)C3)cc12] | (M + H)⁺: 488.442 |
| 203 | 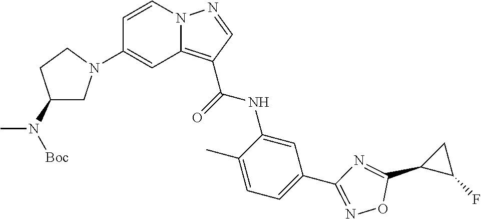 [SMILES: Cc1ccc(-c2noc([C@H]3C[C@@H]3F)n2)cc1NC(=O)c1cnn2ccc(N3CC[C@H](N(C)C(=O)OC(C)(C)C)C3)cc12] | (M + H)⁺: 576.271 |
| 204 | 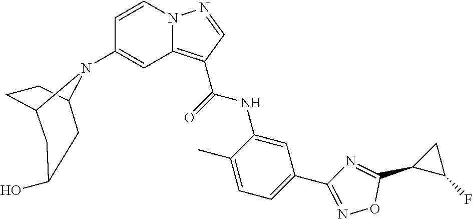 [SMILES: Cc1ccc(-c2noc([C@H]3C[C@@H]3F)n2)cc1NC(=O)c1cnn2ccc(N3C4CCC3CC(O)C4)cc12] | (M + H)⁺: 503.371 |
| 205 | 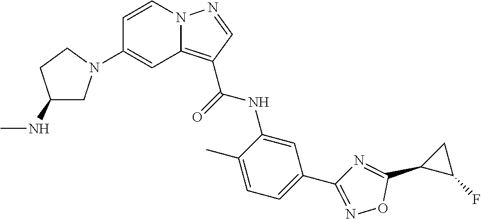 [SMILES: CN[C@H]1CCN(c2ccn3ncc(C(=O)Nc4cc(-c5noc([C@H]6C[C@@H]6F)n5)ccc4C)c3c2)C1] | (M + H)⁺: 476.202 |
| 206 | 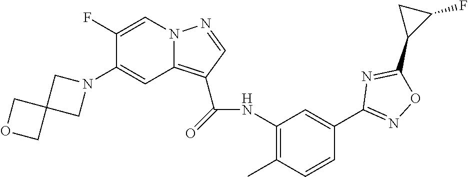 [SMILES: Cc1ccc(-c2noc([C@H]3C[C@@H]3F)n2)cc1NC(=O)c1cnn2cc(F)c(N3CC4(COC4)C3)cc12] | (M + H)⁺: 493.338 |
| 207 | 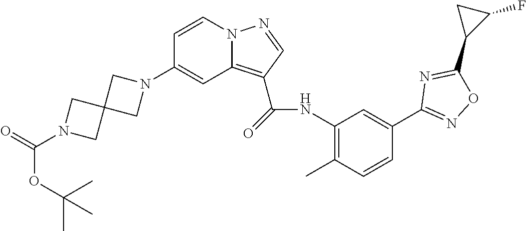 [SMILES: Cc1ccc(-c2noc([C@H]3C[C@@H]3F)n2)cc1NC(=O)c1cnn2ccc(N3CC4(CN(C(=O)OC(C)(C)C)C4)C3)cc12] | (M + H)⁺: 574.364 |
| 208 | 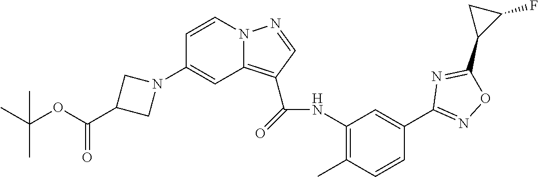 [SMILES: Cc1ccc(-c2noc([C@H]3C[C@@H]3F)n2)cc1NC(=O)c1cnn2ccc(N3CC(C(=O)OC(C)(C)C)C3)cc12] | (M + H)⁺: 533.227 |
| 209 | 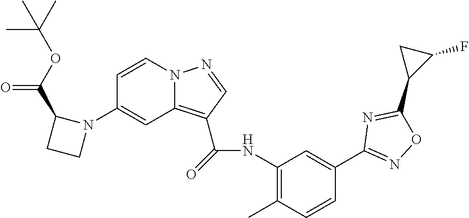 [SMILES: Cc1ccc(-c2noc([C@H]3C[C@@H]3F)n2)cc1NC(=O)c1cnn2ccc(N3CC[C@H]3C(=O)OC(C)(C)C)cc12] | (M + H)⁺: 533.275 |
| 210 | 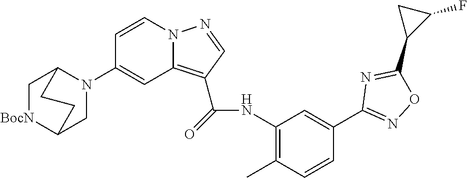 [SMILES: Cc1ccc(-c2noc([C@H]3C[C@@H]3F)n2)cc1NC(=O)c1cnn2ccc(N3CC4CCC3CN4C(=O)OC(C)(C)C)cc12] | (M + H)⁺: 588.380 |
| 211 | 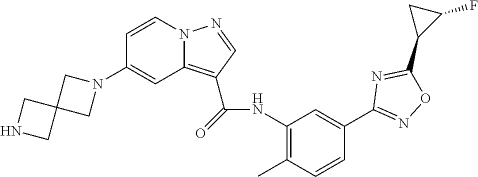 [SMILES: Cc1ccc(-c2noc([C@H]3C[C@@H]3F)n2)cc1NC(=O)c1cnn2ccc(N3CC4(CNC4)C3)cc12] | (M + H)⁺: 474.426 |
| 212 | 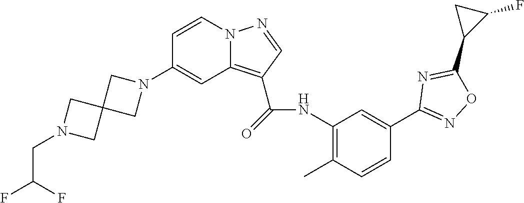 [SMILES: Cc1ccc(-c2noc([C@H]3C[C@@H]3F)n2)cc1NC(=O)c1cnn2ccc(N3CC4(CN(CC(F)F)C4)C3)cc12] | (M + H)⁺: 538.459 |
| 213 | 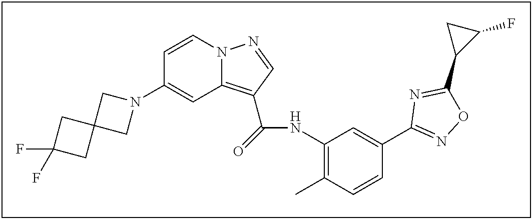 [SMILES: Cc1ccc(-c2noc([C@H]3C[C@@H]3F)n2)cc1NC(=O)c1cnn2ccc(N3CC4(C3)CC(F)(F)C4)cc12] | (M + H)⁺: 509.17 |
| 214 | 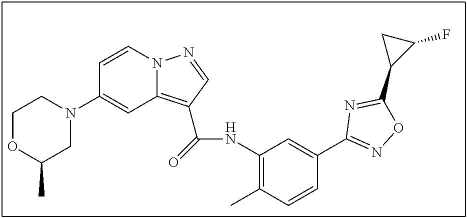 [SMILES: Cc1ccc(-c2noc([C@H]3C[C@@H]3F)n2)cc1NC(=O)c1cnn2ccc(N3CCO[C@H](C)C3)cc12] | (M + H)⁺: 477.25 |
| 215 | 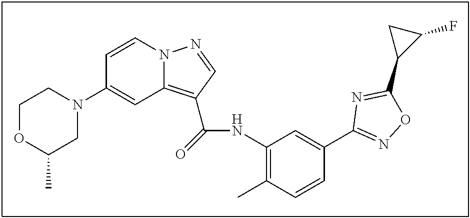 [SMILES: Cc1ccc(-c2noc([C@H]3C[C@@H]3F)n2)cc1NC(=O)c1cnn2ccc(N3CCO[C@@H](C)C3)cc12] | (M + H)⁺: 477.21 |
| 216 | 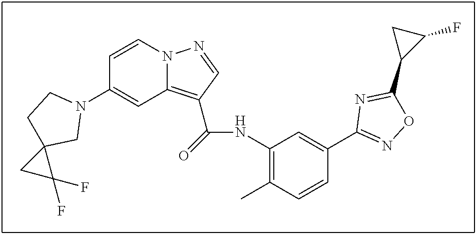 [SMILES: Cc1ccc(-c2noc([C@H]3C[C@@H]3F)n2)cc1NC(=O)c1cnn2ccc(N3CCC4(C3)CC4(F)F)cc12] | (M + H)⁺: 509.07 |
| 217 | 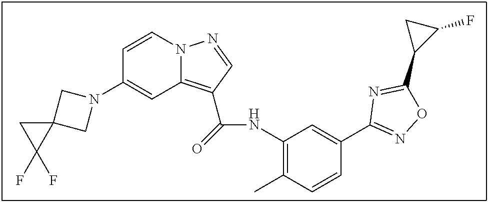 [SMILES: Cc1ccc(-c2noc([C@H]3C[C@@H]3F)n2)cc1NC(=O)c1cnn2ccc(N3CC4(C3)CC4(F)F)cc12] | (M + H)⁺: 495.06 |
| 218 | 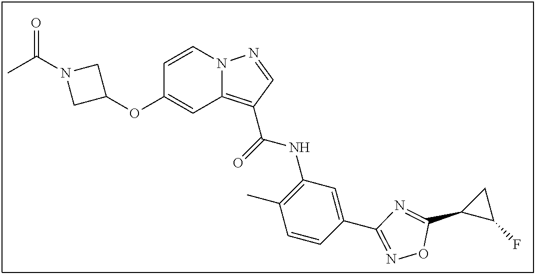 [SMILES: CC(=O)N1CC(Oc2ccn3ncc(C(=O)Nc4cc(-c5noc([C@H]6C[C@@H]6F)n5)ccc4C)c3c2)C1] | (M + H)⁺: 491.17 |
| 219 | 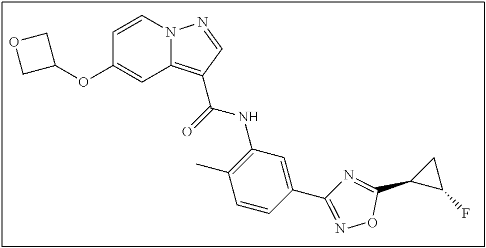 [SMILES: Cc1ccc(-c2noc([C@H]3C[C@@H]3F)n2)cc1NC(=O)c1cnn2ccc(OC3COC3)cc12] | (M + H)⁺: 450.19 |
| 220 | 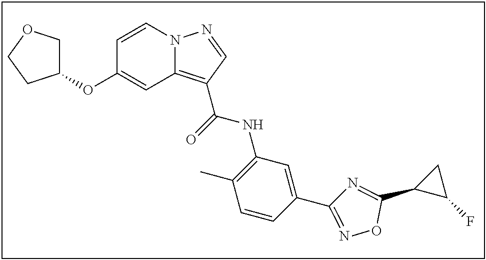 [SMILES: Cc1ccc(-c2noc([C@H]3C[C@@H]3F)n2)cc1NC(=O)c1cnn2ccc(O[C@@H]3CCOC3)cc12] | (M + H)⁺: 464.18 |
| 221 | 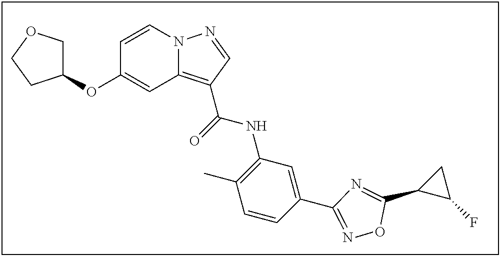 [SMILES: Cc1ccc(-c2noc([C@H]3C[C@@H]3F)n2)cc1NC(=O)c1cnn2ccc(O[C@H]3CCOC3)cc12] | (M + H)⁺: 464.18 |
| 222 | 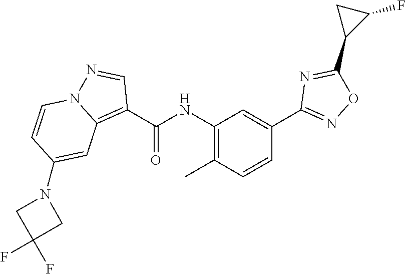 [SMILES: Cc1ccc(-c2noc([C@H]3C[C@@H]3F)n2)cc1NC(=O)c1cnn2ccc(N3CC(F)(F)C3)cc12] | (M + H)⁺: 469.1 |
| 223 | 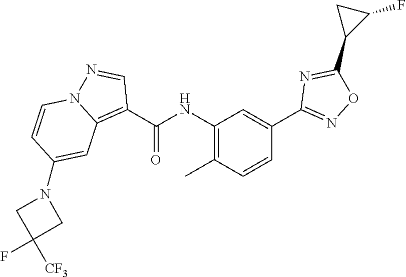 [SMILES: Cc1ccc(-c2noc([C@H]3C[C@@H]3F)n2)cc1NC(=O)c1cnn2ccc(N3CC(F)(C(F)(F)F)C3)cc12] | (M + H)⁺: 519.0 |
| 224 | 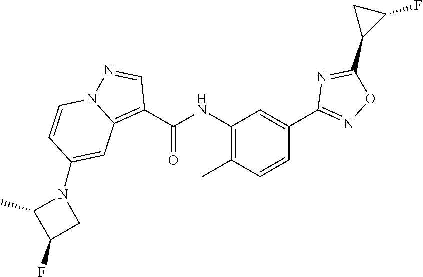 [SMILES: Cc1ccc(-c2noc([C@H]3C[C@@H]3F)n2)cc1NC(=O)c1cnn2ccc(N3C[C@@H](F)[C@@H]3C)cc12] | (M + H)⁺: 465.2 |
| 225 | 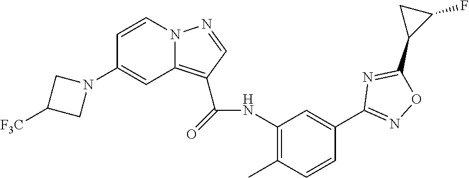 [SMILES: Cc1ccc(-c2noc([C@H]3C[C@@H]3F)n2)cc1NC(=O)c1cnn2ccc(N3CC(C(F)(F)F)C3)cc12] | (M + H)⁺: 501.1 |
| 226 | 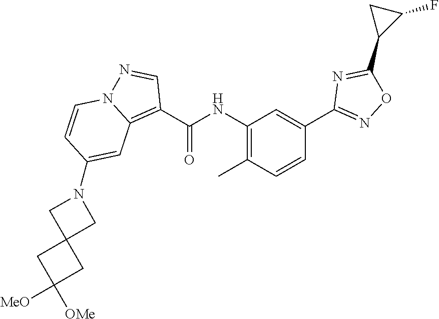 [SMILES: COC1(OC)CC2(CN(c3ccn4ncc(C(=O)Nc5cc(-c6noc([C@H]7C[C@@H]7F)n6)ccc5C)c4c3)C2)C1] | (M + H)⁺: 533.19 |
| 227 | 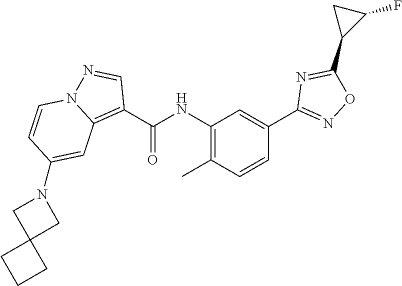 [SMILES: Cc1ccc(-c2noc([C@H]3C[C@@H]3F)n2)cc1NC(=O)c1cnn2ccc(N3CC4(CCC4)C3)cc12] | (M + H)⁺: 473.3 |
| 228 | 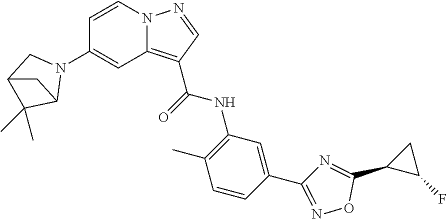 [SMILES: Cc1ccc(-c2noc([C@H]3C[C@@H]3F)n2)cc1NC(=O)c1cnn2ccc(N3CC4CC3C4(C)C)cc12] | (M + H)⁺: 487.338 |
| 229 | 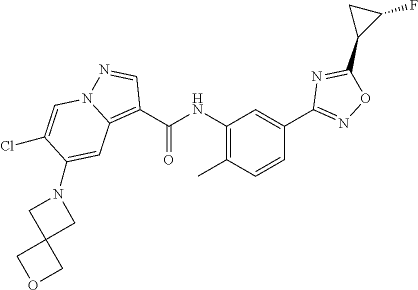 [SMILES: Cc1ccc(-c2noc([C@H]3C[C@@H]3F)n2)cc1NC(=O)c1cnn2cc(Cl)c(N3CC4(COC4)C3)cc12] | (M + H)⁺: 508.9 |
| 230 | 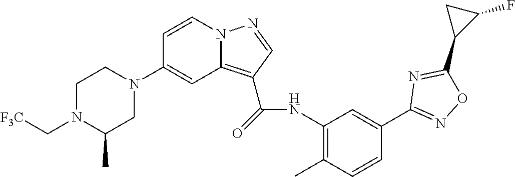 [SMILES: Cc1ccc(-c2noc([C@H]3C[C@@H]3F)n2)cc1NC(=O)c1cnn2ccc(N3CCN(CC(F)(F)F)[C@H](C)C3)cc12] | (M + H)⁺: 558.32 |
| 231 | 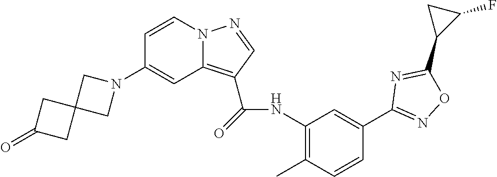 [SMILES: Cc1ccc(-c2noc([C@H]3C[C@@H]3F)n2)cc1NC(=O)c1cnn2ccc(N3CC4(CC(=O)C4)C3)cc12] | (M + H)⁺: 487.04 |
| 232 | 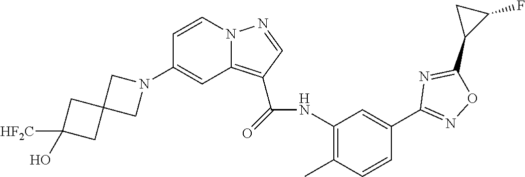 | (M + H)⁺: 539.07 |
| 233 | 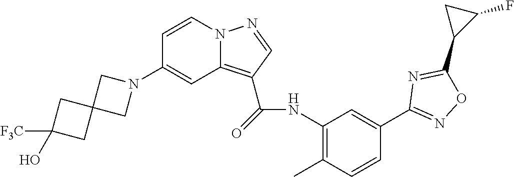 [SMILES: Cc1ccc(-c2noc([C@H]3C[C@@H]3F)n2)cc1NC(=O)c1cnn2ccc(N3CC4(C3)CC(O)(C(F)(F)F)C4)cc12] | (M + H)⁺: 557.07 |
| 234 | 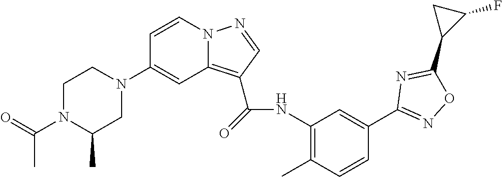 [SMILES: CC(=O)N1CCN(c2ccn3ncc(C(=O)Nc4cc(-c5noc([C@H]6C[C@@H]6F)n5)ccc4C)c3c2)C[C@H]1C] | (M + H)⁺: 518.05 |
| 235 | 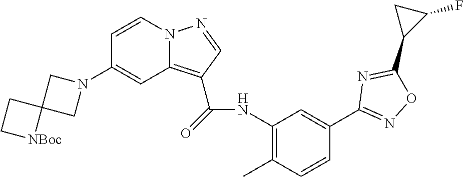 [SMILES: Cc1ccc(-c2noc([C@H]3C[C@@H]3F)n2)cc1NC(=O)c1cnn2ccc(N3CC4(CCN4C(=O)OC(C)(C)C)C3)cc12] | (M + H)⁺: 574.21 |
| 236 | 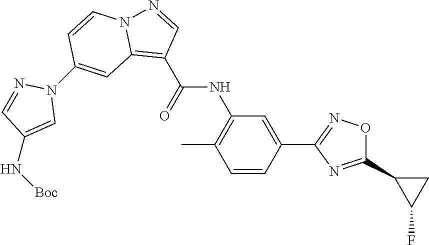 [SMILES: Cc1ccc(-c2noc([C@H]3C[C@@H]3F)n2)cc1NC(=O)c1cnn2ccc(-n3cc(NC(=O)OC(C)(C)C)cn3)cc12] | (M + H)⁺: 559.22 |
| 237 | 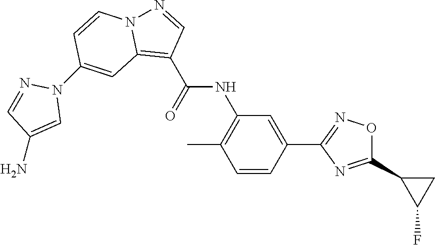 [SMILES: Cc1ccc(-c2noc([C@H]3C[C@@H]3F)n2)cc1NC(=O)c1cnn2ccc(-n3cc(N)cn3)cc12] | (M + H)⁺: 459.29 |
| 238 | 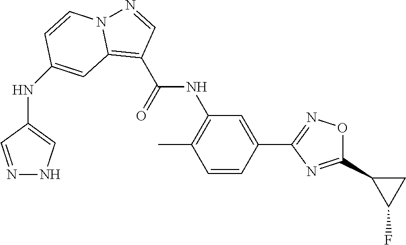 [SMILES: Cc1ccc(-c2noc([C@H]3C[C@@H]3F)n2)cc1NC(=O)c1cnn2ccc(Nc3cn[nH]c3)cc12] | (M + H)⁺: 459.22 |
| 239 | 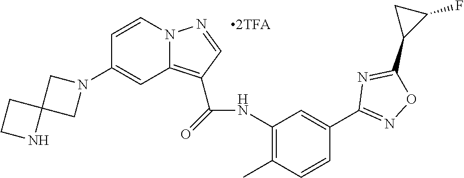 [SMILES: Cc1ccc(-c2noc([C@H]3C[C@@H]3F)n2)cc1NC(=O)c1cnn2ccc(N3CC4(CCN4)C3)cc12] | (M + H)⁺: 474.18 |
| 240 | 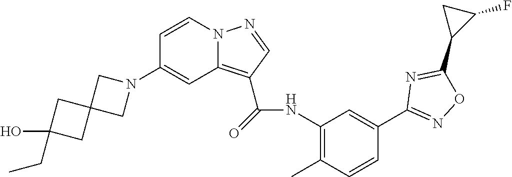 [SMILES: CCC1(O)CC2(CN(c3ccn4ncc(C(=O)Nc5cc(-c6noc([C@H]7C[C@@H]7F)n6)ccc5C)c4c3)C2)C1] | (M + H)⁺: 517.18 |
| 241 | 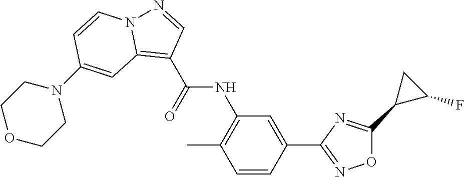 [SMILES: Cc1ccc(-c2noc([C@H]3C[C@@H]3F)n2)cc1NC(=O)c1cnn2ccc(N3CCOCC3)cc12] | (M + H)⁺: 463.20 |
| 241-1 | 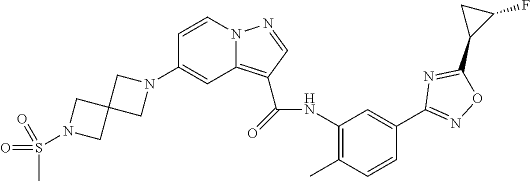 [SMILES: Cc1ccc(-c2noc([C@H]3C[C@@H]3F)n2)cc1NC(=O)c1cnn2ccc(N3CC4(C3)CN(S(C)(=O)=O)C4)cc12] | (M + H)⁺: 551.93 |
| 241-2 | 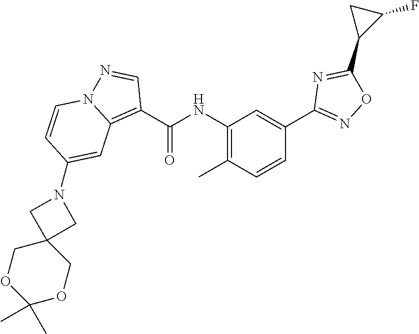 [SMILES: Cc1ccc(-c2noc([C@H]3C[C@@H]3F)n2)cc1NC(=O)c1cnn2ccc(N3CC4(COC(C)(C)OC4)C3)cc12] | (M + H)⁺: 533.16 |
| 241-3 | 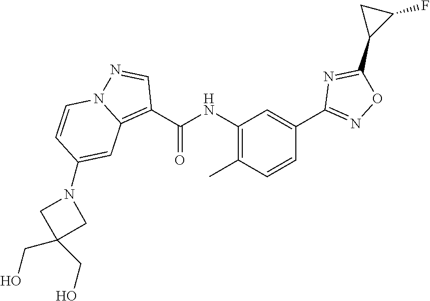 [SMILES: Cc1ccc(-c2noc([C@H]3C[C@@H]3F)n2)cc1NC(=O)c1cnn2ccc(N3CC(CO)(CO)C3)cc12] | (M + H)⁺: 493.07 |
| 241-4 | 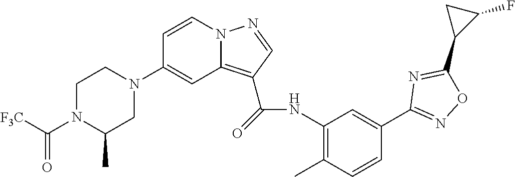 [SMILES: Cc1ccc(-c2noc([C@H]3C[C@@H]3F)n2)cc1NC(=O)c1cnn2ccc(N3CCN(C(=O)C(F)(F)F)[C@H](C)C3)cc12] | (M + H)⁺: 572.00 |
| 241-5 | 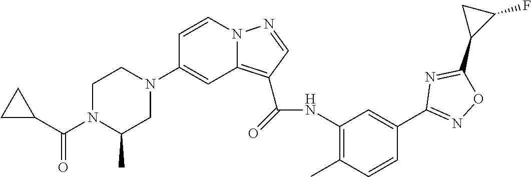 [SMILES: Cc1ccc(-c2noc([C@H]3C[C@@H]3F)n2)cc1NC(=O)c1cnn2ccc(N3CCN(C(=O)C4CC4)[C@H](C)C3)cc12] | (M + H)⁺: 544.06 |
| 241-6 | 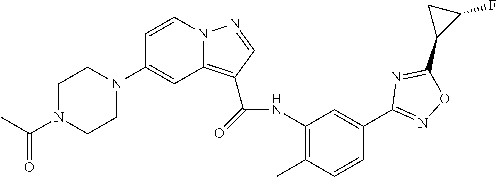 [SMILES: CC(=O)N1CCN(c2ccn3ncc(C(=O)Nc4cc(-c5noc([C@H]6C[C@@H]6F)n5)ccc4C)c3c2)CC1] | (M + H)⁺: 503.98 |
| 241-7 | 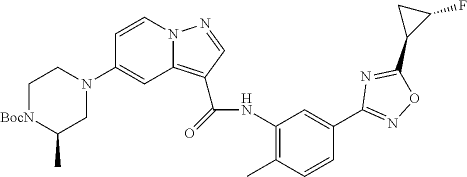 [SMILES: Cc1ccc(-c2noc([C@H]3C[C@@H]3F)n2)cc1NC(=O)c1cnn2ccc(N3CCN(C(=O)OC(C)(C)C)[C@H](C)C3)cc12] | (M + H)⁺: 576.13 |
| 241-8 | 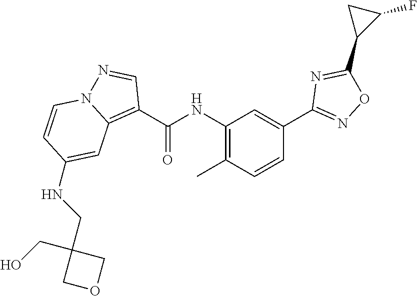 [SMILES: Cc1ccc(-c2noc([C@H]3C[C@@H]3F)n2)cc1NC(=O)c1cnn2ccc(NCC3(CO)COC3)cc12] | (M + H)⁺: 493.11 |
| 241-9 | 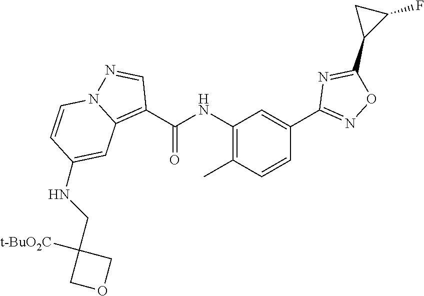 | (M + H)⁺: 563.12 |
| 241-10 | 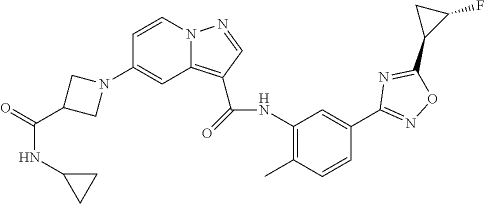 [SMILES: Cc1ccc(-c2noc([C@H]3C[C@@H]3F)n2)cc1NC(=O)c1cnn2ccc(N3CC(C(=O)NC4CC4)C3)cc12] | (M + H)⁺: 516.091 |
| 241-11 | 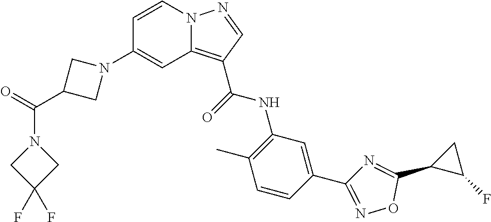 [SMILES: Cc1ccc(-c2noc([C@H]3C[C@@H]3F)n2)cc1NC(=O)c1cnn2ccc(N3CC(C(=O)N4CC(F)(F)C4)C3)cc12] | (M + H)⁺: 552.139 |
| 241-12 | 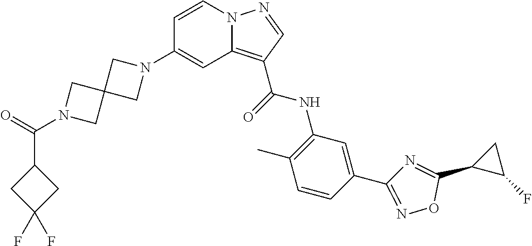 [SMILES: Cc1ccc(-c2noc([C@H]3C[C@@H]3F)n2)cc1NC(=O)c1cnn2ccc(N3CC4(CN(C(=O)C5CC(F)(F)C5)C4)C3)cc12] | (M + H)⁺: 592.124 |
| 241-13 | 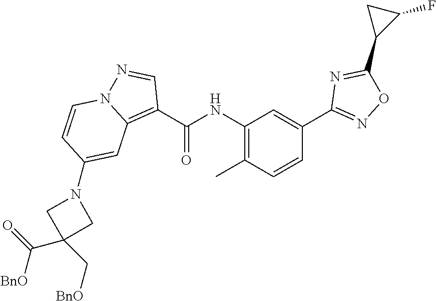 [SMILES: Cc1ccc(-c2noc([C@H]3C[C@@H]3F)n2)cc1NC(=O)c1cnn2ccc(N3CC(COCc4ccccc4)(C(=O)OCc4ccccc4)C3)cc12] | (M + H)⁺: 687.19 |
| 241-14 | 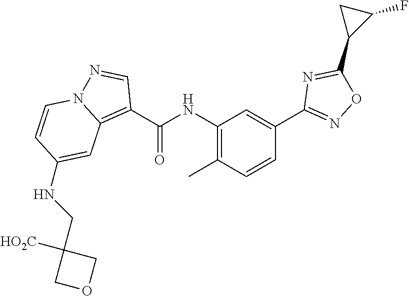 [SMILES: Cc1ccc(-c2noc([C@H]3C[C@@H]3F)n2)cc1NC(=O)c1cnn2ccc(NCC3(C(=O)O)COC3)cc12] | (M + H)⁺: 506.97 |
| 241-15 | 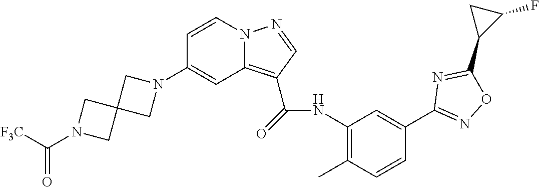 [SMILES: Cc1ccc(-c2noc([C@H]3C[C@@H]3F)n2)cc1NC(=O)c1cnn2ccc(N3CC4(CN(C(=O)C(F)(F)F)C4)C3)cc12] | (M + H)⁺: 570.044 |
| 241-16 | 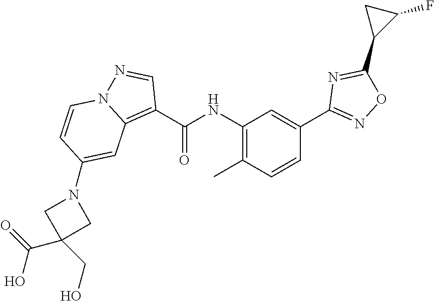 [SMILES: Cc1ccc(-c2noc([C@H]3C[C@@H]3F)n2)cc1NC(=O)c1cnn2ccc(N3CC(CO)(C(=O)O)C3)cc12] | (M + H)⁺: 506.95 |

### Example 242

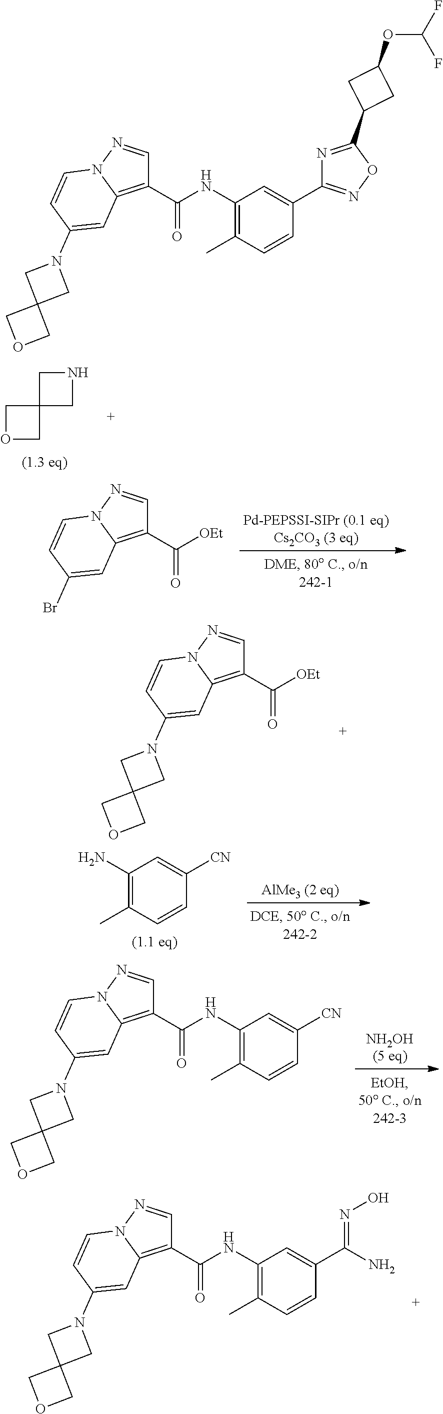

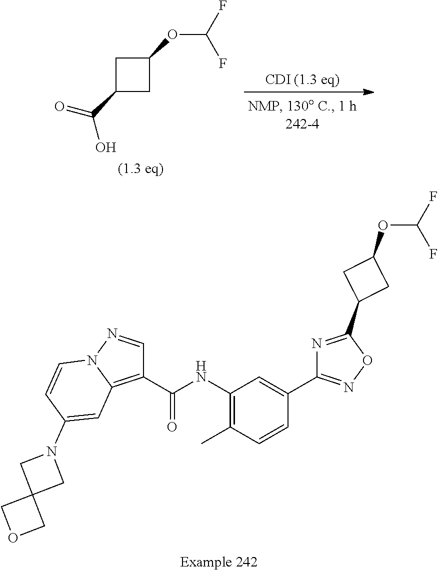 [SMILES: O=C(O)[C@H]1C[C@@H](OC(F)F)C1 ; Cc1ccc(-c2noc([C@H]3C[C@@H](OC(F)F)C3)n2)cc1NC(=O)c1cnn2ccc(N3CC4(COC4)C3)cc12]

Step 242-1: A suspension of ethyl 5-bromopyrazolo[1,5-a]pyridine-3-carboxylate (1.07 g, 1 Eq, 3.98 mmol), Cs₂CO₃ (3.87 g, 2.99 Eq, 11.9 mmol) and Pd-PEPPSI™—SIPr catalyst (270 mg, 0.0996 Eq, 396 mol) in 1,2-Dimethoxyethane (10 mL) was treated with 2-oxa-6-azaspiro[3.3]heptane (504 mg, 450 μL, 1.28 Eq, 5.08 mmol) under N₂. The reaction was warmed to 80° C. and stirred overnight. The mixture was cooled down to room temperature, filtered through celite and rinsed with acetone. The filtrate was concentrated in vacuo, added to a 24 g silica gel column and eluted by acetone/cyclohexane from 0% to 50% to give the desired product (0.91 g, 3.2 mmol, 80%) as an off-white solid. LC-MS, ES+: 288.32 [M+H]⁺.
Step 242-2: A solution of the compound from Step 242-1 (200 mg, 1 Eq, 696 mol) and 3-amino-4-methylbenzonitrile (105 mg, 1.14 Eq, 794 mol) in 1,2-dichloroethane (3 mL) was treated with trimethylaluminum (101 mg, 700 μL, 2 molar, 2.01 Eq, 1.40 mmol) in toluene dropwise at 25° C. over 2 min. The reaction was warmed to 50° C. and stirred overnight. The reaction mixture was quenched with a saturated solution of potassium sodium tartrate slowly at 0° C. and then stirred at 25° C. for 2 hrs. The mixture was filtered and the solid was rinsed with water, acetone and dried over high vacuum to provide the desired product (243 mg, 651 mol, 93.5%) as a white solid. LC-MS, ES+: 374.18 [M+H]⁺.
Step 242-3: A suspension of the compound from Step 242-2 (243 mg, 1 Eq, 651 μmol) in EtOH (3 mL) was treated with hydroxylamine in water (227 mg, 5000 Wt, 5.28 Eq, 3.44 mmol). The reaction was warmed to 55° C. and stirred overnight. The mixture was concentrated in vacuo and dried under high vacuum to give the desired product (243 mg, 598 μmol, 91.9%) as a white solid. LC-MS, ES+: 407.40 [M+H]⁺.
Step 242-4: A solution of (1s,3s)-3-(difluoromethoxy)cyclobutane-1-carboxylic acid (65 mg, 1.3 Eq, 0.39 mmol) in NMP (0.6 mL) was treated with CDI (62 mg, 1.3 Eq, 0.38 mmol). The reaction was stirred at 25° C. for 15 min, and then treated with the compound from Step 242-3 (120 mg, 1 Eq, 295 μmol). The reaction was stirred at 25° C. for additional 15 min, and then warmed to 130° C., and stirred for 1 hour under microwave irradiation. The mixture was diluted with ethyl acetate. The organic layer was washed with water, brine and dried over sodium sulfate. The mixture was filtered and concentrated in vacuo. The crude was added to a 12 g silica gel column and eluted by acetone/cyclohexane from 0% to 10000 to give Example 242 (131 mg, 244 μmol, 82.7%) as a white solid. LC-MS, ES+: 537.17 [M+H]⁺.

The following examples were prepared using procedures similar to those described above:

| Example | Structure | ESI-MS |
|---|---|---|
| 243 | 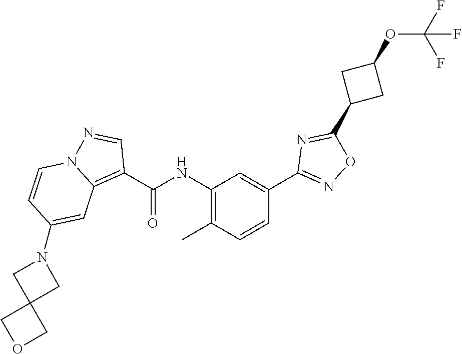 [SMILES: Cc1ccc(-c2noc([C@H]3C[C@@H](OC(F)(F)F)C3)n2)cc1NC(=O)c1cnn2ccc(N3CC4(COC4)C3)cc12] | (M + H)⁺: 555.22 |
| 244 | 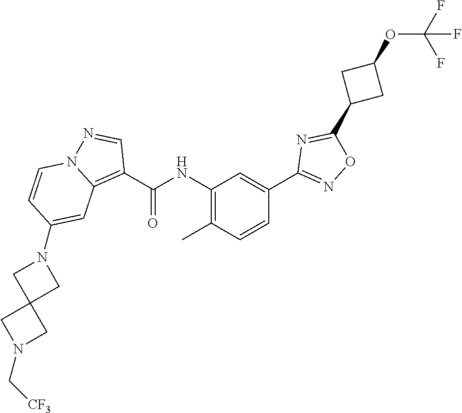 [SMILES: Cc1ccc(-c2noc([C@H]3C[C@@H](OC(F)(F)F)C3)n2)cc1NC(=O)c1cnn2ccc(N3CC4(CN(CC(F)(F)F)C4)C3)cc12] | (M + H)⁺: 636.34 |
| 245 | 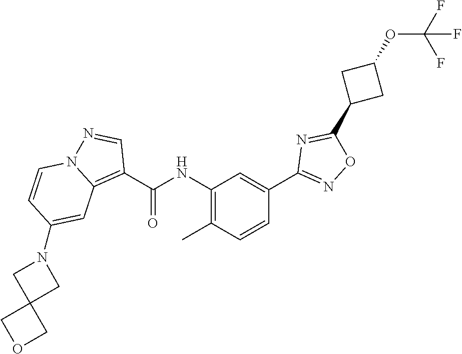 [SMILES: Cc1ccc(-c2noc([C@H]3C[C@H](OC(F)(F)F)C3)n2)cc1NC(=O)c1cnn2ccc(N3CC4(COC4)C3)cc12] | (M + H)⁺: 555.23 |
| 246 | 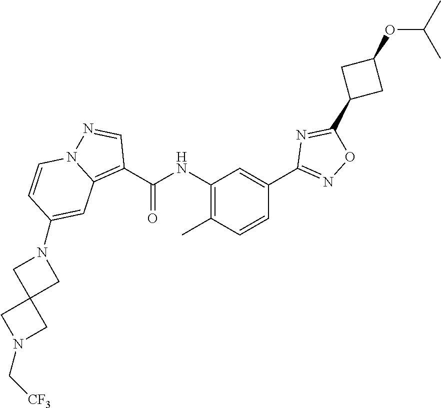 [SMILES: Cc1ccc(-c2noc([C@H]3C[C@@H](OC(C)C)C3)n2)cc1NC(=O)c1cnn2ccc(N3CC4(CN(CC(F)(F)F)C4)C3)cc12] | (M + H)⁺: 610.39 |
| 247 | 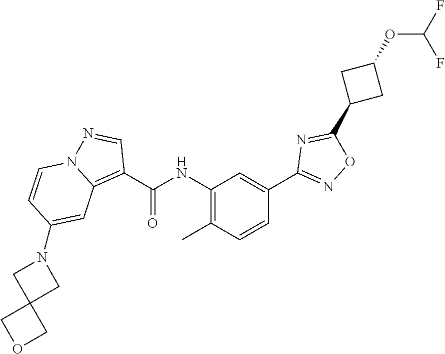 [SMILES: Cc1ccc(-c2noc([C@H]3C[C@H](OC(F)F)C3)n2)cc1NC(=O)c1cnn2ccc(N3CC4(COC4)C3)cc12] | (M + H)⁺: 537.19 |
| 248 | 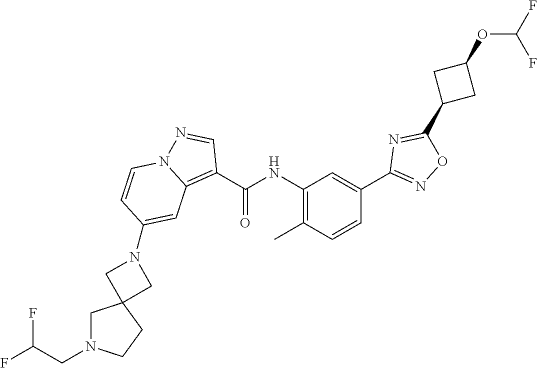 [SMILES: Cc1ccc(-c2noc([C@H]3C[C@@H](OC(F)F)C3)n2)cc1NC(=O)c1cnn2ccc(N3CC4(CCN(CC(F)F)C4)C3)cc12] | (M + H)⁺: 614.42 |
| 249 | 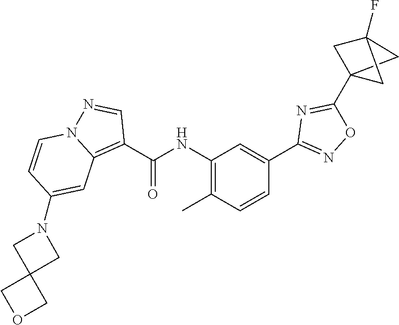 [SMILES: Cc1ccc(-c2noc(C34CC(F)(C3)C4)n2)cc1NC(=O)c1cnn2ccc(N3CC4(COC4)C3)cc12] | (M + H)⁺: 501.17 |
| 250 | 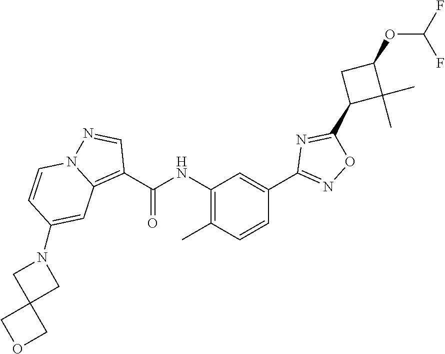 [SMILES: Cc1ccc(-c2noc([C@H]3C[C@@H](OC(F)F)C3(C)C)n2)cc1NC(=O)c1cnn2ccc(N3CC4(COC4)C3)cc12] | (M + H)⁺: 565.22 |
| 251 | 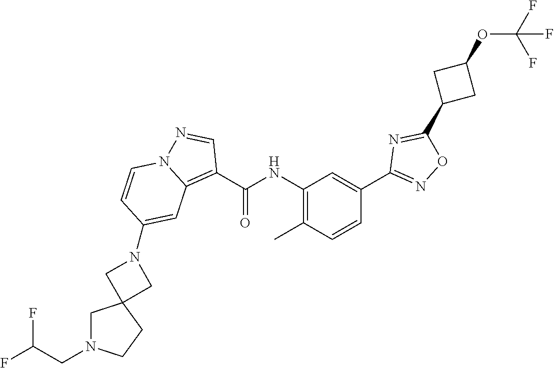 [SMILES: Cc1ccc(-c2noc([C@H]3C[C@@H](OC(F)(F)F)C3)n2)cc1NC(=O)c1cnn2ccc(N3CC4(CCN(CC(F)F)C4)C3)cc12] | (M + H)⁺: 632.44 |
| 252 | 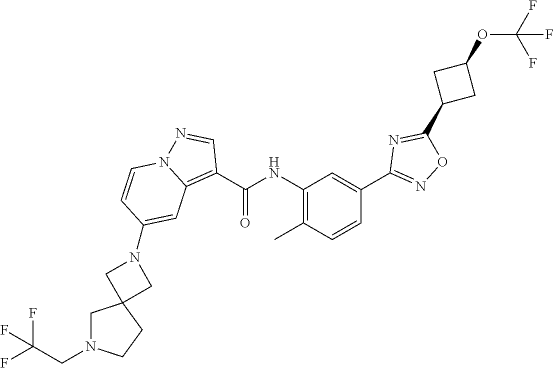 [SMILES: Cc1ccc(-c2noc([C@H]3C[C@@H](OC(F)(F)F)C3)n2)cc1NC(=O)c1cnn2ccc(N3CC4(CCN(CC(F)(F)F)C4)C3)cc12] | (M + H)⁺: 650.29 |
| 253 | 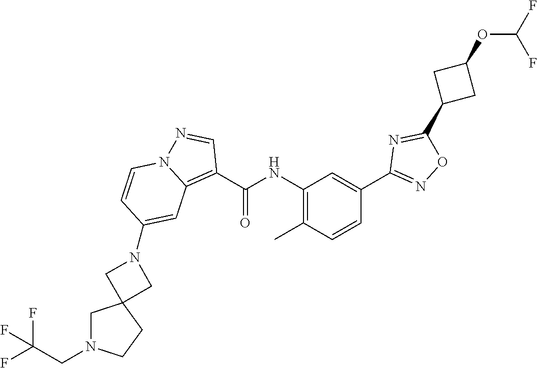 [SMILES: Cc1ccc(-c2noc([C@H]3C[C@@H](OC(F)F)C3)n2)cc1NC(=O)c1cnn2ccc(N3CC4(CCN(CC(F)(F)F)C4)C3)cc12] | (M + H)⁺: 632.27 |
| 254 | 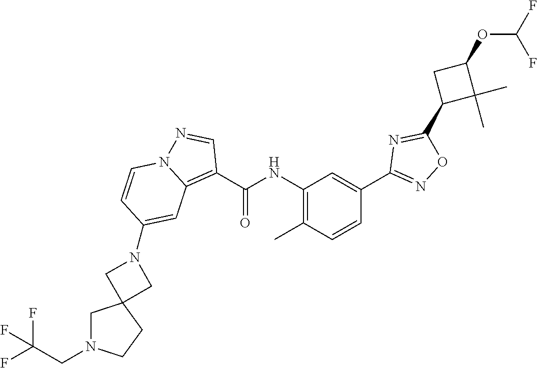 [SMILES: Cc1ccc(-c2noc([C@H]3C[C@@H](OC(F)F)C3(C)C)n2)cc1NC(=O)c1cnn2ccc(N3CC4(CCN(CC(F)(F)F)C4)C3)cc12] | (M + H)⁺: 660.39 |
| 255 | 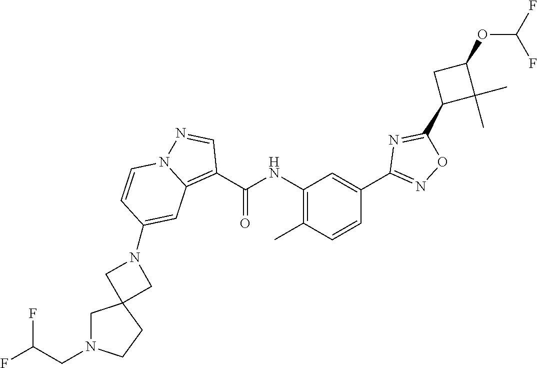 [SMILES: Cc1ccc(-c2noc([C@H]3C[C@@H](OC(F)F)C3(C)C)n2)cc1NC(=O)c1cnn2ccc(N3CC4(CCN(CC(F)F)C4)C3)cc12] | (M + H)⁺: 642.47 |
| 256 | 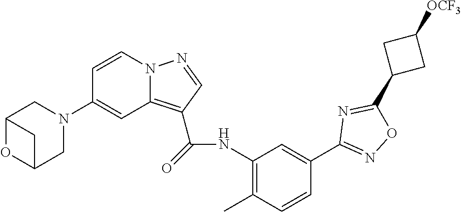 [SMILES: Cc1ccc(-c2noc([C@H]3C[C@@H](OC(F)(F)F)C3)n2)cc1NC(=O)c1cnn2ccc(N3CC4CC(C3)O4)cc12] | (M + H)⁺: 555.163 |
| 257 | 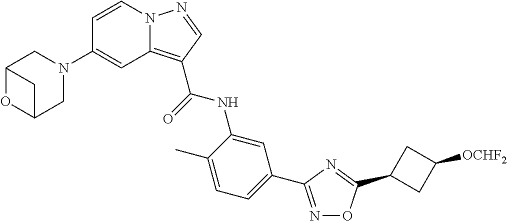 [SMILES: Cc1ccc(-c2noc([C@H]3C[C@@H](OC(F)F)C3)n2)cc1NC(=O)c1cnn2ccc(N3CC4CC(C3)O4)cc12] | (M + H)⁺: 537.211 |
| 258 | 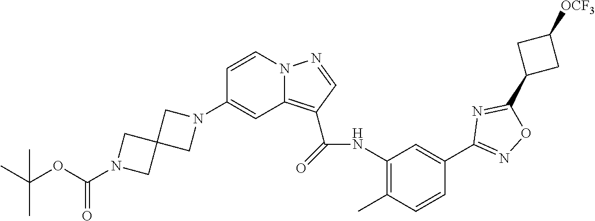 [SMILES: Cc1ccc(-c2noc([C@H]3C[C@@H](OC(F)(F)F)C3)n2)cc1NC(=O)c1cnn2ccc(N3CC4(CN(C(=O)OC(C)(C)C)C4)C3)cc12] | (M + H)⁺: 654.383 |
| 259 | 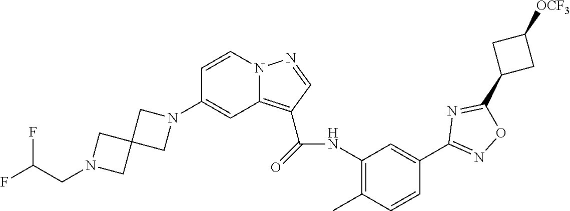 [SMILES: Cc1ccc(-c2noc([C@H]3C[C@@H](OC(F)(F)F)C3)n2)cc1NC(=O)c1cnn2ccc(N3CC4(CN(CC(F)F)C4)C3)cc12] | (M + H)⁺: 618.333 |
| 260 | 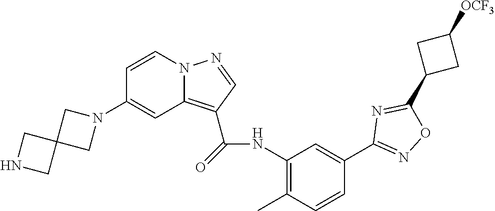 [SMILES: Cc1ccc(-c2noc([C@H]3C[C@@H](OC(F)(F)F)C3)n2)cc1NC(=O)c1cnn2ccc(N3CC4(CNC4)C3)cc12] | (M + H)⁺: 554.395 |
| 261 | 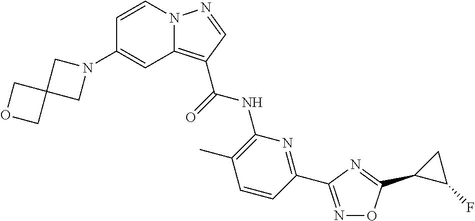 [SMILES: Cc1ccc(-c2noc([C@H]3C[C@@H]3F)n2)nc1NC(=O)c1cnn2ccc(N3CC4(COC4)C3)cc12] | (M + H)⁺: 476.154 |
| 262 | 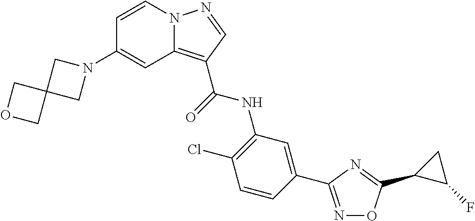 [SMILES: O=C(Nc1cc(-c2noc([C@H]3C[C@@H]3F)n2)ccc1Cl)c1cnn2ccc(N3CC4(COC4)C3)cc12] | (M + H)⁺: 495.162 |
| 263 | 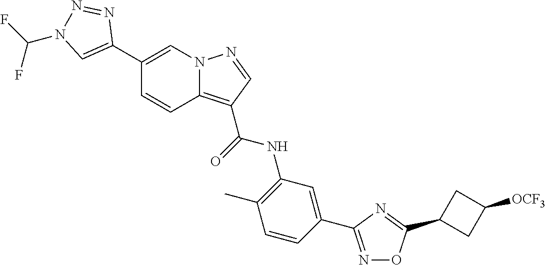 [SMILES: Cc1ccc(-c2noc([C@H]3C[C@@H](OC(F)(F)F)C3)n2)cc1NC(=O)c1cnn2cc(-c3cn(C(F)F)nn3)ccc12] | (M + H)⁺: 575.07 |
| 264 | 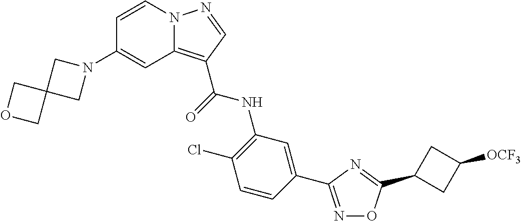 [SMILES: O=C(Nc1cc(-c2noc([C@H]3C[C@@H](OC(F)(F)F)C3)n2)ccc1Cl)c1cnn2ccc(N3CC4(COC4)C3)cc12] | (M + H)⁺: 575.132 |
| 265 | 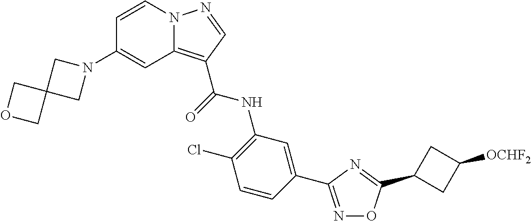 [SMILES: O=C(Nc1cc(-c2noc([C@H]3C[C@@H](OC(F)F)C3)n2)ccc1Cl)c1cnn2ccc(N3CC4(COC4)C3)cc12] | (M + H)⁺: 557.131 |
| 266 | 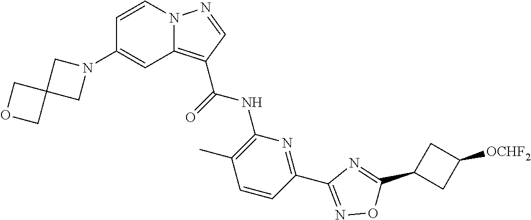 [SMILES: Cc1ccc(-c2noc([C@H]3C[C@@H](OC(F)F)C3)n2)nc1NC(=O)c1cnn2ccc(N3CC4(COC4)C3)cc12] | (M + H)⁺: 538.219 |
| 267 | 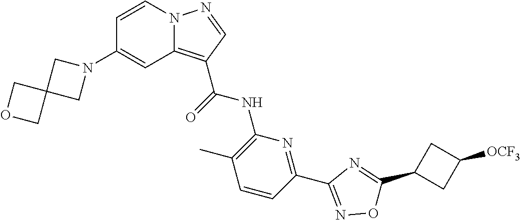 [SMILES: Cc1ccc(-c2noc([C@H]3C[C@@H](OC(F)(F)F)C3)n2)nc1NC(=O)c1cnn2ccc(N3CC4(COC4)C3)cc12] | (M + H)⁺: 556.363 |
| 268 | 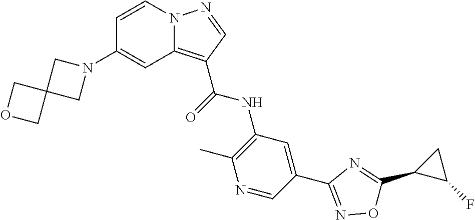 [SMILES: Cc1ncc(-c2noc([C@H]3C[C@@H]3F)n2)cc1NC(=O)c1cnn2ccc(N3CC4(COC4)C3)cc12] | (M + H)⁺: 476.298 |
| 269 | 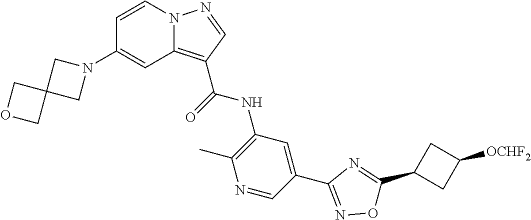 [SMILES: Cc1ncc(-c2noc([C@H]3C[C@@H](OC(F)F)C3)n2)cc1NC(=O)c1cnn2ccc(N3CC4(COC4)C3)cc12] | (M + H)⁺: 538.315 |
| 270 | 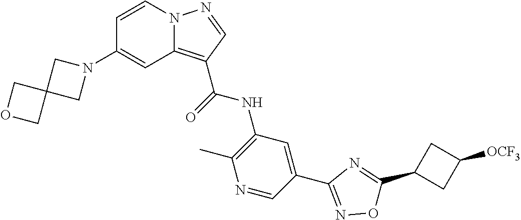 [SMILES: Cc1ncc(-c2noc([C@H]3C[C@@H](OC(F)(F)F)C3)n2)cc1NC(=O)c1cnn2ccc(N3CC4(COC4)C3)cc12] | (M + H)⁺: 556.315 |
| 271 | 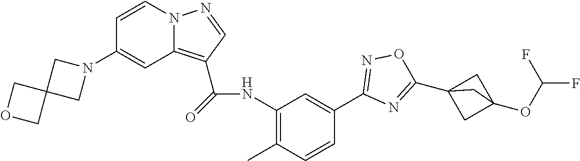 [SMILES: Cc1ccc(-c2noc(C34CC(OC(F)F)(C3)C4)n2)cc1NC(=O)c1cnn2ccc(N3CC4(COC4)C3)cc12] | (M + H)⁺: 549.15 |
| 272 | 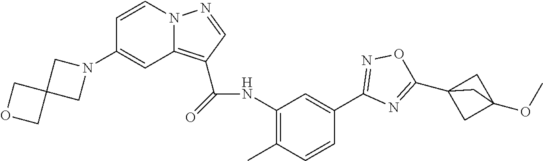 [SMILES: COC12CC(c3nc(-c4ccc(C)c(NC(=O)c5cnn6ccc(N7CC8(COC8)C7)cc56)c4)no3)(C1)C2] | (M + H)⁺: 513.20 |
| 273 | 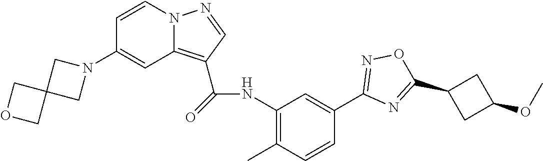 [SMILES: CO[C@H]1C[C@@H](c2nc(-c3ccc(C)c(NC(=O)c4cnn5ccc(N6CC7(COC7)C6)cc45)c3)no2)C1] | (M + H)⁺: 501.11 |
| 274 | 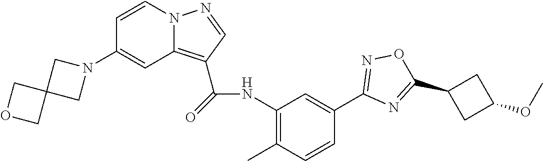 [SMILES: CO[C@H]1C[C@H](c2nc(-c3ccc(C)c(NC(=O)c4cnn5ccc(N6CC7(COC7)C6)cc45)c3)no2)C1] | (M + H)⁺: 501.06 |
| 275 | 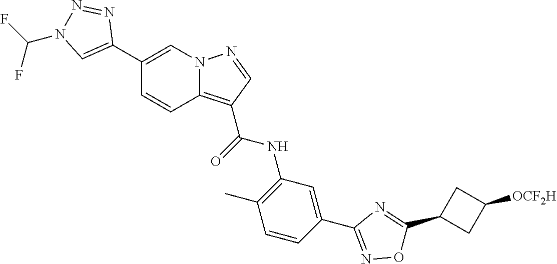 | (M + H)⁺: 557.07 |
| 276 | 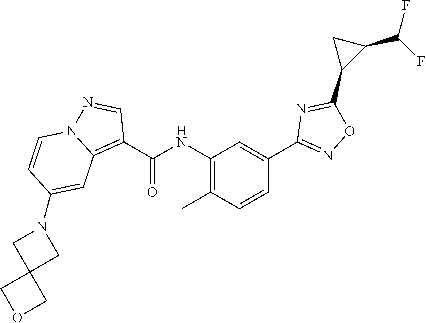 [SMILES: Cc1ccc(-c2noc([C@H]3C[C@H]3C(F)F)n2)cc1NC(=O)c1cnn2ccc(N3CC4(COC4)C3)cc12] | (M + H)⁺: 507.06 |
| 277 | 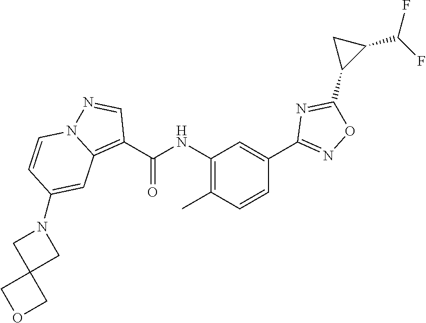 [SMILES: Cc1ccc(-c2noc([C@@H]3C[C@@H]3C(F)F)n2)cc1NC(=O)c1cnn2ccc(N3CC4(COC4)C3)cc12] | (M + H)⁺: 507.09 |
| 278 | 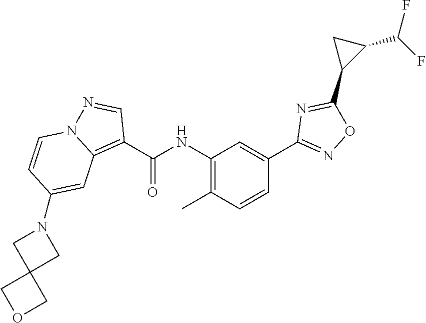 [SMILES: Cc1ccc(-c2noc([C@H]3C[C@@H]3C(F)F)n2)cc1NC(=O)c1cnn2ccc(N3CC4(COC4)C3)cc12] | (M + H)⁺: 507.07 |
| 279 | 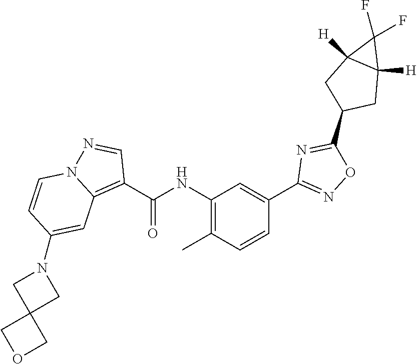 [SMILES: Cc1ccc(-c2noc([C@@H]3C[C@@H]4[C@H](C3)C4(F)F)n2)cc1NC(=O)c1cnn2ccc(N3CC4(COC4)C3)cc12] | (M + H)⁺: 532.82 |
| 280 | 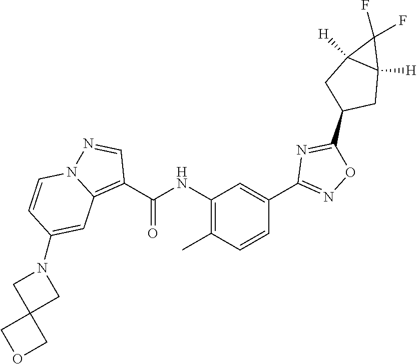 [SMILES: Cc1ccc(-c2noc([C@H]3C[C@@H]4[C@H](C3)C4(F)F)n2)cc1NC(=O)c1cnn2ccc(N3CC4(COC4)C3)cc12] | (M + H)⁺: 533.00 |
| 281 | 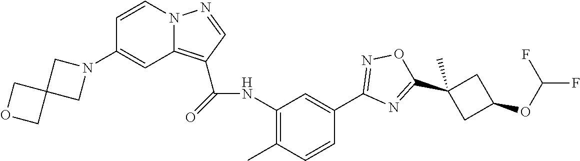 [SMILES: Cc1ccc(-c2noc([C@]3(C)C[C@H](OC(F)F)C3)n2)cc1NC(=O)c1cnn2ccc(N3CC4(COC4)C3)cc12] | (M + H)⁺: 551.12 |
| 281-1 | 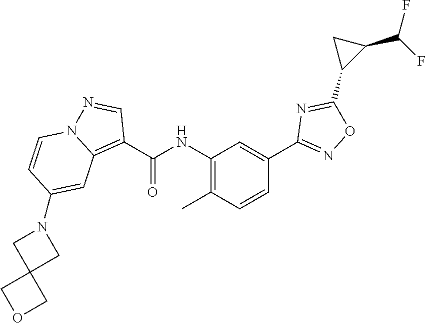 [SMILES: Cc1ccc(-c2noc([C@@H]3C[C@H]3C(F)F)n2)cc1NC(=O)c1cnn2ccc(N3CC4(COC4)C3)cc12] | (M + H)⁺: 507.05 |
| 281-2 | 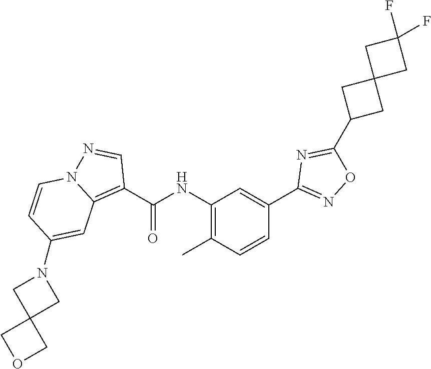 [SMILES: Cc1ccc(-c2noc(C3CC4(C3)CC(F)(F)C4)n2)cc1NC(=O)c1cnn2ccc(N3CC4(COC4)C3)cc12] | (M + H)⁺: 547.18 |
| 281-3 | 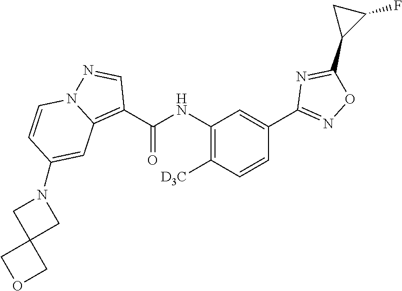 [SMILES: [2H]C([2H])([2H])c1ccc(-c2noc([C@H]3C[C@@H]3F)n2)cc1NC(=O)c1cnn2ccc(N3CC4(COC4)C3)cc12] | (M + H)⁺: 478.13 |
| 281-4 | 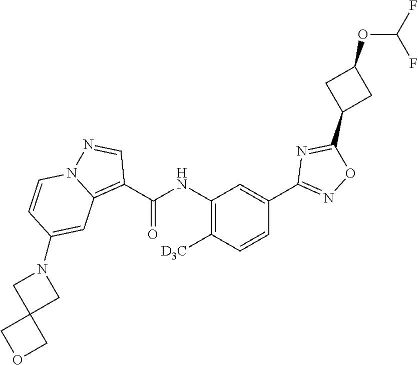 [SMILES: [2H]C([2H])([2H])c1ccc(-c2noc([C@H]3C[C@@H](OC(F)F)C3)n2)cc1NC(=O)c1cnn2ccc(N3CC4(COC4)C3)cc12] | (M + H)⁺: 540.10 |
| 281-5 | 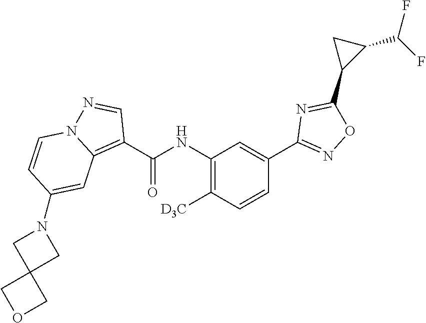 [SMILES: [2H]C([2H])([2H])c1ccc(-c2noc([C@H]3C[C@@H]3C(F)F)n2)cc1NC(=O)c1cnn2ccc(N3CC4(COC4)C3)cc12] | (M + H)⁺: 510.09 |
| 281-6 | 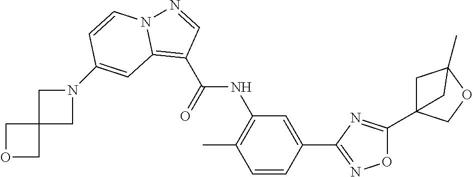 [SMILES: Cc1ccc(-c2noc(C34COC(C)(C3)C4)n2)cc1NC(=O)c1cnn2ccc(N3CC4(COC4)C3)cc12] | (M + H)⁺: 513.05 |
| 281-7 | 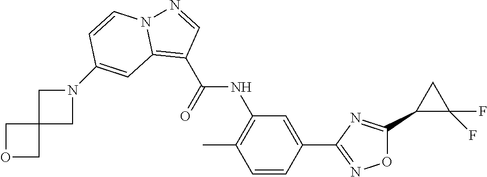 [SMILES: Cc1ccc(-c2noc([C@H]3CC3(F)F)n2)cc1NC(=O)c1cnn2ccc(N3CC4(COC4)C3)cc12] | (M + H)⁺: 493.05 |
| 281-8 | 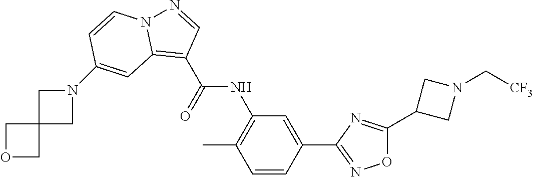 [SMILES: Cc1ccc(-c2noc(C3CN(CC(F)(F)F)C3)n2)cc1NC(=O)c1cnn2ccc(N3CC4(COC4)C3)cc12] | (M + H)⁺: 554.14 |
| 281-9 | 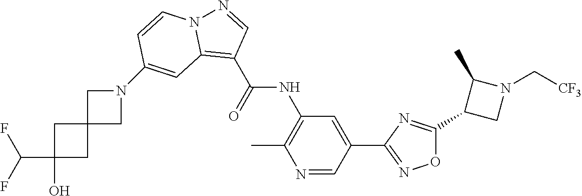 [SMILES: Cc1ncc(-c2noc([C@H]3CN(CC(F)(F)F)[C@@H]3C)n2)cc1NC(=O)c1cnn2ccc(N3CC4(C3)CC(O)(C(F)F)C4)cc12] | (M + H)⁺: 633.24 |
| 281-10 | 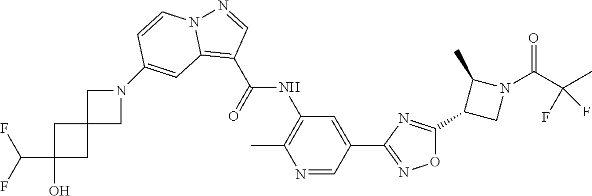 [SMILES: Cc1ncc(-c2noc([C@H]3CN(C(=O)C(C)(F)F)[C@@H]3C)n2)cc1NC(=O)c1cnn2ccc(N3CC4(C3)CC(O)(C(F)F)C4)cc12] | (M + H)⁺: 643.23 |
| 281-11 | 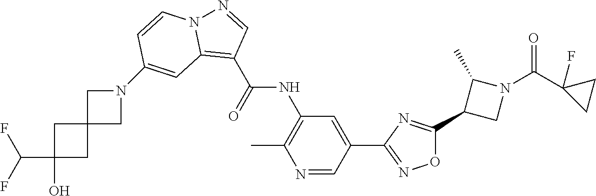 [SMILES: Cc1ncc(-c2noc([C@@H]3CN(C(=O)C4(F)CC4)[C@H]3C)n2)cc1NC(=O)c1cnn2ccc(N3CC4(C3)CC(O)(C(F)F)C4)cc12] | (M + H)⁺: 637.30 |
| 281-12 | 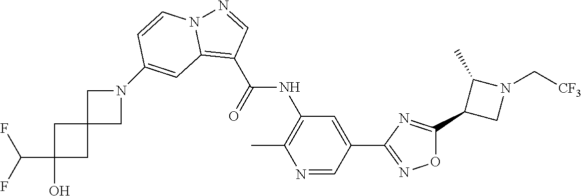 [SMILES: Cc1ncc(-c2noc([C@@H]3CN(CC(F)(F)F)[C@H]3C)n2)cc1NC(=O)c1cnn2ccc(N3CC4(C3)CC(O)(C(F)F)C4)cc12] | (M + H)⁺: 633.24 |
| 281-13 | 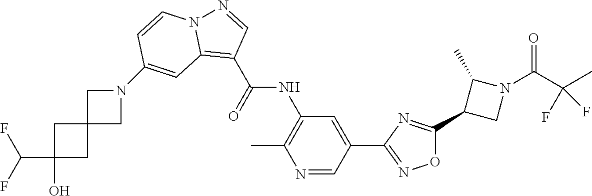 [SMILES: Cc1ncc(-c2noc([C@@H]3CN(C(=O)C(C)(F)F)[C@H]3C)n2)cc1NC(=O)c1cnn2ccc(N3CC4(C3)CC(O)(C(F)F)C4)cc12] | (M + H)⁺: 643.24 |
| 281-14 | 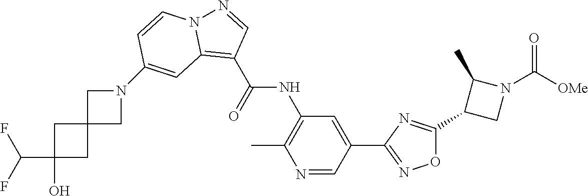 [SMILES: COC(=O)N1C[C@H](c2nc(-c3cnc(C)c(NC(=O)c4cnn5ccc(N6CC7(C6)CC(O)(C(F)F)C7)cc45)c3)no2)[C@H]1C] | (M + H)⁺: 609.24 |
| 281-15 | 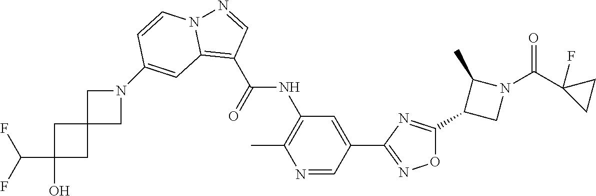 [SMILES: Cc1ncc(-c2noc([C@H]3CN(C(=O)C4(F)CC4)[C@@H]3C)n2)cc1NC(=O)c1cnn2ccc(N3CC4(C3)CC(O)(C(F)F)C4)cc12] | (M + H)⁺: 637.25 |
| 281-16 | 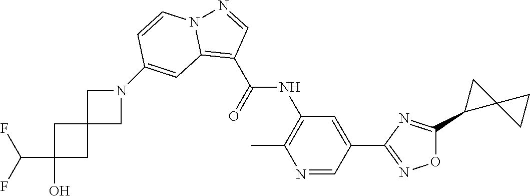 [SMILES: Cc1ncc(-c2noc([C@H]3CC34CC4)n2)cc1NC(=O)c1cnn2ccc(N3CC4(C3)CC(O)(C(F)F)C4)cc12] | (M + H)⁺: 548.22 |
| 281-17 | 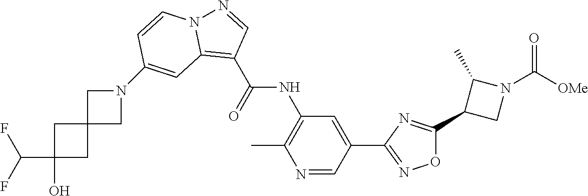 [SMILES: COC(=O)N1C[C@@H](c2nc(-c3cnc(C)c(NC(=O)c4cnn5ccc(N6CC7(C6)CC(O)(C(F)F)C7)cc45)c3)no2)[C@@H]1C] | (M + H)⁺: 609.27 |
| 281-18 | 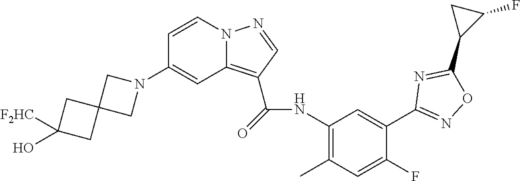 [SMILES: Cc1cc(F)c(-c2noc([C@H]3C[C@@H]3F)n2)cc1NC(=O)c1cnn2ccc(N3CC4(C3)CC(O)(C(F)F)C4)cc12] | (M + H)⁺: 557.0 |
| 281-19 | 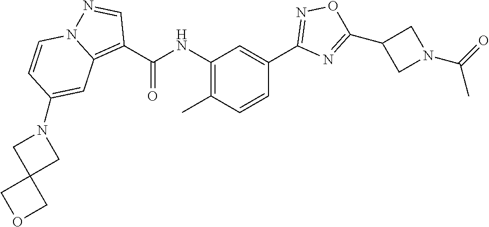 [SMILES: CC(=O)N1CC(c2nc(-c3ccc(C)c(NC(=O)c4cnn5ccc(N6CC7(COC7)C6)cc45)c3)no2)C1] | (M + H)⁺: 514.2 |
| 281-20 | 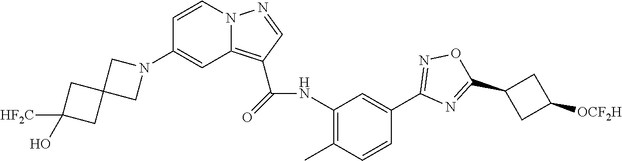 | (M + H)⁺: 601.13 |
| 281-21 | 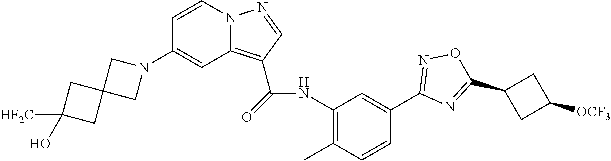 | (M + H)⁺: 619.04 |
| 281-22 | 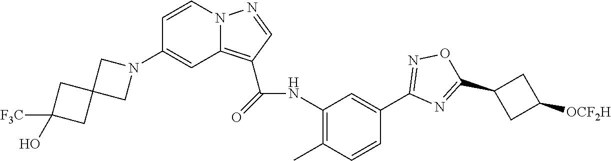 | (M + H)⁺: 619.04 |
| 281-23 | 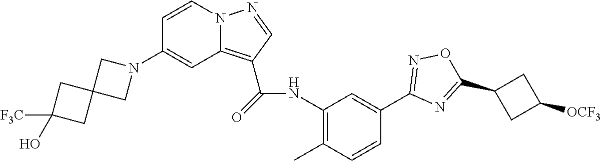 [SMILES: Cc1ccc(-c2noc([C@H]3C[C@@H](OC(F)(F)F)C3)n2)cc1NC(=O)c1cnn2ccc(N3CC4(C3)CC(O)(C(F)(F)F)C4)cc12] | (M + H)⁺: 637.04 |
| 281-24 | 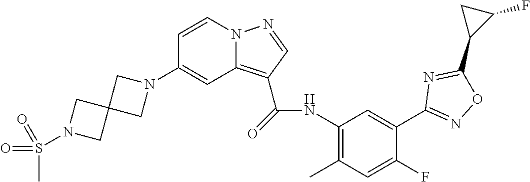 [SMILES: Cc1cc(F)c(-c2noc([C@H]3C[C@@H]3F)n2)cc1NC(=O)c1cnn2ccc(N3CC4(C3)CN(S(C)(=O)=O)C4)cc12] | (M + H)⁺: 569.93 |
| 281-25 | 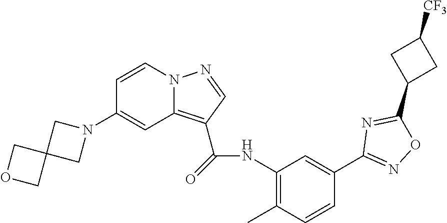 [SMILES: Cc1ccc(-c2noc([C@H]3C[C@@H](C(F)(F)F)C3)n2)cc1NC(=O)c1cnn2ccc(N3CC4(COC4)C3)cc12] | (M + H)⁺: 539.22 |
| 281-26 | 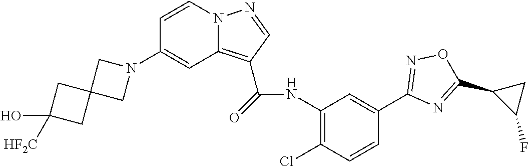 | (M + H)⁺: 558.99 |
| 281-27 | 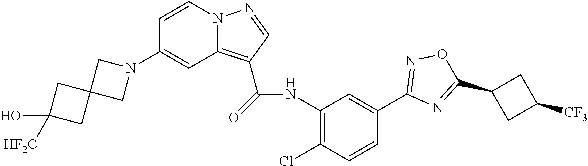 | (M + H)⁺: 623.23 |
| 281-28 | 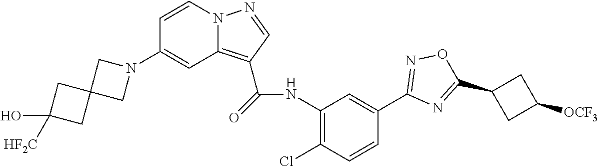 | (M + H)⁺: 639.01 |
| 281-29 | 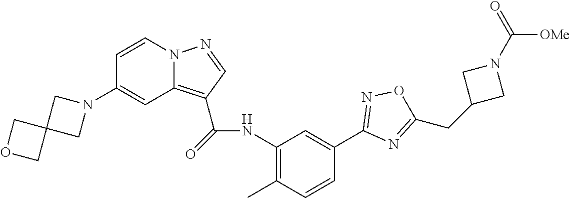 [SMILES: COC(=O)N1CC(Cc2nc(-c3ccc(C)c(NC(=O)c4cnn5ccc(N6CC7(COC7)C6)cc45)c3)no2)C1] | (M + H)⁺: 544.02 |
| 281-30 | 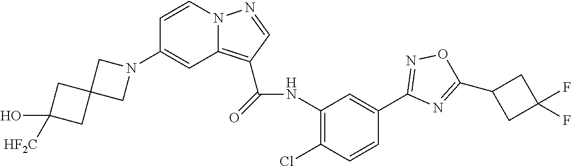 | (M + H)⁺: 590.91 |
| 281-31 | 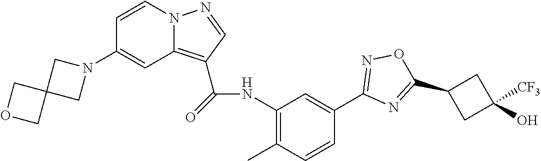 [SMILES: Cc1ccc(-c2noc([C@H]3C[C@](O)(C(F)(F)F)C3)n2)cc1NC(=O)c1cnn2ccc(N3CC4(COC4)C3)cc12] | (M + H)⁺: 554.91 |
| 281-32 | 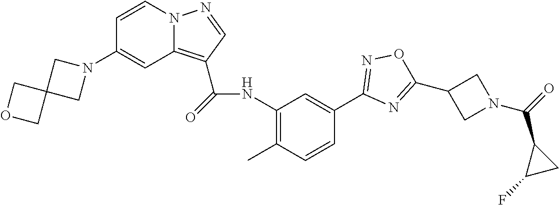 [SMILES: Cc1ccc(-c2noc(C3CN(C(=O)[C@H]4C[C@@H]4F)C3)n2)cc1NC(=O)c1cnn2ccc(N3CC4(COC4)C3)cc12] | (M + H)⁺: 558.03 |
| 281-33 | 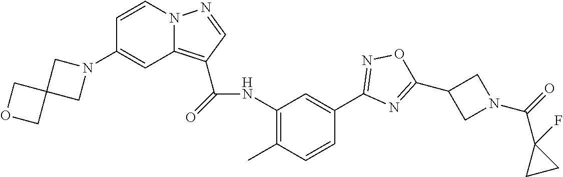 [SMILES: Cc1ccc(-c2noc(C3CN(C(=O)C4(F)CC4)C3)n2)cc1NC(=O)c1cnn2ccc(N3CC4(COC4)C3)cc12] | (M + H)⁺: 558.08 |
| 281-34 | 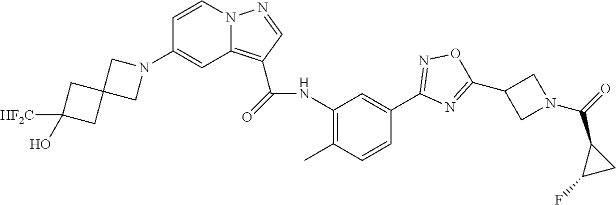 | (M + H)⁺: 622.11 |
| 281-35 | 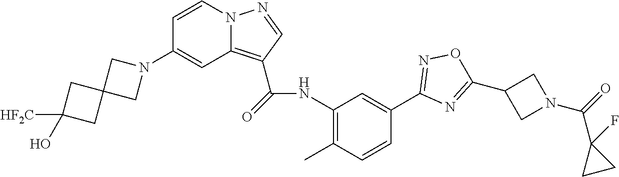 | (M + H)⁺: 622.11 |
| 281-36 | 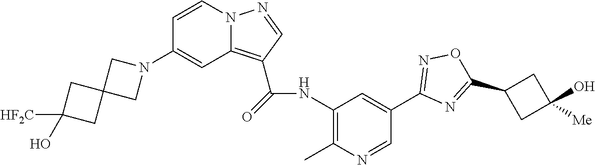 | (M + H)⁺: 565.95 |
| 281-37 | 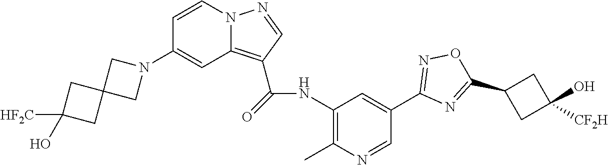 | (M + H)⁺: 601.95 |
| 281-38 | 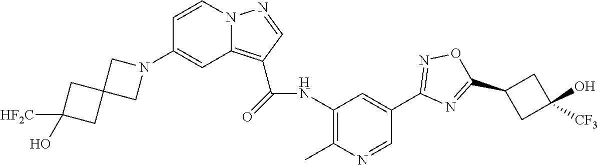 | (M + H)⁺: 620.09 |
| 281-39 | 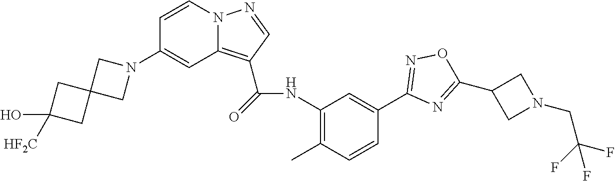 | (M + H)⁺: 618.22 |
| 281-40 | 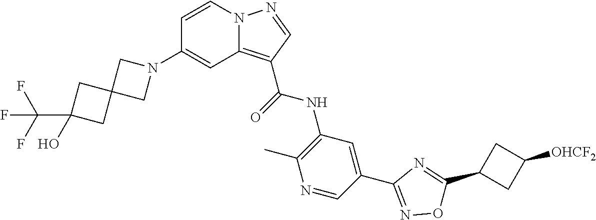 | (M + H)⁺: 620.349 |
| 281-41 | 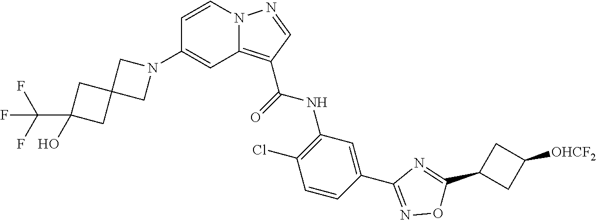 | (M + H)⁺: 639.262, 641.086 |
| 281-42 | 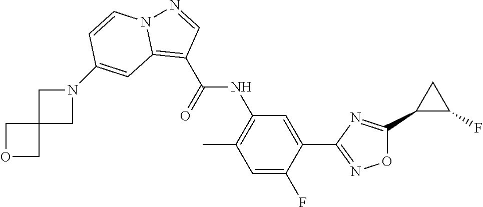 [SMILES: Cc1cc(F)c(-c2noc([C@H]3C[C@@H]3F)n2)cc1NC(=O)c1cnn2ccc(N3CC4(COC4)C3)cc12] | (M + H)⁺: 493.10 |
| 281-43 | 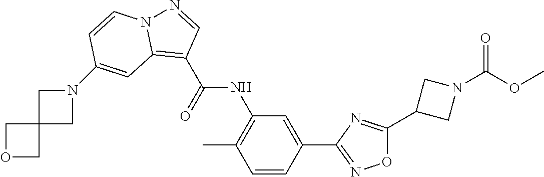 [SMILES: COC(=O)N1CC(c2nc(-c3ccc(C)c(NC(=O)c4cnn5ccc(N6CC7(COC7)C6)cc45)c3)no2)C1] | (M + H)⁺: 530.25 |
| 281-44 | 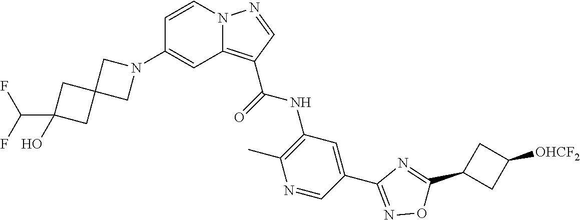 | (M + H)⁺: 602.301 |
| 281-45 | 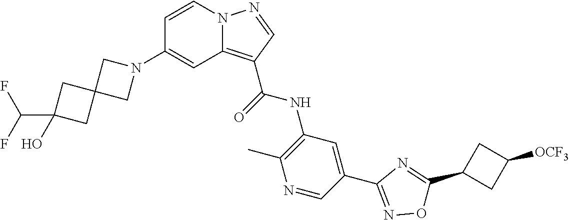 [SMILES: Cc1ncc(-c2noc([C@H]3C[C@@H](OC(F)(F)F)C3)n2)cc1NC(=O)c1cnn2ccc(N3CC4(C3)CC(O)(C(F)F)C4)cc12] | (M + H)⁺: 620.157 |
| 281-46 | 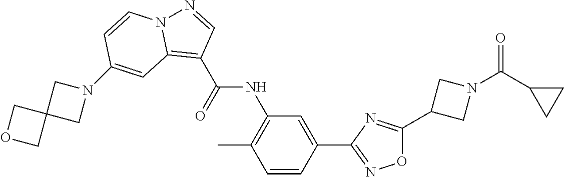 [SMILES: Cc1ccc(-c2noc(C3CN(C(=O)C4CC4)C3)n2)cc1NC(=O)c1cnn2ccc(N3CC4(COC4)C3)cc12] | (M + H)⁺: 540.25 |
| 281-47 | 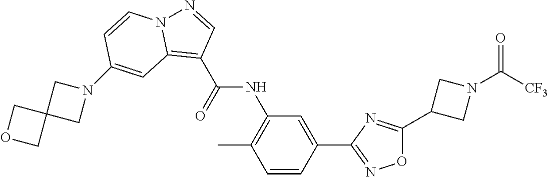 [SMILES: Cc1ccc(-c2noc(C3CN(C(=O)C(F)(F)F)C3)n2)cc1NC(=O)c1cnn2ccc(N3CC4(COC4)C3)cc12] | (M + H)⁺: 568.25 |
| 281-48 | 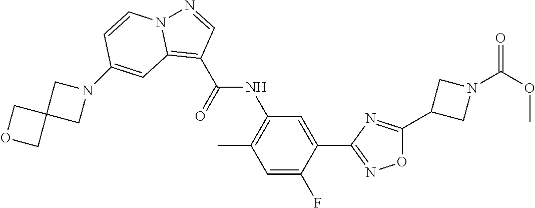 [SMILES: COC(=O)N1CC(c2nc(-c3cc(NC(=O)c4cnn5ccc(N6CC7(COC7)C6)cc45)c(C)cc3F)no2)C1] | (M + H)⁺: 548.20 |
| 281-49 | 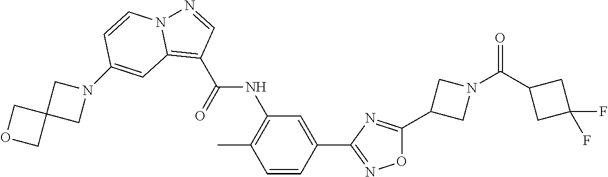 [SMILES: Cc1ccc(-c2noc(C3CN(C(=O)C4CC(F)(F)C4)C3)n2)cc1NC(=O)c1cnn2ccc(N3CC4(COC4)C3)cc12] | (M + H)⁺: 590.30 |
| 281-50 | 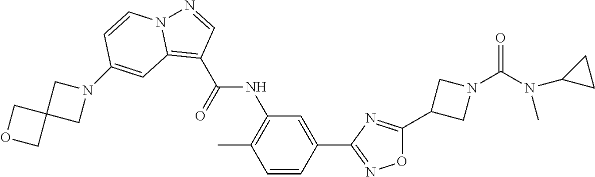 [SMILES: Cc1ccc(-c2noc(C3CN(C(=O)N(C)C4CC4)C3)n2)cc1NC(=O)c1cnn2ccc(N3CC4(COC4)C3)cc12] | (M + H)⁺: 569.35 |
| 281-51 | 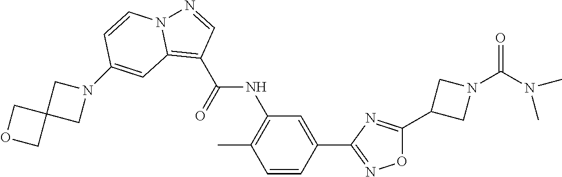 [SMILES: Cc1ccc(-c2noc(C3CN(C(=O)N(C)C)C3)n2)cc1NC(=O)c1cnn2ccc(N3CC4(COC4)C3)cc12] | (M + H)⁺: 543.30 |
| 281-52 | 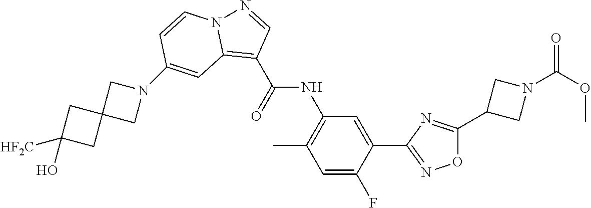 | (M + H)⁺: 612.15 |
| 281-53 | 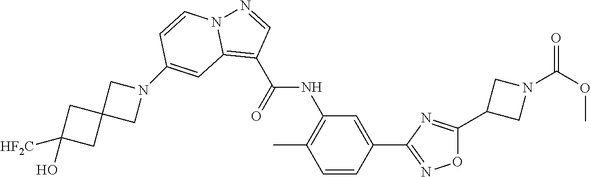 | (M + H)⁺: 594.20 |
| 281-54 | 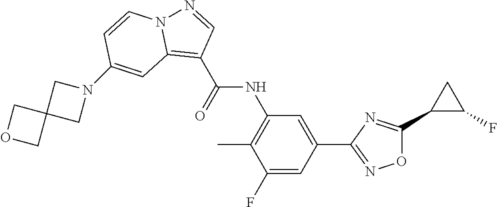 [SMILES: Cc1c(F)cc(-c2noc([C@H]3C[C@@H]3F)n2)cc1NC(=O)c1cnn2ccc(N3CC4(COC4)C3)cc12] | (M + H)⁺: 493.20 |
| 281-55 | 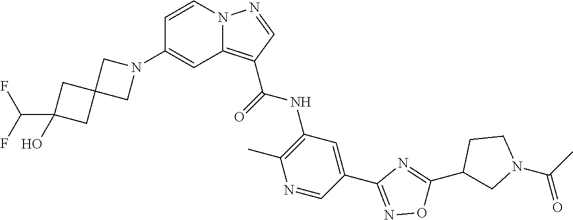 [SMILES: CC(=O)N1CCC(c2nc(-c3cnc(C)c(NC(=O)c4cnn5ccc(N6CC7(C6)CC(O)(C(F)F)C7)cc45)c3)no2)C1] | (M + H)⁺: 593.180 |
| 281-56 | 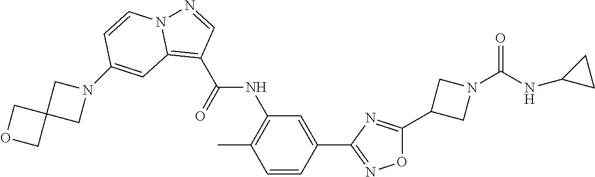 [SMILES: Cc1ccc(-c2noc(C3CN(C(=O)NC4CC4)C3)n2)cc1NC(=O)c1cnn2ccc(N3CC4(COC4)C3)cc12] | (M + H)⁺: 555.35 |
| 281-57 | 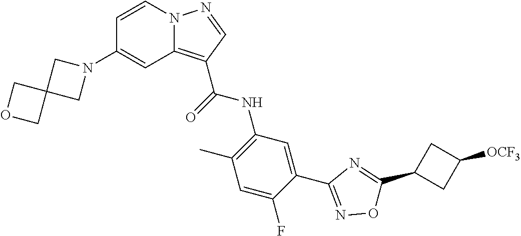 [SMILES: Cc1cc(F)c(-c2noc([C@H]3C[C@@H](OC(F)(F)F)C3)n2)cc1NC(=O)c1cnn2ccc(N3CC4(COC4)C3)cc12] | (M + H)⁺: 573.020 |
| 281-58 | 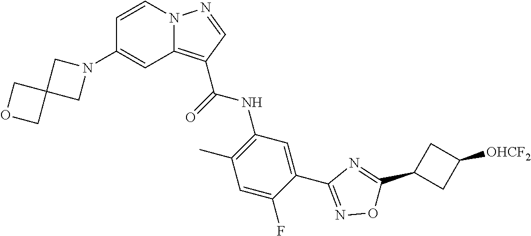 | (M + H)⁺: 555.115 |
| 281-59 | 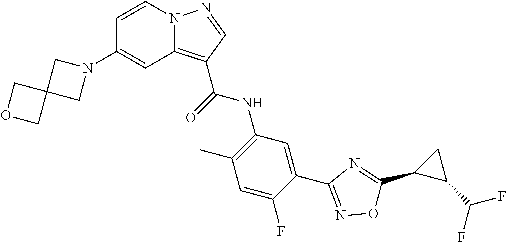 [SMILES: Cc1cc(F)c(-c2noc([C@H]3C[C@@H]3C(F)F)n2)cc1NC(=O)c1cnn2ccc(N3CC4(COC4)C3)cc12] | (M + H)⁺: 525.067 |
| 281-60 | 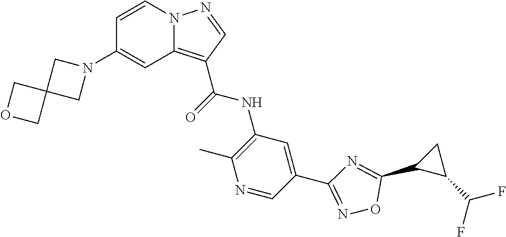 | (M + H)⁺: 508.171 |
| 281-61 | 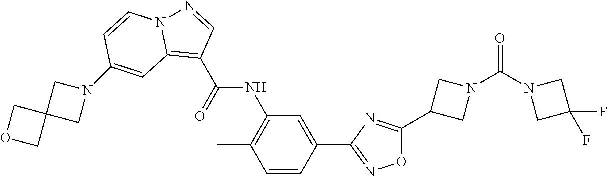 [SMILES: Cc1ccc(-c2noc(C3CN(C(=O)N4CC(F)(F)C4)C3)n2)cc1NC(=O)c1cnn2ccc(N3CC4(COC4)C3)cc12] | (M + H)⁺: 591.30 |
| 281-62 | 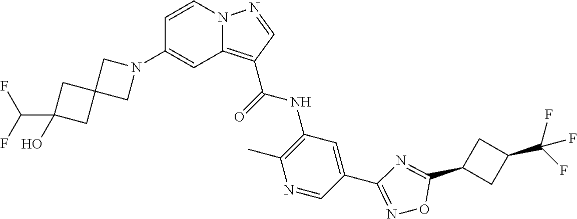 [SMILES: Cc1ncc(-c2noc([C@H]3C[C@@H](C(F)(F)F)C3)n2)cc1NC(=O)c1cnn2ccc(N3CC4(C3)CC(O)(C(F)F)C4)cc12] | (M + H)⁺: 604.269 |
| 281-63 | 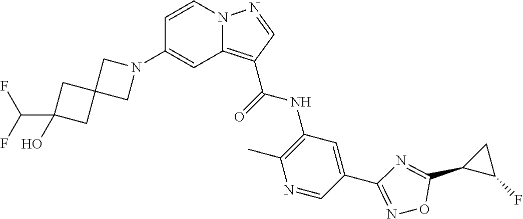 [SMILES: Cc1ncc(-c2noc([C@H]3C[C@@H]3F)n2)cc1NC(=O)c1cnn2ccc(N3CC4(C3)CC(O)(C(F)F)C4)cc12] | (M + H)⁺: 539.995 |
| 281-64 | 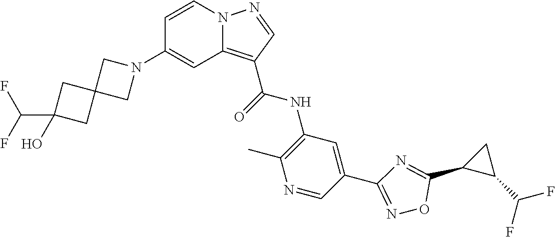 [SMILES: Cc1ncc(-c2noc([C@H]3C[C@@H]3C(F)F)n2)cc1NC(=O)c1cnn2ccc(N3CC4(C3)CC(O)(C(F)F)C4)cc12] | (M + H)⁺: 572.060 |
| 281-65 | 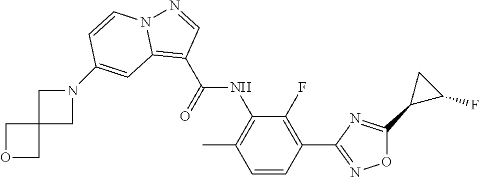 [SMILES: Cc1ccc(-c2noc([C@H]3C[C@@H]3F)n2)c(F)c1NC(=O)c1cnn2ccc(N3CC4(COC4)C3)cc12] | (M + H)⁺: 493.10 |
| 281-66 | 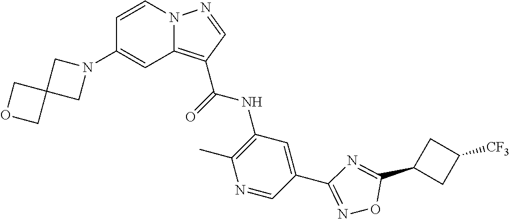 [SMILES: Cc1ncc(-c2noc([C@H]3C[C@H](C(F)(F)F)C3)n2)cc1NC(=O)c1cnn2ccc(N3CC4(COC4)C3)cc12] | (M + H)⁺: 540.187 |
| 281-67 | 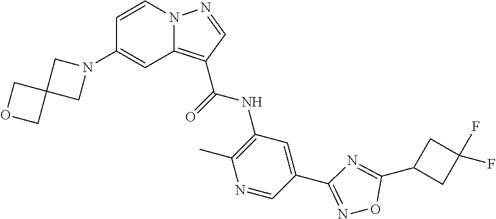 [SMILES: Cc1ncc(-c2noc(C3CC(F)(F)C3)n2)cc1NC(=O)c1cnn2ccc(N3CC4(COC4)C3)cc12] | (M + H)⁺: 508.219 |
| 281-68 | 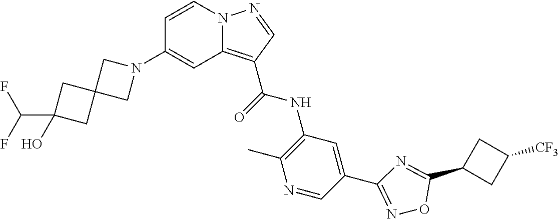 [SMILES: Cc1ncc(-c2noc([C@H]3C[C@H](C(F)(F)F)C3)n2)cc1NC(=O)c1cnn2ccc(N3CC4(C3)CC(O)(C(F)F)C4)cc12] | (M + H)⁺: 604.269 |
| 281-69 | 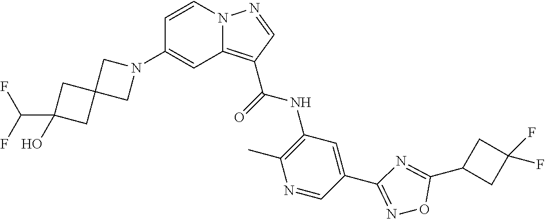 [SMILES: Cc1ncc(-c2noc(C3CC(F)(F)C3)n2)cc1NC(=O)c1cnn2ccc(N3CC4(C3)CC(O)(C(F)F)C4)cc12] | (M + H)⁺: 572.252 |
| 281-70 | 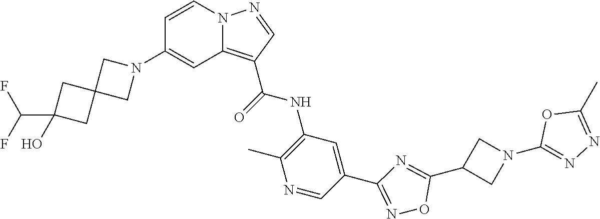 [SMILES: Cc1nnc(N2CC(c3nc(-c4cnc(C)c(NC(=O)c5cnn6ccc(N7CC8(C7)CC(O)(C(F)F)C8)cc56)c4)no3)C2)o1] | (M + H)⁺: 619.149 |
| 281-71 | 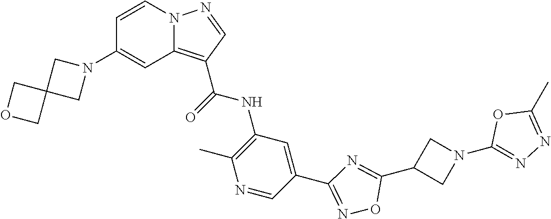 [SMILES: Cc1nnc(N2CC(c3nc(-c4cnc(C)c(NC(=O)c5cnn6ccc(N7CC8(COC8)C7)cc56)c4)no3)C2)o1] | (M + H)⁺: 555.067 |
| 281-72 | 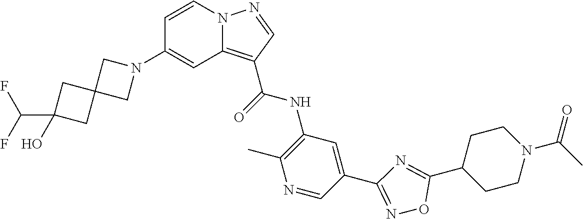 [SMILES: CC(=O)N1CCC(c2nc(-c3cnc(C)c(NC(=O)c4cnn5ccc(N6CC7(C6)CC(O)(C(F)F)C7)cc45)c3)no2)CC1] | (M + H)⁺: 607.245 |
| 281-73 | 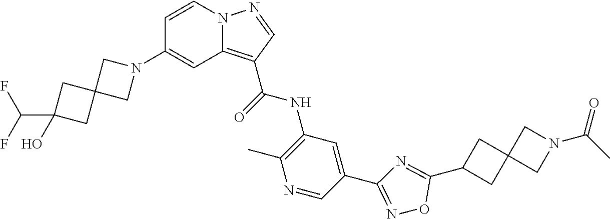 [SMILES: CC(=O)N1CC2(CC(c3nc(-c4cnc(C)c(NC(=O)c5cnn6ccc(N7CC8(C7)CC(O)(C(F)F)C8)cc56)c4)no3)C2)C1] | (M + H)⁺: 619.389 |
| 281-74 | 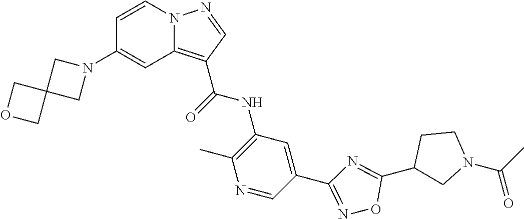 [SMILES: CC(=O)N1CCC(c2nc(-c3cnc(C)c(NC(=O)c4cnn5ccc(N6CC7(COC7)C6)cc45)c3)no2)C1] | (M + H)⁺: 529.243 |
| 281-75 | 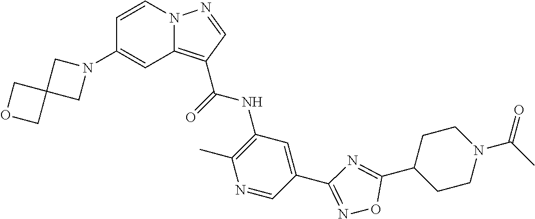 [SMILES: CC(=O)N1CCC(c2nc(-c3cnc(C)c(NC(=O)c4cnn5ccc(N6CC7(COC7)C6)cc45)c3)no2)CC1] | (M + H)⁺: 543.211 |
| 281-76 | 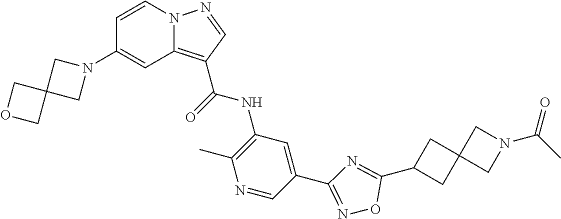 [SMILES: CC(=O)N1CC2(CC(c3nc(-c4cnc(C)c(NC(=O)c5cnn6ccc(N7CC8(COC8)C7)cc56)c4)no3)C2)C1] | (M + H)⁺: 555.067 |
| 281-77 | 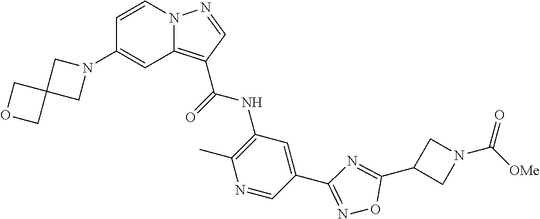 [SMILES: COC(=O)N1CC(c2nc(-c3cnc(C)c(NC(=O)c4cnn5ccc(N6CC7(COC7)C6)cc45)c3)no2)C1] | (M + H)⁺: 531.25 |
| 281-78 | 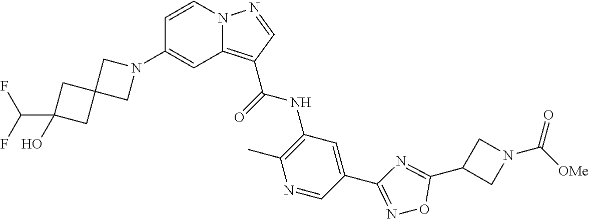 [SMILES: COC(=O)N1CC(c2nc(-c3cnc(C)c(NC(=O)c4cnn5ccc(N6CC7(C6)CC(O)(C(F)F)C7)cc45)c3)no2)C1] | (M + H)⁺: 595.15 |
| 281-79 | 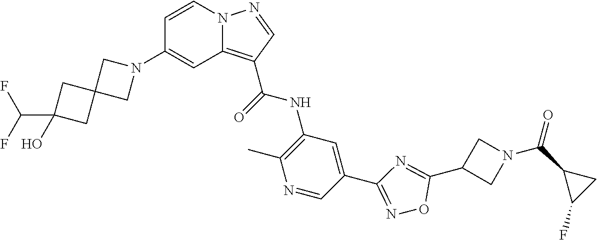 [SMILES: Cc1ncc(-c2noc(C3CN(C(=O)[C@H]4C[C@@H]4F)C3)n2)cc1NC(=O)c1cnn2ccc(N3CC4(C3)CC(O)(C(F)F)C4)cc12] | (M + H)⁺: 623.182 |
| 281-80 | 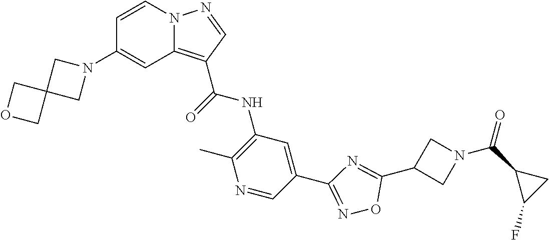 [SMILES: Cc1ncc(-c2noc(C3CN(C(=O)[C@H]4C[C@@H]4F)C3)n2)cc1NC(=O)c1cnn2ccc(N3CC4(COC4)C3)cc12] | (M + H)⁺: 559.148 |
| 281-81 | 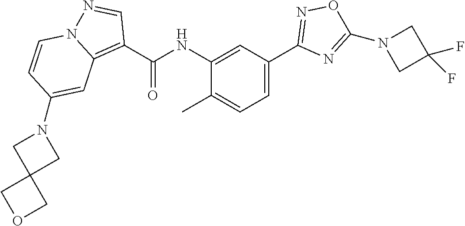 [SMILES: Cc1ccc(-c2noc(N3CC(F)(F)C3)n2)cc1NC(=O)c1cnn2ccc(N3CC4(COC4)C3)cc12] | (M + H)⁺: 508.15 |
| 281-82 | 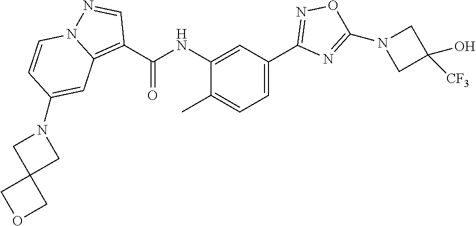 [SMILES: Cc1ccc(-c2noc(N3CC(O)(C(F)(F)F)C3)n2)cc1NC(=O)c1cnn2ccc(N3CC4(COC4)C3)cc12] | (M + H)⁺: 556.20 |
| 281-83 | 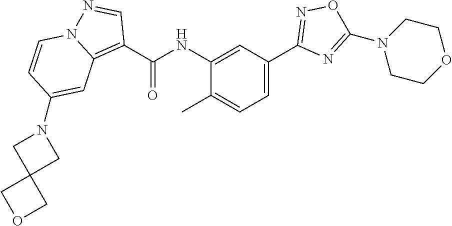 [SMILES: Cc1ccc(-c2noc(N3CCOCC3)n2)cc1NC(=O)c1cnn2ccc(N3CC4(COC4)C3)cc12] | (M + H)⁺: 502.20 |
| 281-84 | 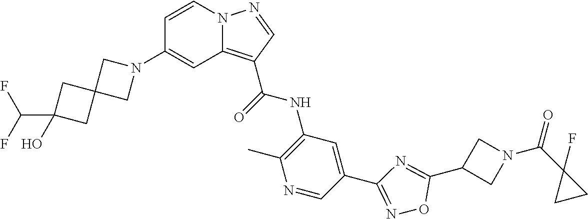 [SMILES: Cc1ncc(-c2noc(C3CN(C(=O)C4(F)CC4)C3)n2)cc1NC(=O)c1cnn2ccc(N3CC4(C3)CC(O)(C(F)F)C4)cc12] | (M + H)⁺: 623.229 |
| 281-85 | 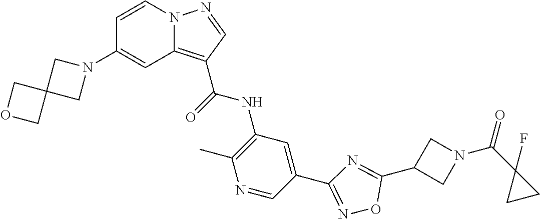 [SMILES: Cc1ncc(-c2noc(C3CN(C(=O)C4(F)CC4)C3)n2)cc1NC(=O)c1cnn2ccc(N3CC4(COC4)C3)cc12] | (M + H)⁺: 559.100 |
| 281-86 | 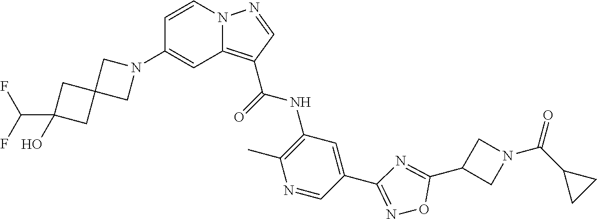 [SMILES: Cc1ncc(-c2noc(C3CN(C(=O)C4CC4)C3)n2)cc1NC(=O)c1cnn2ccc(N3CC4(C3)CC(O)(C(F)F)C4)cc12] | (M + H)⁺: 605.085 |
| 281-87 | 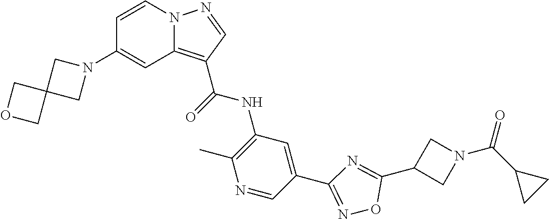 [SMILES: Cc1ncc(-c2noc(C3CN(C(=O)C4CC4)C3)n2)cc1NC(=O)c1cnn2ccc(N3CC4(COC4)C3)cc12] | (M + H)⁺: 541.243 |
| 281-88 | 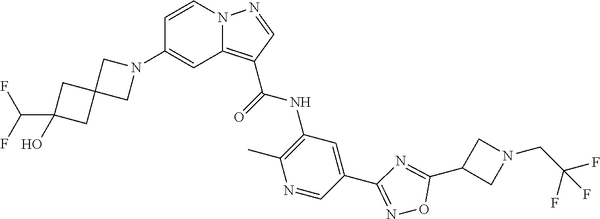 [SMILES: Cc1ncc(-c2noc(C3CN(CC(F)(F)F)C3)n2)cc1NC(=O)c1cnn2ccc(N3CC4(C3)CC(O)(C(F)F)C4)cc12] | (M + H)⁺: 619.149 |
| 281-89 | 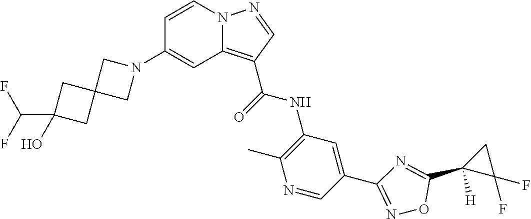 [SMILES: Cc1ncc(-c2noc([C@H]3CC3(F)F)n2)cc1NC(=O)c1cnn2ccc(N3CC4(C3)CC(O)(C(F)F)C4)cc12] | (M + H)⁺: 557.996 |
| 281-90 | 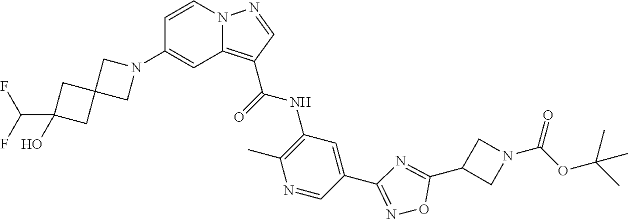 [SMILES: Cc1ncc(-c2noc(C3CN(C(=O)OC(C)(C)C)C3)n2)cc1NC(=O)c1cnn2ccc(N3CC4(C3)CC(O)(C(F)F)C4)cc12] | (M + H)⁺: 637.342, 581.084 |
| 281-91 | 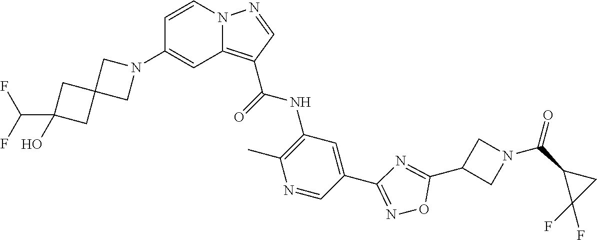 [SMILES: Cc1ncc(-c2noc(C3CN(C(=O)[C@H]4CC4(F)F)C3)n2)cc1NC(=O)c1cnn2ccc(N3CC4(C3)CC(O)(C(F)F)C4)cc12] | (M + H)⁺: 641.374 |
| 281-92 | 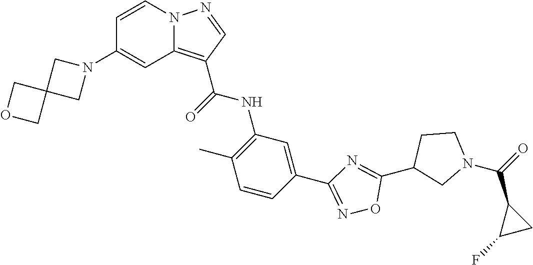 [SMILES: Cc1ccc(-c2noc(C3CCN(C(=O)[C@H]4C[C@@H]4F)C3)n2)cc1NC(=O)c1cnn2ccc(N3CC4(COC4)C3)cc12] | (M + H)⁺: 572.20 |
| 281-93 | 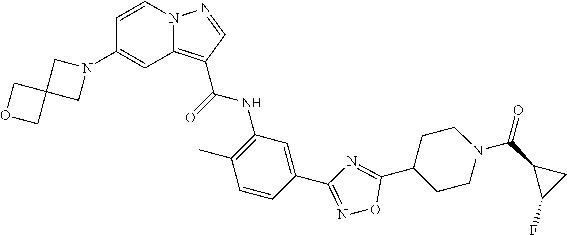 [SMILES: Cc1ccc(-c2noc(C3CCN(C(=O)[C@H]4C[C@@H]4F)CC3)n2)cc1NC(=O)c1cnn2ccc(N3CC4(COC4)C3)cc12] | (M + H)⁺: 586.25 |
| 281-94 | 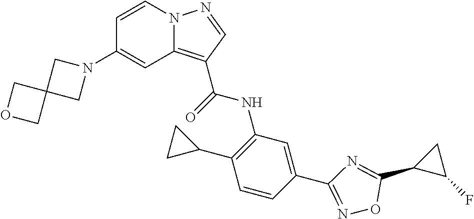 [SMILES: O=C(Nc1cc(-c2noc([C@H]3C[C@@H]3F)n2)ccc1C1CC1)c1cnn2ccc(N3CC4(COC4)C3)cc12] | (M + H)⁺: 501.25 |
| 281-95 | 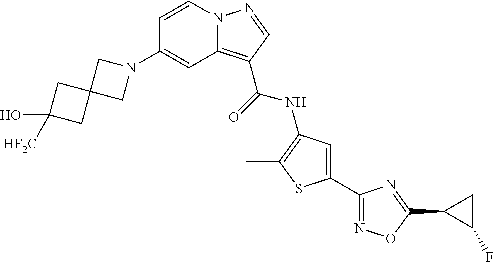 | (M + H)⁺: 545.20 |
| 281-96 | 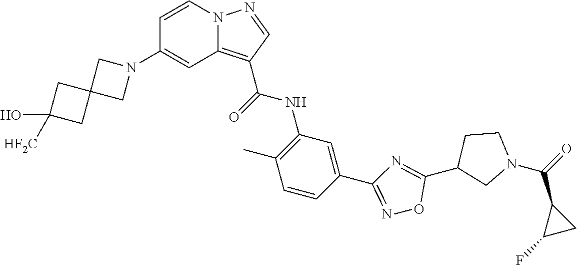 | (M + H)⁺: 636.45 |
| 281-97 | 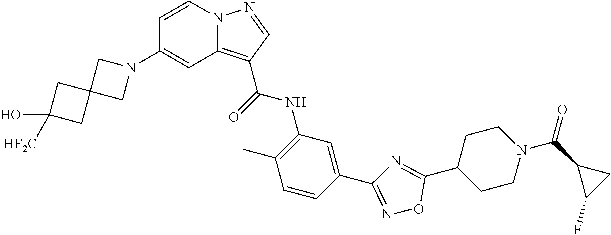 | (M + H)⁺: 650.25 |
| 281-98 | 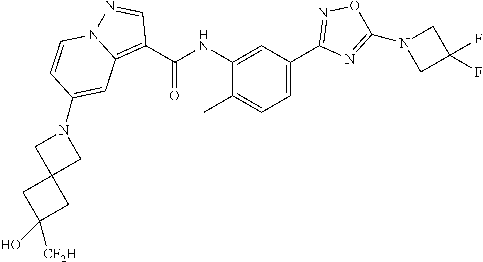 [SMILES: Cc1ccc(-c2noc(N3CC(F)(F)C3)n2)cc1NC(=O)c1cnn2ccc(N3CC4(C3)CC(O)(C(F)F)C4)cc12] | (M + H)⁺: 572.15 |
| 281-99 | 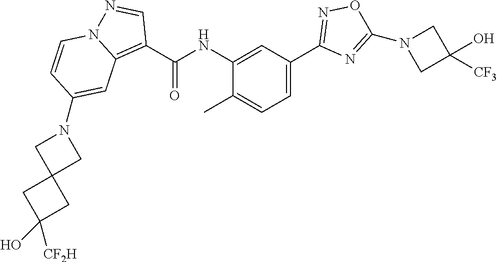 [SMILES: Cc1ccc(-c2noc(N3CC(O)(C(F)(F)F)C3)n2)cc1NC(=O)c1cnn2ccc(N3CC4(C3)CC(O)(C(F)F)C4)cc12] | (M + H)⁺: 620.25 |
| 281-100 | 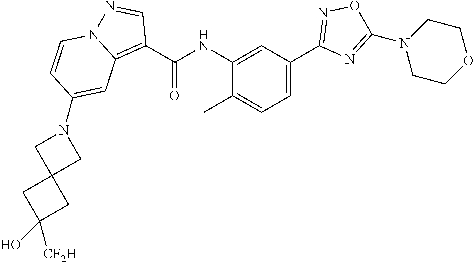 [SMILES: Cc1ccc(-c2noc(N3CCOCC3)n2)cc1NC(=O)c1cnn2ccc(N3CC4(C3)CC(O)(C(F)F)C4)cc12] | (M + H)⁺: 566.30 |
| 281-101 | 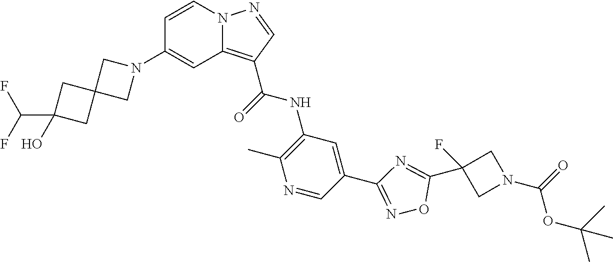 [SMILES: Cc1ncc(-c2noc(C3(F)CN(C(=O)OC(C)(C)C)C3)n2)cc1NC(=O)c1cnn2ccc(N3CC4(C3)CC(O)(C(F)F)C4)cc12] | (M + H)⁺: 655.295 |
| 281-102 | 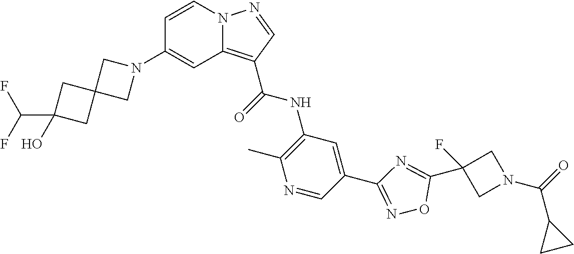 [SMILES: Cc1ncc(-c2noc(C3(F)CN(C(=O)C4CC4)C3)n2)cc1NC(=O)c1cnn2ccc(N3CC4(C3)CC(O)(C(F)F)C4)cc12] | (M + H)⁺: 624.286 |
| 281-103 | 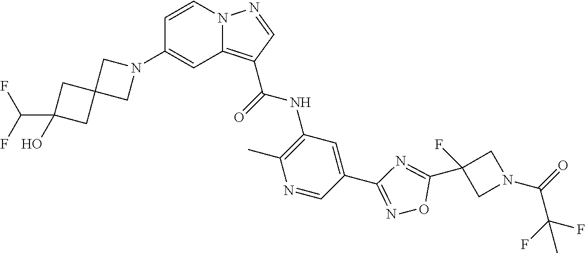 [SMILES: Cc1ncc(-c2noc(C3(F)CN(C(=O)C(C)(F)F)C3)n2)cc1NC(=O)c1cnn2ccc(N3CC4(C3)CC(O)(C(F)F)C4)cc12] | (M + H)⁺: 647.134 |
| 281-104 | 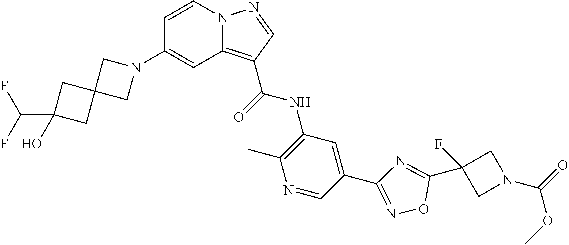 [SMILES: COC(=O)N1CC(F)(c2nc(-c3cnc(C)c(NC(=O)c4cnn5ccc(N6CC7(C6)CC(O)(C(F)F)C7)cc45)c3)no2)C1] | (M + H)⁺: 613.197 |
| 281-105 | 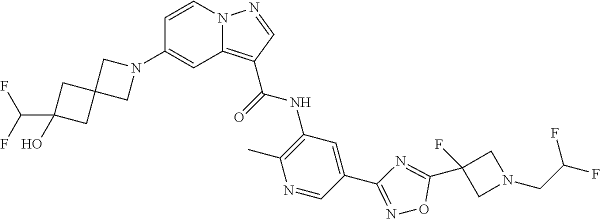 [SMILES: Cc1ncc(-c2noc(C3(F)CN(CC(F)F)C3)n2)cc1NC(=O)c1cnn2ccc(N3CC4(C3)CC(O)(C(F)F)C4)cc12] | (M + H)⁺: 618.957 |
| 281-106 | 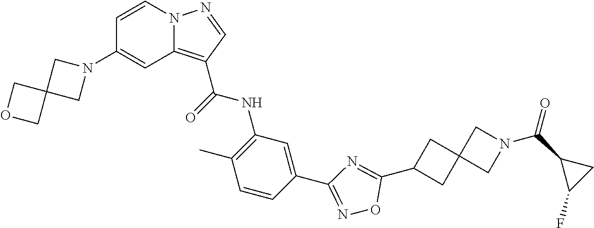 [SMILES: Cc1ccc(-c2noc(C3CC4(C3)CN(C(=O)[C@H]3C[C@@H]3F)C4)n2)cc1NC(=O)c1cnn2ccc(N3CC4(COC4)C3)cc12] | (M + H)⁺: 598.40 |
| 281-107 | 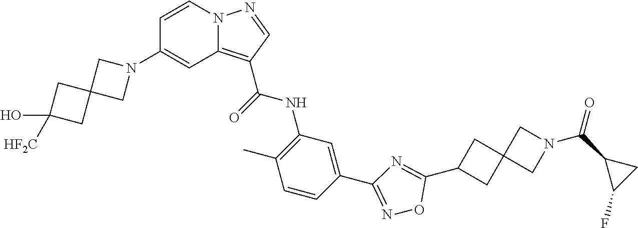 | (M − H)⁺: 662.35 |
| 281-108 | 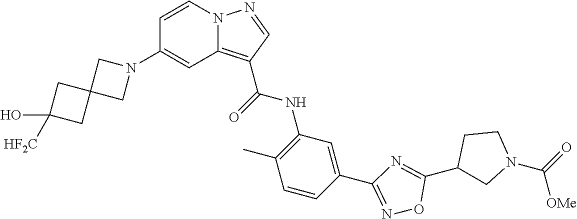 | (M + H)⁺: 608.20 |
| 281-109 | 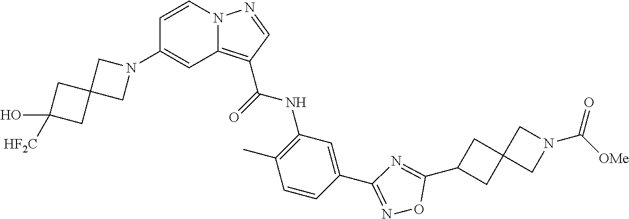 | (M + H)⁺: 634.20 |
| 281-110 | 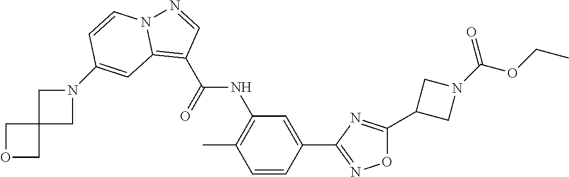 [SMILES: CCOC(=O)N1CC(c2nc(-c3ccc(C)c(NC(=O)c4cnn5ccc(N6CC7(COC7)C6)cc45)c3)no2)C1] | (M + H)⁺: 544.35 |
| 281-111 | 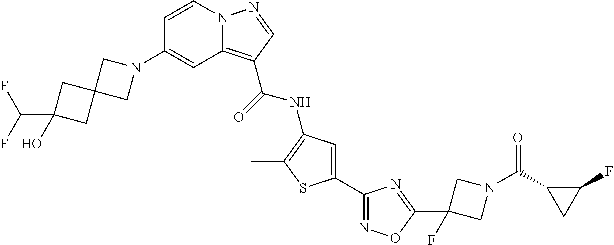 [SMILES: Cc1sc(-c2noc(C3(F)CN(C(=O)[C@H]4C[C@@H]4F)C3)n2)cc1NC(=O)c1cnn2ccc(N3CC4(C3)CC(O)(C(F)F)C4)cc12] | (M + H)⁺: 646.222 |
| 281-112 | 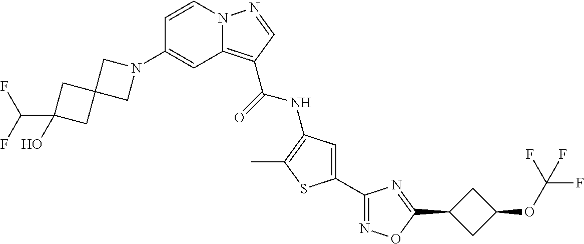 [SMILES: Cc1sc(-c2noc([C@H]3C[C@@H](OC(F)(F)F)C3)n2)cc1NC(=O)c1cnn2ccc(N3CC4(C3)CC(O)(C(F)F)C4)cc12] | (M + H)⁺: 625.166 |
| 281-113 | 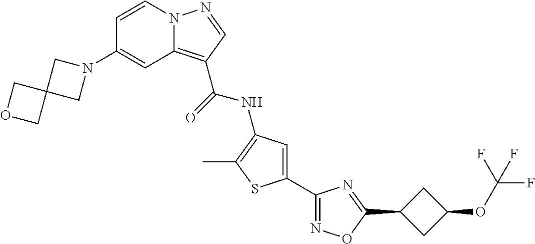 [SMILES: Cc1sc(-c2noc([C@H]3C[C@@H](OC(F)(F)F)C3)n2)cc1NC(=O)c1cnn2ccc(N3CC4(COC4)C3)cc12] | (M + H)⁺: 561.156 |
| 281-114 | 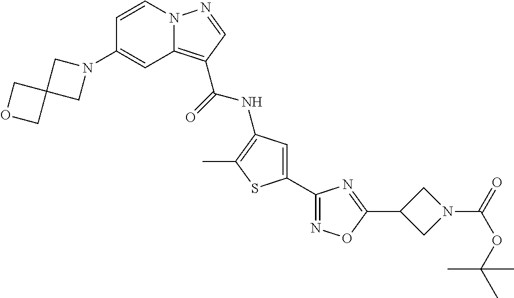 [SMILES: Cc1sc(-c2noc(C3CN(C(=O)OC(C)(C)C)C3)n2)cc1NC(=O)c1cnn2ccc(N3CC4(COC4)C3)cc12] | (M + H)⁺: 578.300, 522.187 |
| 281-115 | 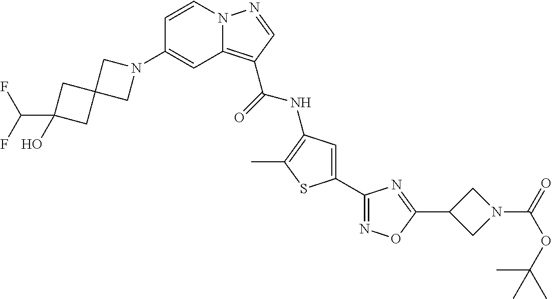 [SMILES: Cc1sc(-c2noc(C3CN(C(=O)OC(C)(C)C)C3)n2)cc1NC(=O)c1cnn2ccc(N3CC4(C3)CC(O)(C(F)F)C4)cc12] | (M + H)⁺: 642.286, 586.268 |
| 281-116 | 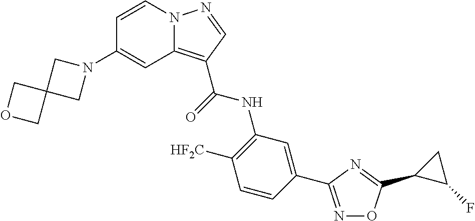 | (M + H)⁺: 511.10 |
| 281-117 | 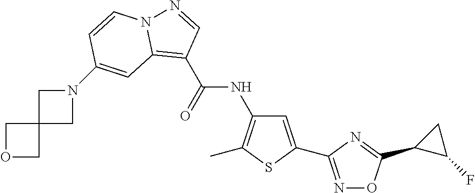 [SMILES: Cc1sc(-c2noc([C@H]3C[C@@H]3F)n2)cc1NC(=O)c1cnn2ccc(N3CC4(COC4)C3)cc12] | (M + H)⁺: 481.098 |
| 281-118 | 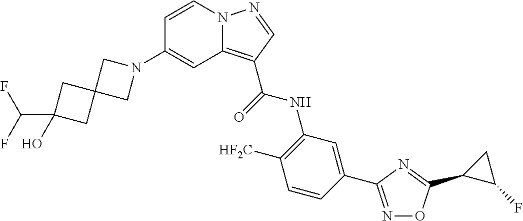 | (M + H)⁺: 575.15 |
| 281-119 | 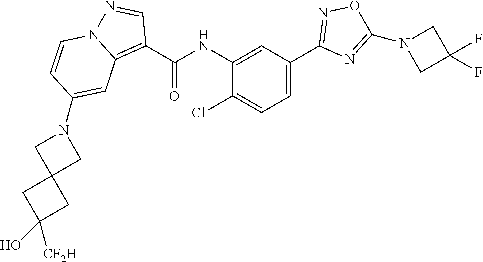 [SMILES: O=C(Nc1cc(-c2noc(N3CC(F)(F)C3)n2)ccc1Cl)c1cnn2ccc(N3CC4(C3)CC(O)(C(F)F)C4)cc12] | (M + H)⁺: 592.25 |
| 281-120 | 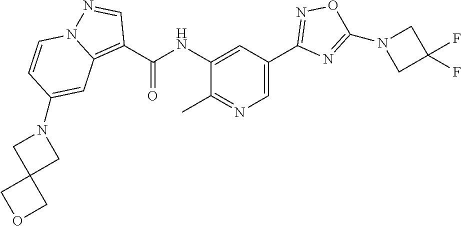 [SMILES: Cc1ncc(-c2noc(N3CC(F)(F)C3)n2)cc1NC(=O)c1cnn2ccc(N3CC4(COC4)C3)cc12] | (M + H)⁺: 509.25 |
| 281-121 | 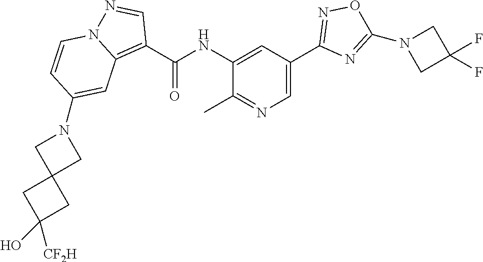 [SMILES: Cc1ncc(-c2noc(N3CC(F)(F)C3)n2)cc1NC(=O)c1cnn2ccc(N3CC4(C3)CC(O)(C(F)F)C4)cc12] | (M + H)⁺: 573.35 |
| 281-122 | 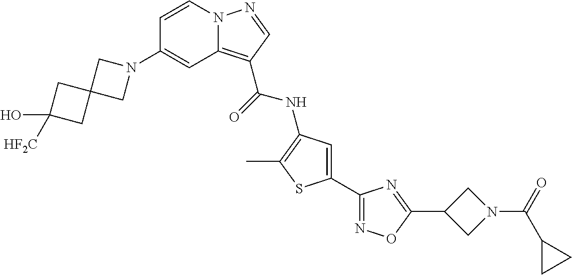 | (M + H)⁺: 610.25 |
| 281-123 | 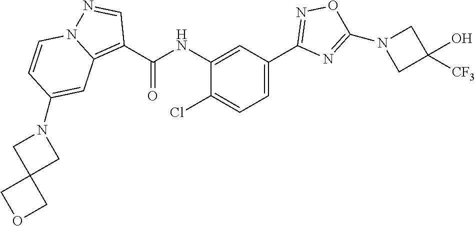 [SMILES: O=C(Nc1cc(-c2noc(N3CC(O)(C(F)(F)F)C3)n2)ccc1Cl)c1cnn2ccc(N3CC4(COC4)C3)cc12] | (M + H)⁺: 576.25 |
| 281-124 | 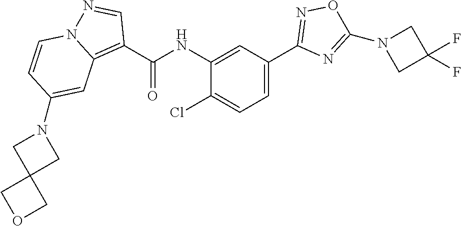 [SMILES: O=C(Nc1cc(-c2noc(N3CC(F)(F)C3)n2)ccc1Cl)c1cnn2ccc(N3CC4(COC4)C3)cc12] | (M + H)⁺: 528.25 |
| 281-125 | 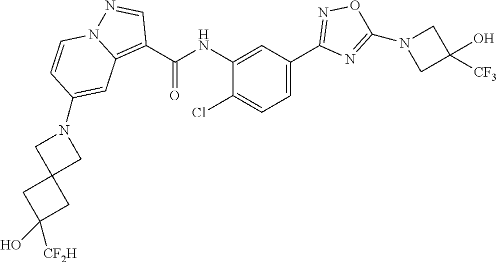 [SMILES: O=C(Nc1cc(-c2noc(N3CC(O)(C(F)(F)F)C3)n2)ccc1Cl)c1cnn2ccc(N3CC4(C3)CC(O)(C(F)F)C4)cc12] | (M + H)⁺: 640.35 |
| 281-126 | 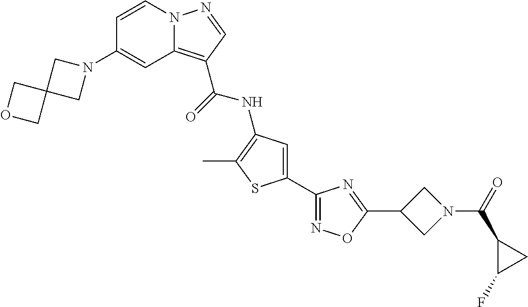 [SMILES: Cc1sc(-c2noc(C3CN(C(=O)[C@H]4C[C@@H]4F)C3)n2)cc1NC(=O)c1cnn2ccc(N3CC4(COC4)C3)cc12] | (M + H)⁺: 564.236 |
| 281-127 | 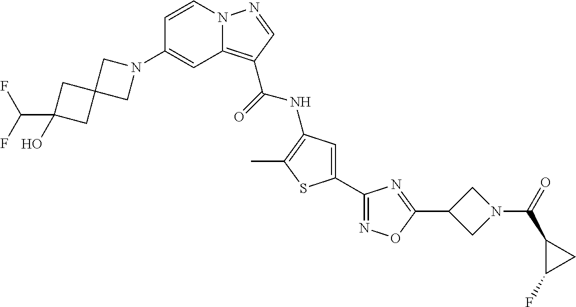 [SMILES: Cc1sc(-c2noc(C3CN(C(=O)[C@H]4C[C@@H]4F)C3)n2)cc1NC(=O)c1cnn2ccc(N3CC4(C3)CC(O)(C(F)F)C4)cc12] | (M + H)⁺: 628.174 |
| 281-128 | 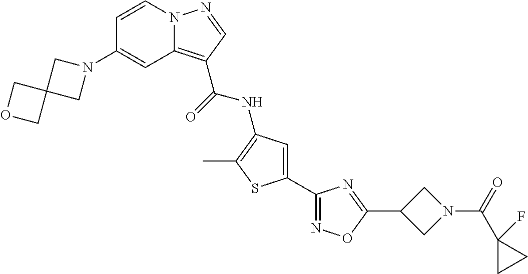 [SMILES: Cc1sc(-c2noc(C3CN(C(=O)C4(F)CC4)C3)n2)cc1NC(=O)c1cnn2ccc(N3CC4(COC4)C3)cc12] | (M + H)⁺: 564.140 |
| 281-129 | 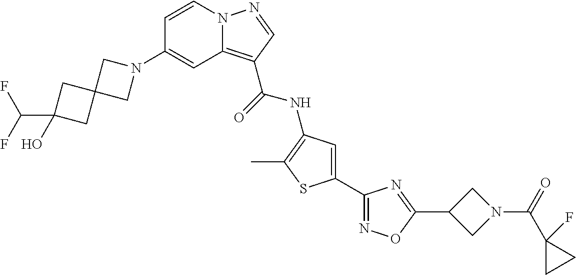 [SMILES: Cc1sc(-c2noc(C3CN(C(=O)C4(F)CC4)C3)n2)cc1NC(=O)c1cnn2ccc(N3CC4(C3)CC(O)(C(F)F)C4)cc12] | (M + H)⁺: 628.270 |
| 281-130 | 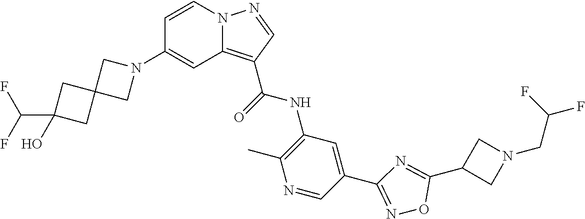 [SMILES: Cc1ncc(-c2noc(C3CN(CC(F)F)C3)n2)cc1NC(=O)c1cnn2ccc(N3CC4(C3)CC(O)(C(F)F)C4)cc12] | (M + H)⁺: 601.245 |
| 281-131 | 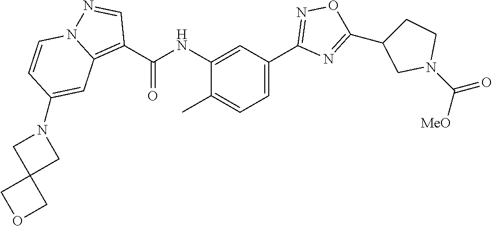 [SMILES: COC(=O)N1CCC(c2nc(-c3ccc(C)c(NC(=O)c4cnn5ccc(N6CC7(COC7)C6)cc45)c3)no2)C1] | (M + H)⁺: 544.45 |
| 281-132 | 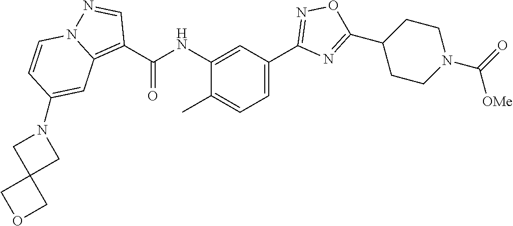 [SMILES: COC(=O)N1CCC(c2nc(-c3ccc(C)c(NC(=O)c4cnn5ccc(N6CC7(COC7)C6)cc45)c3)no2)CC1] | (M + H)⁺: 558.30 |
| 281-133 | 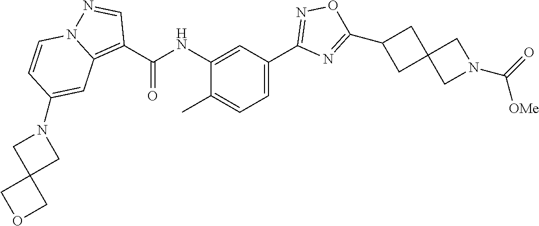 [SMILES: COC(=O)N1CC2(CC(c3nc(-c4ccc(C)c(NC(=O)c5cnn6ccc(N7CC8(COC8)C7)cc56)c4)no3)C2)C1] | (M + H)⁺: 570.40 |
| 281-134 | 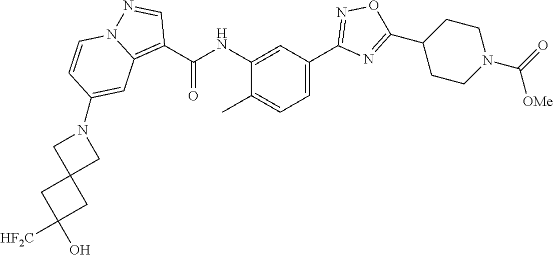 | (M + H)⁺: 622.35 |

### Example 282

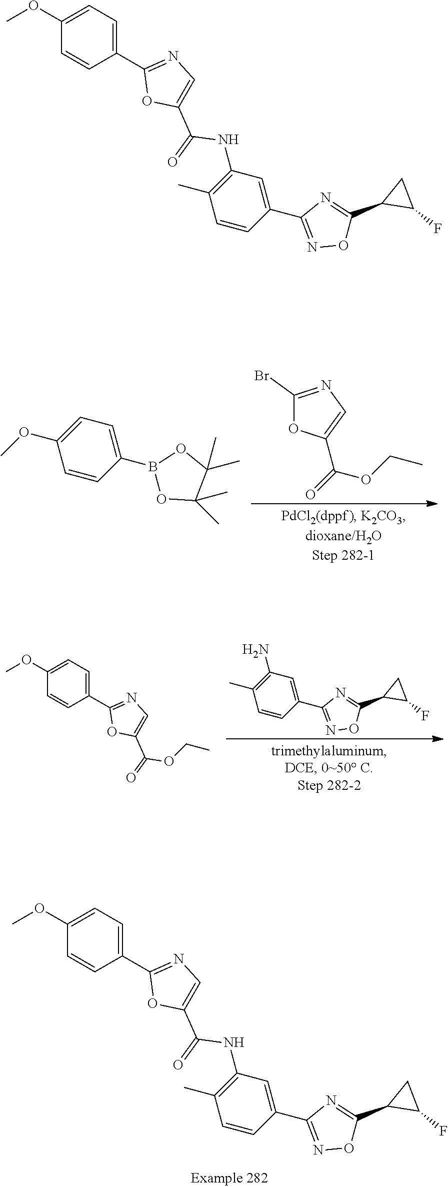

Step 282-1: A solution of 2-(4-methoxyphenyl)-4,4,5,5-tetramethyl-1,3,2-dioxaborolane (100 mg, 0.427 mmol, 1 equiv), ethyl 2-bromooxazole-5-carboxylate (103.38 mg, 0.470 mmol, 1.1 equiv), Pd(dppf)Cl₂—CH₂Cl₂ adduct (34.80 mg, 0.043 mmol, 0.1 equiv) and K₂CO₃ (177.11 mg, 1.281 mmol, 3 equiv) in H₂O (0.2 mL) and dioxane (2 mL) was stirred for 2 h at 100° C. under nitrogen atmosphere. The reaction was quenched by the addition of water (2 mL) at room temperature. The resulting mixture was concentrated under reduced pressure. The residue was purified by reversed-phase flash chromatography with the following conditions: column, C18 silica gel; mobile phase, water with 10 mmol/L NH₄HCO₃ in MeCN, 0% to 100% gradient in 16 min; detector, UV 254 nm. This resulted in the desired product (40 mg, 37.87%) as a yellow solid. (ES, m/z): [M+H]⁺=248.00.
Step 282-2: To a stirred solution of the compound from Step 282-1 (40 mg, 0.162 mmol, 1 equiv) and 5-(5-((1R,2S)-2-fluorocyclopropyl)-1,2,4-oxadiazol-3-yl)-2-methylaniline (37.73 mg, 0.162 mmol, 1 equiv) in 1,2-dichloroethane (3 mL) were added trimethylaluminium (34.99 mg, 0.486 mmol, 3 equiv) dropwise at 0° C. under nitrogen atmosphere. The reaction was stirred for 10 min at room temperature. Then, the reaction was stirred for another 2 h at 50° C. The reaction was quenched by the addition of sat. NH₄Cl (aq.) (3 mL) at room temperature. The mixture was extracted with DCM (2×8 mL). The organic phase was concentrated under a vacuum. The residue was purified by reversed-phase flash chromatography with the following conditions: column, C18 silica gel; mobile phase, MeCN in Water (0.1% FA), 0% to 100% gradient in 10 min; detector, UV 254 nm. The crude product was purified by Prep-HPLC. This resulted in Example 282 (31.0 mg, 43.98%) as a white solid. (ES, m/z): [M+H]⁺=435.10 ¹H NMR (400 MHz, DMSO-d₆) δ 10.24 (s, 1H), 8.17-8.07 (m, 2H), 8.07-7.92 (m, 2H), 7.80 (dd, J=1.8, 7.9 Hz, 1H), 7.50 (d, J=8.0 Hz, 1H), 7.23-7.06 (m, 2H), 5.29 (dddd, J=1.8, 3.5, 5.8, 62.8 Hz, 1H), 3.87 (s, 3H), 3.06 (dddd, J=1.9, 6.9, 11.2, 16.6 Hz, 1H), 2.34 (s, 3H), 1.95 (dddd, J=3.5, 7.1, 10.9, 21.9 Hz, 1H), 1.59 (dq, J=6.8, 13.3 Hz, 1H).

The following examples were prepared using procedures similar to those described above:

| Example | Structure | ESI-MS |
|---|---|---|
| 283 | 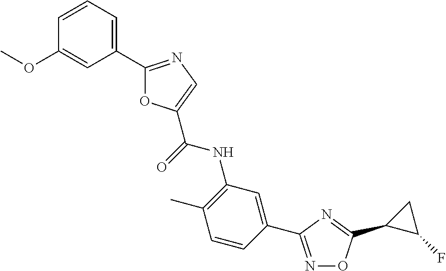 [SMILES: COc1cccc(-c2ncc(C(=O)Nc3cc(-c4noc([C@H]5C[C@@H]5F)n4)ccc3C)o2)c1] | (M + H)⁺: 435.15 |
| 284 | 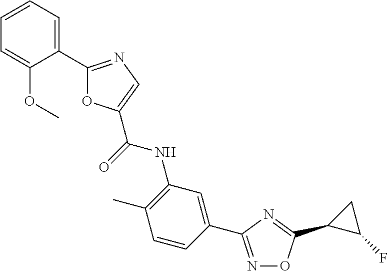 [SMILES: COc1ccccc1-c1ncc(C(=O)Nc2cc(-c3noc([C@H]4C[C@@H]4F)n3)ccc2C)o1] | (M + H)⁺: 435.10 |
| 285 | 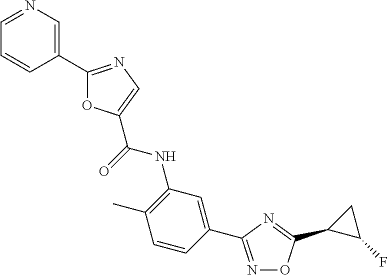 [SMILES: Cc1ccc(-c2noc([C@H]3C[C@@H]3F)n2)cc1NC(=O)c1cnc(-c2cccnc2)o1] | (M + H)⁺: 406.10 |
| 286 | 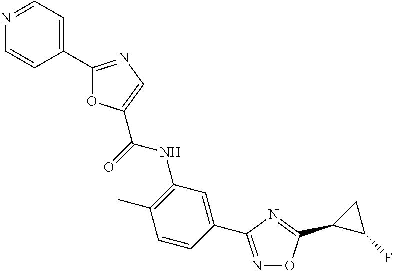 [SMILES: Cc1ccc(-c2noc([C@H]3C[C@@H]3F)n2)cc1NC(=O)c1cnc(-c2ccncc2)o1] | (M + H)⁺: 406.10 |
| 287 | 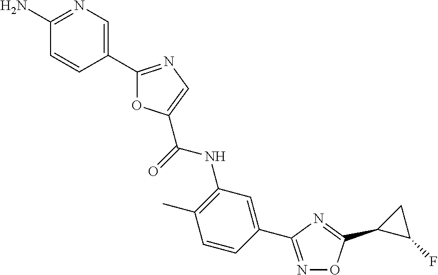 [SMILES: Cc1ccc(-c2noc([C@H]3C[C@@H]3F)n2)cc1NC(=O)c1cnc(-c2ccc(N)nc2)o1] | (M + H)⁺: 421.10 |
| 288 | 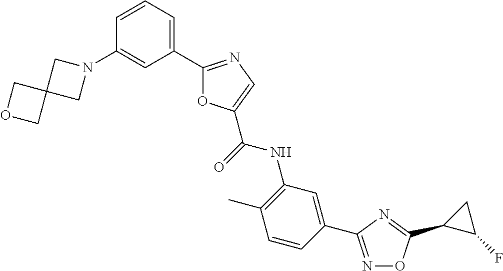 [SMILES: Cc1ccc(-c2noc([C@H]3C[C@@H]3F)n2)cc1NC(=O)c1cnc(-c2cccc(N3CC4(COC4)C3)c2)o1] | (M + H)⁺: 502.15 |
| 289 | 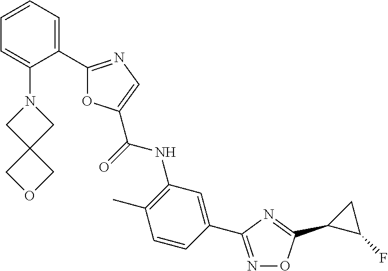 [SMILES: Cc1ccc(-c2noc([C@H]3C[C@@H]3F)n2)cc1NC(=O)c1cnc(-c2ccccc2N2CC3(COC3)C2)o1] | (M + H)⁺: 502.10 |
| 290 | 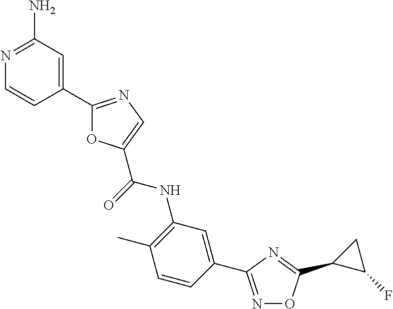 [SMILES: Cc1ccc(-c2noc([C@H]3C[C@@H]3F)n2)cc1NC(=O)c1cnc(-c2ccnc(N)c2)o1] | (M + H)⁺: 421.15 |
| 291 | 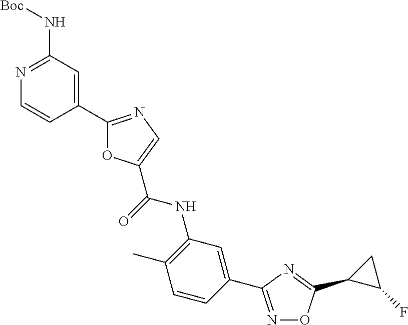 [SMILES: Cc1ccc(-c2noc([C@H]3C[C@@H]3F)n2)cc1NC(=O)c1cnc(-c2ccnc(NC(=O)OC(C)(C)C)c2)o1] | (M + H)⁺: 521.15 |
| 292 | 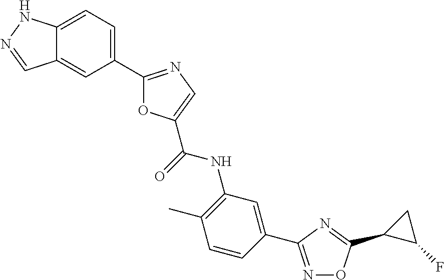 [SMILES: Cc1ccc(-c2noc([C@H]3C[C@@H]3F)n2)cc1NC(=O)c1cnc(-c2ccc3[nH]ncc3c2)o1] | (M + H)⁺: 445.15 |
| 293 | 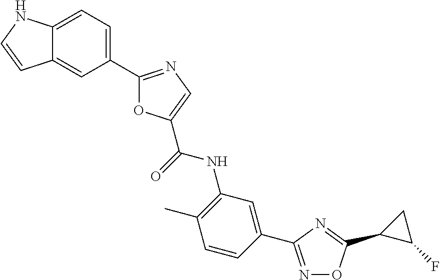 [SMILES: Cc1ccc(-c2noc([C@H]3C[C@@H]3F)n2)cc1NC(=O)c1cnc(-c2ccc3[nH]ccc3c2)o1] | (M + H)⁺: 444.05 |
| 294 | 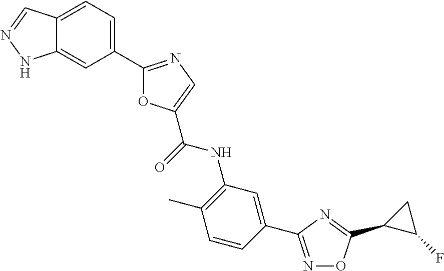 [SMILES: Cc1ccc(-c2noc([C@H]3C[C@@H]3F)n2)cc1NC(=O)c1cnc(-c2ccc3cn[nH]c3c2)o1] | (M + H)⁺: 445.10 |
| 295 | 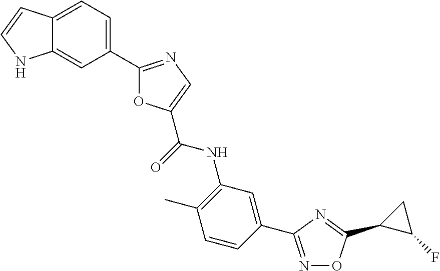 [SMILES: Cc1ccc(-c2noc([C@H]3C[C@@H]3F)n2)cc1NC(=O)c1cnc(-c2ccc3cc[nH]c3c2)o1] | (M + H)⁺: 444.15 |
| 296 | 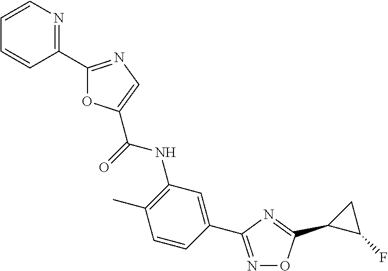 [SMILES: Cc1ccc(-c2noc([C@H]3C[C@@H]3F)n2)cc1NC(=O)c1cnc(-c2ccccn2)o1] | (M + H)⁺: 406.15 |
| 297 | 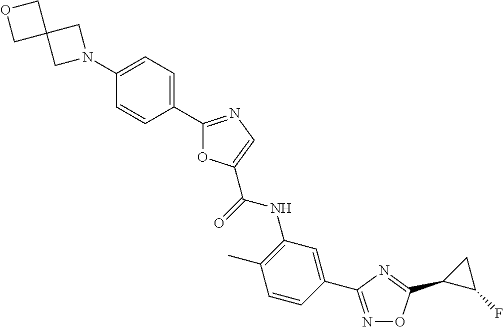 [SMILES: Cc1ccc(-c2noc([C@H]3C[C@@H]3F)n2)cc1NC(=O)c1cnc(-c2ccc(N3CC4(COC4)C3)cc2)o1] | (M + H)⁺: 502.20 |
| 298 | 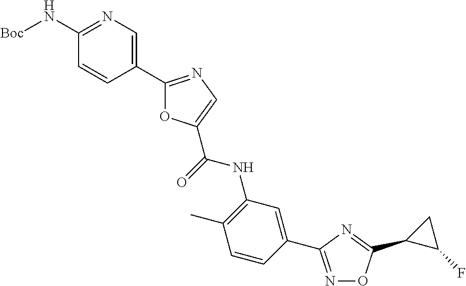 [SMILES: Cc1ccc(-c2noc([C@H]3C[C@@H]3F)n2)cc1NC(=O)c1cnc(-c2ccc(NC(=O)OC(C)(C)C)nc2)o1] | (M + H)⁺: 521.40 |
| 299 | 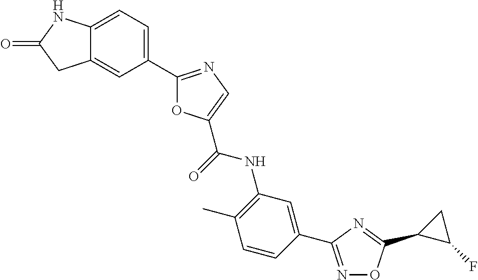 [SMILES: Cc1ccc(-c2noc([C@H]3C[C@@H]3F)n2)cc1NC(=O)c1cnc(-c2ccc3c(c2)CC(=O)N3)o1] | (M + H)⁺: 460.15 |
| 300 | 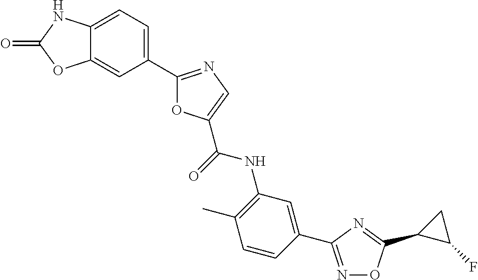 [SMILES: Cc1ccc(-c2noc([C@H]3C[C@@H]3F)n2)cc1NC(=O)c1cnc(-c2ccc3[nH]c(=O)oc3c2)o1] | (M − H)⁻: 460.05 |
| 301 | 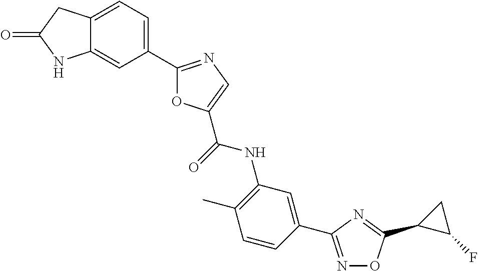 [SMILES: Cc1ccc(-c2noc([C@H]3C[C@@H]3F)n2)cc1NC(=O)c1cnc(-c2ccc3c(c2)NC(=O)C3)o1] | (M + H)⁺: 460.05 |
| 302 | 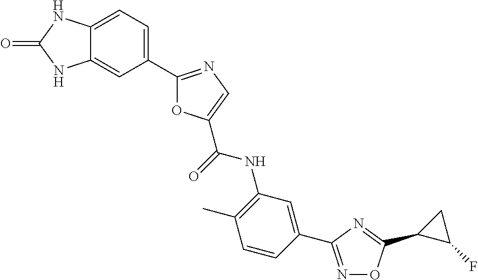 [SMILES: Cc1ccc(-c2noc([C@H]3C[C@@H]3F)n2)cc1NC(=O)c1cnc(-c2ccc3[nH]c(=O)[nH]c3c2)o1] | (M + H)⁺: 461.05 |
| 303 | 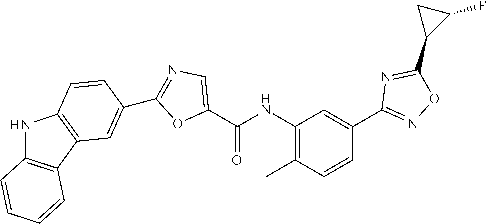 [SMILES: Cc1ccc(-c2noc([C@H]3C[C@@H]3F)n2)cc1NC(=O)c1cnc(-c2ccc3[nH]c4ccccc4c3c2)o1] | (M + H)⁺: 494.02 |
| 304 | 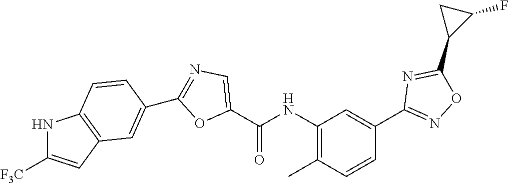 [SMILES: Cc1ccc(-c2noc([C@H]3C[C@@H]3F)n2)cc1NC(=O)c1cnc(-c2ccc3[nH]c(C(F)(F)F)cc3c2)o1] | (M + H)⁺: 511.94 |
| 305 | 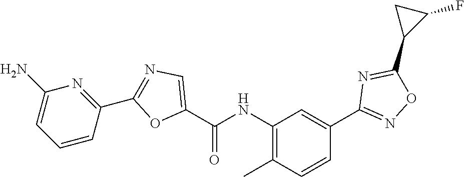 [SMILES: Cc1ccc(-c2noc([C@H]3C[C@@H]3F)n2)cc1NC(=O)c1cnc(-c2cccc(N)n2)o1] | (M + H)⁺: 421.10 |
| 306 | 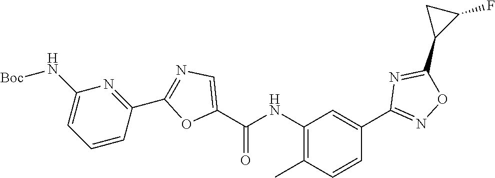 [SMILES: Cc1ccc(-c2noc([C@H]3C[C@@H]3F)n2)cc1NC(=O)c1cnc(-c2cccc(NC(=O)OC(C)(C)C)n2)o1] | (M + H)⁺: 521.15 |
| 307 | 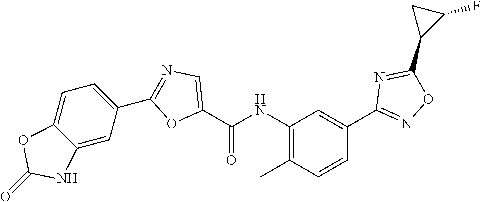 [SMILES: Cc1ccc(-c2noc([C@H]3C[C@@H]3F)n2)cc1NC(=O)c1cnc(-c2ccc3oc(=O)[nH]c3c2)o1] | (M − H)⁻: 460.05 |
| 308 | 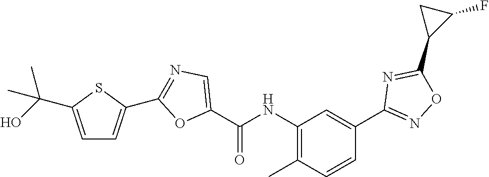 [SMILES: Cc1ccc(-c2noc([C@H]3C[C@@H]3F)n2)cc1NC(=O)c1cnc(-c2ccc(C(C)(C)O)s2)o1] | (M + H)⁺: 469.15 |
| 308-1 | 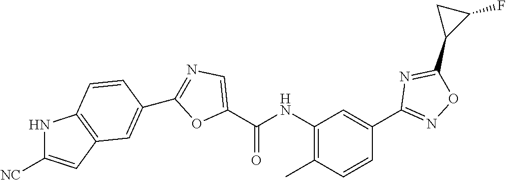 [SMILES: Cc1ccc(-c2noc([C@H]3C[C@@H]3F)n2)cc1NC(=O)c1cnc(-c2ccc3[nH]c(C#N)cc3c2)o1] | (M + H)⁺: 468.98 |
| 308-2 | 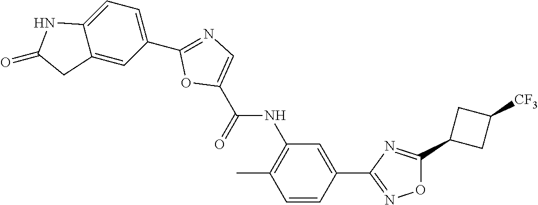 [SMILES: Cc1ccc(-c2noc([C@H]3C[C@@H](C(F)(F)F)C3)n2)cc1NC(=O)c1cnc(-c2ccc3c(c2)CC(=O)N3)o1] | (M + H)⁺: 524.01 |
| 308-3 | 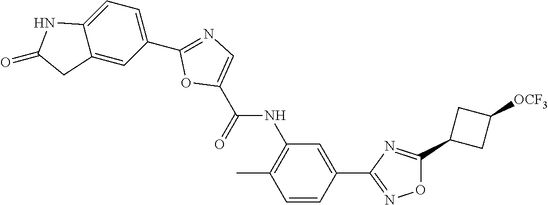 [SMILES: Cc1ccc(-c2noc([C@H]3C[C@@H](OC(F)(F)F)C3)n2)cc1NC(=O)c1cnc(-c2ccc3c(c2)CC(=O)N3)o1] | (M + H)⁺: 539.79 |
| 308-4 | 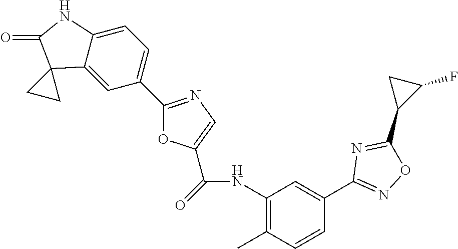 [SMILES: Cc1ccc(-c2noc([C@H]3C[C@@H]3F)n2)cc1NC(=O)c1cnc(-c2ccc3c(c2)C2(CC2)C(=O)N3)o1] | (M + H)⁺: 485.9 |
| 308-5 | 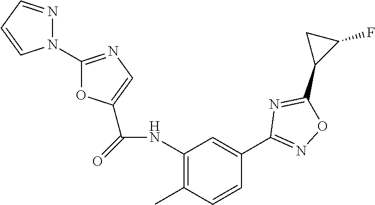 [SMILES: Cc1ccc(-c2noc([C@H]3C[C@@H]3F)n2)cc1NC(=O)c1cnc(-n2cccn2)o1] | (M + H)⁺: 395.0 |
| 308-6 | 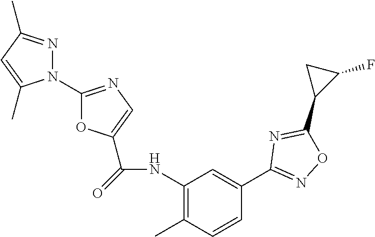 [SMILES: Cc1cc(C)n(-c2ncc(C(=O)Nc3cc(-c4noc([C@H]5C[C@@H]5F)n4)ccc3C)o2)n1] | (M + H)⁺: 423.1 |
| 308-7 | 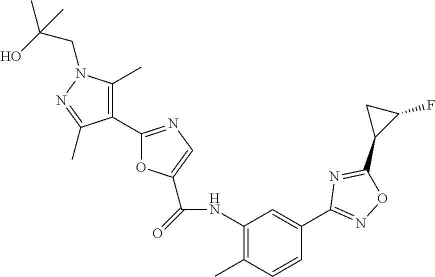 [SMILES: Cc1ccc(-c2noc([C@H]3C[C@@H]3F)n2)cc1NC(=O)c1cnc(-c2c(C)nn(CC(C)(C)O)c2C)o1] | (M + H)⁺: 495.0 |
| 308-8 | 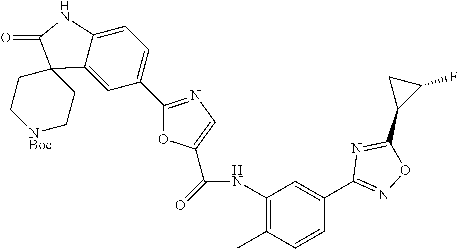 [SMILES: Cc1ccc(-c2noc([C@H]3C[C@@H]3F)n2)cc1NC(=O)c1cnc(-c2ccc3c(c2)C2(CCN(C(=O)OC(C)(C)C)CC2)C(=O)N3)o1] | (M + H)⁺: 629.0 |
| 308-9 | 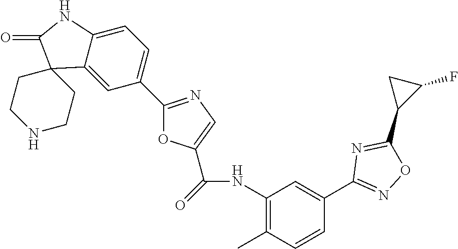 [SMILES: Cc1ccc(-c2noc([C@H]3C[C@@H]3F)n2)cc1NC(=O)c1cnc(-c2ccc3c(c2)C2(CCNCC2)C(=O)N3)o1] | (M + H)⁺: 528.9 |
| 308-10 | 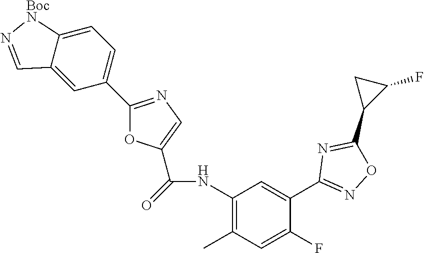 [SMILES: Cc1cc(F)c(-c2noc([C@H]3C[C@@H]3F)n2)cc1NC(=O)c1cnc(-c2ccc3c(cnn3C(=O)OC(C)(C)C)c2)o1] | (M + H)⁺: 563.0 |
| 308-11 | 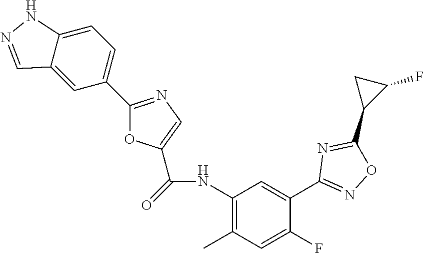 [SMILES: Cc1cc(F)c(-c2noc([C@H]3C[C@@H]3F)n2)cc1NC(=O)c1cnc(-c2ccc3[nH]ncc3c2)o1] | (M + H)⁺: 462.8 |
| 308-12 | 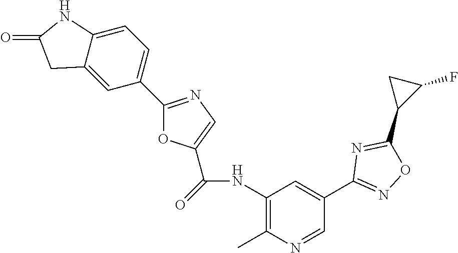 [SMILES: Cc1ncc(-c2noc([C@H]3C[C@@H]3F)n2)cc1NC(=O)c1cnc(-c2ccc3c(c2)CC(=O)N3)o1] | (M + H)⁺: 460.8 |
| 308-13 | 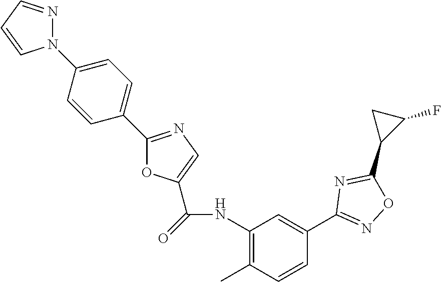 [SMILES: Cc1ccc(-c2noc([C@H]3C[C@@H]3F)n2)cc1NC(=O)c1cnc(-c2ccc(-n3cccn3)cc2)o1] | (M + H)⁺: 470.87 |
| 308-14 | 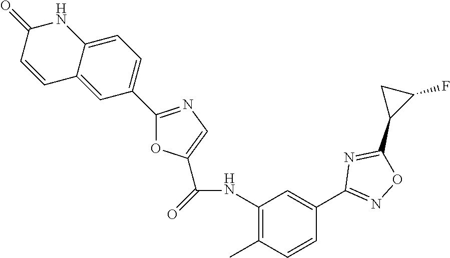 [SMILES: Cc1ccc(-c2noc([C@H]3C[C@@H]3F)n2)cc1NC(=O)c1cnc(-c2ccc3[nH]c(=O)ccc3c2)o1] | (M + H)⁺: 471.83 |
| 308-15 | 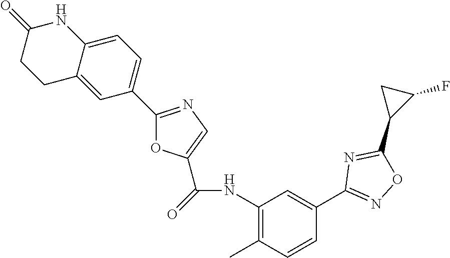 [SMILES: Cc1ccc(-c2noc([C@H]3C[C@@H]3F)n2)cc1NC(=O)c1cnc(-c2ccc3c(c2)CCC(=O)N3)o1] | (M + H)⁺: 474.04 |
| 308-16 | 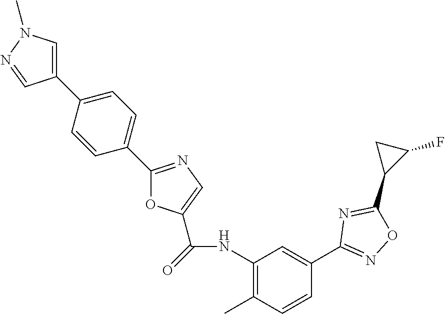 [SMILES: Cc1ccc(-c2noc([C@H]3C[C@@H]3F)n2)cc1NC(=O)c1cnc(-c2ccc(-c3cnn(C)c3)cc2)o1] | (M + H)⁺: 485.12 |
| 308-17 | 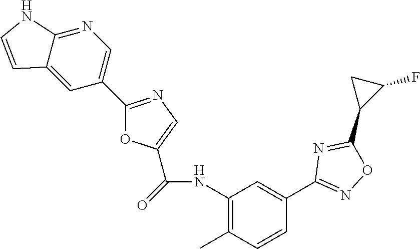 [SMILES: Cc1ccc(-c2noc([C@H]3C[C@@H]3F)n2)cc1NC(=O)c1cnc(-c2cnc3[nH]ccc3c2)o1] | (M + H)⁺: 445.09 |
| 308-18 | 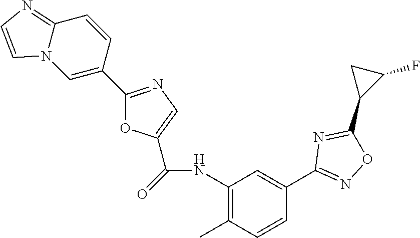 [SMILES: Cc1ccc(-c2noc([C@H]3C[C@@H]3F)n2)cc1NC(=O)c1cnc(-c2ccc3nccn3c2)o1] | (M + H)⁺: 445.10 |
| 308-19 | 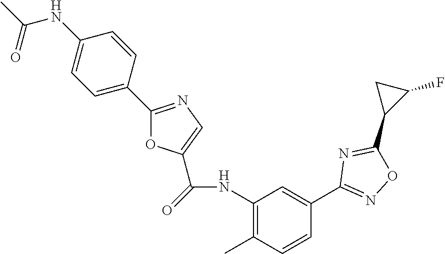 [SMILES: CC(=O)Nc1ccc(-c2ncc(C(=O)Nc3cc(-c4noc([C@H]5C[C@@H]5F)n4)ccc3C)o2)cc1] | (M + H)⁺: 462.04 |
| 308-20 | 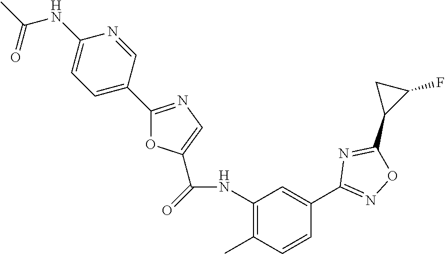 [SMILES: CC(=O)Nc1ccc(-c2ncc(C(=O)Nc3cc(-c4noc([C@H]5C[C@@H]5F)n4)ccc3C)o2)cn1] | (M + H)⁺: 463.24 |
| 308-21 | 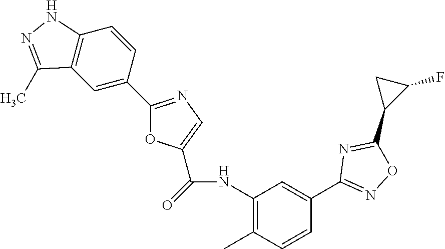 [SMILES: Cc1ccc(-c2noc([C@H]3C[C@@H]3F)n2)cc1NC(=O)c1cnc(-c2ccc3[nH]nc(C)c3c2)o1] | (M + H)⁺: 458.96 |
| 308-22 | 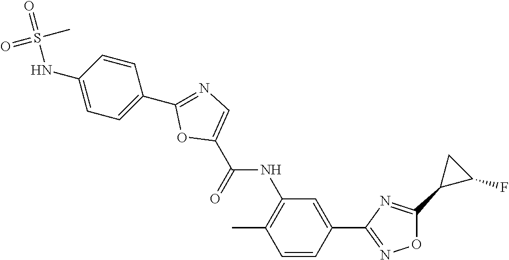 [SMILES: Cc1ccc(-c2noc([C@H]3C[C@@H]3F)n2)cc1NC(=O)c1cnc(-c2ccc(NS(C)(=O)=O)cc2)o1] | (M + H)⁺: 498.05 |
| 308-23 | 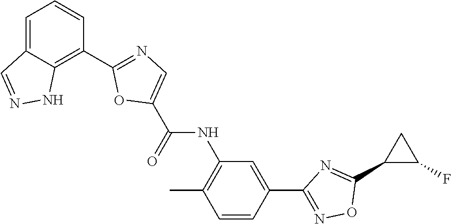 [SMILES: Cc1ccc(-c2noc([C@H]3C[C@@H]3F)n2)cc1NC(=O)c1cnc(-c2cccc3cn[nH]c23)o1] | (M + H)⁺: 445.05 |
| 308-24 | 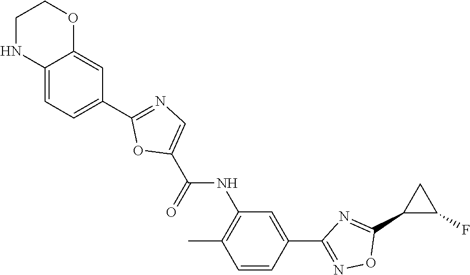 [SMILES: Cc1ccc(-c2noc([C@H]3C[C@@H]3F)n2)cc1NC(=O)c1cnc(-c2ccc3c(c2)OCCN3)o1] | (M + H)⁺: 462.15 |
| 308-25 | 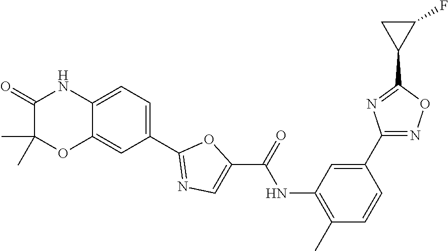 [SMILES: Cc1ccc(-c2noc([C@H]3C[C@@H]3F)n2)cc1NC(=O)c1cnc(-c2ccc3c(c2)OC(C)(C)C(=O)N3)o1] | (M + H)⁺: 504.187 |
| 308-26 | 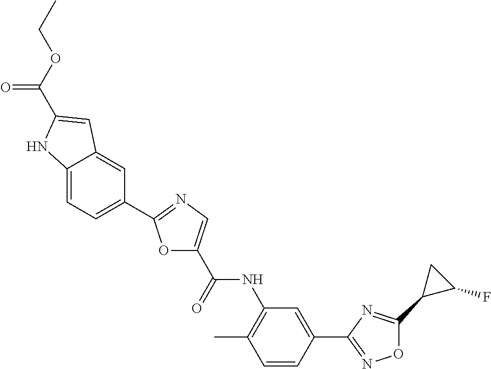 [SMILES: CCOC(=O)c1cc2cc(-c3ncc(C(=O)Nc4cc(-c5noc([C@H]6C[C@@H]6F)n5)ccc4C)o3)ccc2[nH]1] | (M + H)⁺: 516.20 |
| 308-27 | 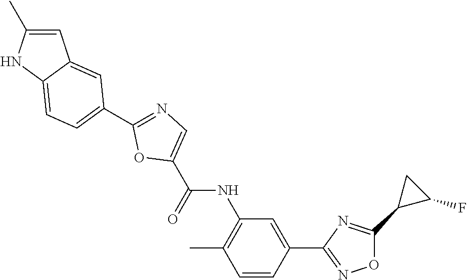 [SMILES: Cc1cc2cc(-c3ncc(C(=O)Nc4cc(-c5noc([C@H]6C[C@@H]6F)n5)ccc4C)o3)ccc2[nH]1] | (M + H)⁺: 458.20 |
| 308-28 | 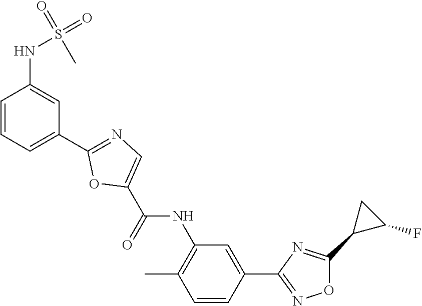 [SMILES: Cc1ccc(-c2noc([C@H]3C[C@@H]3F)n2)cc1NC(=O)c1cnc(-c2cccc(NS(C)(=O)=O)c2)o1] | (M + H)⁺: 498.15 |
| 308-29 | 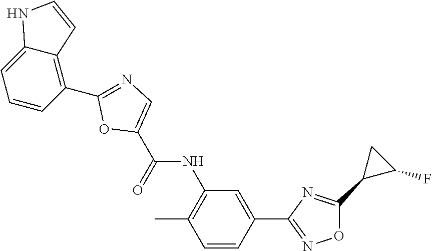 [SMILES: Cc1ccc(-c2noc([C@H]3C[C@@H]3F)n2)cc1NC(=O)c1cnc(-c2cccc3[nH]ccc23)o1] | (M + H)⁺: 444.20 |
| 308-30 | 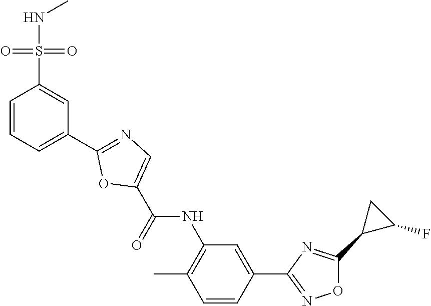 [SMILES: CNS(=O)(=O)c1cccc(-c2ncc(C(=O)Nc3cc(-c4noc([C@H]5C[C@@H]5F)n4)ccc3C)o2)c1] | (M + H)⁺: 498.00 |
| 308-31 | 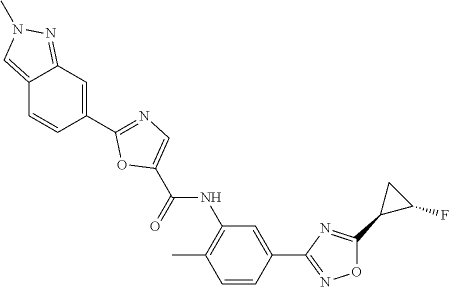 [SMILES: Cc1ccc(-c2noc([C@H]3C[C@@H]3F)n2)cc1NC(=O)c1cnc(-c2ccc3cn(C)nc3c2)o1] | (M + H)⁺: 459.20, 460.20 |
| 308-32 | 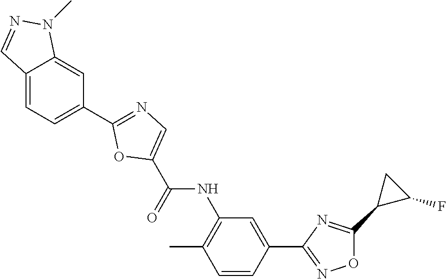 [SMILES: Cc1ccc(-c2noc([C@H]3C[C@@H]3F)n2)cc1NC(=O)c1cnc(-c2ccc3cnn(C)c3c2)o1] | (M + H)⁺: 459.20 |
| 308-33 | 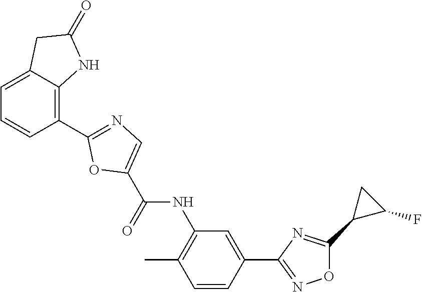 [SMILES: Cc1ccc(-c2noc([C@H]3C[C@@H]3F)n2)cc1NC(=O)c1cnc(-c2cccc3c2NC(=O)C3)o1] | (M + H)⁺: 460.00 |
| 308-34 | 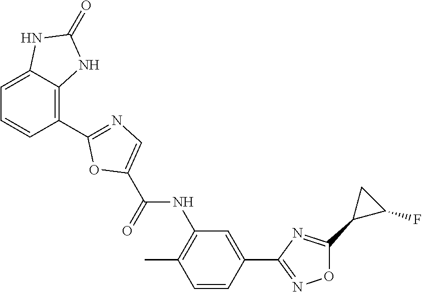 [SMILES: Cc1ccc(-c2noc([C@H]3C[C@@H]3F)n2)cc1NC(=O)c1cnc(-c2cccc3[nH]c(=O)[nH]c23)o1] | (M + H)⁺: 461.15 |
| 308-35 | 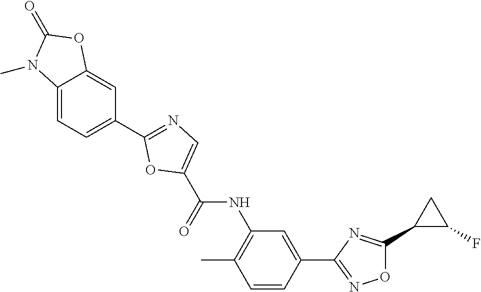 [SMILES: Cc1ccc(-c2noc([C@H]3C[C@@H]3F)n2)cc1NC(=O)c1cnc(-c2ccc3c(c2)oc(=O)n3C)o1] | (M + H)⁺: 476.15 |
| 308-36 | 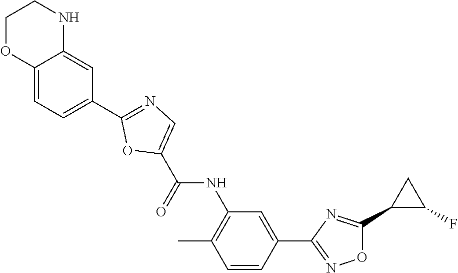 [SMILES: Cc1ccc(-c2noc([C@H]3C[C@@H]3F)n2)cc1NC(=O)c1cnc(-c2ccc3c(c2)NCCO3)o1] | (M + H)⁺: 462.20 |
| 308-37 | 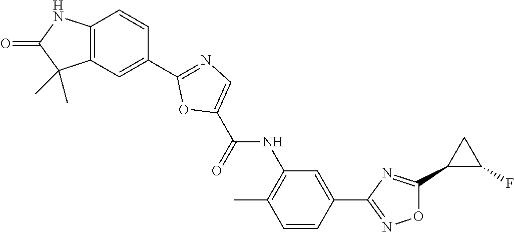 [SMILES: Cc1ccc(-c2noc([C@H]3C[C@@H]3F)n2)cc1NC(=O)c1cnc(-c2ccc3c(c2)C(C)(C)C(=O)N3)o1] | (M + H)⁺: 488.20 |
| 308-38 | 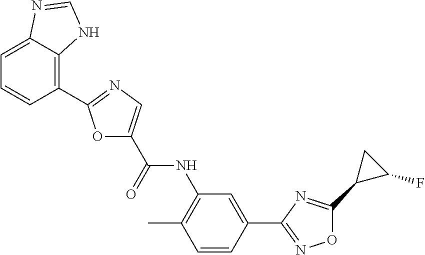 [SMILES: Cc1ccc(-c2noc([C@H]3C[C@@H]3F)n2)cc1NC(=O)c1cnc(-c2cccc3nc[nH]c23)o1] | (M + H)⁺: 445.00 |
| 308-39 | 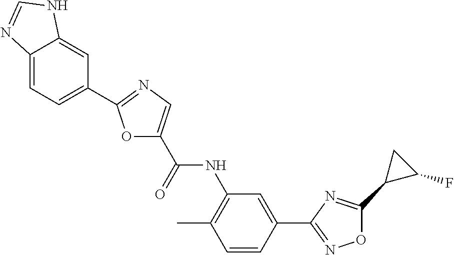 [SMILES: Cc1ccc(-c2noc([C@H]3C[C@@H]3F)n2)cc1NC(=O)c1cnc(-c2ccc3nc[nH]c3c2)o1] | (M + H)⁺: 445.15 |
| 308-40 | 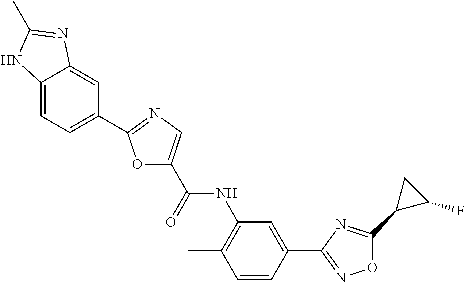 [SMILES: Cc1nc2cc(-c3ncc(C(=O)Nc4cc(-c5noc([C@H]6C[C@@H]6F)n5)ccc4C)o3)ccc2[nH]1] | (M + H)⁺: 459.20 |
| 308-41 | 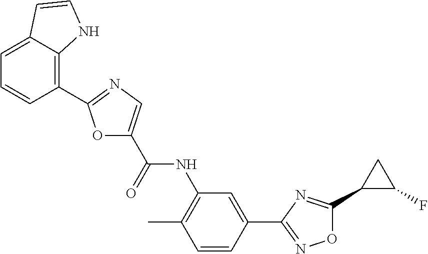 [SMILES: Cc1ccc(-c2noc([C@H]3C[C@@H]3F)n2)cc1NC(=O)c1cnc(-c2cccc3cc[nH]c23)o1] | (M + H)⁺: 444.20 |
| 308-42 | 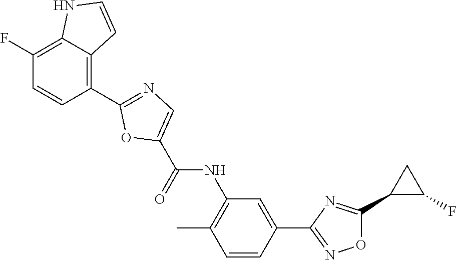 [SMILES: Cc1ccc(-c2noc([C@H]3C[C@@H]3F)n2)cc1NC(=O)c1cnc(-c2ccc(F)c3[nH]ccc23)o1] | (M + H)⁺: 462.15 |
| 308-43 | 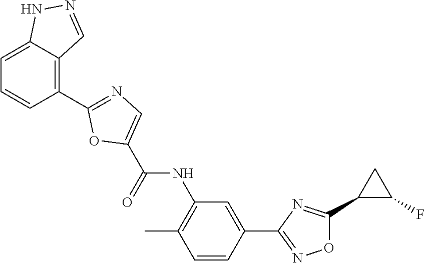 [SMILES: Cc1ccc(-c2noc([C@H]3C[C@@H]3F)n2)cc1NC(=O)c1cnc(-c2cccc3[nH]ncc23)o1] | (M + H)⁺: 445.15 |
| 308-44 | 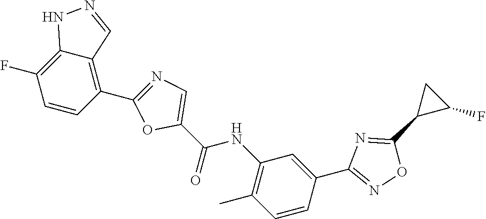 [SMILES: Cc1ccc(-c2noc([C@H]3C[C@@H]3F)n2)cc1NC(=O)c1cnc(-c2ccc(F)c3[nH]ncc23)o1] | (M + H)⁺: 463.05 |
| 308-45 | 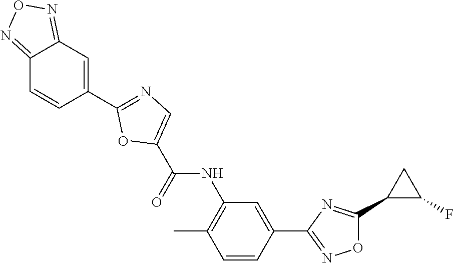 [SMILES: Cc1ccc(-c2noc([C@H]3C[C@@H]3F)n2)cc1NC(=O)c1cnc(-c2ccc3nonc3c2)o1] | (M + H)⁺: 445.00 |
| 308-46 | 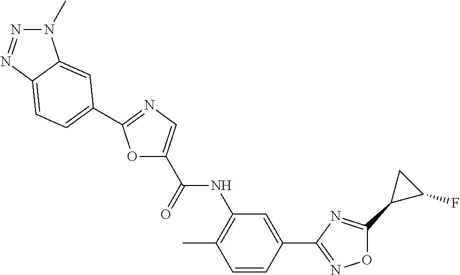 [SMILES: Cc1ccc(-c2noc([C@H]3C[C@@H]3F)n2)cc1NC(=O)c1cnc(-c2ccc3nnn(C)c3c2)o1] | (M + H)⁺: 460.20 |
| 308-47 | 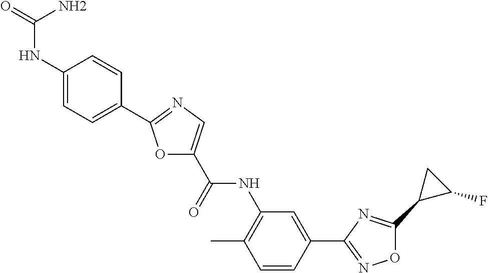 [SMILES: Cc1ccc(-c2noc([C@H]3C[C@@H]3F)n2)cc1NC(=O)c1cnc(-c2ccc(NC(N)=O)cc2)o1] | (M + H)⁺: 463.15 |
| 308-48 | 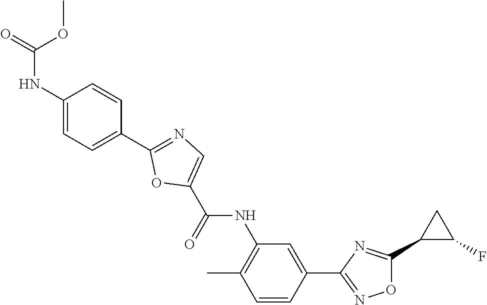 [SMILES: COC(=O)Nc1ccc(-c2ncc(C(=O)Nc3cc(-c4noc([C@H]5C[C@@H]5F)n4)ccc3C)o2)cc1] | (M + H)⁺: 478.20 |
| 308-49 | 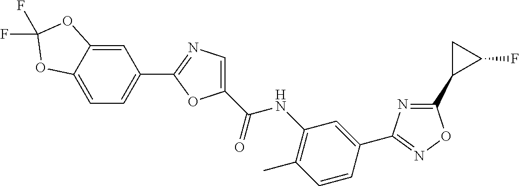 [SMILES: Cc1ccc(-c2noc([C@H]3C[C@@H]3F)n2)cc1NC(=O)c1cnc(-c2ccc3c(c2)OC(F)(F)O3)o1] | (M + H)⁺: 483.05 |
| 308-50 | 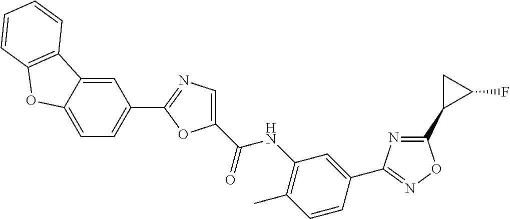 | (M + H)⁺: 459.15 |
| 308-51 | 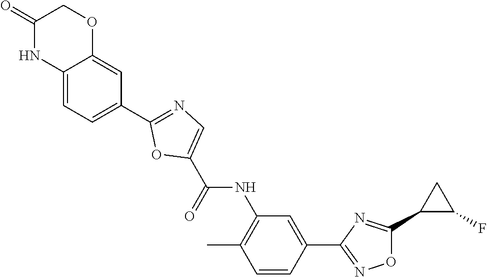 [SMILES: Cc1ccc(-c2noc([C@H]3C[C@@H]3F)n2)cc1NC(=O)c1cnc(-c2ccc3c(c2)OCC(=O)N3)o1] | (M + H)⁺: 474.10 |
| 308-52 | 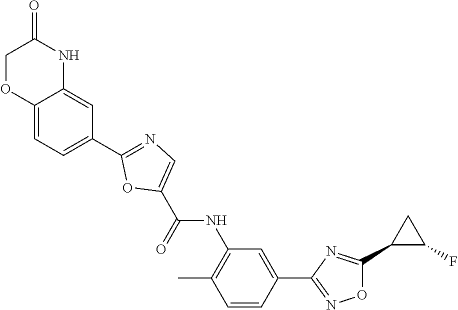 [SMILES: Cc1ccc(-c2noc([C@H]3C[C@@H]3F)n2)cc1NC(=O)c1cnc(-c2ccc3c(c2)NC(=O)CO3)o1] | (M + H)⁺: 476.05 |
| 308-53 | 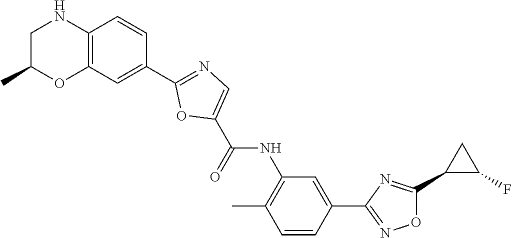 [SMILES: Cc1ccc(-c2noc([C@H]3C[C@@H]3F)n2)cc1NC(=O)c1cnc(-c2ccc3c(c2)O[C@@H](C)CN3)o1] | (M + H)⁺: 476.20 |
| 308-54 | 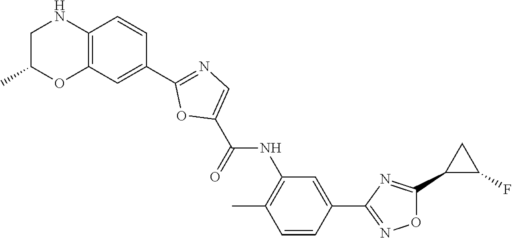 [SMILES: Cc1ccc(-c2noc([C@H]3C[C@@H]3F)n2)cc1NC(=O)c1cnc(-c2ccc3c(c2)O[C@H](C)CN3)o1] | (M + H)⁺: 476.20 |
| 308-55 | 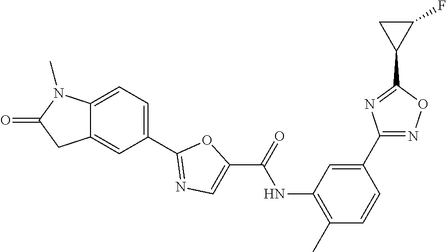 [SMILES: Cc1ccc(-c2noc([C@H]3C[C@@H]3F)n2)cc1NC(=O)c1cnc(-c2ccc3c(c2)CC(=O)N3C)o1] | (M + H)⁺: 474.090 |
| 308-56 | 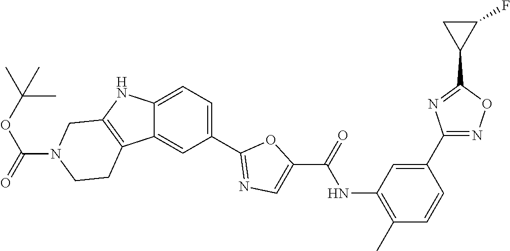 [SMILES: Cc1ccc(-c2noc([C@H]3C[C@@H]3F)n2)cc1NC(=O)c1cnc(-c2ccc3[nH]c4c(c3c2)CCN(C(=O)OC(C)(C)C)C4)o1] | (M + H)⁺: 599.229 |
| 308-57 | 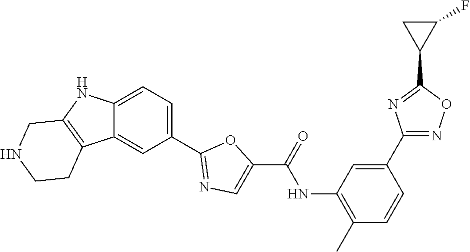 [SMILES: Cc1ccc(-c2noc([C@H]3C[C@@H]3F)n2)cc1NC(=O)c1cnc(-c2ccc3[nH]c4c(c3c2)CCNC4)o1] | (M + H)⁺: 499.338 |
| 308-58 | 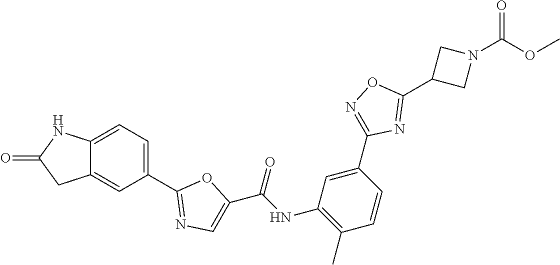 [SMILES: COC(=O)N1CC(c2nc(-c3ccc(C)c(NC(=O)c4cnc(-c5ccc6c(c5)CC(=O)N6)o4)c3)no2)C1] | (M + H)⁺: 515.10 |
| 308-59 | 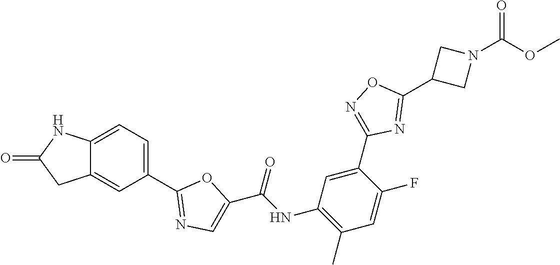 [SMILES: COC(=O)N1CC(c2nc(-c3cc(NC(=O)c4cnc(-c5ccc6c(c5)CC(=O)N6)o4)c(C)cc3F)no2)C1] | (M + H)⁺: 533.10 |
| 308-60 | 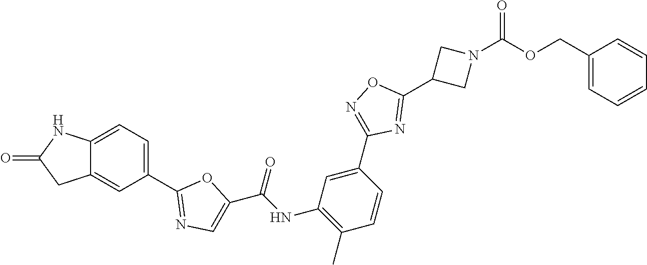 [SMILES: Cc1ccc(-c2noc(C3CN(C(=O)OCc4ccccc4)C3)n2)cc1NC(=O)c1cnc(-c2ccc3c(c2)CC(=O)N3)o1] | (M + H)⁺: 591.30 |
| 308-61 | 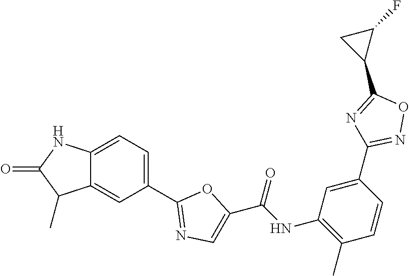 [SMILES: Cc1ccc(-c2noc([C@H]3C[C@@H]3F)n2)cc1NC(=O)c1cnc(-c2ccc3c(c2)C(C)C(=O)N3)o1] | (M + H)⁺: 473.994 |
| 308-62 | 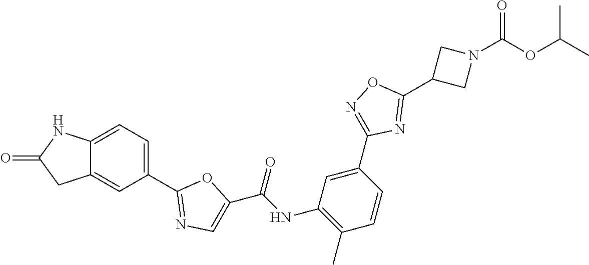 [SMILES: Cc1ccc(-c2noc(C3CN(C(=O)OC(C)C)C3)n2)cc1NC(=O)c1cnc(-c2ccc3c(c2)CC(=O)N3)o1] | (M + H)⁺: 543.15 |
| 308-63 | 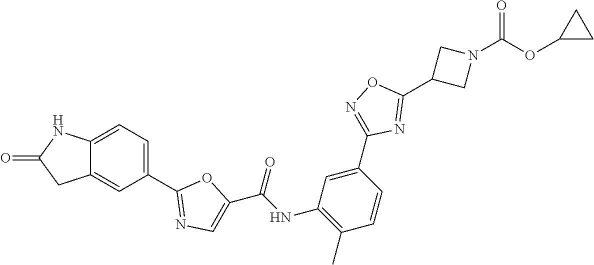 [SMILES: Cc1ccc(-c2noc(C3CN(C(=O)OC4CC4)C3)n2)cc1NC(=O)c1cnc(-c2ccc3c(c2)CC(=O)N3)o1] | (M + H)⁺: 541.15 |
| 308-64 | 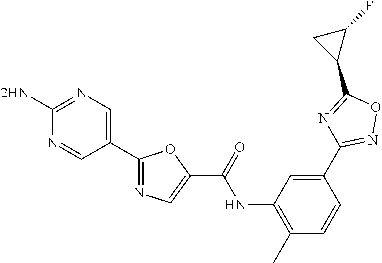 | (M + H)⁺: 422.298 |
| 308-65 | 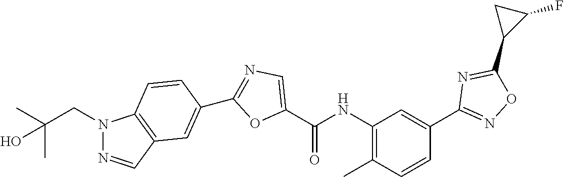 [SMILES: Cc1ccc(-c2noc([C@H]3C[C@@H]3F)n2)cc1NC(=O)c1cnc(-c2ccc3c(cnn3CC(C)(C)O)c2)o1] | (M + H)⁺: 517.17 |
| 308-66 | 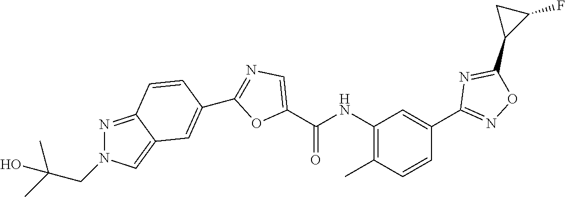 [SMILES: Cc1ccc(-c2noc([C@H]3C[C@@H]3F)n2)cc1NC(=O)c1cnc(-c2ccc3nn(CC(C)(C)O)cc3c2)o1] | (M + H)⁺: 517.24 |
| 308-67 | 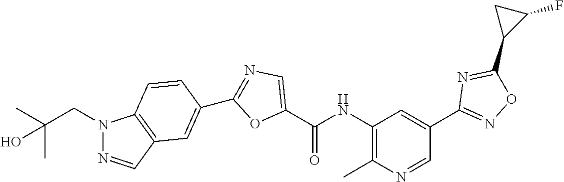 [SMILES: Cc1ncc(-c2noc([C@H]3C[C@@H]3F)n2)cc1NC(=O)c1cnc(-c2ccc3c(cnn3CC(C)(C)O)c2)o1] | (M + H)⁺: 518.32 |
| 308-68 | 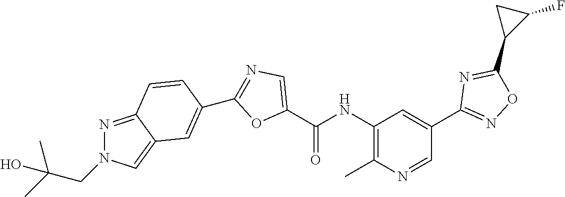 [SMILES: Cc1ncc(-c2noc([C@H]3C[C@@H]3F)n2)cc1NC(=O)c1cnc(-c2ccc3nn(CC(C)(C)O)cc3c2)o1] | (M + H)⁺: 518.24 |

### Example 309

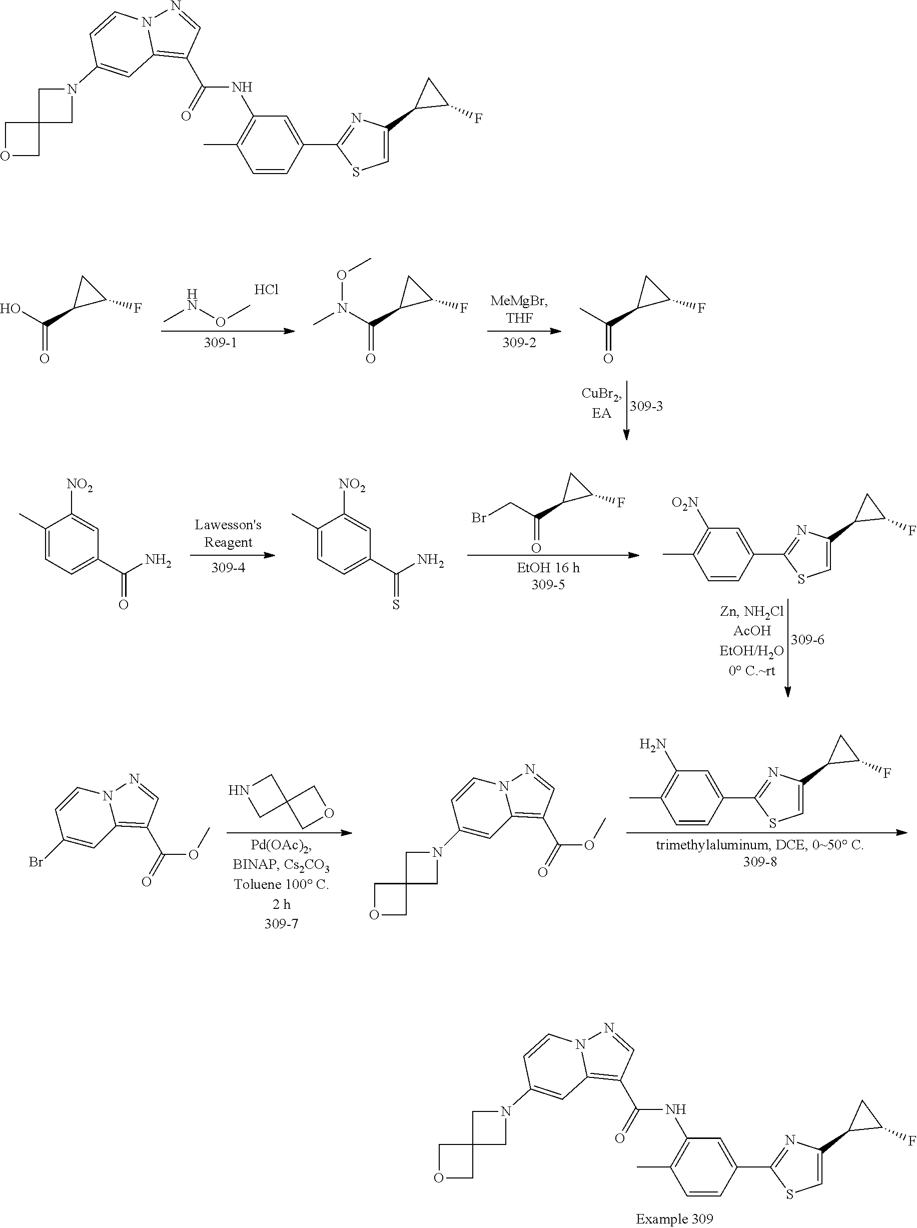

Step 309-1: A solution of (1R,2S)-2-fluorocyclopropane-1-carboxylic acid (500 mg, 4.804 mmol, 1 equiv), N,O-dimethylhydroxylamine hydrochloride (515.46 mg, 5.284 mmol, 1.1 equiv), HATU (2.01 g, 5.284 mmol, 1.1 equiv) and DIPEA (2.48 g, 19.216 mmol, 4 equiv) in THF (5 mL) at 0° C. The resulting mixture was stirred for 3 h at room temperature. Desired product could be detected by LCMS. The resulting mixture was extracted with CH₂Cl₂ (3×30 mL). The combined organic layers were washed with deionized water (2×30 mL), dried over anhydrous Na₂SO₄. After filtration, the filtrate was concentrated under reduced pressure. The residue was purified by silica gel column chromatography, eluted with CH₂Cl₂/PE (1:5) to afford the desired product (500 mg, 70.73%) as a light yellow oil. (ES, m/z): [M+H]³⁰=148.25.
Step 309-2: To a stirred solution the compound from Step 309-1 (200 mg, 1.359 mmol, 1 equiv) in THE (3 mL) were added MeMgBr (4.08 mL, 4.077 mmol, 3 equiv) dropwise at 0° C. The resulting mixture was stirred for an additional 2 h at room temperature. The reaction was quenched by the addition of Na₂SO₄·10H₂O at 0° C. The resulting mixture was filtered, the filter cake was washed with CH₂Cl₂ (3×20 mL). The filtrate was concentrated under reduced pressure. This resulted in the desired product (100 mg, 72.06%) as a light yellow oil. The crude product was used in the next step directly without further purification.
Step 309-3: A solution of the compound from Step 309-2 (100 mg, 0.979 mmol, 1 equiv) and CuBr₂ (1.09 g, 4.895 mmol, 5 equiv) in EtOAc (10 mL) was stirred for overnight at room temperature The resulting mixture was filtered, the filter cake was washed with CH₂Cl₂ (3×50 mL). The filtrate was concentrated under reduced pressure. The resulting mixture was washed with 2×2 100 mL of sat. NH₄Cl (aq.). The filtrate was concentrated under reduced pressure. The resulted in the desired product (120 mg, 67.69%) as a brown oil. The crude product was used in the next step directly without further purification.
Step 309-4: A solution of the 4-methyl-3-nitrobenzamide (200 mg, 1.110 mmol, 1 equiv) and Lawesson's Reagent (538.80 mg, 1.332 mmol, 1.2 equiv) in THE (2 mL) was stirred for 4 h at 60° C. under nitrogen atmosphere. Desired product could be detected by LCMS. The resulting mixture was diluted with EtOAc (200 mL). The reaction was quenched by the addition of sat. NaHCO₃(aq.) (200 mL) at room temperature. The resulting mixture was washed with 2×2 100 mL of sat. NaHCO₃(aq.). The resulting mixture was concentrated under reduced pressure. This resulted in the desired product (170 mg, 78.04%) as a yellow solid. (ES, m/z): [M+H]⁺=197.05.
Step 309-5: A solution of the compound from Step 309-4 (216.81 mg, 1.105 mmol, 1 equiv) and the compound from Step 309-3 (200 mg, 1.105 mmol, 1.00 equiv) in EtOH (3 mL) was stirred for 16 h at room temperature. The resulting mixture was concentrated under reduced pressure. The crude product was purified by reversed-phase flash chromatography with the following conditions: column, C18 silica gel; mobile phase, water with 1% NH₄HCO₃ in MeCN, 0% to 100% gradient in 20 min; detector, UV 254 nm. This resulted in the desired product (50 mg, 16.26%) as a yellow green solid. (ES, m/z): [M+H]⁺=279.00.
Step 309-6: A solution of the compound from Step 309-5 (170 mg, 0.611 mmol, 1 equiv) and Zn (199.69 mg, 3.055 mmol, 5 equiv) in EtOH/H₂O (2 mL/0.5 mL) was added NH₄Cl (163.37 mg, 3.055 mmol, 5 equiv) in portions at 0° C. The solution was stirred for 10 min at 0° C. and stirred for 1 h at room temperature. Then, AcOH (0.10 mL, 1.821 mmol, 3 equiv) was added into the above solution at room temperature and stirred for another 1 h at room temperature. The resulting mixture was filtered, and the filter cake was washed with EA (3×15 mL). The filtrate was concentrated under reduced pressure. The crude product was purified by reversed-phase flash chromatography with the following conditions: column, C18 silica gel; mobile phase, water with 1% NH₄HCO₃ in MeCN, 0% to 100% gradient in 15 min; detector, UV 254 nm. This resulted in the desired product (50 mg, 32.96%) as a brown yellow solid. (ES, m/z): [M+H]⁺=249.20.
Step 309-7: A solution of methyl 5-bromopyrazolo[1,5-a]pyridine-3-carboxylate (400 mg, 1.568 mmol, 1 equiv), 2-oxa-6-azaspiro[3.3]heptane (310.92 mg, 3.136 mmol, 2 equiv), Pd(OAc)₂ (35.21 mg, 0.157 mmol, 0.1 equiv), BINAP (97.65 mg, 0.157 mmol, 0.1 equiv) and Cs₂CO₃ (1021.89 mg, 3.136 mmol, 2 equiv) in Toluene (6 mL) was stirred for 2 h at 100° C. under nitrogen atmosphere. The reaction was quenched by the addition of Water (3 mL) at room temperature. The aqueous layer was extracted with CH₂C₂ (2×20 mL). The organic phase was concentrated under a vacuum. The residue was purified by reversed-phase flash chromatography with the following conditions: column, C18 silica gel; mobile phase, MeCN in Water (0.1% FA), 0% to 100% gradient in 20 min; detector, UV 254 nm. This resulted in the desired product (140 mg, 32.67%) as a yellow solid. (ES, m/z): [M+H]⁺=274.15.
Step 309-8: To a stirred solution of the compound from Step 309-6 (50 mg, 0.201 mmol, 1 equiv) and the compound from Step 309-7 (57.27 mg, 0.199 mmol, 0.9 equiv) in DCE (3 mL) were added AlMe₃ (0.30 mL, 0.603 mmol, 3 equiv) dropwise at 0° C. under nitrogen atmosphere. The reaction was stirred for another 2 h at 50° C. The reaction was quenched with sat. NH₄Cl (aq.) at 0° C. The resulting mixture was extracted with EtOAc (3×50 mL). The combined organic layers were washed with H₂O (3×30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 0% to 100% gradient in 10 min; detector, UV 254 nm. The crude product was purified by Prep-HPLC. This resulted Example 309 (40.1 mg, 40.68%) as a white solid. (ES, m/z): [M+H]⁺=490.20.

¹H-NMR (400 MHz, DMSO-d₆) δ 9.40 (s, 1H), 8.55 (t, J=3.8 Hz, 2H), 7.93 (d, J=1.9 Hz, 1H), 7.64 (dd, J=7.9, 1.9 Hz, 1H), 7.43 (s, 1H), 7.36 (d, J=8.0 Hz, 1H), 6.85 (d, J=2.6 Hz, 1H), 6.42 (dd, J=7.5, 2.6 Hz, 1H), 5.00 (ddt, J=64.3, 6.0, 2.4 Hz, 1H), 4.74 (s, 4H), 4.15 (s, 4H), 2.74-2.60 (m, 1H), 2.30 (s, 3H), 1.56 (dddd, J=22.9, 11.2, 6.7, 2.9 Hz, 1H), 1.30 (dq, J=11.5, 6.6 Hz, 1H).

The following examples were prepared using procedures similar to those described above:

| Example | Structure | ESI-MS |
|---|---|---|
| 310 | 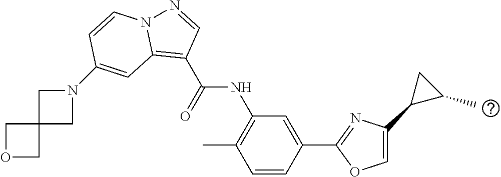 | (M + H)⁺: 474.05 |
| 311 | 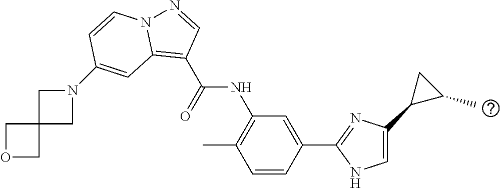 | (M + H)⁺: 473.15 |

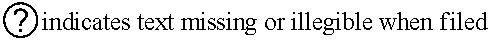

### Example 312

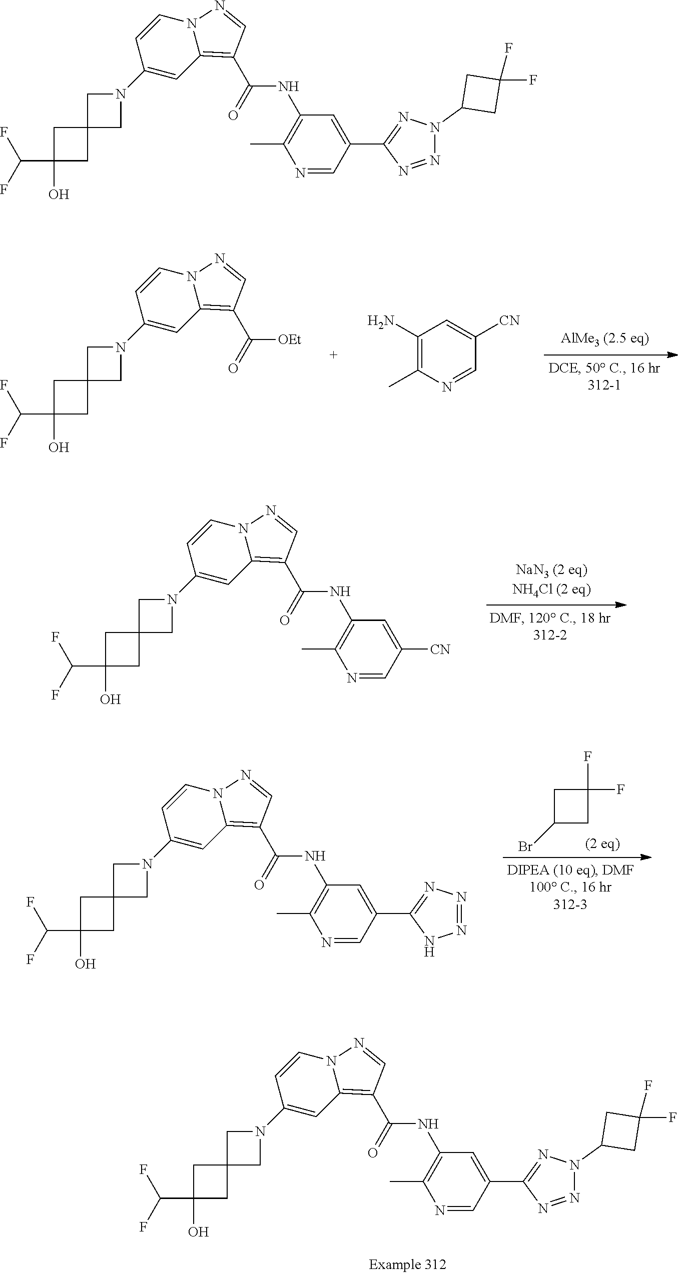

### Step 312-1:

To a solution of the compound from 313-1 (1000 mg, 1 Eq, 2.846 mmol) and 5-amino-6-methylnicotinonitrile (416.9 mg, 1.10 Eq, 3.131 mmol) in anhydrous ClCH₂CH₂Cl (9.487 mL) at rt under N₂ was added trimethylaluminum in toluene (513.0 mg, 3.558 mL, 2 molar, 2.5 Eq, 7.115 mmol). The reaction mixture was stirred at rt for 20 min, and the mixture was heated to 60° C. and stirred overnight. Upon the completion, the reaction was cooled to 0 degree, quenched with Brine solution (4 mL) and sat. NaHCO₃ solution (4 mL). The mixture was then concentrated. The solid was washed with ACN (20 mL) and DMF (5 mL). The filtrate was lyophilized to afford the desired product (1.1 g, 2.4 mmol, 86%, 950 Purity) as yellowish solid. (ES, m/z): [M+H]=439.242.

Step 312-2: To the solution of the compound from Step 312-1 (200 mg, 1 Eq, 456 μmol) in DMF (2.0 mL) were added ammonium chloride (48.8 mg, 2 Eq, 912 m 2 mol) and sodium azide (59.3 mg, 2 Eq, 912 μmol) at room temperature. The reaction mixture was then heated to 120° C. for 18 hours. After the completion of the reaction, 3 mL of water was added. The mixture was then acidified with 1N HCl solution (1 mL). Off-white solid was precipitated out. The solid was filtered out and dried under vacuum to afford the desired product (209 mg, 434 μmol, 95) as an off-white solid. (ES, m/z): [M+H]=482.15.
Step 312-3: To the solution of the compound from Step 312-2 (30 mg, 1 Eq, 62 mol) and 3-bromo-1,1-difluorocyclobutane (21 mg, 13 μL, 2 Eq, 0.12 mmol) in DMF (0.62 mL) was added DIPEA (81 mg, 0.11 mL, 10 Eq, 0.62 mmol) at rt. The reaction mixture was then heated to 100° C. for 16 hours. After completion, the reaction mixture was subjected to prep-TIPLC directly to afford Example 312 (8 mg, 0.01 mmol, 20%, 95% Purity) as a white solid. (ES, m/z): [M+H]⁺=572.300.

The following examples were prepared using procedures similar to those described above: F

| Example | Structure | ESI-MS |
|---|---|---|
| 312-1 | 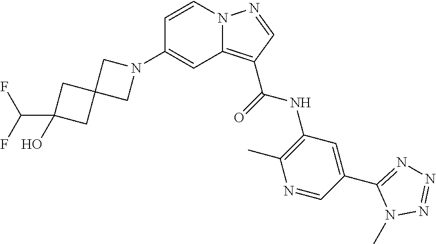 [SMILES: Cc1ncc(-c2nnnn2C)cc1NC(=O)c1cnn2ccc(N3CC4(C3)CC(O)(C(F)F)C4)cc12] | (M + H)⁺: 496.1 |
| 312-2 | 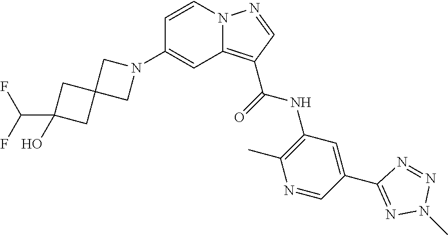 [SMILES: Cc1ncc(-c2nnn(C)n2)cc1NC(=O)c1cnn2ccc(N3CC4(C3)CC(O)(C(F)F)C4)cc12] | (M + H)⁺: 496.1 |
| 312-3 | 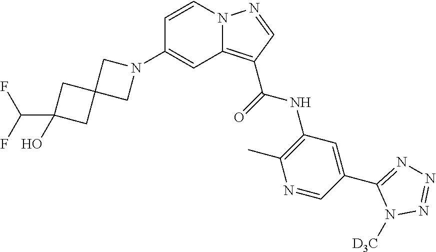 [SMILES: [2H]C([2H])([2H])n1nnnc1-c1cnc(C)c(NC(=O)c2cnn3ccc(N4CC5(C4)CC(O)(C(F)F)C5)cc23)c1] | (M + H)⁺: 499.0 |
| 312-4 | 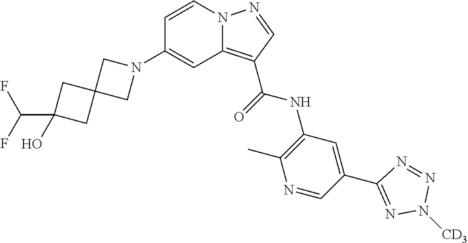 [SMILES: [2H]C([2H])([2H])n1nnc(-c2cnc(C)c(NC(=O)c3cnn4ccc(N5CC6(C5)CC(O)(C(F)F)C6)cc34)c2)n1] | (M + H)⁺: 499.3 |
| 312-5 | 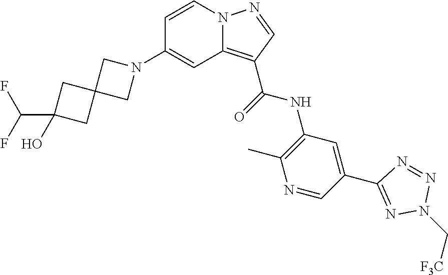 [SMILES: Cc1ncc(-c2nnn(CC(F)(F)F)n2)cc1NC(=O)c1cnn2ccc(N3CC4(C3)CC(O)(C(F)F)C4)cc12] | (M + H)⁺: 564.1 |
| 312-6 | 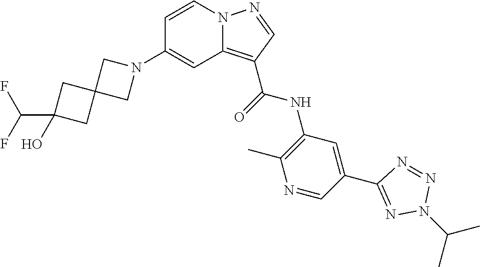 [SMILES: Cc1ncc(-c2nnn(C(C)C)n2)cc1NC(=O)c1cnn2ccc(N3CC4(C3)CC(O)(C(F)F)C4)cc12] | (M + H)⁺: 524.1 |
| 312-7 | 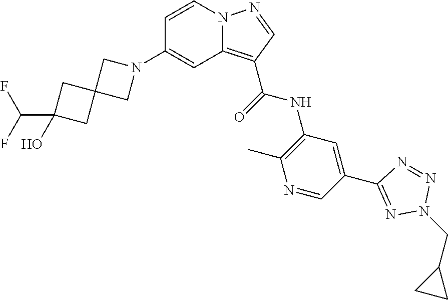 [SMILES: Cc1ncc(-c2nnn(CC3CC3)n2)cc1NC(=O)c1cnn2ccc(N3CC4(C3)CC(O)(C(F)F)C4)cc12] | (M + H)⁺: 536.3 |
| 312-8 | 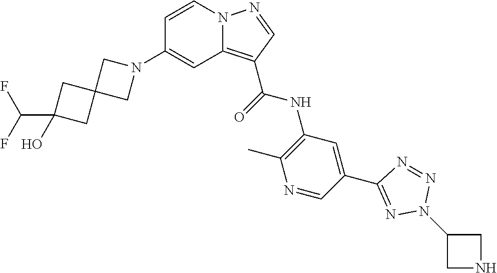 [SMILES: Cc1ncc(-c2nnn(C3CNC3)n2)cc1NC(=O)c1cnn2ccc(N3CC4(C3)CC(O)(C(F)F)C4)cc12] | (M + H)⁺: 537.211 |
| 312-9 | 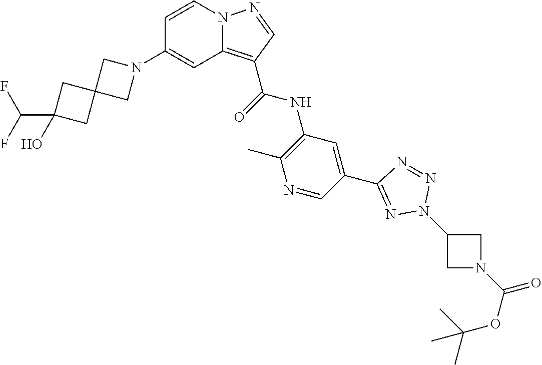 [SMILES: Cc1ncc(-c2nnn(C3CN(C(=O)OC(C)(C)C)C3)n2)cc1NC(=O)c1cnn2ccc(N3CC4(C3)CC(O)(C(F)F)C4)cc12] | (M + H)⁺: 637.438 |
| 312-10 | 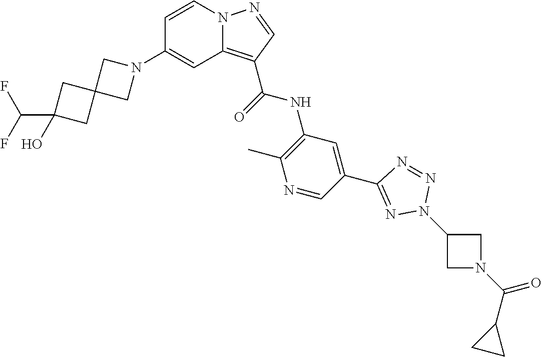 [SMILES: Cc1ncc(-c2nnn(C3CN(C(=O)C4CC4)C3)n2)cc1NC(=O)c1cnn2ccc(N3CC4(C3)CC(O)(C(F)F)C4)cc12] | (M + H)⁺: 605.277 |
| 312-11 | 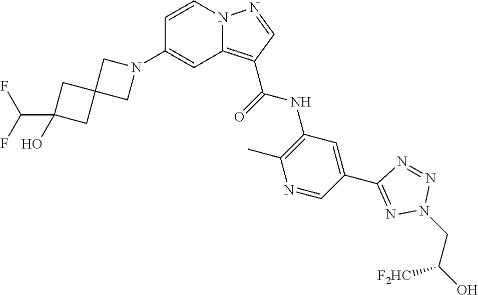 [SMILES: Cc1ncc(-c2nnn(C[C@H](O)C(F)F)n2)cc1NC(=O)c1cnn2ccc(N3CC4(C3)CC(O)(C(F)F)C4)cc12] | (M + H)⁺: 576.1 |
| 312-12 | 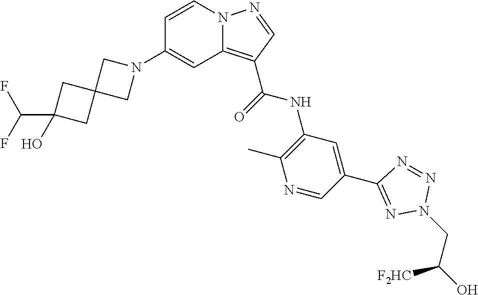 [SMILES: Cc1ncc(-c2nnn(C[C@@H](O)C(F)F)n2)cc1NC(=O)c1cnn2ccc(N3CC4(C3)CC(O)(C(F)F)C4)cc12] | (M + H)⁺: 576.1 |
| 312-13 | 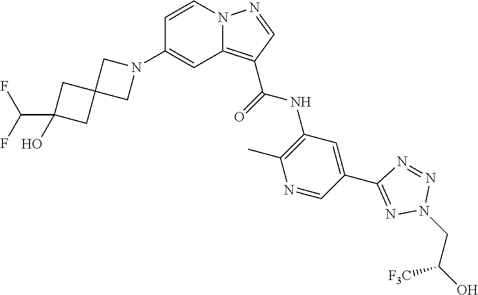 [SMILES: Cc1ncc(-c2nnn(C[C@H](O)C(F)(F)F)n2)cc1NC(=O)c1cnn2ccc(N3CC4(C3)CC(O)(C(F)F)C4)cc12] | (M + H)⁺: 594.2 |
| 312-14 | 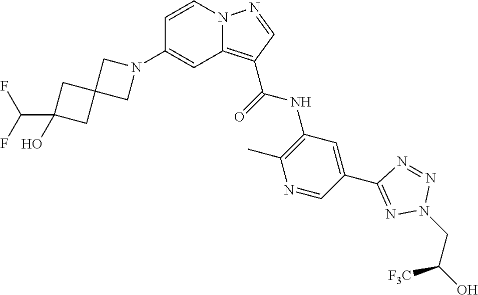 [SMILES: Cc1ncc(-c2nnn(C[C@@H](O)C(F)(F)F)n2)cc1NC(=O)c1cnn2ccc(N3CC4(C3)CC(O)(C(F)F)C4)cc12] | (M + H)⁺: 594.2 |

### Example 313

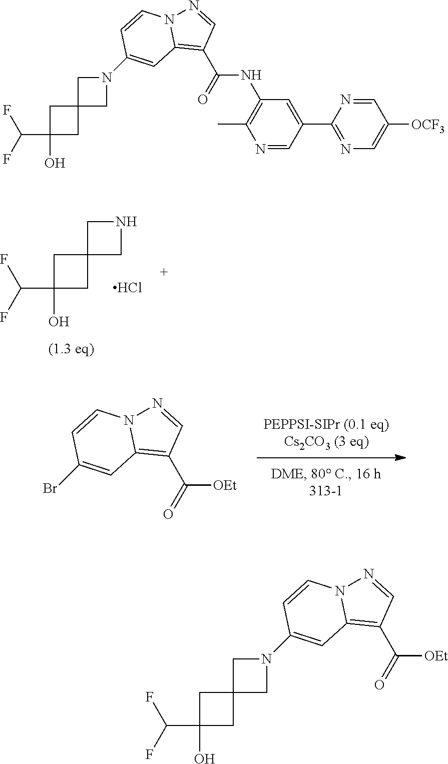 [SMILES: Cc1ncc(-c2ncc(OC(F)(F)F)cn2)cc1NC(=O)c1cnn2ccc(N3CC4(C3)CC(O)(C(F)F)C4)cc12 ; OC1(C(F)F)CC2(CNC2)C1 ; Cl ; CCOC(=O)c1cnn2ccc(Br)cc12 ; CCOC(=O)c1cnn2ccc(N3CC4(C3)CC(O)(C(F)F)C4)cc12]

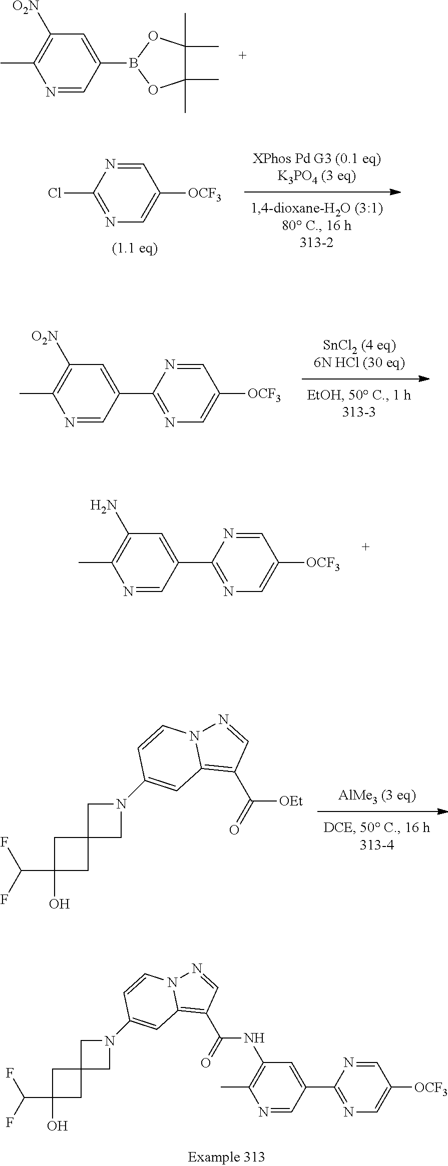

Step 313-1: 6-(difluoromethyl)-2-azaspiro[3.3]heptan-6-ol hydrochloride (482 mg, 1.3 eq, 2.42 mmol), ethyl 5-bromopyrazolo[1,5-a]pyridine-3-carboxylate (500 mg, 1.0 eq, 1.86 mmol), PEPPSI™—SIPr catalyst (127 mg, 0.1 Eq, 186 mol) and cesium carbonate (1.82 g, 3 Eq, 5.57 mmol) were loaded in a round bottom flask equipped with a magnetic stir bar. The flask was sealed, evacuated and refilled with nitrogen. Anhydrous DME (10 mL) was added via syringe, and the reaction was heated at 80° C. over 16 hrs. The flask was cooled to room temperature, and the crude mixture was filtered through celite plug, washing with DCM. After removal of solvent, the crude was purified over silica gel chromatography (0-20% MeOH in DCM) to reveal the desired product (480 mg, 1.37 mmol, 74% yield) as a light yellow powder. (ES, m/z): [M+H]⁺=352.16.
Step 313-2: A solution of 2-methyl-3-nitro-5-(4,4,5,5-tetramethyl-1,3,2-dioxaborolan-2-yl)pyridine (141 mg, 1 Eq, 534 mol) and 2-chloro-5-(trifluoromethoxy)pyrimidine (116 mg, 1.09 Eq, 584 mol) in 1,4-Dioxane (1 mL) and water (0.3 mL) was treated with tripotassium phosphate (348 mg, 3.07 Eq, 1.64 mmol) and XPhos Pd G3 (59 mg, 0.13 Eq, 70 mol) under N₂. The reaction was warmed to 80° C. and stirred for 16 hours. The mixture was concentrated in vacuo and diluted with dichloromethane. The mixture was filtered and rinsed with dichloromethane and acetone. The filtrate was concentrated in vacuo. The crude was added to a 24 g silica gel column and eluted by ethyl acetate/cyclohexane from 0% to 100% to give the desired product (155 mg, 516 mol, 97%) as a white solid. (ES, m/z): [M+H]⁺=300.85.
Step 313-3: A solution of the compound from Step 313-2 (155 mg, 1 Eq, 516 mol) in EtOH (0.5 mL) was treated with HCl (0.55 g, 2.5 mL, 6 molar, 29 Eq, 15 mmol) and tin(II) chloride (392 mg, 4 Eq, 2.07 mmol). The reaction was warmed to 50° C. and stirred for 1 hour. The reaction was neutralized with 5 N NaOH aqueous solution. The aqueous layer was extracted with ethyl acetate over 3 times. The combined organic layer was dried over sodium sulfate, filtered and concentrated in vacuo. The crude was added to a 12 g silica gel column and eluted by ethyl acetate/cyclohexane from 0% to 100% to give the desired product (68 mg, 0.25 mmol, 49%) as a white solid. (ES, m/z): [M+H]⁺=270.89.
Step 313-4: A solution of the compound from Step 313-1 (34 mg, 1.3 Eq, 97 mol) and the compound from Step 313-3 (20 mg, 1 Eq, 74 mol) in 1,2-Dichloroethane (0.5 mL) was treated with a 2 M solution of trimethylaluminum in toluene (15.9 mg, 110 μL, 2 molar, 3.0 Eq, 220 mol). The reaction was warmed to 50° C. and stirred for 16 hours. The reaction was quenched with a saturated solution of potassium sodium tartrate. The mixture was stirred at room temperature for 2 hours. The aqueous layer was extracted with dichloromethane over 3 times. The combined organic layer was dried over sodium sulfate, filtered and concentrated in vacuo. The crude was added to a 4 g silica gel column and eluted by acetone/cyclohexane from 0% to 100% to give Example 313 (29 mg, 50 mol, 68%) as a white solid. (ES, m/z): [M+H]⁺=576.29.

¹H NMR (500 MHz, DMSO) δ 9.48 (s, 1H), 9.20 (d, J=2.0 Hz, 1H), 9.14 (d, J=0.9 Hz, 2H), 8.79 (d, J=2.1 Hz, 1H), 8.57 (s, 1H), 8.54 (d, J=7.5 Hz, 1H), 6.83 (d, J=2.5 Hz, 1H), 6.41 (dd, J=7.5, 2.6 Hz, 1H), 5.91 (s, 1H), 5.82 (s, 1H), 4.01 (s, 2H), 3.95 (s, 2H), 2.58 (s, 3H), 2.55 (s, 1H), 2.28 (d, J=13.3 Hz, 2H).

The following examples were prepared using procedures similar to those described above:

| Example | Structure | ESI-MS |
|---|---|---|
| 314 | 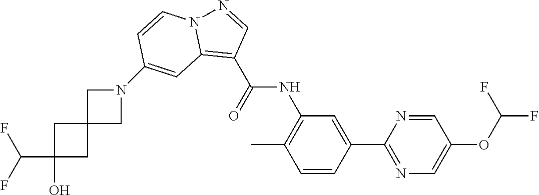 [SMILES: Cc1ccc(-c2ncc(OC(F)F)cn2)cc1NC(=O)c1cnn2ccc(N3CC4(C3)CC(O)(C(F)F)C4)cc12] | (M + H)⁺: 557.04 |
| 315 | 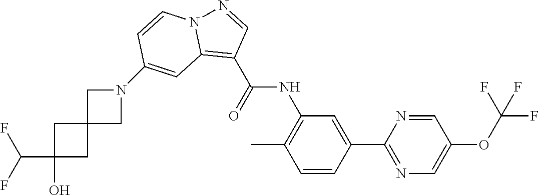 [SMILES: Cc1ccc(-c2ncc(OC(F)(F)F)cn2)cc1NC(=O)c1cnn2ccc(N3CC4(C3)CC(O)(C(F)F)C4)cc12] | (M + H)⁺: 575.07 |
| 316 | 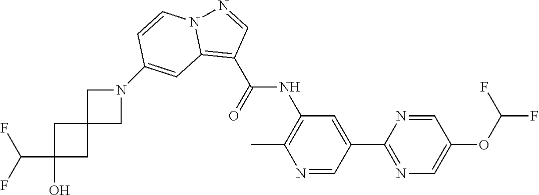 [SMILES: Cc1ncc(-c2ncc(OC(F)F)cn2)cc1NC(=O)c1cnn2ccc(N3CC4(C3)CC(O)(C(F)F)C4)cc12] | (M + H)⁺: 558.16 |
| 317 | 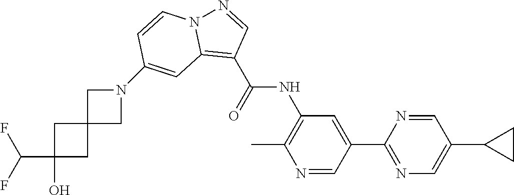 [SMILES: Cc1ncc(-c2ncc(C3CC3)cn2)cc1NC(=O)c1cnn2ccc(N3CC4(C3)CC(O)(C(F)F)C4)cc12] | (M + H)⁺: 532.18 |
| 318 | 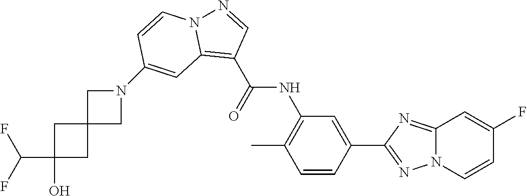 [SMILES: Cc1ccc(-c2nc3cc(F)ccn3n2)cc1NC(=O)c1cnn2ccc(N3CC4(C3)CC(O)(C(F)F)C4)cc12] | (M + H)⁺: 548.19 |
| 319 | 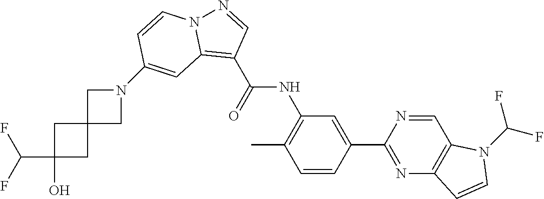 [SMILES: Cc1ccc(-c2ncc3c(ccn3C(F)F)n2)cc1NC(=O)c1cnn2ccc(N3CC4(C3)CC(O)(C(F)F)C4)cc12] | (M + H)⁺: 580.25 |
| 320 | 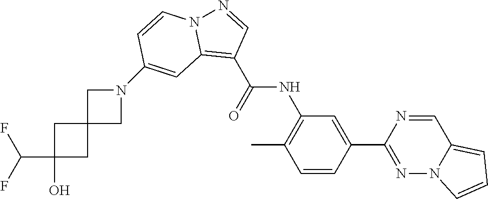 [SMILES: Cc1ccc(-c2ncc3cccn3n2)cc1NC(=O)c1cnn2ccc(N3CC4(C3)CC(O)(C(F)F)C4)cc12] | (M + H)⁺: 530.31 |
| 321 | 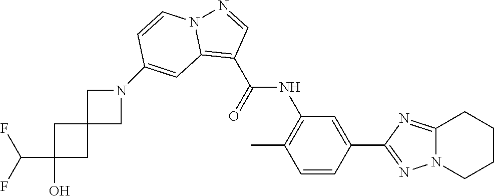 [SMILES: Cc1ccc(-c2nc3n(n2)CCCC3)cc1NC(=O)c1cnn2ccc(N3CC4(C3)CC(O)(C(F)F)C4)cc12] | (M + H)⁺: 534.17 |
| 322 | 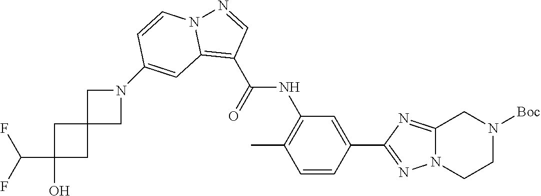 [SMILES: Cc1ccc(-c2nc3n(n2)CCN(C(=O)OC(C)(C)C)C3)cc1NC(=O)c1cnn2ccc(N3CC4(C3)CC(O)(C(F)F)C4)cc12] | (M + H)⁺: 635.45 |
| 323 | 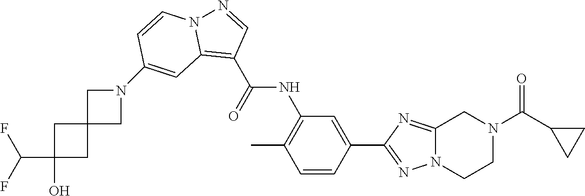 [SMILES: Cc1ccc(-c2nc3n(n2)CCN(C(=O)C2CC2)C3)cc1NC(=O)c1cnn2ccc(N3CC4(C3)CC(O)(C(F)F)C4)cc12] | (M + H)⁺: 603.44 |
| 324 | 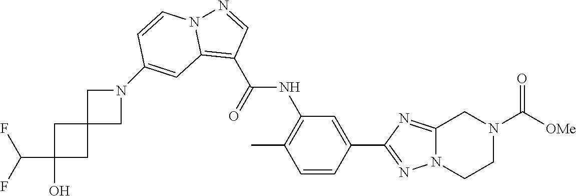 [SMILES: COC(=O)N1CCn2nc(-c3ccc(C)c(NC(=O)c4cnn5ccc(N6CC7(C6)CC(O)(C(F)F)C7)cc45)c3)nc2C1] | (M + H)⁺: 593.41 |
| 325 | 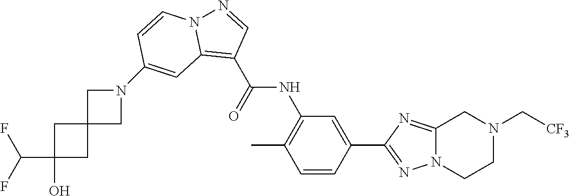 | (M + H)⁺: 535.20 |
| 326 | 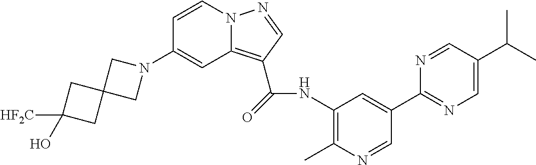 | (M + H)⁺: 534.22 |
| 327 | 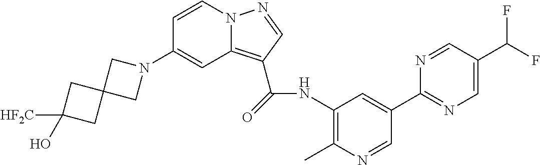 | (M + H)⁺: 542.09 |
| 328 | 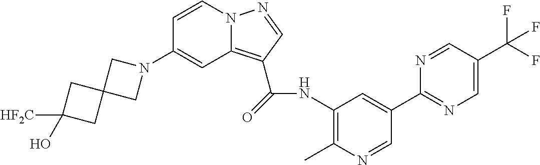 | (M + H)⁺: 560.24 |
| 329 | 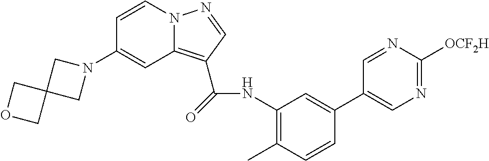 | (M + H)⁺: 493.18 |
| 330 | 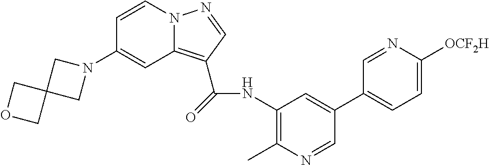 | (M + H)⁺: 493.33 |
| 331 | 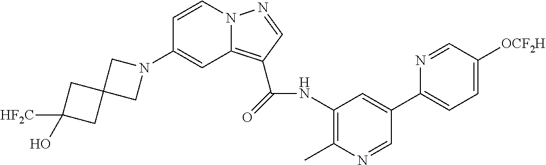 | (M + H)⁺: 557.31 |
| 332 | 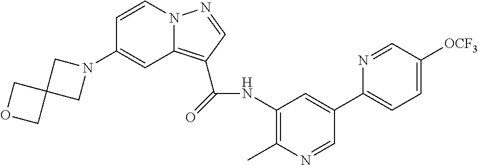 [SMILES: Cc1ncc(-c2ccc(OC(F)(F)F)cn2)cc1NC(=O)c1cnn2ccc(N3CC4(COC4)C3)cc12] | (M + H)⁺: 511.38 |
| 333 | 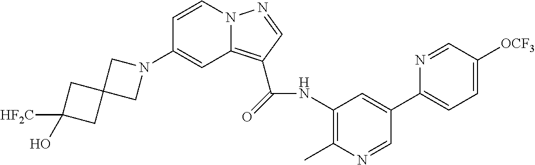 | (M + H)⁺: 575.36 |
| 334 | 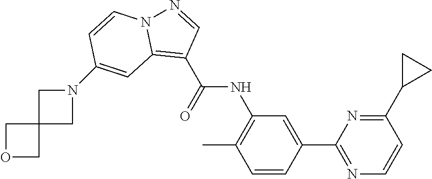 [SMILES: Cc1ccc(-c2nccc(C3CC3)n2)cc1NC(=O)c1cnn2ccc(N3CC4(COC4)C3)cc12] | (M + H)⁺: 467.25 |
| 335 | 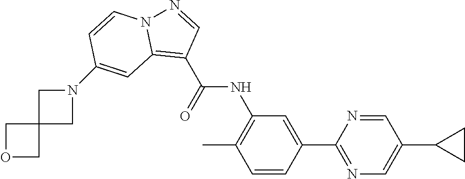 [SMILES: Cc1ccc(-c2ncc(C3CC3)cn2)cc1NC(=O)c1cnn2ccc(N3CC4(COC4)C3)cc12] | (M + H)⁺: 467.30 |
| 336 | 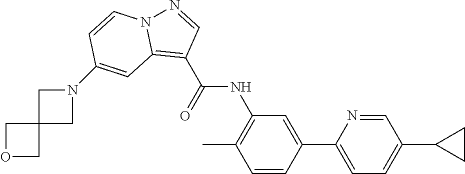 [SMILES: Cc1ccc(-c2ccc(C3CC3)cn2)cc1NC(=O)c1cnn2ccc(N3CC4(COC4)C3)cc12] | (M + H)⁺: 466.20 |
| 337 | 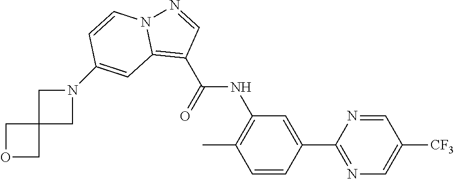 [SMILES: Cc1ccc(-c2ncc(C(F)(F)F)cn2)cc1NC(=O)c1cnn2ccc(N3CC4(COC4)C3)cc12] | (M + H)⁺: 495.25 |
| 338 | 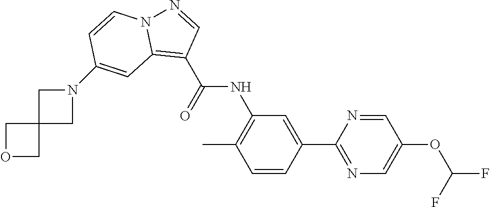 [SMILES: Cc1ccc(-c2ncc(OC(F)F)cn2)cc1NC(=O)c1cnn2ccc(N3CC4(COC4)C3)cc12] | (M + H)⁺: 493.25 |
| 339 | 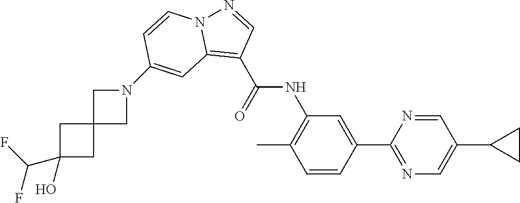 [SMILES: Cc1ccc(-c2ncc(C3CC3)cn2)cc1NC(=O)c1cnn2ccc(N3CC4(C3)CC(O)(C(F)F)C4)cc12] | (M + H)⁺: 531.20 |
| 340 | 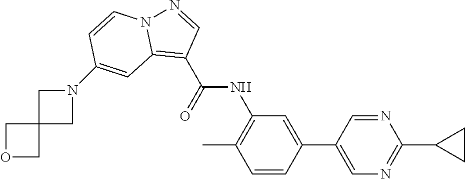 [SMILES: Cc1ccc(-c2cnc(C3CC3)nc2)cc1NC(=O)c1cnn2ccc(N3CC4(COC4)C3)cc12] | (M + H)⁺: 467.25 |
| 341 | 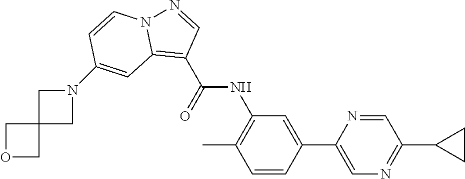 [SMILES: Cc1ccc(-c2cnc(C3CC3)cn2)cc1NC(=O)c1cnn2ccc(N3CC4(COC4)C3)cc12] | (M + H)⁺: 467.10 |
| 342 | 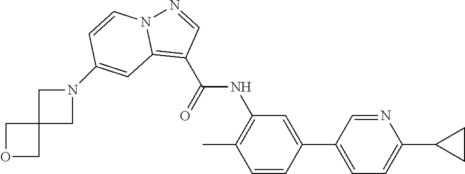 [SMILES: Cc1ccc(-c2ccc(C3CC3)nc2)cc1NC(=O)c1cnn2ccc(N3CC4(COC4)C3)cc12] | (M + H)⁺: 466.25 |
| 343 | 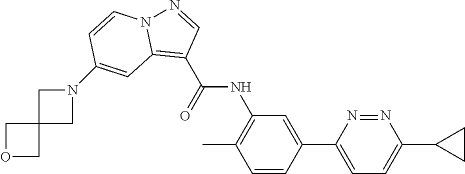 [SMILES: Cc1ccc(-c2ccc(C3CC3)nn2)cc1NC(=O)c1cnn2ccc(N3CC4(COC4)C3)cc12] | (M + H)⁺: 467.20 |
| 344 | 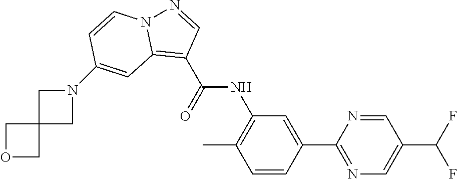 [SMILES: Cc1ccc(-c2ncc(C(F)F)cn2)cc1NC(=O)c1cnn2ccc(N3CC4(COC4)C3)cc12] | (M + H)⁺: 477.15 |
| 345 | 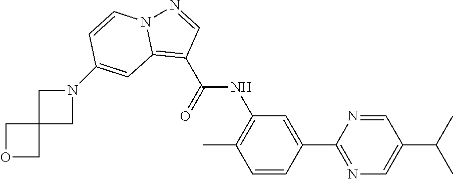 [SMILES: Cc1ccc(-c2ncc(C(C)C)cn2)cc1NC(=O)c1cnn2ccc(N3CC4(COC4)C3)cc12] | (M + H)⁺: 469.20 |
| 346 | 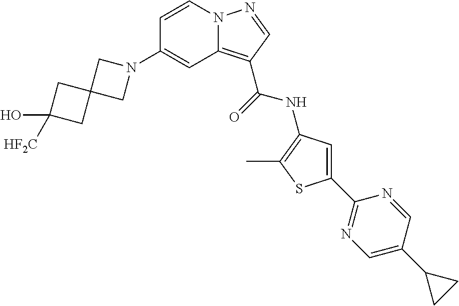 | (M + H)⁺: 537.15 |
| 347 | 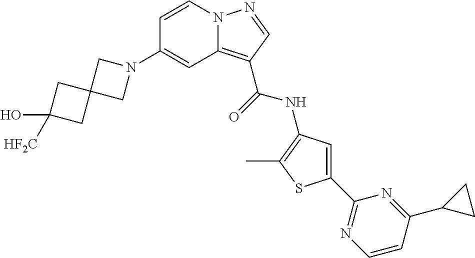 | (M + H)⁺: 537.25 |
| 348 | 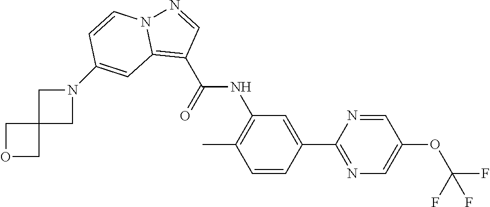 [SMILES: Cc1ccc(-c2ncc(OC(F)(F)F)cn2)cc1NC(=O)c1cnn2ccc(N3CC4(COC4)C3)cc12] | (M + H)⁺: 511.25 |
| 349 | 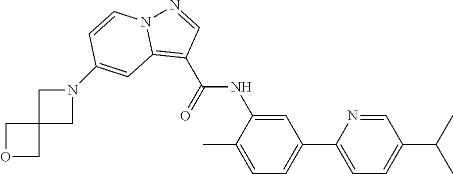 [SMILES: Cc1ccc(-c2ccc(C(C)C)cn2)cc1NC(=O)c1cnn2ccc(N3CC4(COC4)C3)cc12] | (M + H)⁺: 468.30 |
| 350 | 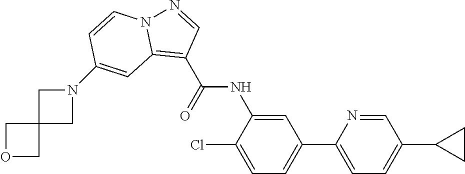 [SMILES: O=C(Nc1cc(-c2ccc(C3CC3)cn2)ccc1Cl)c1cnn2ccc(N3CC4(COC4)C3)cc12] | (M + H)⁺: 486.10 |
| 351 | 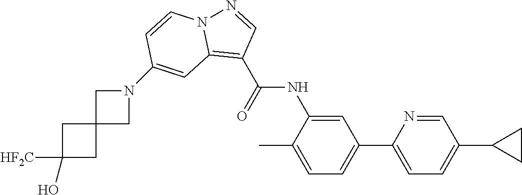 | (M + H)⁺: 530.20 |
| 352 | 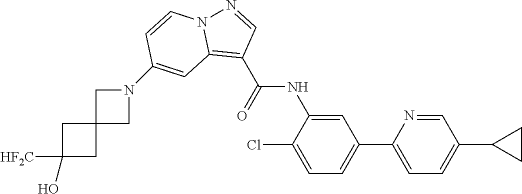 | (M + H)⁺: 550.15 |
| 353 | 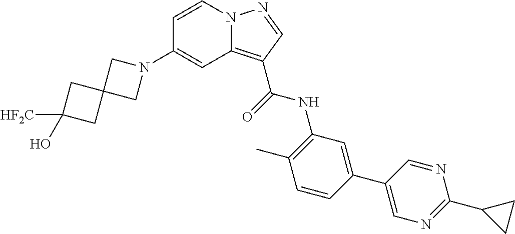 | (M + H)⁺: 531.30 |
| 354 | 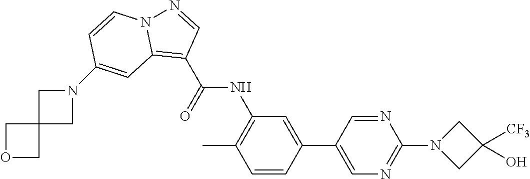 [SMILES: Cc1ccc(-c2cnc(N3CC(O)(C(F)(F)F)C3)nc2)cc1NC(=O)c1cnn2ccc(N3CC4(COC4)C3)cc12] | (M + H)⁺: 566.15 |
| 355 | 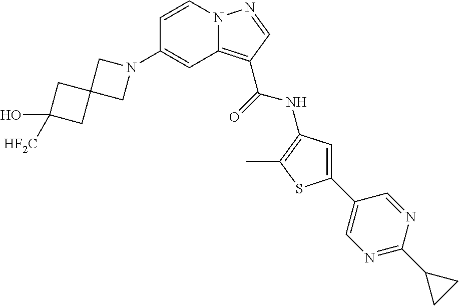 | (M + H)⁺: 537.15 |
| 356 | 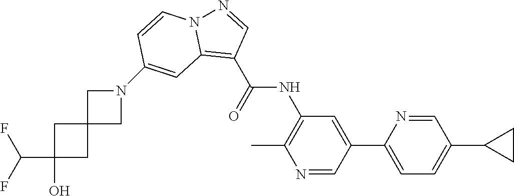 [SMILES: Cc1ncc(-c2ccc(C3CC3)cn2)cc1NC(=O)c1cnn2ccc(N3CC4(C3)CC(O)(C(F)F)C4)cc12] | (M + H)⁺: 531.30 |
| 357 | 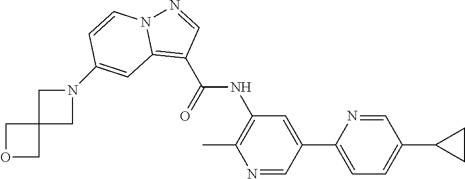 [SMILES: Cc1ncc(-c2ccc(C3CC3)cn2)cc1NC(=O)c1cnn2ccc(N3CC4(COC4)C3)cc12] | (M + H)⁺: 467.25 |

### Example 358

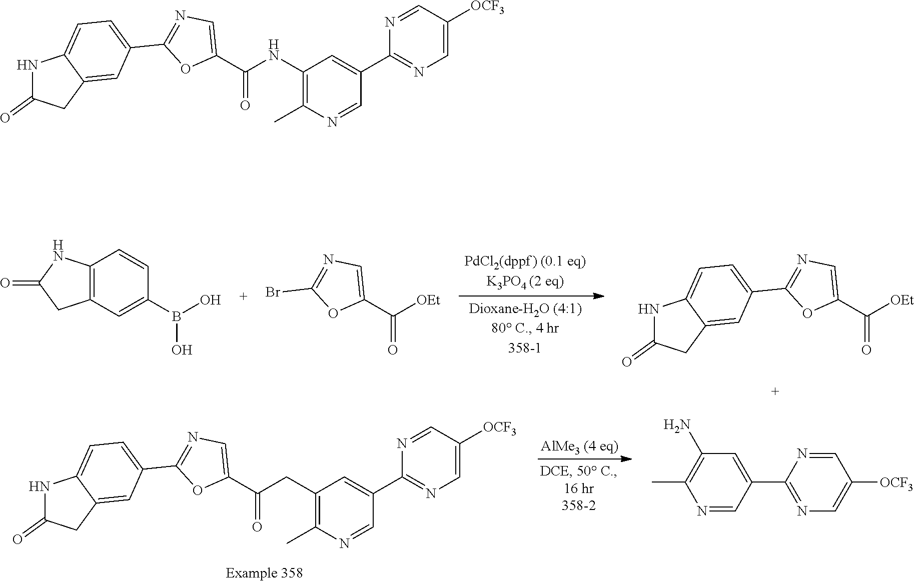 [SMILES: Cc1ncc(-c2ncc(OC(F)(F)F)cn2)cc1NC(=O)c1cnc(-c2ccc3c(c2)CC(=O)N3)o1 ; O=C1Cc2cc(B(O)O)ccc2N1 ; CCOC(=O)c1cnc(Br)o1 ; CCOC(=O)c1cnc(-c2ccc3c(c2)CC(=O)N3)o1 ; Cc1ncc(-c2ncc(OC(F)(F)F)cn2)cc1CC(=O)c1cnc(-c2ccc3c(c2)CC(=O)N3)o1 ; Cc1ncc(-c2ncc(OC(F)(F)F)cn2)cc1N]

Step 358-1: A solution of 5-(4,4,5,5-tetramethyl-1,3,2-dioxaborolan-2-yl)indolin-2-one (2.11 g, 1 Eq, 8.14 mmol) and ethyl 2-bromooxazole-5-carboxylate (2.35 g, 1.31 Eq, 10.7 mmol) in 1,4-Dioxane (32 mL) and Water (8 mL) was treated with [1,1′-Bis(di-tert-butylphosphino)ferrocene]dichloropalladium(II) (558 mg, 0.105 Eq, 856 mol) and tripotassium phosphate (3.52 g, 2.04 Eq, 16.6 mmol) under N₂. The reaction was warmed to 80° C. and stirred for 4 hours. The mixture was filtered through celite and concentrated in vacuo. The crude was added to a 24 g silica gel column and eluted by ethyl acetate/cyclohexane from 0% to 100% to give ethyl 2-(2-oxoindolin-5-yl)oxazole-5-carboxylate (336 mg, 1.23 mmol, 15%) as a brownish solid. (ES, m/z): [M+H]⁺=272.86.
Step 358-2: A solution of the compound from Step 358-1 (18 mg, 1.1 Eq, 66 mol) and the compound from Step 313-3 (16 mg, 1 Eq, 59 mol) in 1,2-Dichloroethane (0.5 mL) was treated with a 2 M solution of trimethylaluminum in toluene (15.9 mg, 110 μL, 2 molar, 3.7 Eq, 220 mol). The reaction was warmed to 50° C. and stirred for 16 hours. The mixture was diluted with DMSO and then quenched with methanol slowly. The mixture was filtered and purified by preparative reverse phase HPLC to give N-(2-methyl-5-(5-(trifluoromethoxy)pyrimidin-2-yl)pyridin-3-yl)-2-(2-oxoindolin-5-yl)oxazole-5-carboxamide (15 mg, 30 mol, 51%) as an off-white solid. (ES, m/z): [M+H]⁺=497.18.

The following examples were prepared using procedures similar to those described above:

| Example | Structure | ESI-MS |
|---|---|---|
| 359 | 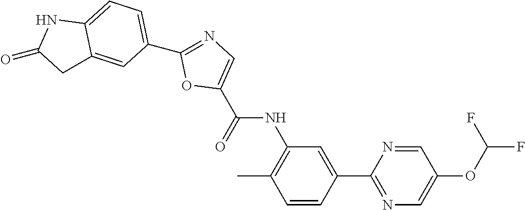 [SMILES: Cc1ccc(-c2ncc(OC(F)F)cn2)cc1NC(=O)c1cnc(-c2ccc3c(c2)CC(=O)N3)o1] | (M + H)⁺: 477.76 |
| 360 | 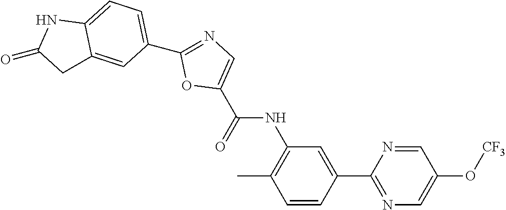 [SMILES: Cc1ccc(-c2ncc(OC(F)(F)F)cn2)cc1NC(=O)c1cnc(-c2ccc3c(c2)CC(=O)N3)o1] | (M + H)⁺: 495.80 |
| 361 | 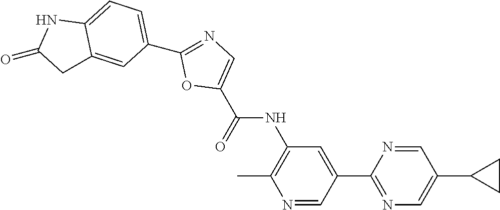 [SMILES: Cc1ncc(-c2ncc(C3CC3)cn2)cc1NC(=O)c1cnc(-c2ccc3c(c2)CC(=O)N3)o1] | (M + H)⁺: 453.04 |
| 362 | 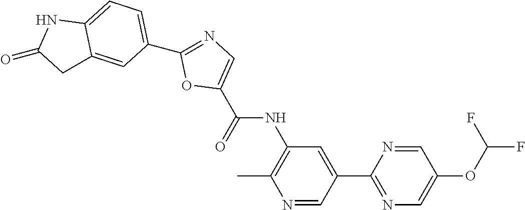 [SMILES: Cc1ncc(-c2ncc(OC(F)F)cn2)cc1NC(=O)c1cnc(-c2ccc3c(c2)CC(=O)N3)o1] | (M + H)⁺: 479.19 |
| 363 | 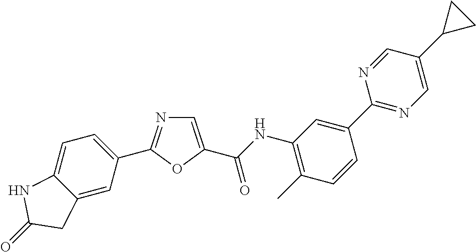 [SMILES: Cc1ccc(-c2ncc(C3CC3)cn2)cc1NC(=O)c1cnc(-c2ccc3c(c2)CC(=O)N3)o1] | (M + H)⁺: 452.10 |

### Example 364

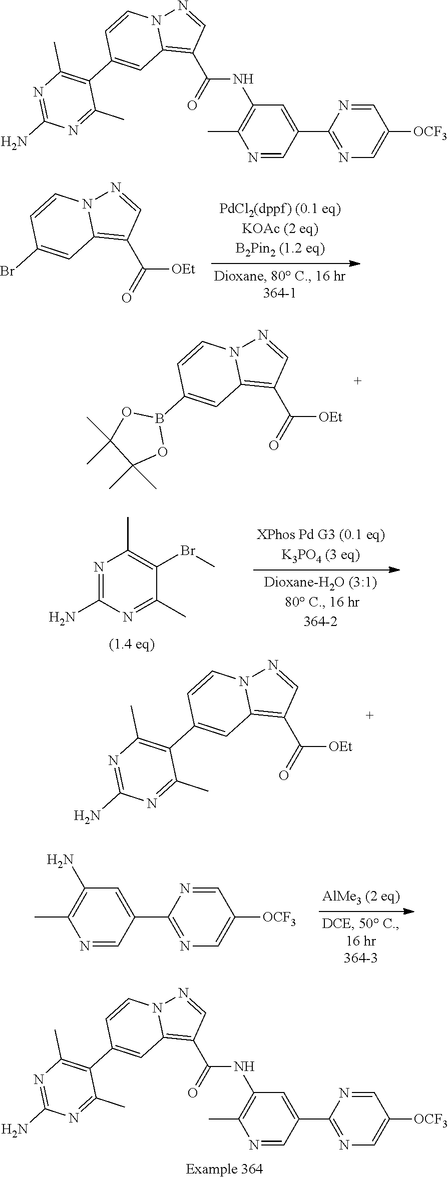

Step 364-1: A solution of ethyl 5-bromopyrazolo[1,5-a]pyridine-3-carboxylate (4.4 g, 1 Eq, 16 mmol) and bis(pinacolato)diboron (5.1 g, 1.2 Eq, 20 mmol) in 1,4-Dioxane (40 mL) was treated with potassium acetate (4.98 g, 3.1 Eq, 50.7 mmol) and PdCl₂(dppf) (1.15 g, 0.096 Eq, 1.57 mmol) under N₂. The reaction was warmed to 80° C. and stirred for 16 hours. The mixture was filtered through celite and concentrated in vacuo. The crude was added to an ethyl acetate/cyclohexane from 0% to 20% to give the desired product (3.77 g, 11.9 mmol, 73%) as an off-white solid. (ES, m/z): [M+H]⁺=316.96.
Step 364-2: A solution of 5-bromo-4,6-dimethylpyrimidin-2-amine (580 mg, 1.45 Eq, 2.87 mmol) and the compound from Step 364-1 (628 mg, 1 Eq, 1.99 mmol) in 1,4-Dioxane (6 mL) and Water (2 mL) was treated with tripotassium phosphate (1.28 g, 3.04 Eq, 6.03 mmol) and XPhos Pd G3 (174 mg, 0.103 Eq, 206 mol) under N₂. The reaction was warmed to 80° C. and stirred for 16 hours. The reaction was quenched with water. The aqueous layer was extracted with ethyl acetate over 3 times. The combined organic layer was dried over sodium sulfate, filtered and concentrated in vacuo. The crude was added to a 24 g silica gel column and eluted by methanol/dichloromethane from 0% to 20% to give the desired product (420 mg, 1.35 mmol, 68%) as a yellow solid. (ES, m/z): [M+H]⁺=312.14.
Step 364-3: To a solution of the compound from Step 364-2 (30 mg, 1 Eq, 96 mol) and the compound from Step 313-3 (29 mg, 1.1 Eq, 0.11 mmol) in anhydrous DCE at rt under N₂ was added trimethylaluminum in toluene (14 mg, 96 μL, 2 molar, 2 Eq, 0.19 mmol), the reaction mixture was stirred at rt for 20 min, and the mixture was heated to 50° C. and stirred overnight, cooled to 0° C., quenched with Brine solution (2 mL), filtered through celite, the filtrate was separated, and the organic layer was concentrated. The crude mixture was purified by chromatography on silica gel using Cyclohexane/EA (100:0 to 0:100) to give Example 364 as a white solid. (ES, m/z): [M+H]⁺=536.1.

The following examples were prepared using procedures similar to those described above:

| Example | Structure | ESI-MS |
|---|---|---|
| 365 | 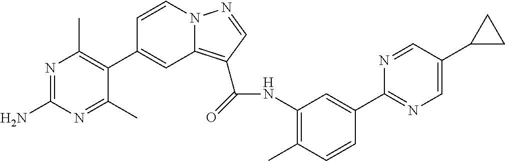 [SMILES: Cc1ccc(-c2ncc(C3CC3)cn2)cc1NC(=O)c1cnn2ccc(-c3c(C)nc(N)nc3C)cc12] | (M + H)⁺: 491.4 |
| 366 | 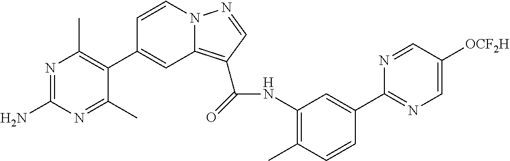 | (M + H)⁺: 517.1 |
| 367 | 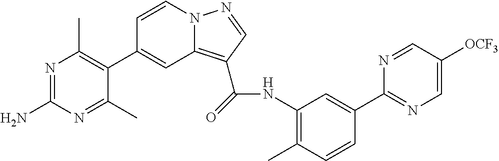 [SMILES: Cc1ccc(-c2ncc(OC(F)(F)F)cn2)cc1NC(=O)c1cnn2ccc(-c3c(C)nc(N)nc3C)cc12] | (M + H)⁺: 535.4 |
| 368 | 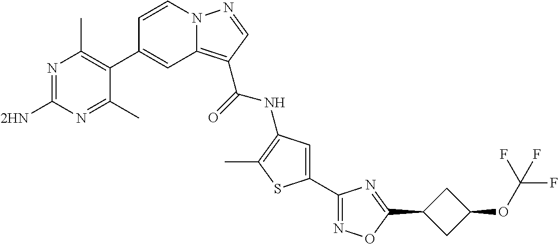 | (M + H)⁺: 585.169, 586.171 |
| 369 | 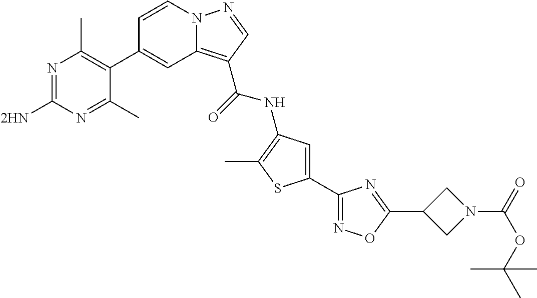 | (M + H)⁺: 602.541, 546.139 |
| 370 | 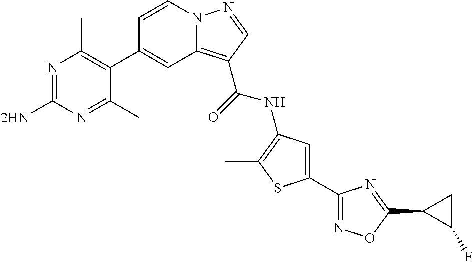 | (M + H)⁺: 505.483 |
| 371 | 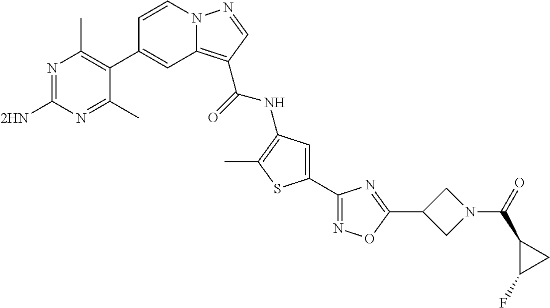 | (M + H)⁺: 588.332 |
| 371-1 | 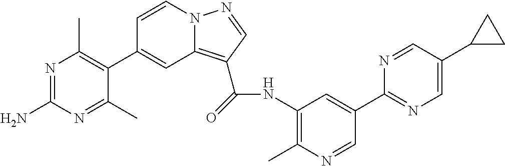 [SMILES: Cc1ncc(-c2ncc(C3CC3)cn2)cc1NC(=O)c1cnn2ccc(-c3c(C)nc(N)nc3C)cc12] | (M + H)⁺: 492.7 |

### Example 372

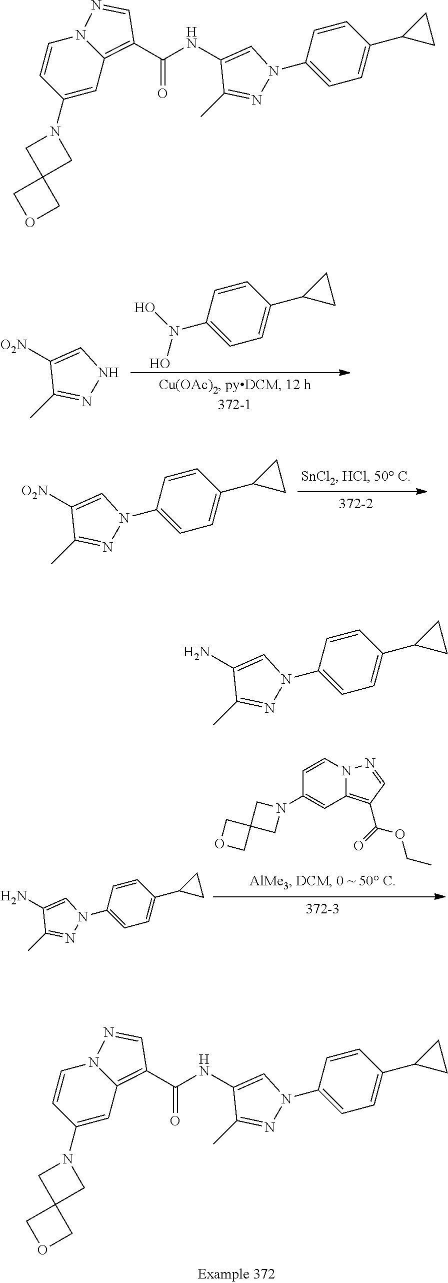

Step 372-1: To a stirred solution of 3-methyl-4-nitro-1H-pyrazole (200 mg, 1.574 mmol, 1 equiv) and (4-cyclopropylphenyl)boronic acid (510 mg, 3.148 mmol, 2 equiv) in DCM (3 mL) was added Cu(OAc)₂ (429 mg, 2.361 mmol, 1.5 equiv), pyridine (93 mg, 1.180 mmol, 5 equiv) dropwise at room temperature under air atmosphere. The resulting mixture was stirred at room temperature for 12 h under air atmosphere. The aqueous layer was extracted with CH₂Cl₂ (3×20 mL). The residue was purified by Prep-TLC (PE/EA 5:1) to afford the desired product (245 mg, 90% purity, 58% yield) as a white solid. (ES, m/z): [M+H]=244.15.
Step 372-2: To a stirred solution of the compound from Step 372-1 (245 mg, 1.007 mmol, 1 equiv) and O₂ (955 mg, 5.035 mmol, 5 equiv) in EtOH (4 mL) and H₂O (4 mL) were added HCl (37 mg, 1.007 mmol, 1 equiv) dropwise at 0° C. under nitrogen atmosphere. The reaction was stirred 12 h at 50° C. The resulting mixture was concentrated under reduced pressure. The mixture was neutralized to pH 8 with saturated NaHCO₃(aq.). The aqueous layer was extracted with EtOAc (3×10 mL). The residue was purified by reversed-phase flash chromatography with the following conditions: column, C18 silica gel; mobile phase, MeCN in Water, 10% to 50% gradient in 10 min; detector, UV 254 nm. This resulted in the desired product (160 mg, 90% purity, 67% yield) as off-white solid. (ES, m/z): [M+H]⁺=214.15.
Step 372-3: To a stirred mixture of the compound from Step 372-2 (60 mg, 0.209 mmol, 1 equiv) and the compound from Step 242-1 (45 mg, 0.209 mmol, 1 equiv) in DCE (2 mL) was added AlMe₃ in Tol. (0.28 mL, 0.627 mmol, 3 equiv) dropwise at 0° C. under nitrogen atmosphere. The resulting mixture was stirred at 50° C. for 4 h under nitrogen atmosphere. The reaction was quenched with sat. NH₄Cl (aq.) at room temperature. The aqueous layer was extracted with EtOAc (3×20 mL). The crude product (90 mg) was purified by Prep-HPLC with the following conditions (Column: Xselect CSH C18 OBD Column 19*250 mm 5 m; Mobile Phase A: Water (0.1% FA), Mobile Phase B: ACN; Flow rate: 30 mL/min mL/min; Gradient: 40% B to 62% B in 8 min; Wave Length: 254 nm/220 nm nm; RT1(min): 7.72; Number of Runs: 4) to afford Example 372 (19.8 mg, 99.7% purity, 21% yield) as a white solid. (ES, m/z): [M+H]⁺=455.30.

¹H NMR (400 MHz, DMSO) δ 9.25 (s, 1H), 8.58-8.50 (m, 3H), 7.69-7.62 (m, 2H), 7.20-7.13 (m, 2H), 6.88 (d, J=2.6 Hz, 1H), 6.41 (dd, J=2.6, 7.5 Hz, 1H), 4.75 (s, 4H), 4.16 (s, 4H), 2.28 (s, 3H), 1.95 (td, J=4.3, 8.5 Hz, 1H), 1.01-0.92 (m, 2H), 0.73-0.65 (m, 2H).

The following examples were prepared using procedures similar to those described above:

| Example | Structure | ESI-MS |
|---|---|---|
| 373 | 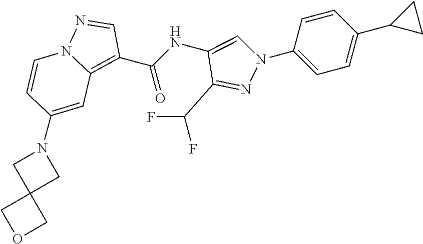 [SMILES: O=C(Nc1cn(-c2ccc(C3CC3)cc2)nc1C(F)F)c1cnn2ccc(N3CC4(COC4)C3)cc12] | (M + H)⁺: 491.15 |

### Example 374

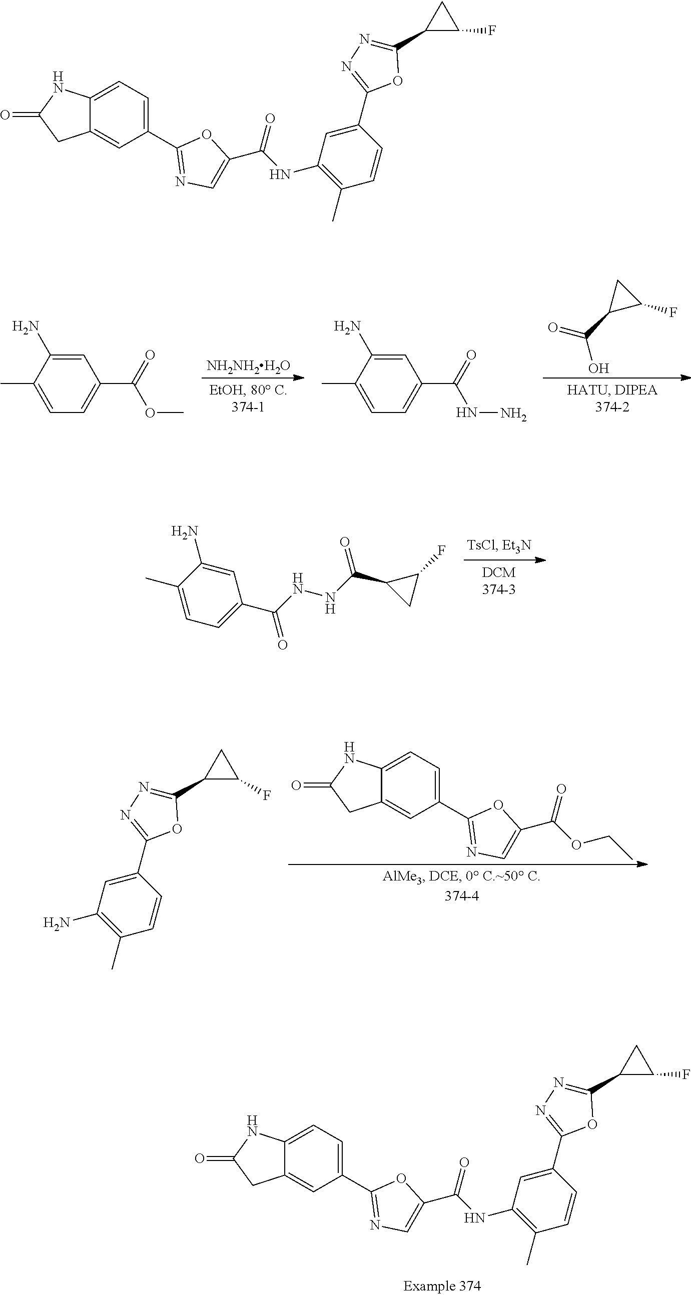

Step 374-1: A mixture of methyl 3-amino-4-methylbenzoate (1.5 g, 9.080 mmol, 1 equiv) and hydrazine monohydrate (1.14 g, 22.700 mmol, 2.5 equiv) in EtOH (10 mL) was stirred at 80° C. for 16 h under air atmosphere. Desired product could be detected by LCMS. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in the desired product (1 g, 67% yield, 80% purity) as a brown solid. (ES, m/z): [M+H]⁺=166.09.
Step 374-2: To a stirred solution of the compound from Step 374-1 (500 mg, 3.027 mmol, 1 equiv) and (1R,2S)-2-fluorocyclopropane-1-carboxylic acid (346.52 mg, 3.330 mmol, 1.1 equiv) in DMF (5 mL) were added HATU (1.73 g, 4.540 mmol, 1.5 equiv) and DIPEA (1.56 g, 12.108 mmol, 4 equiv) in portions at 0° C. under air atmosphere. The resulting mixture was stirred at room temperature for 3 h under air atmosphere. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in the desired product (500 mg, 66% yield, 80% purity) as an off-white solid. (ES, m/z): [M+H]=252.11.
Step 374-3: To a stirred mixture of the compound from Step 374-2 (300 mg, 1.194 mmol, 1 equiv) and TsCl (227 mg, 1.194 mmol, 1 equiv), Et₃N (362 mg, 3.582 mmol, 3 equiv) in DCM (3 mL) at 0° C. under air atmosphere. The resulting mixture was stirred at room temperature for 3 h under air atmosphere. The resulting mixture was concentrated under reduced pressure. The residue was purified by reversed-phase flash chromatography with the following conditions: column, C18 silica gel; mobile phase, MeCN in Water (0.1% FA), 20% to 40% gradient in 10 min; detector, UV 254 nm. This resulted in the desired product (80 mg, 26% yield, 90% purity) as an off-white solid. (ES, m/z): [M+H]⁺=234.10.
Step 374-4: To a stirred solution of the compound from Step 374-3 (50 mg, 0.184 mmol, 1.00 equiv) and the compound from Step 358-1 (43 mg, 0.184 mmol, 1 equiv) in DCE (1 mL) were AlMe₃ (2.0M in toluene) (0.28 mL, 0.552 mmol, 3 equiv) dropwise at 0° C. under nitrogen atmosphere. The reaction was stirred for another 2 h at 50° C. The reaction was quenched with sat. NH₄Cl (aq.) at 0° C. The resulting mixture was extracted with CH₂Cl₂ (3×20 mL). The combined organic layers were washed with H₂O (3×20 mL), dried over anhydrous Na₂SO₄. The crude product (60 mg) was purified by Prep-HPLC with the following conditions (Column: Xselect CSH C18 OBD Column 30*150 mm 5 m; Mobile Phase A: Water (0.1% FA), Mobile Phase B: ACN; Flow rate: 60 mL/min mL/min; Gradient: 16% B to 43% B in 7 min; Wave Length: 254 nm/220 nm nm; RT1(min): 7.73; Number of Runs: 2) to afford Example 374 (4.9 mg, 6% yield, 99% purity) as an off-white solid. (ES, m/z): [M+H]+=460.05.

¹H NMR (400 MHz, DMSO-d6) δ 10.77 (s, 1H), 10.27 (s, 1H), 8.04 (d, J=9.0 Hz, 3H), 7.98 (d, J=1.8 Hz, 1H), 7.81 (dd, J=8.0, 1.9 Hz, 1H), 7.53 (d, J=8.0 Hz, 1H), 7.01 (d, J=8.0 Hz, 1H), 5.40-5.02 (m, 1H), 3.63 (s, 2H), 2.99-2.86 (m, 1H), 2.35 (s, 3H), 1.91-1.73 (m, 1H), 1.53 (dq, J=13.4, 6.8 Hz, 1H).

### Example 375

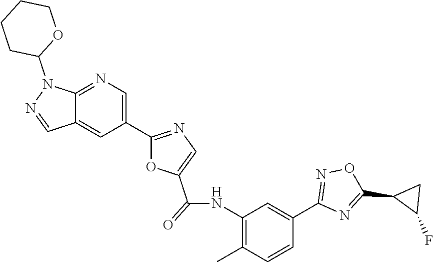 [SMILES: Cc1ccc(-c2noc([C@H]3C[C@@H]3F)n2)cc1NC(=O)c1cnc(-c2cnc3c(cnn3C3CCCCO3)c2)o1]

(ES, m/z): [M+H]⁺=530.05.

### Example 376

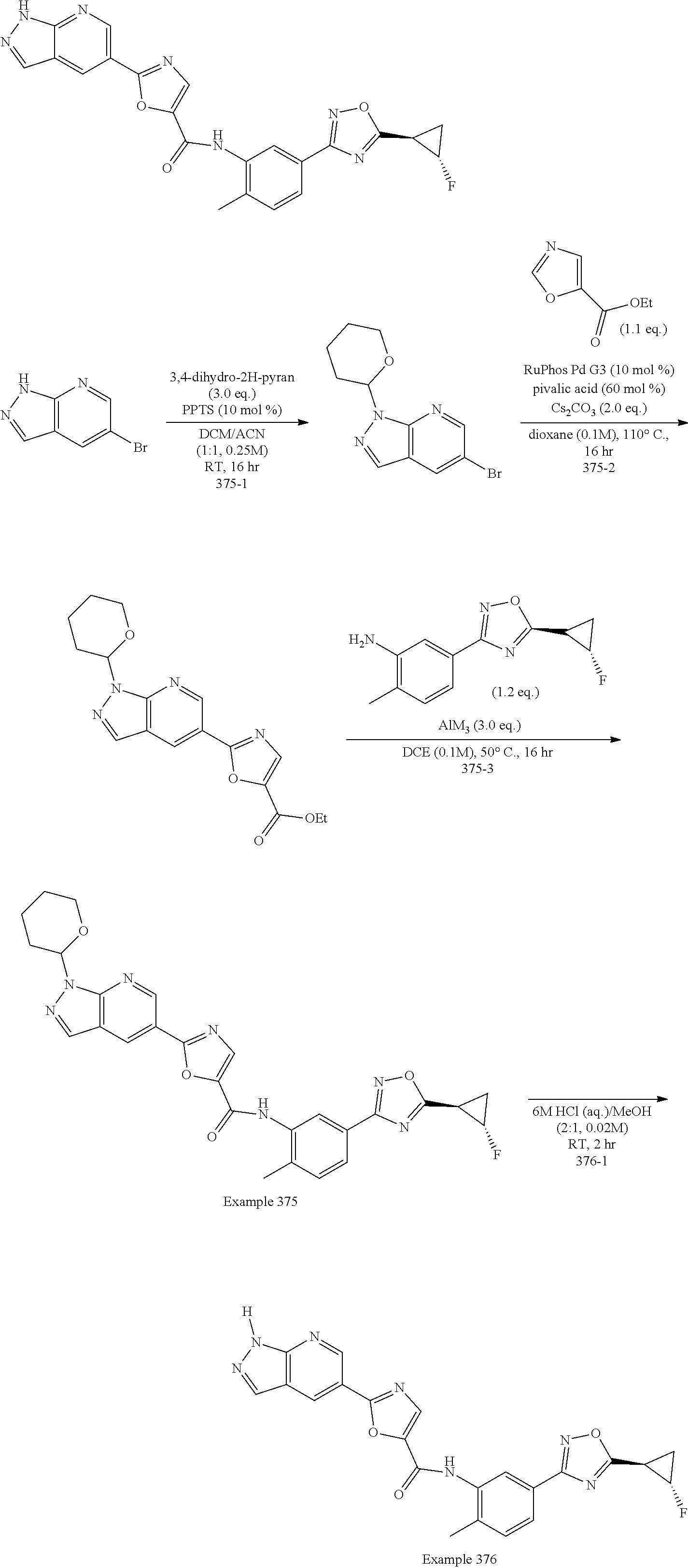

Step 375-1: A mixture of 5-bromo-1H-pyrazolo[3,4-b]pyridine (500 mg, 1 Eq, 2.52 mmol), 3,4-dihydro-2H-pyran (637 mg, 686 μL, 3 Eq, 7.57 mmol) and PPTS (63.5 mg, 0.1 Eq, 252 mol) in DCM (5 mL)/ACN (5 mL) was stirred at RT for 16 hrs. The resulting mixture was concentrated and purified using silica gel column chromatography (0-80% EtOAc/cHex) to afford the desired product (0.71 g, quant.).
Step 375-2: In a 2-dram vial was loaded the compound from Step 375-1 (30 mg, 1 Eq, 0.11 mmol), RuPhos Pd G3 (8.9 mg, 0.1 Eq, 11 mol) and cesium carbonate (69 mg, 2 Eq, 0.21 mmol). The vial was evacuated and refilled with N₂, followed by the addition of 1,4-Dioxane (1 mL) and ethyl oxazole-5-carboxylate (17 mg, 1.1 Eq, 0.12 mmol). The mixture was heated at 110° C. over 16 hrs before being filtered through a celite plug, washing with EtOAc. The filtrate was concentrated and purified over silica gel column chromatography (0-80% EtOAc/cHex) to afford the desired product (23 mg, 63% yield).
Step 375-3: To a solution of the compound from Step 375-2 (20 mg, 1 Eq, 58 mol) and 5-(5-((1R,2S)-2-fluorocyclopropyl)-1,2,4-oxadiazol-3-yl)-2-methylaniline (16 mg, 1.2 Eq, 70 mol) in anhydrous DCE (1 mL) at rt under N₂ was added trimethylaluminum in toluene (13 mg, 88 μL, 2 molar, 3 Eq, 0.18 mmol). The mixture was heated at 50° C. over 16 hours. The mixture was diluted with DCM, quenched with water (3 mL), and then filtered through a celite plug. The filtrate was separated, extracted with DCM (3 mL*3) and the organic layer was concentrated. The crude was purified over prep-HPLC to yield Example 375 as a white solid (18 mg, 58% yield). (ES, m/z): [M+H]=445.96
Step 376-1: To Example 375 (13 mg, 1 Eq, 25 mol) was added HCl (6M) (1 mL) and methanol (0.5 mL). The mixture was stirred at RT over 2 hrs. NaOH (1M) was added and the pH was adjusted to 8-9. The mixture was extracted with EtOAc. The solvent was removed, and the resulting crude was purified over prep-HPLC to reveal Example 376 as a white solid (3.8 mg, 35% yield). (ES, m/z): [M+H]=445.96

### Example 377

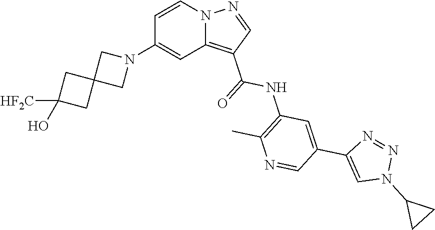

(ES, m/z): [M+H]⁺=521.17.

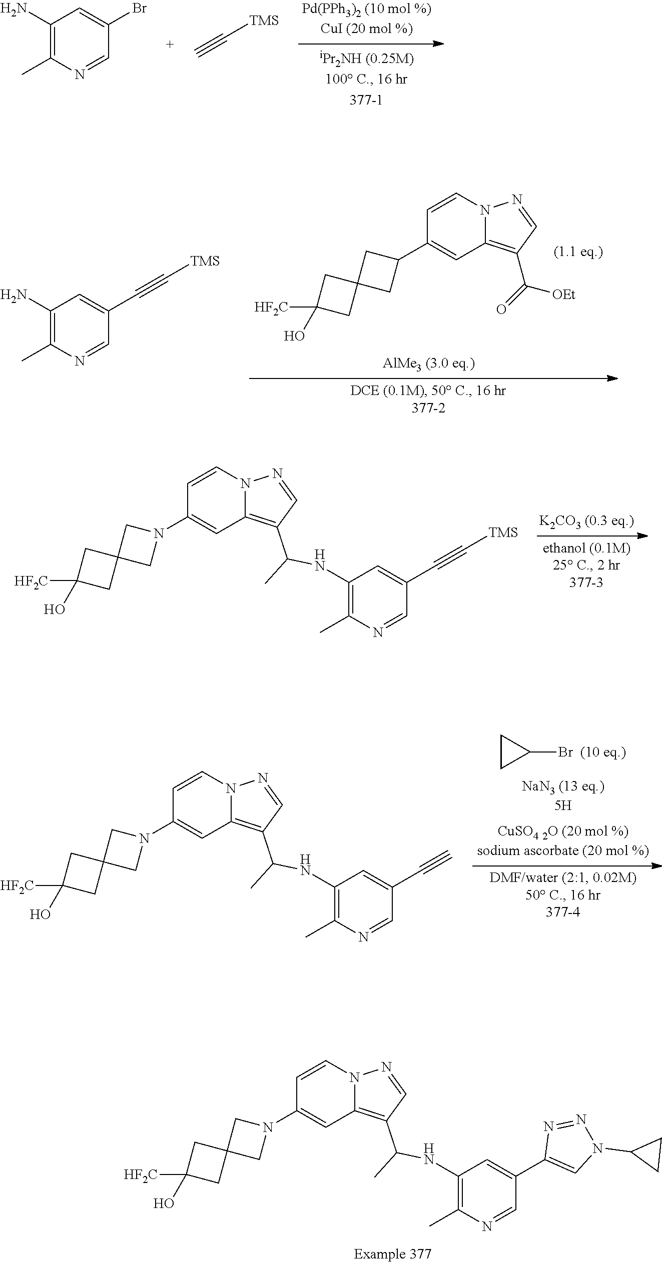

Step 377-1: In a 40 mL vial, 5-bromo-2-methylpyridin-3-amine (500 mg, 1 Eq, 2.67 mmol), bis-(triphenylphosphino)-palladous chloride (188 mg, 0.1 Eq, 267 mol) and CuI (102 mg, 0.2 Eq, 535 mol) was loaded. The vial was evacuated and refilled with N₂, followed by the addition of diisopropylamine (10 mL) and ethynyltrimethylsilane (788 mg, 1.1 mL, 3 Eq, 8.02 mmol). The mixture was heated to 100° C. and stirred overnight before being filtered through a celite plug and concentrated. The crude was then purified over silica gel column chromatography (EtOAc/cHex 0-50%) to afford the desired product (480 mg, 88% yield).
Step 377-2: To a solution of the compound from Step 313-1 (0.13 g, 1.1 Eq, 0.38 mmol) and the compound from Step 377-1 (70 mg, 1 Eq, 0.34 mmol) in anhydrous DCE (1 mL) at rt under N₂ was added trimethylaluminum in toluene (74 mg, 0.51 mL, 2 molar, 3 Eq, 1.0 mmol). The mixture was heated at 50° C. over 16 hours. The mixture was diluted with DCM, quenched with water (3 mL), and then filtered through a celite plug. The filtrate was separated, extracted with DCM (3 mL*3) and the organic layer was concentrated. The crude was purified over silica gel column chromatography (EtOAc/cHex 0-100%) to yield the desired product as a yellow solid (73 mg, 42% yield).
Step 377-3: The compound from Step 377-2 (73 mg, 1 Eq, 0.14 mmol) was dissolved in ethanol (2 mL) and potassium carbonate (5.9 mg, 0.3 Eq, 43 mol) was added. The suspension was stirred over 2 hrs at RT. The mixture was filtered through a celite plug, washing with EtOAc. The solvent was removed, and the resulting crude was purified over silica gel column chromatography (0-100% EtOAc in Hex) to afford the desired product (30 mg, 48% yield).
Step 377-4: In a 20 ml microwave vial, bromocyclopropane (41 mg, 26 μL, 10 Eq, 0.34 mmol) and sodium azide (29 mg, 13 Eq, 0.45 mmol) was suspended in water (0.5 mL). The vial was sealed and heated in a microwave reactor at 120° C. over 30 mins. To the resulting mixture was added the compound from Step 377-3 (15 mg, 1 Eq, 34 μmol), copper(II) sulfate pentahydrate (1.7 mg, 0.2 Eq, 6.9 μmol), sodium ascorbate (1.4 mg, 0.2 Eq, 6.9 μmol) and DMF (1 mL). The resulting mixture was stirred overnight at 80° C. before being filtered through a celite plug, washing with acetone. The filtrate was concentrated, and the crude was purified over prep-TIPLC to afford Example 377 as an off-white solid (5.9 mg, 3300 yield) (ES, m/z): [M+H]⁺=521.17.

The following examples are prepared using procedures similar to those described above:

| Example | Structure |
|---|---|
| 3a | 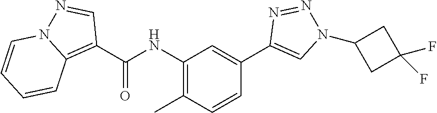 [SMILES: Cc1ccc(-c2cn(C3CC(F)(F)C3)nn2)cc1NC(=O)c1cnn2ccccc12] |
| 4a | 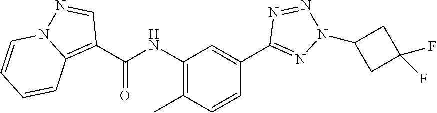 [SMILES: Cc1ccc(-c2nnn(C3CC(F)(F)C3)n2)cc1NC(=O)c1cnn2ccccc12] |
| 5a | 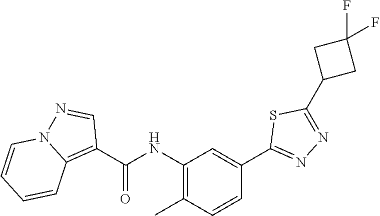 [SMILES: Cc1ccc(-c2nnc(C3CC(F)(F)C3)s2)cc1NC(=O)c1cnn2ccccc12] |
| 6a | 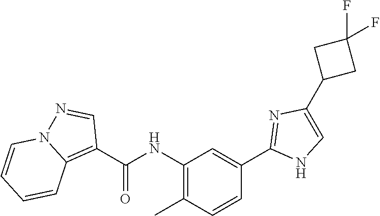 [SMILES: Cc1ccc(-c2nc(C3CC(F)(F)C3)c[nH]2)cc1NC(=O)c1cnn2ccccc12] |
| 7a | 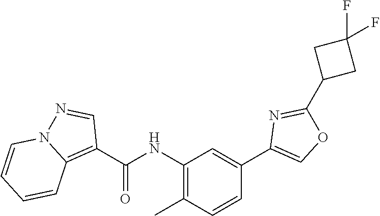 [SMILES: Cc1ccc(-c2coc(C3CC(F)(F)C3)n2)cc1NC(=O)c1cnn2ccccc12] |
| 8a | 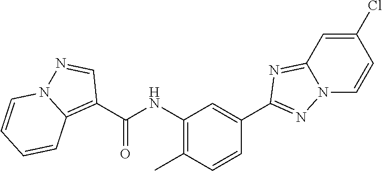 [SMILES: Cc1ccc(-c2nc3cc(Cl)ccn3n2)cc1NC(=O)c1cnn2ccccc12] |
| 9a | 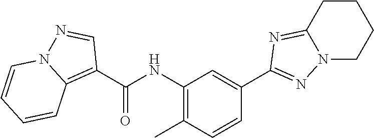 [SMILES: Cc1ccc(-c2nc3n(n2)CCCC3)cc1NC(=O)c1cnn2ccccc12] |
| 10a | 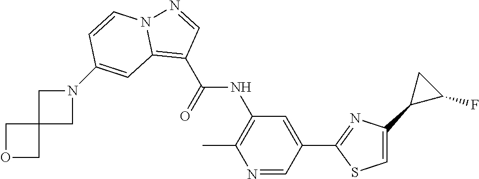 [SMILES: Cc1ncc(-c2nc([C@H]3C[C@@H]3F)cs2)cc1NC(=O)c1cnn2ccc(N3CC4(COC4)C3)cc12] |
| 11a | 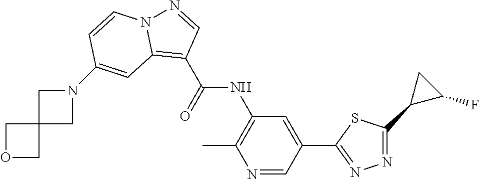 [SMILES: Cc1ncc(-c2nnc([C@H]3C[C@@H]3F)s2)cc1NC(=O)c1cnn2ccc(N3CC4(COC4)C3)cc12] |
| 12a | 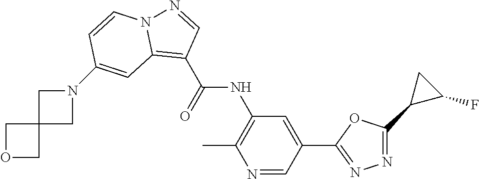 [SMILES: Cc1ncc(-c2nnc([C@H]3C[C@@H]3F)o2)cc1NC(=O)c1cnn2ccc(N3CC4(COC4)C3)cc12] |
| 13a | 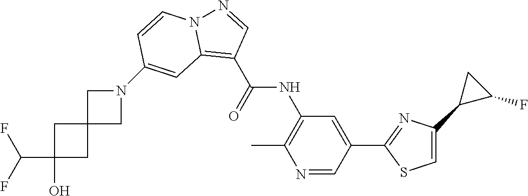 [SMILES: Cc1ncc(-c2nc([C@H]3C[C@@H]3F)cs2)cc1NC(=O)c1cnn2ccc(N3CC4(C3)CC(O)(C(F)F)C4)cc12] |
| 14a | 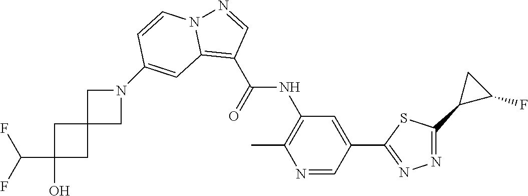 [SMILES: Cc1ncc(-c2nnc([C@H]3C[C@@H]3F)s2)cc1NC(=O)c1cnn2ccc(N3CC4(C3)CC(O)(C(F)F)C4)cc12] |
| 15a | 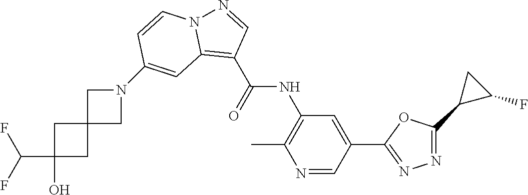 [SMILES: Cc1ncc(-c2nnc([C@H]3C[C@@H]3F)o2)cc1NC(=O)c1cnn2ccc(N3CC4(C3)CC(O)(C(F)F)C4)cc12] |
| 16a | 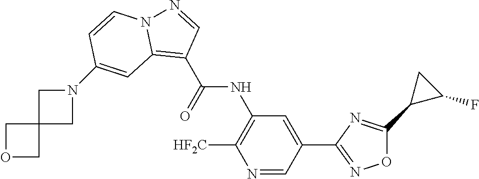 |
| 17a | 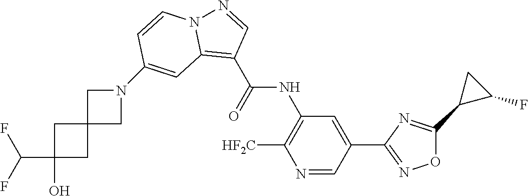 |
| 18a | 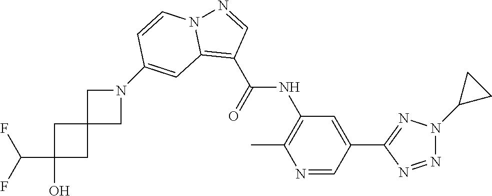 [SMILES: Cc1ncc(-c2nnn(C3CC3)n2)cc1NC(=O)c1cnn2ccc(N3CC4(C3)CC(O)(C(F)F)C4)cc12] |
| 19a | 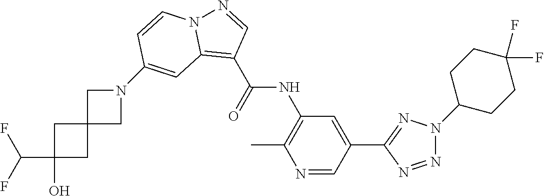 [SMILES: Cc1ncc(-c2nnn(C3CCC(F)(F)CC3)n2)cc1NC(=O)c1cnn2ccc(N3CC4(C3)CC(O)(C(F)F)C4)cc12] |
| 20a | 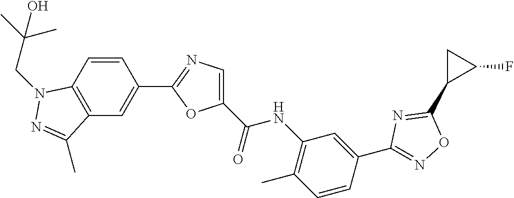 [SMILES: Cc1ccc(-c2noc([C@H]3C[C@@H]3F)n2)cc1NC(=O)c1cnc(-c2ccc3c(c2)c(C)nn3CC(C)(C)O)o1] |
| 21a | 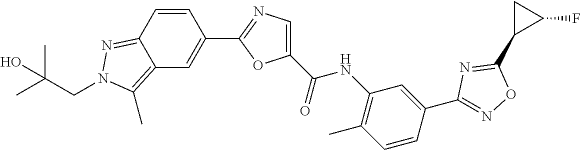 [SMILES: Cc1ccc(-c2noc([C@H]3C[C@@H]3F)n2)cc1NC(=O)c1cnc(-c2ccc3nn(CC(C)(C)O)c(C)c3c2)o1] |
| 22a | 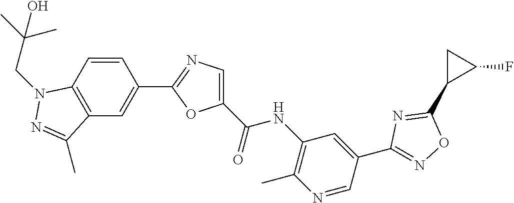 [SMILES: Cc1ncc(-c2noc([C@H]3C[C@@H]3F)n2)cc1NC(=O)c1cnc(-c2ccc3c(c2)c(C)nn3CC(C)(C)O)o1] |
| 23a | 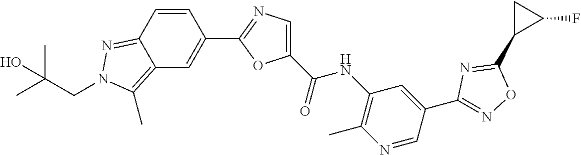 [SMILES: Cc1ncc(-c2noc([C@H]3C[C@@H]3F)n2)cc1NC(=O)c1cnc(-c2ccc3nn(CC(C)(C)O)c(C)c3c2)o1] |

### Biological Activity

1. c-KIT Kinase Assay

### Materials and Methods:

### Materials and Equipment:

Staurosporine (AM-2282) and Imatinib (STI571) were purchased from Selleck Chemicals LLC (Houston, TX). The c-KIT Kinase Enzyme System (V4498) and ADP-Glo™ Kinase Assay kit (V6930) were purchased from Promega (Madison, WI). DMSO (D2650) was purchased from Sigma-Aldrich (Burlington, MA). ECHO-650 acoustic liquid handling system (67492212) and 384-well LDV ECHO source plates (LP-0200) were purchased from Labcyte, Inc (San Jose, CA). Envision Multimode Plate Reader (2104/464466) and ProxiPlate-384 Plus White 384-shallow well microplates (6008280) were purchased from PerkinElmer (Waltham, MA).

c-KIT Kinase Chemiluminescence Assay:

IC₅₀ determination. Test compounds were dispersed into a 384-well low volume while ProxiPlate microplate from a DMSO solution using an ECHO 650 acoustic dispenser to generate a 11-point, 3.162-fold dilution, concentration curve starting at 10 μM in duplicate.

Kinase Reaction: The kinase assay was based on the recommended protocol by c-KIT Kinase Enzyme System from Promega. Recombinant human c-KIT kinase in 3 μL of assay buffer (40 mM Tris pH 7.5, 20 mM MgCl₂, 0.1 mg/mL BSA and 50 μM DTT) was added to the test or high control wells and 3 μL of assay buffer was added to the low control wells. The microplate was centrifuged at 800 rotations per minute (rpm) for 60 s and incubated at room temperature (RT) for 30 min. Next, 2 μL of buffered ATP and polyEY substrate solution was added to all wells. The microplate was centrifuged at 800 rpm for 60 s and incubated at RT for 2 h. The final assay contained c-KIT (25 ng), ATP (50 μM), polyEY substrate (0.2 μg/μL), test compounds (0-10 μM) and DMSO (1.7%) in 5 μL assay buffer.

ADP detection with ADP-Glo™ Kinase Assay: After the kinase reaction incubation, 5 μL of ADP-Glo™ reagent was added to all wells. The microplate was centrifuged at 800 rpm for 60 s and incubated at RT for 40 min. Ten microliter (10 μL) of Kinase Detection Reagent (Luciferin-Luciferase system) was then added to all wells. The microplate was centrifuged at 800 rpm for 60 s and incubated at RT for 60 min. Kinase activity was measured as increase in luminescence at RT in an Envision plate reader equipped with 560 nm filters and operating in endpoint mode. Data Analysis: Endpoint chemiluminescence from high and low control wells were used to calculate normalized c-KIT activity at various concentrations of test compounds. The normalized activities were fitted to inhibitor-versus-normalized response fit in GraphPad Prism 7 to determine half-maximal inhibitory concentration (IC₅₀) and hill coefficient values (H).

c-KIT activity (%)=100/(I+I^{H}/IC₅₀H). Where I is the inhibitor concentration and H is the Hill coefficient. IC₅₀ ranges are reported as follows: A<100 nM; B 0.1-1 μM; C>1 μM.

### 2. M-07e Cellular Assay

M-07e cells (Creative Bioarray, CSC-C0249) which endogenously express human c-KIT were used to evaluate the effect of compounds on stem cell factor (SCF)-mediated cell proliferation. M-07e cells were cultured in growth media (RPMI+10% FBS+1% penicillin/streptomycin+10 ng/mL GM-CSF) at 5E5-1.5E6 cells/mL, replenishing media every 3-4 days. 1.5E4 cells were seeded per well in 384 well tissue culture treated plates in 20 μL assay media (RPMI+1000 FBS+1% penicillin/streptomycin) without growth factor stimulation and incubated at 37° C. in a CO₂ humidity-controlled incubator. Twenty-four hours later, three-fold serially diluted test compounds in media containing SCF (at the previously determined EC₉₀) were added to the plated M-07e cells and incubated for an additional 72 hours. Cell viability was determined by adding 25 μL CellTiter Glo (Promega) to the cells and the luminescence was measured using a Perkin Elmer Envision plate reader. The EC₅₀ was determined using GraphPad Prism. Percent cell growth was determined after normalizing the curve to the mean RLU high control (no compound control wells) at 100% and the mean RLU low control (no SCF control wells) at 0%. EC₅₀ curves were generated using a variable slope four-parameter logistic model with equation Y=100/(1+X\hat{ }HillSlope)/(EC50\hat{ }HillSlope). EC₅₀ ranges are reported as follows: A<100 nM; B 0.1-1 μM; C>1 μM.

TABLE 1
Summary of Biological Activities

| Example # | c-Kit IC₅₀ | M-07e EC₅₀ |
|---|---|---|
| 1 | B | — |
| 2 | C | C |
| 3 | C | C |
| 4 | C | C |
| 5 | C | C |
| 6 | A | A |
| 7 | C | C |
| 8 | C | — |
| 9 | C | — |
| 10 | B | — |
| 11 | C | — |
| 12 | C | — |
| 13 | — | — |
| 14 | B | C |
| 15 | B | B |
| 16 | C | — |
| 17 | C | — |
| 18 | C | — |
| 19 | C | — |
| 20 | C | — |
| 21 | C | — |
| 22 | C | — |
| 23 | B | B |
| 24 | B | — |
| 25 | C | — |
| 26 | C | — |
| 27 | C | C |
| 28 | C | — |
| 29 | C | — |
| 30 | A | B |
| 31 | C | — |
| 32 | C | — |
| 33 | C | — |
| 34 | C | C |
| 35 | C | C |
| 36 | C | C |
| 37 | C | C |
| 38 | B | B |
| 39 | C | C |
| 40 | C | C |
| 41 | A | B |
| 42 | A | B |
| 43 | A | A |
| 44 | A | A |
| 45 | C | — |
| 46 | C | — |
| 47 | C | — |
| 48 | C | — |
| 49 | C | — |
| 50 | C | — |
| 51 | C | — |
| 52 | B | B |
| 53 | C | — |
| 54 | A | A |
| 55 | A | B |
| 56 | A | B |
| 57 | A | B |
| 58 | A | A |
| 59 | A | B |
| 60 | A | A |
| 61 | A | A |
| 62 | B | C |
| 63 | C | — |
| 64 | C | — |
| 65 | C | — |
| 66 | C | — |
| 67 | C | — |
| 68 | C | — |
| 69 | A | A |
| 70 | A | B |
| 71 | A | B |
| 72 | A | B |
| 73 | C | C |
| 74 | B | C |
| 75 | B | — |
| 76 | C | — |
| 77 | C | — |
| 78 | C | — |
| 79 | B | — |
| 80 | B | — |
| 81 | C | — |
| 82 | C | — |
| 83 | C | — |
| 84 | B | — |
| 85 | B | B |
| 86 | C | — |
| 87 | C | — |
| 88 | B | — |
| 89 | C | — |
| 90 | B | B |
| 91 | B | — |
| 92 | A | A |
| 93 | B | B |
| 94 | B | B |
| 95 | B | B |
| 96 | B | B |
| 97 | C | C |
| 98 | C | C |
| 99 | A | A |
| 100 | A | B |
| 101 | B | — |
| 102 | A | B |
| 103 | B | B |
| 104 | C | C |
| 105 | B | B |
| 106 | A | A |
| 107 | A | A |
| 108 | A | A |
| 109 | A | A |
| 110 | B | A |
| 111 | B | A |
| 112 | A | A |
| 113 | A | A |
| 114 | A | A |
| 115 | A | A |
| 116 | A | A |
| 117 | A | A |
| 118 | A | A |
| 119 | A | A |
| 120 | B | B |
| 121 | C | C |
| 122 | A | A |
| 123 | B | A |
| 124 | A | A |
| 125 | B | A |
| 126 | A | A |
| 127 | B | B |
| 128 | C | B |
| 129 | B | C |
| 130 | B | C |
| 131 | B | A |
| 132 | A | A |
| 133 | A | — |
| 134 | A | — |
| 135 | A | B |
| 136 | A | A |
| 137 | A | — |
| 138 | A | A |
| 139 | A | — |
| 140 | A | A |
| 141 | A | — |
| 142 | A | A |
| 143 | A | — |
| 144 | A | A |
| 145 | A | A |
| 146 | A | A |
| 147 | A | A |
| 148 | B | B |
| 149 | A | A |
| 150 | A | A |
| 151 | A | A |
| 152 | A | A |
| 153 | A | A |
| 154 | A | A |
| 155 | B | B |
| 156 | A | A |
| 157 | B | B |
| 158 | B | A |
| 159 | A | A |
| 160 | A | A |
| 161 | A | A |
| 162 | A | A |
| 163 | A | A |
| 164 | B | B |
| 165 | C | B |
| 166 | C | B |
| 167 | C | C |
| 168 | A | A |
| 169 | B | B |
| 170 | A | A |
| 171 | A | A |
| 172 | A | A |
| 173 | A | A |
| 174 | A | A |
| 175 | A | A |
| 176 | A | A |
| 177 | A | A |
| 178 | A | A |
| 179 | A | A |
| 180 | A | A |
| 181 | A | A |
| 182 | A | A |
| 183 | A | A |
| 184 | A | A |
| 185 | A | A |
| 186 | A | A |
| 187 | A | A |
| 188 | B | B |
| 189 | A | A |
| 190 | B | B |
| 191 | A | A |
| 192 | B | B |
| 193 | B | B |
| 194 | A | A |
| 195 | A | B |
| 196 | A | A |
| 197 | B | A |
| 198 | A | B |
| 199 | A | A |
| 200 | A | A |
| 201 | A | A |
| 202 | B | B |
| 203 | A | B |
| 204 | A | A |
| 205 | A | B |
| 206 | B | B |
| 207 | A | B |
| 208 | B | B |
| 209 | B | B |
| 210 | B | B |
| 211 | A | B |
| 212 | A | A |
| 213 | A | A |
| 214 | A | A |
| 215 | A | A |
| 216 | A | A |
| 217 | A | A |
| 218 | B | B |
| 219 | A | A |
| 220 | A | B |
| 221 | A | B |
| 222 | A | B |
| 223 | B | B |
| 224 | A | A |
| 225 | A | B |
| 226 | A | A |
| 227 | A | B |
| 228 | A | B |
| 229 | B | — |
| 230 | A | A |
| 231 | A | A |
| 232 | A | A |
| 233 | A | A |
| 234 | A | A |
| 235 | C | C |
| 236 | B | B |
| 237 | A | B |
| 238 | A | A |
| 239 | A | B |
| 240 | A | A |
| 241 | A | A |
| 241-1 | A | A |
| 242 | A | A |
| 243 | A | A |
| 244 | A | A |
| 245 | A | A |
| 246 | B | A |
| 247 | A | A |
| 248 | B | A |
| 249 | A | A |
| 250 | A | B |
| 251 | B | B |
| 252 | B | B |
| 253 | B | B |
| 254 | B | B |
| 255 | A | B |
| 256 | A | A |
| 257 | A | B |
| 258 | B | B |
| 259 | A | A |
| 260 | A | B |
| 261 | B | B |
| 262 | A | A |
| 263 | A | A |
| 264 | A | A |
| 265 | A | A |
| 266 | B | B |
| 267 | B | B |
| 268 | B | B |
| 269 | B | A |
| 270 | B | A |
| 271 | A | A |
| 272 | A | A |
| 273 | B | A |
| 274 | B | A |
| 275 | A | A |
| 276 | A | A |
| 277 | A | A |
| 278 | A | A |
| 279 | A | A |
| 280 | A | A |
| 281 | A | A |
| 281-1 | A | A |
| 282 | B | A |
| 283 | B | A |
| 284 | C | C |
| 285 | C | C |
| 286 | C | B |
| 287 | B | B |
| 288 | B | B |
| 289 | C | C |
| 290 | B | B |
| 291 | C | C |
| 292 | A | A |
| 293 | A | A |
| 294 | A | B |
| 295 | A | B |
| 296 | C | C |
| 297 | B | B |
| 298 | C | B |
| 299 | A | A |
| 300 | B | B |
| 301 | B | C |
| 302 | A | C |
| 303 | B | B |
| 304 | A | A |
| 305 | C | C |
| 306 | C | C |
| 307 | C | B |
| 308 | C | C |
| 309 | A | A |
| 1a | B | B |
| 2a | A | A |
| 98-1 | A | A |
| 98-2 | C | C |
| 98-3 | C | C |
| 98-4 | C | C |
| 98-5 | C | C |
| 98-6 | C | C |
| 98-7 | C | C |
| 98-8 | C | C |
| 132-1 | A | B |
| 132-2 | B | B |
| 132-3 | A | C |
| 132-4 | A | C |
| 132-5 | B | A |
| 132-6 | B | A |
| 132-7 | B | B |
| 132-8 | A | A |
| 132-9 | A | A |
| 132-10 | B | A |
| 132-11 | A | A |
| 132-12 | A | A |
| 132-13 | A | A |
| 132-14 | A | A |
| 132-15 | A | A |
| 132-16 | B | B |
| 132-17 | A | A |
| 167-1 | A | A |
| 171-1 | A | A |
| 171-2 | C | C |
| 171-3 | C | C |
| 171-4 | B | B |
| 171-5 | B | B |
| 171-6 | B | A |
| 171-7 | B | B |
| 171-8 | B | B |
| 171-9 | A | A |
| 171-10 | C | C |
| 171-11 | B | B |
| 171-12 | B | B |
| 171-13 | B | B |
| 171-14 | B | B |
| 171-15 | A | A |
| 171-16 | A | A |
| 171-17 | A | A |
| 171-18 | A | A |
| 171-19 | A | A |
| 171-20 | A | A |
| 171-21 | A | A |
| 171-22 | B | B |
| 171-23 | C | C |
| 171-24 | A | A |
| 171-25 | A | A |
| 171-26 | A | A |
| 171-28 | A | A |
| 171-29 | A | A |
| 171-30 | A | A |
| 171-31 | B | A |
| 241-2 | A | C |
| 241-3 | A | A |
| 241-4 | A | A |
| 241-5 | A | A |
| 241-6 | A | A |
| 241-7 | B | A |
| 241-8 | A | A |
| 241-9 | B | B |
| 241-10 | A | A |
| 241-11 | A | A |
| 241-12 | A | A |
| 241-13 | C | C |
| 241-14 | B | C |
| 241-15 | A | A |
| 241-16 | B | C |
| 281-2 | A | A |
| 281-3 | A | A |
| 281-4 | A | A |
| 281-5 | A | A |
| 281-6 | B | A |
| 281-7 | B | A |
| 281-8 | A | A |
| 281-9 | B | B |
| 281-10 | B | B |
| 281-11 | B | B |
| 281-12 | A | B |
| 281-13 | B | B |
| 281-14 | B | B |
| 281-15 | B | B |
| 281-16 | A | A |
| 281-17 | B | A |
| 281-18 | A | A |
| 281-19 | B | C |
| 281-20 | A | A |
| 281-21 | A | A |
| 281-22 | A | A |
| 281-23 | A | A |
| 281-24 | A | B |
| 281-25 | A | A |
| 281-26 | B | A |
| 281-27 | A | A |
| 281-28 | A | A |
| 281-29 | B | B |
| 281-30 | A | A |
| 281-31 | A | A |
| 281-32 | B | B |
| 281-33 | B | B |
| 281-34 | B | B |
| 281-35 | A | A |
| 281-36 | A | B |
| 281-37 | B | B |
| 281-38 | B | A |
| 281-39 | A | A |
| 281-40 | B | A |
| 281-41 | C | B |
| 281-42 | A | A |
| 281-43 | A | A |
| 281-44 | A | A |
| 281-45 | A | A |
| 281-46 | B | B |
| 281-47 | B | C |
| 281-48 | A | B |
| 281-49 | C | C |
| 281-50 | B | B |
| 281-51 | C | C |
| 281-52 | A | A |
| 281-53 | A | A |
| 281-54 | A | A |
| 281-55 | B | C |
| 281-56 | C | C |
| 281-57 | A | B |
| 281-58 | B | B |
| 281-59 | A | B |
| 281-60 | B | B |
| 281-61 | C | B |
| 281-62 | A | A |
| 281-63 | B | A |
| 281-64 | A | A |
| 281-65 | A | A |
| 281-66 | B | A |
| 281-67 | B | A |
| 281-68 | A | A |
| 281-69 | B | A |
| 281-70 | C | C |
| 281-71 | C | C |
| 281-72 | C | C |
| 281-73 | C | C |
| 281-74 | C | C |
| 281-75 | C | C |
| 281-76 | C | C |
| 281-77 | B | B |
| 281-78 | A | A |
| 281-79 | C | C |
| 281-80 | C | C |
| 281-81 | B | A |
| 281-82 | A | A |
| 281-83 | B | A |
| 281-84 | C | B |
| 281-85 | C | B |
| 281-86 | C | C |
| 281-87 | C | C |
| 281-88 | B | A |
| 281-89 | B | A |
| 281-90 | C | B |
| 281-91 | C | C |
| 281-92 | B | B |
| 281-93 | B | B |
| 281-94 | C | C |
| 281-95 | B | A |
| 281-96 | B | B |
| 281-97 | B | B |
| 281-98 | A | A |
| 281-99 | A | A |
| 281-100 | B | A |
| 281-101 | C | B |
| 281-102 | C | C |
| 281-103 | B | C |
| 281-104 | C | C |
| 281-105 | B | B |
| 281-106 | B | B |
| 281-107 | B | B |
| 281-108 | A | A |
| 281-109 | A | B |
| 281-110 | A | A |
| 281-111 | C | B |
| 281-112 | A | B |
| 281-113 | B | B |
| 281-114 | C | C |
| 281-115 | C | C |
| 281-116 | B | B |
| 281-117 | B | B |
| 281-118 | B | A |
| 281-119 | B | A |
| 281-120 | B | B |
| 281-121 | B | A |
| 281-122 | B | C |
| 281-123 | A | A |
| 281-124 | B | A |
| 281-125 | A | A |
| 281-126 | C | C |
| 281-127 | B | C |
| 281-128 | B | B |
| 281-129 | B | B |
| 281-130 | B | B |
| 281-131 | A | A |
| 281-132 | A | A |
| 281-133 | B | B |
| 281-134 | A | A |
| 308-1 | B | B |
| 308-2 | A | A |
| 308-3 | A | A |
| 308-4 | B | B |
| 308-5 | C | C |
| 308-6 | C | C |
| 308-7 | C | C |
| 308-8 | C | B |
| 308-9 | B | C |
| 308-10 | C | B |
| 308-11 | B | B |
| 308-12 | C | B |
| 308-13 | C | C |
| 308-14 | A | A |
| 308-15 | B | B |
| 308-16 | B | B |
| 308-17 | A | A |
| 308-18 | B | B |
| 308-19 | B | B |
| 308-20 | C | B |
| 308-21 | A | A |
| 308-22 | B | B |
| 308-23 | A | C |
| 308-24 | A | A |
| 308-25 | B | B |
| 308-26 | A | B |
| 308-27 | A | A |
| 308-28 | B | B |
| 308-29 | A | B |
| 308-30 | B | B |
| 308-31 | B | B |
| 308-32 | A | B |
| 308-33 | C | C |
| 308-34 | B | C |
| 308-35 | B | B |
| 308-36 | A | A |
| 308-37 | B | B |
| 308-38 | B | B |
| 308-39 | B | B |
| 308-40 | A | A |
| 308-41 | B | C |
| 308-42 | B | B |
| 308-43 | A | B |
| 308-44 | B | B |
| 308-45 | C | C |
| 308-46 | B | B |
| 308-47 | B | B |
| 308-48 | B | B |
| 308-49 | B | B |
| 308-50 | C | C |
| 308-51 | A | A |
| 308-52 | B | B |
| 308-53 | B | B |
| 308-54 | B | B |
| 308-55 | B | B |
| 308-56 | B | B |
| 308-57 | A | B |
| 308-58 | A | B |
| 308-59 | B | B |
| 308-60 | C | C |
| 308-61 | B | B |
| 308-62 | B | C |
| 308-63 | B | B |
| 308-64 | C | C |
| 308-65 | A | — |
| 308-66 | A | — |
| 310 | A | A |
| 311 | A | A |
| 312 | B | A |
| 312-1 | C | C |
| 312-2 | B | A |
| 312-3 | C | C |
| 312-4 | B | A |
| 312-5 | C | A |
| 312-6 | C | A |
| 312-7 | B | A |
| 312-8 | C | C |
| 312-9 | C | B |
| 312-10 | B | |
| 313 | B | A |
| 314 | A | A |
| 315 | A | A |
| 316 | B | B |
| 317 | B | A |
| 318 | A | A |
| 319 | A | A |
| 320 | A | A |
| 321 | B | B |
| 322 | A | B |
| 323 | B | C |
| 324 | B | B |
| 325 | B | C |
| 326 | A | A |
| 327 | B | B |
| 328 | B | A |
| 329 | A | B |
| 330 | B | A |
| 331 | B | A |
| 332 | B | A |
| 333 | A | A |
| 334 | B | A |
| 335 | A | A |
| 336 | A | A |
| 337 | B | A |
| 338 | B | A |
| 339 | A | A |
| 340 | A | A |
| 341 | A | A |
| 342 | A | A |
| 343 | A | A |
| 344 | B | A |
| 345 | A | A |
| 346 | A | A |
| 347 | B | B |
| 348 | A | A |
| 349 | A | A |
| 350 | C | C |
| 351 | A | A |
| 352 | C | C |
| 353 | A | A |
| 354 | A | A |
| 355 | B | A |
| 356 | A | A |
| 357 | B | A |
| 358 | C | B |
| 359 | B | B |
| 360 | B | B |
| 361 | B | B |
| 362 | B | B |
| 363 | A | A |
| 364 | C | A |
| 365 | B | A |
| 366 | C | B |
| 367 | B | B |
| 368 | C | B |
| 369 | C | C |
| 370 | C | C |
| 371 | C | C |
| 371-1 | C | B |
| 372 | C | C |
| 373 | C | C |
| 374 | B | B |
| 375 | B | |
| 376 | — | — |
| 377 | B | A |

It is understood that the examples and embodiments described herein are for illustrative purposes only and that various modifications or changes in light thereof will be suggested to persons skilled in the art and are to be included within the spirit and purview of this application and scope of the appended claims.

While this invention has been particularly shown and described with references to preferred embodiments thereof, it will be understood by those skilled in the art that various changes in form and details may be made therein without departing from the scope of the invention encompassed by the appended claims.

1. A compound of Formula (I) or pharmaceutically acceptable salt thereof: 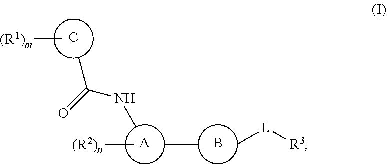
wherein:
each R¹ is independently selected from the group consisting of deuterium, halogen, —CN, optionally substituted —C₁-C₆ alkyl, optionally substituted —C₁-C₆ alkoxy, optionally substituted —C₃-C₁₂ cycloalkyl, optionally substituted —C₅-C₁₂ cycloalkenyl, optionally substituted 3- to 12-membered heterocycloalkyl, optionally substituted aryl, optionally substituted arylalkyl, optionally substituted heteroaryl, optionally substituted heteroarylalkyl, —C(O)R⁴, —C(O)OR⁴, —C(O)NR⁴R⁵, —C(S)NR⁴R⁵, and —NR⁴R⁵;
alternatively, two adjacent R¹ groups are taken together with the atoms to which they are attached to form a fused ring which is optionally substituted C₅-C₈ cycloalkenyl, or optionally substituted 5- to 8-membered heterocycloalkyl;
m is selected from the group consisting of 0, 1, 2, 3, and 4;
each R⁴ and R⁵ is independently selected from the group consisting of hydrogen, optionally substituted —C₁-C₆ alkyl, optionally substituted —C₃-C₈ cycloalkyl, optionally substituted aryl, optionally substituted arylalkyl, optionally substituted heteroaryl, optionally substituted heteroarylalkyl; alternatively, R⁴ and R⁵ are taken together with the nitrogen atom to which they are attached to form an optionally substituted —C₃-C₁₂ heterocyclic ring;
each R² is independently selected from the group consisting of deuterium, halogen, —CN, optionally substituted —C₁-C₆ alkyl, optionally substituted —C₃-C₈ cycloalkyl, optionally substituted —C₁-C₆ alkoxy, and optionally substituted —C₃-C₈ cycloalkoxy;
n is 0, 1, 2, 3, or 4;
L is absent, —(CR⁶R⁷)ₚ—, —(CR⁷R⁸)_{q}O—, —(CR⁷R⁸)NR⁴—, —(CR⁷R⁸)_{q}C(O)NR⁴—, or —(CR⁷R⁸)_{q}NR⁴C(O)—;
p is selected from the group consisting of 1, 2, 3, or 4;
q is selected from the group consisting of 0, 1, 2, 3 and 4;
R⁶ is selected from the group consisting of hydrogen, optionally substituted —C₁-C₆ alkyl, optionally substituted —C₁-C₆ alkoxy, and —NHC(O)OR⁴;
R⁷ and R⁸ are each independently selected from the group consisting of hydrogen, fluorine, and optionally substituted —C₁-C₆ alkyl;
R³ is selected from the group consisting of optionally substituted —C₁-C₈ alkyl, optionally substituted —C₂-C₈ alkenyl, optionally substituted —C₃-C₁₂ cycloalkyl, optionally substituted —C₅-C₁₂ cycloalkenyl, optionally substituted 3- to 12-membered heterocycloalkyl, optionally substituted aryl, and optionally substituted heteroaryl; 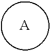
is optionally substituted aryl or optionally substituted heteroaryl; and 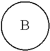
is absent or optionally substituted heteroaryl when L is not absent, and 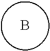
is optionally substituted heteroaryl when L is absent; alternatively, when L is absent, 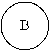
and R² are taken together to form an optionally substituted fused 5- to 8-membered heterocycyl or optionally substituted fused heteroaryl; and 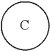
is an optionally substituted heteroaryl; provided that 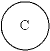
is not

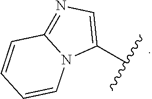 [SMILES: *c1cnc2ccccn12]

2. The compound of Formula (I) is represented by Formula (XIV): 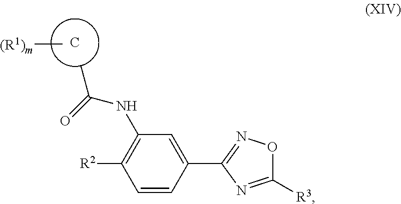
wherein 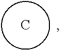
R¹, m, R², and R³ are as defined in claim 1.

3. The compound of Formula (I) is represented by Formula (XXIV-1) or Formula (XXIV-2): 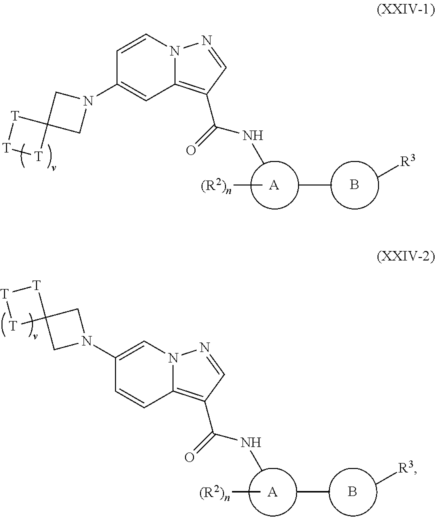
wherein at least one T is CR₂₁R₂₂, and the other Ts are independently O, NR₂₃, —SO₂—, or CR₂₁R₂₂; R₂₁ and R₂₂ are each independently selected from the group consisting of hydrogen, OH, optionally substituted —C₁-C₆ alkyl, optionally substituted —C₁-C₆ alkoxyl, and optionally substituted —C₃-C₈ cycloalkyl; R₂₃ is hydrogen, optionally substituted —C₁-C₆ alkyl, optionally substituted —C₃-C₈ cycloalkyl, —C(O)R⁴, —C(O)OR⁴, or —C(O)NR⁴R⁵; v is 1, 2, 3, or 4; R², R³, R⁴, R⁵, n, 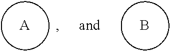
are as defined in claim 1.

4. The compound of Formula (I) is represented by Formula (XXX-1) or Formula (XXX-8): 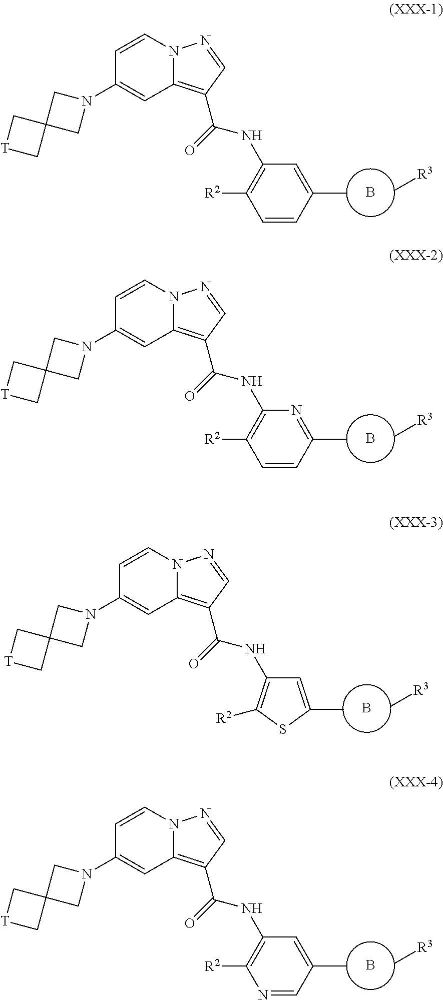 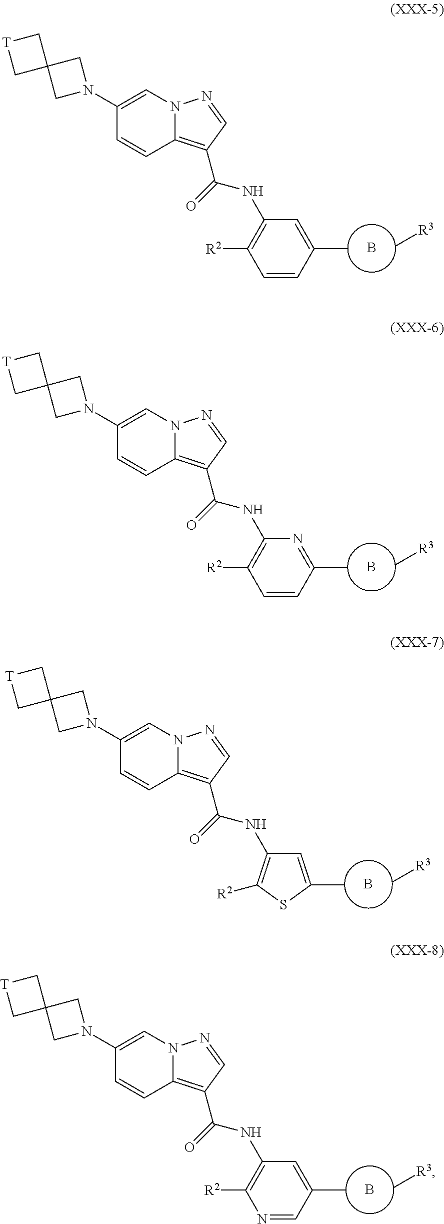
wherein T is O or CR₂₁R₂₂; R₂₁ and R₂₂ are each independently select from the group consisting of hydrogen, OH, optionally substituted —C₁-C₆ alkyl, optionally substituted —C₁-C₆ alkoxyl, and optionally substituted —C₃-C₈ cycloalkyl; R², R³, and 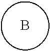
are as defined in claim 1.

5. The compound of Formula (I) is represented by Formula (XXV): 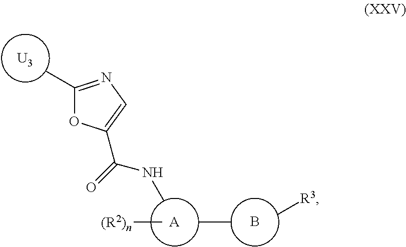
wherein U₃ is optionally substituted 3- to 12-membered heterocycloalkyl, optionally substituted aryl, or optionally substituted heteroaryl; R², R³, n, 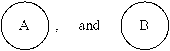
are as defined in claim 1.

6. A pharmaceutical composition comprising the compound of claim 1 and a pharmaceutically acceptable carrier.

7-9. (canceled)

10. A method for treating a kinase-mediated disease or disorder in a subject in need thereof, comprising administering to the subject an effective amount of the compound of claim 1, wherein the kinase is selected from c-kit, CSF1R, PDGFRα and PDGFRβ.

11. The method of claim 10, wherein the disease is a mast-cell associated disease, a respiratory disease, an inflammatory disorder, irritable bowel syndrome (IBS), inflammatory bowel disease (IBD), an autoimmune disorder, a metabolic disease, a fibrosis disease, a dermatological disease, pulmonary arterial hypertension (PAH) or primary pulmonary hypertension (PPH).

12. The method of claim 11, wherein the disease is asthma, allergic rhinitis, pulmonary arterial hypertension (PAH), pulmonary fibrosis, hepatic fibrosis, cardiac fibrosis, scleroderma, irritable bowel syndrome (IBS), inflammatory bowel disease (IBD), uticaria, dermatosis, allergic contact dematitis, rheumatoid arthritis, multiple sclerosis, food allergy, anaphylactic, syndrome, type I diabetes or type II diabetes.

13. A method of modulating kinase activity, comprising administering to a system or a subject in need thereof, an effective amount of the compound of claim 1, wherein the kinase is c-kit, CSF1R, PDGFRα and PDGFRβ.

14-16. (canceled)
